SEQUENCE LISTING (50 sequences)

SEQ ID NO: 1 - DNA, 31 bp, Artificial Sequence
  Description of Artificial Sequence Synthetic primer
acgttggatggttatggtccttaggaaaatg

SEQ ID NO: 2 - DNA, 30 bp, Artificial Sequence
  Description of Artificial Sequence Synthetic primer
acgttggatgacgtctatagatttaccctg

SEQ ID NO: 3 - DNA, 20 bp, Artificial Sequence
  Description of Artificial Sequence Synthetic primer
ctgtacaaactttcttccat

SEQ ID NO: 4 - DNA, 400 bp, Artificial Sequence
  Description of Artificial Sequence Synthetic polynucleotide
ctttgttagtaactttagttcgtcttcagttatacattatttttggatgtttatgcaatcttatttaaatattgtaaaaataattgtaatatactattttgagcaaatttatgtttctcatttactttatttatttatcattgttatggtccttaggaaaatgttattttccttatttggaaaatggatataatcaaaatyatggaagaaagtttgtacagggtaaatctatagacgttgcctgccatcctggctacgctcttccaaaagcgcagaccacagttacatgtatggagaatggctggtctcctactcccagatgcatccgtgtcagtaagtacactactctgaaatcctagcatgttcatgtctttctaagtaacatagatgacattctaag

SEQ ID NO: 5 - DNA, 31 bp, Artificial Sequence
  Description of Artificial Sequence Synthetic primer
acgttggatggaccataaaatgattaaaagg

SEQ ID NO: 6 - DNA, 30 bp, Artificial Sequence
  Description of Artificial Sequence Synthetic primer
acgttggatggtactgatgcagtcttattt

SEQ ID NO: 7 - DNA, 26 bp, Artificial Sequence
  Description of Artificial Sequence Synthetic primer
tatactattttgatcaaattcatgtt

SEQ ID NO: 8 - DNA, 621 bp, Artificial Sequence
  Description of Artificial Sequence Synthetic polynucleotide
tttacagattgactctgtaaagatattccttcatattttgtgttatatccattctccaaataactgagaatacattgtcctaaagaccataaaatgattaaaaggtagattagraacatgaatttgatcaaaatagtatattaaaataattttttgaatatttaaataagactgcatcagtacacaaaaatgacgtatcactgaaggaaaactaaagctactactaaatgtttgtacaaaaaggtcagtattcaatgttacttatctttagtttttatgataaaatatgtttaaattatataggtattctcataaggttcctatatttatttctcatgtgattttcatgaaggtctcataacagaaaagatctagtttggtgtttttgcatgaacaactcttcctttggtaccatctctgtcatataagacaatgtaatcatttgtttgctcttctctctccattctttgcaagttttatgcacatattgttgtaaagaggtttgcttactgaggcatgggactgttggcaaccacccatcttgtgtgcagtgaatgtaatcccagtaacttcctgaaggagtcacaaaattttggtcacagtaataggagtaagattgtc

SEQ ID NO: 9 - DNA, 10 bp, Artificial Sequence
  Description of Artificial Sequence Synthetic primer
acgttggatg

SEQ ID NO: 10 - DNA, 25 bp, Artificial Sequence
  Description of Artificial Sequence Synthetic primer
ttccttatttggaaaatggatataa

SEQ ID NO: 11 - DNA, 24 bp, Artificial Sequence
  Description of Artificial Sequence Synthetic primer
gcaacgtctatagatttaccctgt

SEQ ID NO: 12 - DNA, 18 bp, Artificial Sequence
  Description of Artificial Sequence Synthetic probe
tttcttccatgattttga

SEQ ID NO: 13 - DNA, 20 bp, Artificial Sequence
  Description of Artificial Sequence Synthetic probe
actttcttccataattttga

SEQ ID NO: 14 - DNA, 401 bp, Artificial Sequence
  Description of Artificial Sequence Synthetic polynucleotide
ctttgttagtaactttagttcgtcttcagttatacattatttttggatgtttatgcaatcttatttaaatattgtaaaaataattgtaatatactattttgagcaaatttatgtttctcatttactttatttatttatcattgttatggtccttaggaaaatgttattttccttatttggaaaatggatataatcaaaatyatggaagaaagtttgtacagggtaaatctatagacgttgcctgccatcctggctacgctcttccaaaagcgcagaccacagttacatgtatggagaatggctggtctcctactcccagatgcatccgtgtcagtaagtacactactctgaaatcctagcatgttcatgtctttctaagtaacatagatgacattctaaga

SEQ ID NO: 15 - DNA, 401 bp, Artificial Sequence; identical to SEQ ID NO: 14
  Description of Artificial Sequence Synthetic polynucleotide

SEQ ID NO: 16 - DNA, 52 bp, Homo sapiens
atttggaaaatggatataatcaaaatyatggaagaaagtttgtacagggtaa

SEQ ID NO: 17 - DNA, 52 bp, Homo sapiens
ctttgcttctcagtgcctaaaaaggartaccatacaataacaataatattta

SEQ ID NO: 18 - DNA, 52 bp, Homo sapiens
cataaaatgattaaaaggtagattagraacatgaatttgatcaaaatagtat

SEQ ID NO: 19 - DNA, 52 bp, Homo sapiens
tttgaaactttctgaattaacgttatktaaaaggaaatgtagatgttatttt

SEQ ID NO: 20 - DNA, 52 bp, Homo sapiens
tttctaaattttttttcagtgggatgmtatgttgatagcagctactccatcc

SEQ ID NO: 21 - DNA, 52 bp, Homo sapiens
ccattacttttatttttttcttcacasattaattgaggctaataatatgcct

SEQ ID NO: 22 - DNA, 52 bp, Homo sapiens
tgaggctaataatatgccttgattagwtatgcaatttctcctgatatcaaac

SEQ ID NO: 23 - DNA, 52 bp, Homo sapiens
ttggtttgattttggagctgattgtakaatcaactacttattttttctcttt

SEQ ID NO: 24 - DNA, 52 bp, Homo sapiens
gccagataaatcacagaatatcaattyctcttgacttgtaaaacttgaatta

SEQ ID NO: 25 - DNA, 52 bp, Homo sapiens
ttttatataaacacaaatttattcctyatggatacatatctaagactgggtt

SEQ ID NO: 26 - DNA, 52 bp, Homo sapiens
ctcaaaagaagacatttatgcagtcarcaaatacatgaaaacaagcttattg

SEQ ID NO: 27 - DNA, 52 bp, Homo sapiens
tagtttgaaatcaggtagcgtgatgchtcagctttgtgctttttgattagga

SEQ ID NO: 28 - DNA, 52 bp, Homo sapiens
  modified_base (27)..(27): a, c, t or g
acaaactgttttcctgagcgatcatanattgtaccttcacatactcagtgta

SEQ ID NO: 29 - DNA, 52 bp, Homo sapiens
gctggcaaggttgcgaaacaactggawctcacatacactgagtatgtgaagg

SEQ ID NO: 30 - DNA, 52 bp, Homo sapiens
cagcaggcatacaaatctgacaatctygtaactatttgtggcaagccaggtc

SEQ ID NO: 31 - DNA, 52 bp, Homo sapiens
ctgcctagtgccccctttccactgggrcagacccagagacaaaccctagcag

SEQ ID NO: 32 - DNA, 52 bp, Homo sapiens
ctgggatggaatataaacactggagayagatctaaccttgaacactatcaaa

SEQ ID NO: 33 - DNA, 52 bp, Homo sapiens
gcttactgcaacaggggggacactgartggaaaatttctcatgccctacaaa

SEQ ID NO: 34 - DNA, 52 bp, Homo sapiens
acactgagtggaaaatttctcatgccytacaaagcctgatgttaaacagtca

SEQ ID NO: 35 - DNA, 52 bp, Homo sapiens
gtggcgctgccaaatctaaagcagcgycctttgagagaaaaggcacagcaat

SEQ ID NO: 36 - DNA, 52 bp, Homo sapiens
agtcatgaataatgtataagcatcagktatcaccagataaatggctttgaaa

SEQ ID NO: 37 - DNA, 52 bp, Homo sapiens
  modified_base (27)..(27): a, c, t or g
atagttgtgctggcttctaatatgccntcaacatatataagcaaagtctcat

SEQ ID NO: 38 - DNA, 52 bp, Homo sapiens
ctaaacccatcccttctctctcatgtwtgaacaaatcaaatggctttcttag

SEQ ID NO: 39 - DNA, 52 bp, Homo sapiens
ctgttccaacaaatagcatacacagtrtggggtgtgtacagtgctaggttaa

SEQ ID NO: 40 - DNA, 52 bp, Homo sapiens
aatagcatactcagccttctgttgtcsgtggtgcataaagctgctttcctca

SEQ ID NO: 41 - DNA, 52 bp, Homo sapiens
tcactaggcagagcaggccactaaagrggtgaacagagaaaaaaatgcccaa

SEQ ID NO: 42 - DNA, 52 bp, Homo sapiens
tggaagaagatgtaaagagagaattcrattgagactttgactccaatcaaag

SEQ ID NO: 43 - DNA, 52 bp, Homo sapiens
ggactatgtctttgggacacacttaargaagactatccaaaaaataatttaa

SEQ ID NO: 44 - DNA, 52 bp, Homo sapiens
ttagacaaaacatatgtcttcttgacwactacaatagttactttgagcataa

SEQ ID NO: 45 - DNA, 52 bp, Homo sapiens
gcatgagactatgttagcatggcatakaatgtctagaaaatctataagtcta

SEQ ID NO: 46 - DNA, 52 bp, Homo sapiens
tatgcctggcagtcccttttggagtckgtgggaaaatgggcagaatattttg

SEQ ID NO: 47 - DNA, 52 bp, Homo sapiens
  modified_base (27)..(27): a, c, t or g
gagtgtatactccatagctaggactcntgatgcctagaaggctctgccacca

SEQ ID NO: 48 - DNA, 52 bp, Homo sapiens
ttctttgctgcaaaccctactgtctcmgcgtattggtctattgctaaacagt

SEQ ID NO: 49 - DNA, 188 bp, Homo sapiens
ccttaggaaaatgttattttccttatttggaaaatggatataatcaaaatcatggaagagtttgtacagggtaaatctatagacgttgcctgccatcctggctacgctcttccaaaagcgcagaccacagttacatgtatggagaatggctggtctcctactcccagatgcatccgtgtcagtaagta

SEQ ID NO: 50 - DNA, 188 bp, Homo sapiens
ccttaggaaaatgttattttccttatttggaaaatggatataatcaaaattatggaagagtttgtacagggtaactctacagaagttgcctgccatcctggctacggtcttccaaaagcgcagaccacagttacatgtacggagaaaggctggtctcctactcccagatgcatccgtgtcagtaagta

(19) United States
(12) Patent Application Publication
(10) Pub. No.: US 2012/0202708 A1
(43) Pub. Date: Aug. 9, 2012

(54) COMPLEMENT FACTOR H COPY NUMBER VARIANTS FOUND IN THE RCA LOCUS

(75) Inventors: Lorah T. Perlee, Wilton, CT (US); Paul A. Oeth, San Diego, CA (US); Michael R. Barnes, Thundridge Ware (GB)

(73) Assignee: SEQUENOM, INC., San Diego, CA (US)

(21) Appl. No.: 13/273,195

(22) Filed: Oct. 13, 2011

Related U.S. Application Data
(60) Provisional application No. 61/393,300, filed on Oct. 14, 2010.

(51) Int. Cl. C40B 30/10; C12Q 1/68

(52) U.S. Cl. 506/12; 435/6.11

(57) ABSTRACT

Provided herein is a variant in the RCA locus and methods for detecting the presence, absence or amount of multiple forms of the variant.

### RELATED APPLICATION

This application claims the benefit of U.S. Provisional Patent Application No. 61/393,300, filed Oct. 14, 2010, entitled “Complement Factor H Copy Number Variants Found in the RCA Locus”, naming Lorah Perlee et al. as inventors and assigned attorney docket no. SEQ-6029-PV. The foregoing provisional patent application is incorporated herein by reference in its entirety.

### SEQUENCE LISTING

The instant application contains a Sequence Listing which has been submitted in ASCII format via EFS-Web and is hereby incorporated by reference in its entirety. Said ASCII copy, created on Nov. 30, 2011, is named SEQ6029U.txt and is 11,631 bytes in size.

### FIELD

The technology relates in part to novel variants in the RCA locus and methods for detecting the presence, absence or amount of multiple forms of the variants.

### BACKGROUND

Age-related macular degeneration (AMD) is the leading cause of irreversible blindness in developed countries. AMD is defined as an abnormality of the retinal pigment epithelium (RPE) that leads to overlying photoreceptor degeneration of the macula and consequent loss of central vision. Early AMD is characterized by drusen (>63 um) and hyper- or hypo-pigmentation of the RPE. Intermediate AMD is characterized by the accumulation of focal or diffuse drusen (>120 um) and hyper- or hypo-pigmentation of the RPE. Advanced AMD is associated with vision loss due to either geographic atrophy of the RPE and photoreceptors (dry AMD) or neovascular choriocapillary invasion across Bruch's membrane into the RPE and photoreceptor layers (wet AMD). AMD leads to a loss of central visual acuity, and can progress in a manner that results in severe visual impairment and blindness. Visual loss in wet AMD is more sudden and may be more severe than in dry AMD.

It is estimated that 1.75 million people in the United States alone suffer from advanced AMD (dry and wet AMD). Also in the United States alone, it is estimated that an additional 7.3 million people suffer from intermediate AMD, which puts them at increased risk for developing the advanced forms of the disease. It is projected that such numbers will increase significantly over the next 10 to 15 years.

### SUMMARY

The technology in part relates to the discovery of a subclass of novel CFH H1 risk haplotypes with significant structural variations observed in CFH and downstream CFHR genes that provide the basis for a mechanism associated with the dysfunction observed in the regulation of the alternative complement system. The alternative complement system plays a role in multiple indication areas, including but not limited to age-related macular degeneration (AMD), renal diseases (aHUS, MPGNII), and autoimmune diseases. Thus, the novel “risk” haplotypes provided herein represent new markers for detecting, diagnosing, prognosing, analyzing and/or monitoring diseases and disorders associated with the alternative complement system. It was observed that these haplotypes occurred at a relatively high frequency in the Caucasian population and in a Yoruba subject suggesting that the haplotypes may be ancient and highly dispersed across a range of populations.

The technology also in part relates to the discovery of alleles that are multiplied, and in particular, duplicated. In some embodiments, such alleles include a multiplied region within a Complement Factor H(CFH) locus, which CFH locus includes the CFH gene, CFH-related genes (e.g., CFHR1, CFHR2, CFHR3, CFHR4 and CFHR5 genes) and intergenic regions between the foregoing genes. These alleles are referred to herein as “CFH alleles” and can be present as copy number variants (CNVs). Detecting the presence or absence of a multiplied (e.g., duplicated) CFH allele in nucleic acid from a subject (e.g., on one chromosome or one strand of nucleic acid from the subject) can be useful for identifying the presence or absence of an altered risk (e.g., increased or decreased risk) for a complement-pathway associated condition or disease (e.g., age-related macular degeneration (AMD)).

In some embodiments, a multiplied (e.g., duplicated) CFH allele comprises all of, or a portion of, a region that includes one or more single nucleotide polymorphism (SNP) positions chosen from rs1061170 (SEQ ID NO: 16), rs403846 (SEQ ID NO: 17), rs1409153 (SEQ ID NO: 18), rs10922153 (SEQ ID NO: 19) and rs1750311 (SEQ ID NO: 20). In certain embodiments, a multiplied (e.g., duplicated) CFH allele comprises all of, or a portion of, a region that includes one or more single nucleotide polymorphism (SNP) positions chosen from rs10922094 (SEQ ID NO: 21); rs12124794 (SEQ ID NO: 22); rs12405238 (SEQ ID NO: 23); rs10922096 (SEQ ID NO: 24); rs12041668 (SEQ ID NO: 25); rs514943 (SEQ ID NO: 26); rs579745 (SEQ ID NO: 27); rs10922102 (SEQ ID NO: 28); rs2860102 (SEQ ID NO: 29); rs4658046 (SEQ ID NO: 30); rs10754199 (SEQ ID NO: 31); rs12565418 (SEQ ID NO: 32); rs12038333 (SEQ ID NO: 33); rs12045503 (SEQ ID NO: 34); rs9970784 (SEQ ID NO: 35); rs1831282 (SEQ ID NO: 36); rs203687 (SEQ ID NO: 37); rs2019727 (SEQ ID NO: 38); rs2019724 (SEQ ID NO: 39); rs1887973 (SEQ ID NO: 40); rs6428357 (SEQ ID NO: 41); rs7513157 (SEQ ID NO: 42); rs6695321 (SEQ ID NO: 43); rs10733086 (SEQ ID NO: 44); rs1410997 (SEQ ID NO: 45); rs203685 (SEQ ID NO: 46); rs203684 (SEQ ID NO: 47); and rs10737680 (SEQ ID NO: 48). In certain embodiments, the region includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or 33 of the foregoing SNPs. In some embodiments, a multiplied (e.g., duplicated) CFH allele comprises all of, or a portion of, a region spanning exon 9 of the CFH gene to CFHR4 (e.g., about chromosome position 196,659,237 to about chromosome position 196,887,763 (NCBI Build 37)). In certain embodiments, a multiplied (e.g., duplicated) CFH allele comprises all of, or a portion of, a region spanning intron 9 of the CFH gene to CFHR4 (e.g., about chromosome position 196,679,455 to about chromosome position 196,887,763 (NCBI Build 37)). In some embodiments, a multiplied (e.g., duplicated) CFH allele comprises all of, or a portion of, a region spanning CFHR3 to CFHR4 (e.g., about chromosome position 196,743,930 to about chromosome position 196,887,763 (NCBI Build 37)). In certain embodiments, a multiplied (e.g., duplicated) CFH allele comprises all of, or a portion of, a region spanning intron 9, exon 10 and intron 11 of the CFH gene, which includes SNP rs10737680 (SEQ ID NO: 48) (e.g., CNV1 described herein; e.g., about chromosome position 196,650,000 to about chromosome position 196,680,665 (NCBI Build 37)). In some embodiments, a multiplied (e.g., duplicated) CFH allele comprises all of, or a portion of, an intergenic region between CFHR1 and CFHR4 (e.g., CNV2 described herein; e.g., about chromosome position 196,788,861 to about chromosome position 196,857,212 (NCBI Build 37)). For specific copy number variants CNV1 and CNV2 described herein, CNV2 is homologous and tends to co-occur with CNV1. It is possible that the region spanning CNV1 and CNV2 contain additional CNVs. In some embodiments, a CFH allele haplotype (e.g., H1, H2, H3 or H4 haplotype) is considered in a nucleic acid analysis.

Thus provided herein are methods and materials for detecting multiplied (e.g., duplicated) CFH alleles in mammals. The methods and materials described herein can be used to determine the CFH copy number genotype. The ability to determine CFH copy number genotypes can aid patient care because CFH allele function can regulate the complement pathway. The complement pathway plays a role in a wide range of physiological processes, and has been implicated in a wide range of diseases and disorders including AMD. When more than one CFH copy number allele is present, knowing which allele is duplicated can allow the proper phenotype to be assigned. For example, an individual with two or more copies of the CFH allele can be at greater risk of developing a severe form of AMD (e.g., wet AMD). Thus, subjects at risk of developing (or have developed), progressing, who are progressing, or who have progressed, to a severe form of a complement pathway associated condition or disease (e.g., wet AMD) can be identified by methods described herein, and treatments can be administered to such subjects. Provided herein is a method for identifying the presence or absence of a duplicated or multiplied Complement Factor H(CFH) allele in sample nucleic acid, including: (a) detecting one or more nucleotides at one or more single nucleotide polymorphism (SNP) positions chosen from rs1061170 (SEQ ID NO: 16), rs403846 (SEQ ID NO: 17), rs1409153 (SEQ ID NO: 18), rs10922153 (SEQ ID NO: 19) and rs1750311 (SEQ ID NO: 20) in a nucleic acid containing a CFH allele from a biological sample, thereby providing a genotype; and (b) identifying the presence or absence of a duplicated or multiplied CFH allele based on the genotype.

Also provided herein is a method for identifying the presence or absence of a duplicated or multiplied Complement Factor H(CFH) allele in sample nucleic acid, including: (a) analyzing a polynucleotide including a CFH allele in a nucleic acid from a biological sample, thereby providing an analyzed polynucleotide; and (b) determining from the analyzed polynucleotide whether the CFH allele is present or absent in multiple copies on one chromosome in a region that includes one or more single nucleotide polymorphism (SNP) positions chosen from rs1061170 (SEQ ID NO: 16), rs403846 (SEQ ID NO: 17), rs1409153 (SEQ ID NO: 18), rs10922153 (SEQ ID NO: 19) and rs1750311 (SEQ ID NO: 20).

Provided also herein is a method for identifying the presence or absence of a duplicated or multiplied Complement Factor H(CFH) allele in sample nucleic acid, including: (a) analyzing a polynucleotide including a CFH allele in a nucleic acid from a biological sample, thereby providing an analyzed polynucleotide; and (b) determining from the analyzed polynucleotide whether the CFH allele is present or absent in multiple copies on one chromosome in a region spanning about chr1: 196,659,237 to about chr1:196,887,763, which chromosome positions are according to NCBI Build 37.

Also provided herein is a method for identifying the presence or absence of a duplicated or multiplied Complement Factor H(CFH) allele in sample nucleic acid, including: (a) analyzing a polynucleotide including a CFH allele in a nucleic acid from a biological sample, thereby providing an analyzed polynucleotide; and (b) determining from the analyzed polynucleotide whether the CFH allele is present or absent in multiple copies on one chromosome in a region surrounding exon 10 of the CFH allele.

Provided also herein is a method for identifying the presence or absence of a duplicated or multiplied Complement Factor H(CFH) allele in sample nucleic acid, including: (a) analyzing a polynucleotide including a CFH allele in a nucleic acid from a biological sample, thereby providing an analyzed polynucleotide; and (b) determining from the analyzed polynucleotide whether the CFH allele is present or absent in multiple copies on one chromosome in a region in proximity to coding variant Y402H and extending through intron 9 and intron 14 of the CFH allele.

Also provided herein is a method for identifying the presence or absence of a duplicated or multiplied Complement Factor H(CFH) allele in sample nucleic acid, including: (a) analyzing a polynucleotide including a CFH allele in a nucleic acid from a biological sample, thereby providing an analyzed polynucleotide; and (b) determining from the analyzed polynucleotide whether the CFH allele is present or absent in multiple copies on one chromosome in a region in proximity to coding variant Y402H and extending through CFHR4.

In some embodiments, the one or more SNP positions further are chosen from rs10922094 (SEQ ID NO: 21); rs12124794 (SEQ ID NO: 22); rs12405238 (SEQ ID NO: 23); rs10922096 (SEQ ID NO: 24); rs12041668 (SEQ ID NO: 25); rs514943 (SEQ ID NO: 26); rs579745 (SEQ ID NO: 27); rs10922102 (SEQ ID NO: 28); rs2860102 (SEQ ID NO: 29); rs4658046 (SEQ ID NO: 30); rs10754199 (SEQ ID NO: 31); rs12565418 (SEQ ID NO: 32); rs12038333 (SEQ ID NO: 33); rs12045503 (SEQ ID NO: 34); rs9970784 (SEQ ID NO: 35); rs1831282 (SEQ ID NO: 36); rs203687 (SEQ ID NO: 37); rs2019727 (SEQ ID NO: 38); rs2019724 (SEQ ID NO: 39); rs1887973 (SEQ ID NO: 40); rs6428357 (SEQ ID NO: 41); rs7513157 (SEQ ID NO: 42); rs6695321 (SEQ ID NO: 43); rs10733086 (SEQ ID NO: 44); rs1410997 (SEQ ID NO: 45); rs203685 (SEQ ID NO: 46); rs203684 (SEQ ID NO: 47); rs10737680 (SEQ ID NO: 48); rs11811456; rs12240143; rs2336502; rs6428363; rs6428370; rs6685931; rs6695525, rs2133138, rs6428366, rs10733086 (SEQ ID NO: 44), rs10922094 (SEQ ID NO: 21), and rs1887973 (SEQ ID NO: 40). In certain embodiments, the genotype includes two or more copies of a nucleotide at each SNP position. In some embodiments, the genotype includes a ratio between two of the two or more copies of the nucleotide at each SNP position.

In certain embodiments, the method further includes determining whether the subject from which the sample was obtained is homozygous or heterozygous for a nucleotide at each of the one or more SNP positions. In some embodiments, the method further includes detecting the one or more nucleotides at the one or more SNP positions on a single strand of the nucleic acid. In certain embodiments, the method further includes detecting the presence or absence of an increased risk, decreased risk, or changed or altered risk of developing a severe form of a complement-pathway associated condition or disease based on the identification of the presence or absence of the duplicated or multiplied CFH allele. In some embodiments, the method further includes detecting the presence or absence of age-related macular degeneration (AMD) based on the identification of the presence or absence of the duplicated or multiplied CFH allele.

In some embodiments, the method further includes determining from the analyzed polynucleotide whether the CFH allele is present or absent in multiple copies on one chromosome in a region spanning about chr1: 196,659,237 to about chr1:196,887,763, which chromosome positions are according to NCBI Build 37. In certain embodiments, the method further includes determining from the analyzed polynucleotide whether the CFH allele is present or absent in multiple copies on one chromosome in a region spanning about chr1: 196,679,455 to about chr1:196,887,763, which chromosome positions are according to NCBI Build 37. In some embodiments, the method further includes determining from the analyzed polynucleotide whether the CFH allele is present or absent in multiple copies on one chromosome in a region spanning about chr1:196,743,930 to about chr1:196,887,763, which chromosome positions are according to NCBI Build 37.

In certain embodiments, the analyzing in (a) includes determining the presence or absence of one or more genetic markers associated with the multiple copies on the one chromosome. In some embodiments, the analyzing in (a) includes detecting one or more nucleotides at one or more single nucleotide polymorphism (SNP) positions chosen from rs1061170 (SEQ ID NO: 16), rs403846 (SEQ ID NO: 17), rs1409153 (SEQ ID NO: 18), rs10922153 (SEQ ID NO: 19) and rs1750311 (SEQ ID NO: 20) in the amplified CFH allele, thereby providing a genotype. In certain embodiments, the one or more SNP positions further are chosen from rs10922094 (SEQ ID NO: 21); rs12124794 (SEQ ID NO: 22); rs12405238 (SEQ ID NO: 23); rs10922096 (SEQ ID NO: 24); rs12041668 (SEQ ID NO: 25); rs514943 (SEQ ID NO: 26); rs579745 (SEQ ID NO: 27); rs10922102 (SEQ ID NO: 28); rs2860102 (SEQ ID NO: 29); rs4658046 (SEQ ID NO: 30); rs10754199 (SEQ ID NO: 31); rs12565418 (SEQ ID NO: 32); rs12038333 (SEQ ID NO: 33); rs12045503 (SEQ ID NO: 34); rs9970784 (SEQ ID NO: 35); rs1831282 (SEQ ID NO: 36); rs203687 (SEQ ID NO: 37); rs2019727 (SEQ ID NO: 38); rs2019724 (SEQ ID NO: 39); rs1887973 (SEQ ID NO: 40); rs6428357 (SEQ ID NO: 41); rs7513157 (SEQ ID NO: 42); rs6695321 (SEQ ID NO: 43); rs10733086 (SEQ ID NO: 44); rs1410997 (SEQ ID NO: 45); rs203685 (SEQ ID NO: 46); rs203684 (SEQ ID NO: 47); rs10737680 (SEQ ID NO: 48); rs11811456; rs12240143; rs2336502; rs6428363; rs6428370; rs6685931; rs6695525, rs2133138, rs6428366, rs10733086 (SEQ ID NO: 44), rs10922094 (SEQ ID NO: 21), and rs1887973 (SEQ ID NO: 40).

In some embodiments, the genotype includes two or more copies of a nucleotide at each SNP position. In certain embodiments, the genotype includes a ratio between two of the two or more copies of the nucleotide at each SNP position. In some embodiments, the method further includes determining whether the subject from which the sample was obtained is homozygous or heterozygous for a nucleotide at each of the one or more SNP positions. In certain embodiments, the method further includes detecting the one or more nucleotides at the one or more SNP positions on a single strand of the nucleic acid.

In some embodiments, the method further includes obtaining from a subject the biological sample that contains the nucleic acid including the CFH allele. In certain embodiments, the nucleic acid is double-stranded. In some embodiments, the nucleic acid is deoxyribonucleic acid (DNA). In certain embodiments, the method further includes amplifying the nucleic acid from the biological sample and detecting the one or more nucleotides at the one or more SNP positions in the amplified nucleic acid.

In certain embodiments, the method further includes detecting the presence or absence of an increased risk, decreased risk, or changed or altered risk of developing a complement-pathway associated condition or disease based on whether the CFH allele is present or absent in multiple copies on one chromosome. In some embodiments the method further includes detecting the presence or absence of an increased risk, decreased risk, or changed or altered risk of developing a severe form of a complement-pathway associated condition or disease based on whether the CFH allele is present or absent in multiple copies on one chromosome.

In certain embodiments, the method further includes detecting the presence or absence of age-related macular degeneration (AMD) based on whether the CFH allele is present or absent in multiple copies on one chromosome. In some embodiments, the method further includes detecting the presence or absence of wet age-related macular degeneration (AMD) based on whether the CFH allele is present or absent in multiple copies on one chromosome.

In some embodiments, the method further includes determining the risk of progressing from a less severe to a more severe form of a complement-pathway associated condition or disease based on whether the CFH allele is present or absent in multiple copies on one chromosome. In certain embodiments, the complement-pathway associated condition or disease is wet age-related macular degeneration (AMD). In some embodiments, the method further includes amplifying the nucleic acid from the biological sample and analyzing the amplified nucleic acid in (a).

In some embodiments, the presence of absence of one or more of the following SNP variants is detected: an adenine at rs11811456, a cytosine at rs12240143, a cytosine at rs1409153 (SEQ ID NO: 18), a guanine at rs2133138, a thymine at rs2133138, a thymine at rs23336502, a guanine at rs6428363, an adenine at rs6428366, a cytosine at rs6429366, a guanine at rs6428370, a cytosine at rs6685931, a guanine at rs6695525, an adenine at rs10737680 (SEQ ID NO: 48), a thymine at rs12045503 (SEQ ID NO: 34), a thymine at rs2019724 (SEQ ID NO: 39), an adenine at rs2019727 (SEQ ID NO: 38), an adenine at rs203685 (SEQ ID NO: 46), a cytosine at rs203687 (SEQ ID NO: 37), a thymine at rs2860102 (SEQ ID NO: 29), a thymine at rs4658046 (SEQ ID NO: 30), a thymine at rs514943 (SEQ ID NO: 26), and an adenine at rs6428357 (SEQ ID NO: 41), which are associated with a CFH allele multiplication event. In certain embodiments, the presence or absence of a complementary nucleotide for one or more the SNP variants listed in the previous sentence is detected in a complementary strand (e.g., a thymine at rs11811456). In certain embodiments, the presence of absence of one or more of the following SNP variants is detected: a guanine at rs11811456, a thymine at rs12240143, a thymine at rs1409153 (SEQ ID NO: 18), an adenine at rs2133138, a cytosine at rs2133138, a cytosine at rs23336502, an adenine at rs6428363, a guanine at rs6428366, a thymine at rs6429366, an adenine at rs6428370, a thymine at rs6685931, a thymine at rs6695525, a cytosine at rs10737680 (SEQ ID NO: 48), a cytosine at rs12045503 (SEQ ID NO: 34), a cytosine at rs2019724 (SEQ ID NO: 39), a thymine at rs2019727 (SEQ ID NO: 38), a cytosine at rs203685 (SEQ ID NO: 46), a thymine at rs203687 (SEQ ID NO: 37), an adenine at rs2860102 (SEQ ID NO: 29), a cytosine at rs4658046 (SEQ ID NO: 30), a cytosine at rs514943 (SEQ ID NO: 26), a guanine at rs6428357 (SEQ ID NO: 41), an adenine at rs10733086 (SEQ ID NO: 44), a thymine at rs10733086 (SEQ ID NO: 44), a cytosine at rs10922094 (SEQ ID NO: 21), a guanine at rs10922094 (SEQ ID NO: 21), a cytosine at rs1887973 (SEQ ID NO: 40) and a guanine at rs188793, which are not associated with a CFH allele multiplication event. In certain embodiments, the presence or absence of a complementary nucleotide for one or more the SNP variants listed in the previous sentence is detected in a complementary strand (e.g., a cytosine at rs11811456). In some embodiments, the presence of absence of one or more of the foregoing variants at each SNP position is detected (e.g., 1, 2 or 3 variants are detected at each position), and in certain embodiments, a ratio between two SNP variants is determined. In certain embodiments, it is determined whether a subject is homozygous or heterozygous for one or more of the SNP variants identified.

Certain aspects of the technology are described further in the following description, examples, claims and drawings.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIG. 1 shows the high degree of sequence identity at Y402H in the region flanking the key CFH variant associated with the Y402H (non-synonymous coding SNP rs1061170 (SEQ ID NO: 16)). The query sequence (SEQ ID NO: 49; subject sequence is disclosed as SEQ ID NO: 50) is exon 9 of CFH which is shown here to demonstrate 96% sequence identity with a region in CFHR3. However, the “C” variant found in the CFH reference sequence is not present in any of the sequences in the RCA region demonstrating high identity.

FIG. 2A shows the results from the real-time qPCR assay for relative quantification of the rs1061170 (SEQ ID NO: 16) loci for the C allele using a Taqman probe. Data for 47 HapMap CEPH DNAs is shown. Fold difference was calculated using the ΔΔCₜ method (2001, Pfaffl). The data was generated from quadruplicate reactions per sample and the ΔΔCₜ shown represents the mean of those observations after normalization. The X-axis lists sample ID and genotype and the Y-axis the relative difference between samples based on normalization to PLAC4 then to NA12043 (note its value is 1).

FIG. 2B shows the results from the real-time qPCR assay for relative quantification of the rs1061170 (SEQ ID NO: 16) loci for the T allele using a Taqman probe. Data for 47 HapMap CEPH DNAs is shown. Fold difference was calculated using the ΔΔCₜ method (2001, Pfaffl). The data was generated from quadruplicate reactions per sample and the ΔΔCt shown represents the mean of those observations after normalization. The X-axis lists sample ID and genotype and the Y-axis the relative difference between samples based on normalization to PLAC4 then to NA12043 (note its value is 1).

FIG. 3 shows detection of copy number variants at rs1409153 (SEQ ID NO: 18) using Sequenom® MassARRAY® technology. Cluster plot depiction of MassARRAY primer extension products for rs1409153 (SEQ ID NO: 18) over HapMap CEPH populations DNA Plates 1 & 6 obtained from Coriell Cell Repositories. All Samples were run in quadruplicate. The clusters are based on the amount of each allele from the biallelic SNP converted to a product of specific mass corresponding to each allele or both alleles (heterozygous samples). Two samples, NA11840 and NA10854, clearly deviated from the 1:1 allele ratio exhibited by the core cluster of heterozygotes for all four replicates and were shown to be significant based on a CNV calling algorithm previous described (2009, Oeth et al). The allele ratios clearly show a 2:1 or 1:2 bias indicative of an extra copy, note the change in peak areas for the two alleles.

FIGS. 4A-E show depth of read coverage across the six available subjects. BAM file-size is indicated for each subject, giving a relative measure of chromosome-wide read depth. Overall variability of read depth between subjects is due to variation in draft read depth. Two additional subjects with copy numbers in CFH reported in the DGV database are also included for reference (DGV9384, DGV9385). FIGS. 4A-E disclose “RS1061170” as SEQ ID NO: 16.

FIGS. 5A-D show depth of read coverage across the RCA Cluster for six available subjects. Again the same two possible duplicated regions (CNV1 & CNV2) are shown in the Figures.

FIG. 6 shows depth of read coverage for hapmap subject NA12842 showing key genomic features across CNV1 and CNV2.

FIG. 7 shows depth of read coverage for hapmap subject NA12842 showing key genomic features across CNV1. FIG. 7 discloses “RS1061170” as SEQ ID NO: 16 and “RS10737680” as SEQ ID NO: 48.

FIG. 8 shows depth of read coverage for hapmap subject NA12842 showing key genomic features across CNV2.

Experimental details and results for FIGS. 9-23 are described in Example 5.

FIG. 9 schematically illustrates various genes or portions thereof in the CFH and CFHR regions and digital PCR assays used to detect differences in copy number.

FIG. 10 shows the results from digital PCR assays for various regions in the CFH-CFHR region.

FIG. 11 schematically illustrates the organization of the CFH-CFHR region and a known duplication which confers protection to AMD.

FIGS. 12A-12E show the results of digital PCR assays performed to distinguish CFH haplotypes.

FIG. 13 shows the results of 26 digital PCR SNP assays used to evaluate ratio differences reflective of copy number polymorphisms in CNV2.

FIG. 14 presents a table of copy number differences detected in various samples. FIG. 14 discloses “rs1409153” as SEQ ID NO: 18.

FIG. 15 presents a table of copy number differences detected in various samples across multiple SNPs in CNV1 and CNV2 regions. FIG. 15 discloses “rs10737680” as SEQ ID NO: 48, “rs12045503” as SEQ ID NO: 34, “rs203685” as SEQ ID NO: 46 and “rs6695321” as SEQ ID NO: 43.

FIG. 16 presents a table of different haplotypes deduced from about 1900 clinical samples from patients having late stage AMD, and age matched controls. FIG. 16 discloses “RS1061170” as SEQ ID NO: 16, “RS403846” as SEQ ID NO: 17, “RS1409153” as SEQ ID NO: 18 and “RS10922153” as SEQ ID NO: 19.

FIG. 17 presents linkage disequilibrium values for various SNP. FIG. 17 discloses “rs1061170” as SEQ ID NO: 16, “RS403846” as SEQ ID NO: 17, “rs1409153” as SEQ ID NO: 18, “rs1750311” as SEQ ID NO: 20 and “rs10922153” as SEQ ID NO: 19.

FIG. 18 shows SNP's that can be used to distinguish various haplotype combinations. FIG. 18 discloses “rs1061170” as SEQ ID NO: 16, “RS403846” as SEQ ID NO: 17, “rs1409153” as SEQ ID NO: 18 and “rs10922153” as SEQ ID NO: 19.

FIG. 19 shows the results of digital PCR assays that identify genotypes generated by SNPs that distinguish the 2 most frequent duplications (e.g., H1/H3) observed in clinical samples. FIG. 19 discloses “rs1061170” as SEQ ID NO: 16, “R5403846” as SEQ ID NO: 17, “rs1409153” as SEQ ID NO: 18 and “rs10922153” as SEQ ID NO: 19.

FIG. 20 presents a table of SNP patterns reflective of duplication. FIG. 20 discloses “rs1061170” as SEQ ID NO: 16, “R5403846” as SEQ ID NO: 17, “rs1409153” as SEQ ID NO: 18, “rs10922153” as SEQ ID NO: 19 and “rs1750311” as SEQ ID NO: 20.

FIG. 21 is a schematic illustration of Alu recombination hotspots that map to the exon 9 region of the CFH-CFHR locus.

FIG. 22 provides chromosome position information (NCBI build 37) for CFH and CFHR genes in the CFH-CFHR region.

FIG. 23 is a schematic representation of an intron 9 breakpoint associated with various CFH haplotypes. Also shown in FIG. 23 are the nucleotides associated with various CFH haplotypes.

FIG. 24 illustrates a regional ARMD4 association plot for CFH. FIG. 24 is described in Example 6. FIG. 24 discloses “rs10737680” as SEQ ID NO: 48.

### DETAILED DESCRIPTION

The H1-copy number variant subclass was initially identified through an investigation of a group of HapMap samples that revealed a discordant genotyping at the CFH 1277 “C” position associated with SNP rs1061170 (SEQ ID NO: 16). The HapMap genotyping performed on the Illumina platform generated a CT result in a collection of samples designated “discordant” relative to the CC genotyping obtained on the MassARRAY platform and further confirmed with Sanger sequencing. Subsequently, these samples were evaluated with a real-time PCR assay designed to detect copy number variations at the AMD disease associated SNP rs1061170 (SEQ ID NO: 16). The discordant sample typings obtained on the real-time PCR assay matched the results obtained with the MassARRAY and sequencing platforms. However, the copy number assay also revealed striking differences in copy number across the sample collection with 6 samples demonstrating more than 5 fold difference in the C-variant assay and 4 samples with at least 5 fold difference observed in the T-variant assay. Further testing of these samples was pursued by scanning short read (next-gen) sequencing data across the entire CFH-CFHR5 region to detect the presence or absence of copy number variants/deletions. The CFH variant alleles were shown to contain copy number variants of a segment of DNA in CFH corresponding to the region surrounding exon 10 in addition to a segment upstream of CFHR4, a gene known to harbor copy number variations. The H1-variant identified is described as containing multiple copies of a segment of the CFH gene localized to a region surrounding exon 10, in close proximity to the coding variant Y402H, and extending through intron 9 and exon 10. These regions contain SNPs that have been reported with the highest association to developing advanced stage AMD.

Evaluation of regions of short read next-generation sequencing data across the CFH-CFHR5 region in these variant samples revealed two putative duplicated regions. One copy number variant was observed in CFH in the exon 10 region with boundaries or regions of segmental copy number variant that extend upstream to include CFH exon 9. The second copy number variant observed in these samples was in a region upstream of CFHR4. The observation of a CNV in CFHR4 was also observed on the MassARRAY platform through a query of the region associated with SNP rs1409153 (SEQ ID NO: 18). Data from this locus revealed a copy number variant in HapMap sample NA11840. Copy number variants other than the one described here have been reported in the CFHR4 region and have been shown to influence disease susceptibility by changing the delicate balance of CFH and CFHR proteins reported to be associated with dysfunction of Alternative Complement mediated diseases. The presence of a copy number variant embedded in the region of the key complement control protein CFH, which is central to innate immune function has even greater potential to impact biological pathways and provide the definitive mechanism involved in the development of disease associated with Alternative Complement Pathway dysfunction.

This subclass of H1 haplotypes was identified with an assay that measures the copy number of a segment of DNA containing the upstream and downstream regions flanking the CFH Y402H coding variant and verified through a comprehensive analysis of all publicly available 1000 Genomes Project short read data from 92 HapMap subjects surveyed across the CFH locus.

The CFH Y402H coding variant, found in the region of copy number variant, has been previously identified to have high association with susceptibility to developing age-related macular degeneration. The Tyr402H is polymorphism lies in the center of SCR7 within a cluster of positively charged amino acids mediating binding of heparin, C-reactive protein (CRP) and M protein. The biological consequences of a His instead of a Tyr at position 402 are decreased affinity to glycosaminoglycans, retinal pigment epithelial cells and C-reactive protein. Strikingly, SNP variants downstream of Y402H have demonstrated an even higher association with AMD and described as independent factors for disease risk. Identification of a subclass of H1 risk alleles containing a copy number variant in the region central to the association of advanced stage AMD provides a plausible explanation for a dual function of both kinds of genetic variation for disease causality. Genetic variations in CFH are associated with a range of clinical conditions, including complement factor H deficiency (CFH deficiency) [MIM:609814], and Haemolytic uraemic syndrome atypical type 1 (AHUS1) [MIM:235400], both of which primarily impact renal tissues but also manifest symptoms in the eye. Two clinical conditions associated with CFH variations are known to primarily impact the eye, Basal laminar drusen (BLD) [MIM:126700] and Age-related macular degeneration [MIM:610698]. AMD has been described as an inflammatory disease that results from over activation of the alternative complement pathway as a result of a variant form of CFH, the key inhibitor of the alternative complement pathway. AMD is a multi-factorial eye disease and the most common cause of irreversible vision loss in the developed world. In most patients, the disease manifests as ophthalmoscopically visible yellowish accumulations of protein and lipid (known as drusen) that lie beneath the retinal pigment epithelium and within an elastin-containing structure known as Bruch membrane. Studies have shown a consistently strong association with CFH at the missense Tyr402His variant (rs1061170 (SEQ ID NO: 16)); however a recent high density association study (Chen et al 2010) confirmed association at rs1061170 (SEQ ID NO: 16) while showing strongest association with rs10737680 (SEQ ID NO: 48) in intron 10 of the CFH gene (odds ratio (OR)=3.11 (2.76, 3.51), with P<1.6×10⁻⁷⁵).

Risk conferred by SNP variants could be modified by variability in copy number at the CFH gene or other transcripts in the wider RCA cluster. Hughes et al. (2006) have reported that a CFHR1 and CFHR3 deletion haplotype is protective against age-related macular degeneration. A gene copy number variant embedded in the critical region of CFH, the protein required for concerted or competitive binding of C3b, C-reactive protein, heparin, sialic acid and other polyanions, and interaction with plasma proteins and microorganisms could lead to (i) a disruption/modification of the corresponding transcript resulting in an incompletely transcribed or significantly truncated or modified version of the CFH protein, or (ii) to a shift in the ratio of full length Factor H vs. its shorter isoform Factor H-Like 1 in various tissues or body compartments, or (iii) to a general up- or down regulation of proteins transcribed from this gene as a consequence of a change of cis-acting regulatory elements or a change in RNA stability or translation efficiency.

Similarly, CFHR-4 close to which CNV2 is localized, is structurally and functionally closely related to CFH and modulate its biological function, including but not limited to enhancing the cofactor activity for the factor I-mediated proteolytic inactivation of C3b.

Thus provided herein are methods for determining the presence or absence of an H1-copy number variation. In related embodiments, methods provided herein may also include further determining the presence or absence of other known genetic variants associated with alternative complement pathway diseases or disorders. Examples of genetic variants associated with alternative complement pathway diseases or disorders are known in the art.

A significant portion of CNVs have been identified in regions containing known segmental copy number variants Sharp et al. (2005). CNVs that are associated with segmental copy number variants may be susceptible to structural chromosomal rearrangements via non-allelic homologous recombination (NAHR) mechanisms (Lupski 1998). NAHR is a process whereby segmental copy number variants on the same chromosome can facilitate copy number changes of the segmental duplicated regions along with intervening sequences. In addition to the formation of CNVs in normal individuals, NAHR may also result in large structural polymorphisms and chromosomal rearrangements that directly lead to genomic instability or to early onset, highly penetrant disorders (Lupski 1998). CNVs mediated by segmental copy number variants have also been seen across multiple populations, including African populations, suggesting that these specific genomic imbalances may in some cases either predate the dispersal of modern humans out of Africa or recur independently in different populations. CNV1 and CNV2 as described herein have been seen in the Yoruba subject carrying the known CFH copy number variant DGV9385, suggesting that these CNVs may be ancient and highly dispersed among populations, although copy number may vary between populations.

Recent reports in the literature demonstrating CNV related to the deletion of CFHR3/1 changes competitive binding of CFH to C3b specific to SCR7 (Fritsche et al. HMG 2010). The H1 copy number variant described herein is located in close proximity to SCR7. The deletion of CFHR3/CFHR1 has been shown to have a significant impact on the modulation of alternative complement pathway independent of haplotype tagging SNPs in CFH that tag the haplotype [Fritsche et al HMG 2010]. This provides a basis for proposing that a copy number variant in the region containing/flanking SCR7 in CFH will have a significant impact on disease biology.

Modification of the CFH gene, central to immune modulation, can have significant implications related to modified functionality and subsequent changes in immunological control and concomitant susceptibility/protection to indications that manifest at the individual level as Alternative Complement Pathway Related diseases or disorders. In some embodiments, provided is a subclass of the H1CFH risk alleles referred to as “H1-copy number variant” that specifically influence an individual's disease susceptibility, prognosis (or severity), treatment or outcome. Identification of a subclass of H1 risk haplotypes revealing gross structural modifications in the gene central to inflammation will improve prediction of late stage AMD and potentially have utility in other indication areas (e.g. aHUS, MPGNII) involving CFH/CFHR genetic variants demonstrating strong association with disease. Identification of patients with/without the CFH H1-copy number variant haplotype will substantially improve the positive predictive value of a genetic test that predicts risk of developing late stage AMD.

Also provided herein are methods and materials related to detecting duplicated CFH alleles in mammals. A duplicated CFH allele can be any arrangement of a CFH gene within the RCA locus that includes a copy number variant of a CFH allele or portion thereof. For example, a duplicated CFH allele can have a CFH copy number variant arrangement as shown in Table 13.

Genomic DNA is typically used in an analysis of duplicated CFH alleles. Genomic DNA can be extracted from any biological sample containing nucleated cells, such as a peripheral blood sample or a tissue sample (e.g., mucosal scrapings of the lining of the mouth). Standard methods can be used to extract genomic DNA from a blood or tissue sample, including, for example, phenol extraction. Genomic DNA also can be extracted with kits well known in the art.

A duplicated CFH allele can be detected by any appropriate DNA, RNA (e.g., Northern blotting or RT-PCR), or polypeptide (e.g., Western blotting or protein activity) based method. Non-limiting examples of DNA based methods include PCR methods (e.g., quantitative PCR methods and PCR methods described in the Examples, direct sequencing, fluorescence in situ hybridization (FISH), a Sequenom® MassARRAY®-based allele specific primer extension (ASPE) assay, such as that described in the Examples, and Southern blotting. In some cases, the phase of a duplicated CFH allele can be determined using an ASPE-based algorithm, such as that described in the Examples. In some cases, the phase of a duplicated CFH allele can be determined by isolating and genotyping a non-duplicated CFH allele and a 5′ and 3′ CFH duplicated allele. In some cases, a duplicated CFH allele can be detected based on altered CFH polypeptide function (e.g., decreased or no metabolism of one or more environmental chemicals or drugs). Any combination of such methods also can be used.

PCR refers to a procedure or technique in which target nucleic acids are enzymatically amplified. Sequence information from the ends of the region of interest or beyond typically is employed to design oligonucleotide primers that are identical in sequence to opposite strands of the template to be amplified. PCR can be used to amplify specific sequences from DNA as well as RNA, including sequences from total genomic DNA or total cellular RNA. Primers are typically 14 to 40 nucleotides in length, but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995. When using RNA as a source of template, reverse transcriptase can be used to synthesize complementary DNA (cDNA) strands.

Oligonucleotide primer pairs can be combined with genomic DNA from a mammal and subjected to standard PCR conditions, such as those described in Example 2, to amplify a CFH allele or portion thereof. For example, such a PCR reaction can be performed to amplify an entire duplicated CFH allele, or a portion of a duplicated CYP2D6 allele. The oligonucleotide primers having the nucleotide sequences set forth in SEQ ID NOs:2-8 are examples of primers that can be used to amplify nucleic acids containing duplicated CYP2D6 alleles, or portions thereof.

Amplified products can be separated based on size (e.g., by Mass Spectrometry) and the appropriate detection system used to determine the size of the amplified product. In some cases, detection of an amplification product of a particular size can indicate the presence and/or identity of a duplicated CFH allele.

As is known in the medical arts and sciences, a single diagnostic or prognostic parameter may or may not be relied upon in isolation. A number of different parameters may be considered in combination, including but not limited to patient age, general health status, sex, lifelong health habits, smoking, medication history, and physical or clinical findings. The latter may include macular or extramacular drusen, retinal pigment epithelial changes, subretinal fluid, subretinal hemorrhage, disciform scarring, subretinal exudate, peripheral drusen, and peripheral reticular pigmentary change.

When a risk of neovascular AMD is identified or an early onset of neovascular AMD is identified, patients can be grouped appropriately, i.e., stratified so that appropriate conclusions can be drawn in clinical studies. Additionally, appropriate modifications to lifestyle can be recommended, including, but not limited to diet, supplementation of vitamins and minerals, for example, smoking cessation, drugs, and obesity reduction or control. Supplementation of diet, including but not limited to vitamins C, E, beta carotene, zinc, and/or lutein/zeaxanthin may be recommended. Diets high in these factors may be used as a source of the helpful factors. One particular combination supplement includes: 500 milligrams of vitamin C, 400 milligrams of vitamin E, 15 milligrams of beta-carotene, 80 milligrams of zinc as zinc oxide, two milligrams of copper as cupric oxide. Drugs that may delay onset or reduce a symptoms of disease when it occurs include anti-inflammatory medicaments. Many are known in the art and can be used. Positive dietary recommendations include carrots, corn, kiwi, pumpkin, yellow squash, zucchini squash, red grapes, green peas, cucumber, butternut squash, green bell pepper, celery, cantaloupe, sweet potatoes, dried apricots, tomato and tomato products, dark green leafy vegetables, spinach, kale, turnips, and collard greens.

The association of the genetic variations set forth herein may be employed in methods of identifying subjects at risk for developing one or more diseases or pathologic conditions of the eye associated with a condition selected from the formation of drusen, pathologic neovascularization, vascular leak, and edema in the tissues of the eye, AMD in both its wet and dry forms, DR, ROP, ischemia-induced neovascularization, and macular edema.

Such complement factor H-associated diseases or disorders include eye diseases and disorders, including age-related macular degeneration (AMD), optic nerve disorders, cardiovascular disease, and atypical hemolytic uremic syndrome (aHUS), a complement related disease with renal manifestations.

Nucleic acids, amplification processes primers and detection methodology are described further hereafter.

### Nucleic Acids

Target or sample nucleic acid may be derived from one or more samples or sources. “Sample nucleic acid” as used herein refers to a nucleic acid from a sample. “Target nucleic acid” and “template nucleic acid” are used interchangeably throughout the document and refer to a nucleic acid of interest. The terms “total nucleic acid” or “nucleic acid composition” as used herein, refer to the entire population of nucleic acid species from or in a sample or source. Non-limiting examples of nucleic acid compositions containing “total nucleic acids” include, host and non-host nucleic acid, maternal and fetal nucleic acid, genomic and acellular nucleic acid, or mixed-population nucleic acids isolated from environmental sources. As used herein, “nucleic acid” refers to polynucleotides such as deoxyribonucleic acid (DNA) and ribonucleic acid (RNA), and refers to derivatives, variants and analogs of RNA or DNA made from nucleotide analogs, single (sense or antisense) and double-stranded polynucleotides. The term “nucleic acid” does not refer to or infer a specific length of the polynucleotide chain, thus nucleotides, polynucleotides, and oligonucleotides are also included within “nucleic acid.”

A sample containing nucleic acids may be collected from an organism, mineral or geological site (e.g., soil, rock, mineral deposit, combat theater), forensic site (e.g., crime scene, contraband or suspected contraband), or a paleontological or archeological site (e.g., fossil, or bone) for example. A sample may be a “biological sample,” which refers to any material obtained from a living source or formerly-living source, for example, an animal such as a human or other mammal, a plant, a bacterium, a fungus, a protist or a virus. Template or sample nucleic acid utilized in methods and kits described herein often is obtained and isolated from a subject. A subject can be any living or non-living source, including but not limited to a human, an animal, a plant, a bacterium, a fungus, a protist. Any human or animal can be selected, including but not limited, non-human, mammal, reptile, cattle, cat, dog, goat, swine, pig, monkey, ape, gorilla, bull, cow, bear, horse, sheep, poultry, mouse, rat, fish, dolphin, whale, and shark, or any animal or organism that may have a detectable genetic abnormality. The sample may be heterogeneous, by which is meant that more than one type of nucleic acid species is present in the sample. A sample may be heterogeneous because more than one cell type is present, such as a fetal cell and a maternal cell or a cancer and non-cancer cell.

The biological or subject sample can be in any form, including without limitation umbilical cord blood, chorionic villi, amniotic fluid, cerbrospinal fluid, spinal fluid, lavage fluid (e.g., bronchoalveolar, gastric, peritoneal, ductal, ear, athroscopic), exudate from a region of infection or inflammation, or a mouth wash containing buccal cells, biopsy sample (e.g., from pre-implantation embryo), celocentesis sample, fetal nucleated cells or fetal cellular remnants, washings of female reproductive tract, urine, feces, sputum, saliva, nasal mucous, prostate fluid, lavage, semen, lymphatic fluid, bile, tears, sweat, breast milk, breast fluid, embryonic cells and fetal cells. a solid material such as a tissue, cells, a cell pellet, a cell extract, or a biopsy, or a biological fluid such as urine, blood, saliva, amniotic fluid, urine, cerebral spinal fluid and synovial fluid and organs. In some embodiments, a biological sample may be blood.

As used herein, the term “blood” encompasses whole blood or any fractions of blood, such as serum and plasma as conventionally defined. Blood plasma refers to the fraction of whole blood resulting from centrifugation of blood treated with anticoagulants. Blood serum refers to the watery portion of fluid remaining after a blood sample has coagulated. Fluid or tissue samples often are collected in accordance with standard protocols hospitals or clinics generally follow. For blood, an appropriate amount of peripheral blood (e.g., between 3-40 milliliters) often is collected and can be stored according to standard procedures prior to further preparation in such embodiments. A fluid or tissue sample from which template nucleic acid is extracted may be acellular. In some embodiments, a fluid or tissue sample may contain cellular elements or cellular remnants.

In some embodiments, the nucleic acid composition containing the target nucleic acid or nucleic acids may be collected from a cell free or substantially cell free biological composition, blood plasma, blood serum or urine for example. The term “substantially cell free” as used herein, refers to biologically derived preparations or compositions that contain a substantially small number of cells, or no cells. A preparation intended to be completely cell free, but containing cells or cell debris can be considered substantially cell free. That is, substantially cell free biological preparations can include up to about 50 cells or fewer per milliliter of preparation (e.g., up to about 50 cells per milliliter or less, 45 cells per milliliter or less, 40 cells per milliliter or less, 35 cells per milliliter or less, 30 cells per milliliter or less, 25 cells per milliliter or less, 20 cells per milliliter or less, 15 cells per milliliter or less, 10 cells per milliliter or less, 5 cells per milliliter or less, or up to about 1 cell per milliliter or less).

Template nucleic acid may be derived from one or more sources (e.g., cells, soil, etc.) by methods known in the art. Cell lysis procedures and reagents are commonly known in the art and may generally be performed by chemical, physical, or electrolytic lysis methods. For example, chemical methods generally employ lysing agents to disrupt the cells and extract the nucleic acids from the cells, followed by treatment with chaotropic salts. Physical methods such as freeze/thaw followed by grinding, the use of cell presses and the like are also useful. High salt lysis procedures are also commonly used. For example, an alkaline lysis procedure may be utilized. The latter procedure traditionally incorporates the use of phenol-chloroform solutions, and an alternative phenol-chloroform-free procedure involving three solutions can be utilized. In the latter procedures, solution 1 can contain 15 mM Tris, pH 8.0; 10 mM EDTA and 100 ug/ml Rnase A; solution 2 can contain 0.2N NaOH and 1% SDS; and solution 3 can contain 3M KOAc, pH 5.5. These procedures can be found in Current Protocols in Molecular Biology, John Wiley & Sons, N.Y., 6.3.1-6.3.6 (1989), incorporated herein in its entirety.

A sample also may be isolated at a different time point as compared to another sample, where each of the samples may be from the same or a different source. A sample nucleic acid may be from a nucleic acid library, such as a cDNA or RNA library, for example. A sample nucleic acid may be a result of nucleic acid purification or isolation and/or amplification of nucleic acid molecules from the sample. Sample nucleic acid provided for sequence analysis processes described herein may contain nucleic acid from one sample or from two or more samples (e.g., from 1, 2, 3, 4, 5, 6, 7, 8, 9, 10, 11, 12, 13, 14, 15, 16, 17, 18, 19 or 20 or more samples).

Sample nucleic acid may comprise or consist essentially of any type of nucleic acid suitable for use with processes of the invention, such as sample nucleic acid that can hybridize to solid phase nucleic acid (described hereafter), for example. A sample nucleic in certain embodiments can comprise or consist essentially of DNA (e.g., complementary DNA (cDNA), genomic DNA (gDNA) and the like), RNA (e.g., message RNA (mRNA), short inhibitory RNA (siRNA), microRNA, ribosomal RNA (rRNA), tRNA and the like), and/or DNA or RNA analogs (e.g., containing base analogs, sugar analogs and/or a non-native backbone and the like). A nucleic acid can be in any form useful for conducting processes herein (e.g., linear, circular, supercoiled, single-stranded, double-stranded and the like). A nucleic acid may be, or may be from, a plasmid, phage, autonomously replicating sequence (ARS), centromere, artificial chromosome, chromosome, a cell, a cell nucleus or cytoplasm of a cell in certain embodiments. A sample nucleic acid in some embodiments is from a single chromosome (e.g., a nucleic acid sample may be from one chromosome of a sample obtained from a diploid organism). Deoxyribonucleotides include deoxyadenosine, deoxycytidine, deoxyguanosine and deoxythymidine. For RNA, the uracil base is uridine. A source or sample containing sample nucleic acid(s) may contain one or a plurality of sample nucleic acids. A plurality of sample nucleic acids as described herein refers to at least 2 sample nucleic acids and includes nucleic acid sequences that may be identical or different. That is, the sample nucleic acids may all be representative of the same nucleic acid sequence, or may be representative of two or more different nucleic acid sequences (e.g., from 1, 2, 3, 4, 5, 6, 7, 8, 9, 10, 11, 12, 13, 14, 15, 16, 17, 18, 19, 20, 50, 100, 1000 or more sequences).

Sample or template nucleic acid can include different nucleic acid species, including extracellular nucleic acid, and therefore is referred to herein as “heterogeneous” in certain embodiments. For example, blood serum or plasma from a person having cancer can include nucleic acid from cancer cells and nucleic acid from non-cancer cells. The term “extracellular template or sample nucleic acid” as used herein refers to nucleic acid isolated from a source having substantially no cells (e.g., no detectable cells, or fewer than 50 cells per milliliter or less as described above, or may contain cellular elements or cellular remnants). Examples of acellular sources for extracellular nucleic acid are blood plasma, blood serum and urine. Without being limited by theory, extracellular nucleic acid may be a product of cell apoptosis and cell breakdown, which provides basis for extracellular nucleic acid often having a series of lengths across a large spectrum (e.g., a “ladder”). In some embodiments, the nucleic acids can be cell free nucleic acid.

The term “nucleotides”, as used herein, in reference to the length of nucleic acid chain, refers to a single stranded nucleic acid chain. The term “base pairs”, as used herein, in reference to the length of nucleic acid chain, refers to a double stranded nucleic acid chain.

Sample nucleic acid may be provided for conducting methods described herein without processing of the sample(s) containing the nucleic acid in certain embodiments. In some embodiments, sample nucleic acid is provided for conducting methods described herein after processing of the sample(s) containing the nucleic acid. For example, a sample nucleic acid may be extracted, isolated, purified or amplified from the sample(s). The term “isolated” as used herein refers to nucleic acid removed from its original environment (e.g., the natural environment if it is naturally occurring, or a host cell if expressed exogenously), and thus is altered by human intervention (e.g., “by the hand of man”) from its original environment. An isolated nucleic acid generally is provided with fewer non-nucleic acid components (e.g., protein, lipid) than the amount of components present in a source sample. A composition comprising isolated sample nucleic acid can be substantially isolated (e.g., about 90%, 91%, 92%, 93%, 94%, 95%, 96%, 97%, 98%, 99% or greater than 99% free of non-nucleic acid components). The term “purified” as used herein refers to sample nucleic acid provided that contains fewer nucleic acid species than in the sample source from which the sample nucleic acid is derived. A composition comprising sample nucleic acid may be substantially purified (e.g., about 90%, 91%, 92%, 93%, 94%, 95%, 96%, 97%, 98%, 99% or greater than 99% free of other nucleic acid species). The term “amplified” as used herein refers to subjecting nucleic acid of a sample to a process that linearly or exponentially generates amplicon nucleic acids having the same or substantially the same nucleotide sequence as the nucleotide sequence of the nucleic acid in the sample, or portion thereof.

Sample nucleic acid also may be processed by subjecting nucleic acid to a method that generates nucleic acid fragments, in certain embodiments, before providing sample nucleic acid for a process described herein. In some embodiments, sample nucleic acid subjected to fragmentation or cleavage may have a nominal, average or mean length of about 5 to about 10,000 base pairs, about 100 to about 1,000 base pairs, about 100 to about 500 base pairs, or about 10, 15, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000, 9000 or 10000 base pairs. Fragments can be generated by any suitable method known in the art, and the average, mean or nominal length of nucleic acid fragments can be controlled by selecting an appropriate fragment-generating procedure. In certain embodiments, sample nucleic acid of a relatively shorter length can be utilized to analyze sequences that contain little sequence variation and/or contain relatively large amounts of known nucleotide sequence information. In some embodiments, sample nucleic acid of a relatively longer length can be utilized to analyze sequences that contain greater sequence variation and/or contain relatively small amounts of unknown nucleotide sequence information.

Sample nucleic acid fragments can contain overlapping nucleotide sequences, and such overlapping sequences can facilitate construction of a nucleotide sequence of the previously non-fragmented sample nucleic acid, or a portion thereof. For example, one fragment may have subsequences x and y and another fragment may have subsequences y and z, where x, y and z are nucleotide sequences that can be 5 nucleotides in length or greater. Overlap sequence y can be utilized to facilitate construction of the x-y-z nucleotide sequence in nucleic acid from a sample in certain embodiments. Sample nucleic acid may be partially fragmented (e.g., from an incomplete or terminated specific cleavage reaction) or fully fragmented in certain embodiments.

Sample nucleic acid can be fragmented by various methods known in the art, which include without limitation, physical, chemical and enzymatic processes. Examples of such processes are described in U.S. Patent Application Publication No. 20050112590 (published on May 26, 2005, entitled “Fragmentation-based methods and systems for sequence variation detection and discovery,” naming Van Den Boom et al.). Certain processes can be selected to generate non-specifically cleaved fragments or specifically cleaved fragments. Examples of processes that can generate non-specifically cleaved fragment sample nucleic acid include, without limitation, contacting sample nucleic acid with apparatus that expose nucleic acid to shearing force (e.g., passing nucleic acid through a syringe needle; use of a French press); exposing sample nucleic acid to irradiation (e.g., gamma, x-ray, UV irradiation; fragment sizes can be controlled by irradiation intensity); boiling nucleic acid in water (e.g., yields about 500 base pair fragments) and exposing nucleic acid to an acid and base hydrolysis process.

Sample nucleic acid may be specifically cleaved by contacting the nucleic acid with one or more specific cleavage agents. The term “specific cleavage agent” as used herein refers to an agent, sometimes a chemical or an enzyme that can cleave a nucleic acid at one or more specific sites. Specific cleavage agents often will cleave specifically according to a particular nucleotide sequence at a particular site.

Examples of enzymic specific cleavage agents include without limitation endonucleases (e.g., DNase (e.g., DNase I, II); RNase (e.g., RNase E, F, H, P); Cleavase™ enzyme; Taq DNA polymerase; E. coli DNA polymerase I and eukaryotic structure-specific endonucleases; murine FEN-1 endonucleases; type I, II or III restriction endonucleases such as Acc I, Afl III, Alu I, Alw44 I, Apa I, Asn I, Ava I, Ava II, BamH I, Ban II, Bcl I, Bgl I. Bgl II, Bln I, Bsm I, BssH II, BstE II, Cfo I, Cla I, Dde I, Dpn I, Dra I, EcIX I, EcoR I, EcoR I, EcoR II, EcoR V, Hae II, Hae II, Hind III, Hind III, Hpa I, Hpa II, Kpn I, Ksp I, Mlu I, MIuN I, Msp I, Nci I, Nco I, Nde I, Nde II, Nhe I, Not I, Nru I, Nsi I, Pst I, Pvu I, Pvu II, Rsa I, Sac I, Sal I, Sau3A I, Sca I, ScrF I, Sfi I, Sma I, Spe I, Sph I, Ssp I, Stu I, Sty I, Swa I, Taq I, Xba I, Xho I); glycosylases (e.g., uracil-DNA glycolsylase (UDG), 3-methyladenine DNA glycosylase, 3-methyladenine DNA glycosylase II, pyrimidine hydrate-DNA glycosylase, FaPy-DNA glycosylase, thymine mismatch-DNA glycosylase, hypoxanthine-DNA glycosylase, 5-Hydroxymethyluracil DNA glycosylase (HmUDG), 5-Hydroxymethylcytosine DNA glycosylase, or 1,N6-etheno-adenine DNA glycosylase); exonucleases (e.g., exonuclease III); ribozymes, and DNAzymes. Sample nucleic acid may be treated with a chemical agent, or synthesized using modified nucleotides, and the modified nucleic acid may be cleaved. In non-limiting examples, sample nucleic acid may be treated with (i) alkylating agents such as methylnitrosourea that generate several alkylated bases, including N3-methyladenine and N3-methylguanine, which are recognized and cleaved by alkyl purine DNA-glycosylase; (ii) sodium bisulfite, which causes deamination of cytosine residues in DNA to form uracil residues that can be cleaved by uracil N-glycosylase; and (iii) a chemical agent that converts guanine to its oxidized form, 8-hydroxyguanine, which can be cleaved by formamidopyrimidine DNA N-glycosylase. Examples of chemical cleavage processes include without limitation alkylation, (e.g., alkylation of phosphorothioate-modified nucleic acid); cleavage of acid lability of P3′-N5′-phosphoroamidate-containing nucleic acid; and osmium tetroxide and piperidine treatment of nucleic acid.

As used herein, the term “complementary cleavage reactions” refers to cleavage reactions that are carried out on the same sample nucleic acid using different cleavage reagents or by altering the cleavage specificity of the same cleavage reagent such that alternate cleavage patterns of the same target or reference nucleic acid or protein are generated. In certain embodiments, sample nucleic acid may be treated with one or more specific cleavage agents (e.g., 1, 2, 3, 4, 5, 6, 7, 8, 9, or more specific cleavage agents) in one or more reaction vessels (e.g., sample nucleic acid is treated with each specific cleavage agent in a separate vessel).

Sample nucleic acid also may be exposed to a process that modifies certain nucleotides in the nucleic acid before providing sample nucleic acid for a method described herein. A process that selectively modifies nucleic acid based upon the methylation state of nucleotides therein can be applied to sample nucleic acid, for example. The term “methylation state” as used herein refers to whether a particular nucleotide in a polynucleotide sequence is methylated or not methylated. Methods for modifying a target nucleic acid molecule in a manner that reflects the methylation pattern of the target nucleic acid molecule are known in the art, as exemplified in U.S. Pat. No. 5,786,146 and U.S. patent publications 20030180779 and 20030082600. For example, non-methylated cytosine nucleotides in a nucleic acid can be converted to uracil by bisulfite treatment, which does not modify methylated cytosine. Non-limiting examples of agents that can modify a nucleotide sequence of a nucleic acid include methylmethane sulfonate, ethylmethane sulfonate, diethylsulfate, nitrosoguanidine (N-methyl-N′-nitro-N-nitrosoguanidine), nitrous acid, di-(2-chloroethyl)sulfide, di-(2-chloroethyl)methylamine, 2-aminopurine, t-bromouracil, hydroxylamine, sodium bisulfite, hydrazine, formic acid, sodium nitrite, and 5-methylcytosine DNA glycosylase. In addition, conditions such as high temperature, ultraviolet radiation, x-radiation, can induce changes in the sequence of a nucleic acid molecule.

Sample nucleic acid may be provided in any form useful for conducting a sequence analysis or manufacture process described herein, such as solid or liquid form, for example. In certain embodiments, sample nucleic acid may be provided in a liquid form optionally comprising one or more other components, including without limitation one or more buffers or salts selected.

### Amplification

In some embodiments, one or more nucleic acids are amplified using a suitable amplification process. It may be desirable to amplify a nucleic acid particularly if one or more of the nucleic acid exists at low copy number. In some embodiments amplification of sequences or regions of interest may aid in detection of gene dosage imbalances. An amplification product (amplicon) of a particular nucleic acid is referred to herein as an “amplified nucleic acid.”

Nucleic acid amplification often involves enzymatic synthesis of nucleic acid amplicons (copies), which contain a sequence complementary to a nucleic acid being amplified. Amplifying nucleic acid and detecting the amplicons synthesized, can improve the sensitivity of an assay, since fewer target sequences are needed at the beginning of the assay, and can improve detection of a nucleic acid.

Any suitable amplification technique can be utilized. Amplification of polynucleotides include, but are not limited to, polymerase chain reaction (PCR); ligation amplification (or ligase chain reaction (LCR)); amplification methods based on the use of Q-beta replicase or template-dependent polymerase (see US Patent Publication Number US20050287592); helicase-dependant isothermal amplification (Vincent et al., “Helicase-dependent isothermal DNA amplification”. EMBO reports 5 (8): 795-800 (2004)); strand displacement amplification (SDA); thermophilic SDA nucleic acid sequence based amplification (3SR or NASBA) and transcription-associated amplification (TAA). Non-limiting examples of PCR amplification methods include standard PCR, AFLP-PCR, Allele-specific PCR, Alu-PCR, Asymmetric PCR, Colony PCR, digital PCR, Hot start PCR, Inverse PCR (IPCR), In situ PCR (ISH), Intersequence-specific PCR (ISSR-PCR), Long PCR, Multiplex PCR, Nested PCR, Quantitative PCR, Reverse Transcriptase PCR(RT-PCR), Real Time PCR, Single cell PCR, Solid phase PCR, combinations thereof, and the like. Reagents and hardware for conducting PCR are commercially available.

The terms “amplify”, “amplification”, “amplification reaction”, or “amplifying” refers to any in vitro processes for multiplying the copies of a target sequence of nucleic acid. Amplification sometimes refers to an “exponential” increase in target nucleic acid. However, “amplifying” as used herein can also refer to linear increases in the numbers of a select target sequence of nucleic acid, but is different than a one-time, single primer extension step. In some embodiments a limited amplification reaction, also known as pre-amplification, can be performed. Pre-amplification is a method in which a limited amount of amplification occurs due to a small number of cycles, for example 10 cycles, being performed. Pre-amplification can allow some amplification, but stops amplification prior to the exponential phase, and typically produces about 500 copies of the desired nucleotide sequence(s). Use of pre-amplification may also limit inaccuracies associated with depleted reactants in standard PCR reactions, and also may reduce amplification biases due to nucleotide sequence or species abundance of the target. In some embodiments a one-time primer extension may be used may be performed as a prelude to linear or exponential amplification.

A generalized description of an amplification process is presented herein. Primers and target nucleic acid are contacted, and complementary sequences anneal to one another, for example. Primers can anneal to a target nucleic acid, at or near (e.g., adjacent to, abutting, and the like) a sequence of interest. A reaction mixture, containing components necessary for enzymatic functionality, is added to the primer—target nucleic acid hybrid, and amplification can occur under suitable conditions. Components of an amplification reaction may include, but are not limited to, e.g., primers (e.g., individual primers, primer pairs, primer sets and the like) a polynucleotide template (e.g., target nucleic acid), polymerase, nucleotides, dNTPs and the like. In some embodiments, non-naturally occurring nucleotides or nucleotide analogs, such as analogs containing a detectable label (e.g., fluorescent or colorimetric label), may be used for example. Polymerases can be selected and include polymerases for thermocycle amplification (e.g., Taq DNA Polymerase; Q-Bio™ Taq DNA Polymerase (recombinant truncated form of Taq DNA Polymerase lacking 5′-3′ exo activity); SurePrime™ Polymerase (chemically modified Taq DNA polymerase for “hot start” PCR); Arrow™ Taq DNA Polymerase (high sensitivity and long template amplification)) and polymerases for thermostable amplification (e.g., RNA polymerase for transcription-mediated amplification (TMA) described at World Wide Web URL “gen-probe.com/pdfs/tma_whiteppr.pdf”). Other enzyme components can be added, such as reverse transcriptase for transcription mediated amplification (TMA) reactions, for example.

The terms “near” or “adjacent to” when referring to a nucleotide sequence of interest refers to a distance or region between the end of the primer and the nucleotide or nucleotides of interest. As used herein adjacent is in the range of about 5 nucleotides to about 500 nucleotides (e.g., about 5 nucleotides away from nucleotide of interest, about 10, about 20, about 30, about 40, about 50, about 60, about 70, about 80, about 90, about 100, about 150, about 200, about 250, about 300, abut 350, about 400, about 450 or about 500 nucleotides from a nucleotide of interest). In some embodiments the primers in a set hybridize within about 10 to 30 nucleotides from a nucleic acid sequence of interest and produce amplified products.

Each amplified nucleic acid independently is about 10 to about 500 base pairs in length in some embodiments. In certain embodiments, an amplified nucleic acid is about 20 to about 250 base pairs in length, sometimes is about 50 to about 150 base pairs in length and sometimes is about 100 base pairs in length. Thus, in some embodiments, the length of each of the amplified nucleic acid products independently is about 10, 15, 20, 25, 30, 35, 40, 45, 50, 55, 60, 65, 70, 75, 80, 82, 84, 86, 88, 90, 92, 94, 96, 98, 100, 102, 104, 106, 108, 110, 112, 114, 116, 118, 120, 125, 130, 135, 140, 145, 150, 175, 200, 250, 300, 350, 400, 450, or 500 base pairs (bp) in length.

An amplification product may include naturally occurring nucleotides, non-naturally occurring nucleotides, nucleotide analogs and the like and combinations of the foregoing. An amplification product often has a nucleotide sequence that is identical to or substantially identical to a sample nucleic acid nucleotide sequence or complement thereof. A “substantially identical” nucleotide sequence in an amplification product will generally have a high degree of sequence identity to the nucleic acid being amplified or complement thereof (e.g., about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater than 99% sequence identity), and variations sometimes are a result of infidelity of the polymerase used for extension and/or amplification, or additional nucleotide sequence(s) added to the primers used for amplification.

PCR conditions can be dependent upon primer sequences, target abundance, and the desired amount of amplification, and therefore, one of skill in the art may choose from a number of PCR protocols available (see, e.g., U.S. Pat. Nos. 4,683,195 and 4,683,202; and PCR Protocols: A Guide to Methods and Applications, Innis et al., eds, 1990. Digital PCR is also known to those of skill in the art; see, e.g., US Patent Application Publication Number 20070202525, filed Feb. 2, 2007, which is hereby incorporated by reference). PCR often is carried out as an automated process with a thermostable enzyme. In this process, the temperature of the reaction mixture is cycled through a denaturing region, a primer-annealing region, and an extension reaction region automatically. Machines specifically adapted for this purpose are commercially available. A non-limiting example of a PCR protocol that may be suitable for embodiments described herein is, treating the sample at 95° C. for 5 minutes; repeating forty-five cycles of 95° C. for 1 minute, 59° C. for 1 minute, 10 seconds, and 72° C. for 1 minute 30 seconds; and then treating the sample at 72° C. for 5 minutes. Multiple cycles frequently are performed using a commercially available thermal cycler. Suitable isothermal amplification processes known and selected also may be applied, in certain embodiments.

In some embodiments, multiplex amplification processes may be used to amplify target nucleic acids, such that multiple amplicons are simultaneously amplified in a single, homogenous reaction. As used herein “multiplex amplification” refers to a variant of PCR where simultaneous amplification of many targets of interest in one reaction vessel may be accomplished by using more than one pair of primers (e.g., more than one primer set). Multiplex amplification may be useful for analysis of deletions, mutations, and polymorphisms, or quantitative assays, in some embodiments. In certain embodiments multiplex amplification may be used for detecting paralog sequence imbalance, genotyping applications where simultaneous analysis of multiple markers is required, detection of pathogens or genetically modified organisms, or for microsatellite analyses. In some embodiments multiplex amplification may be combined with another amplification (e.g., PCR) method (e.g., digital PCR, nested PCR or hot start PCR, for example) to increase amplification specificity and reproducibility. In other embodiments multiplex amplification may be done in replicates, for example, to reduce the variance introduced by said amplification.

In certain embodiments, nucleic acid amplification can generate additional nucleic acid species of different or substantially similar nucleic acid sequence. In certain embodiments described herein, contaminating or additional nucleic acid species, which may contain sequences substantially complementary to, or may be substantially identical to, the sequence of interest, can be useful for sequence quantification, with the proviso that the level of contaminating or additional sequences remains constant and therefore can be a reliable marker whose level can be substantially reproduced. Additional considerations that may affect sequence amplification reproducibility are: PCR conditions (number of cycles, volume of reactions, melting temperature difference between primers pairs, and the like), concentration of target nucleic acid in sample, the number of chromosomes on which the nucleotide species of interest resides, variations in quality of prepared sample, and the like. The terms “substantially reproduced” or “substantially reproducible” as used herein refer to a result (e.g., quantifiable amount of nucleic acid) that under substantially similar conditions would occur in substantially the same way about 75% of the time or greater, about 80%, about 85%, about 90%, about 95%, or about 99% of the time or greater.

In some embodiments where a target nucleic acid is RNA, prior to the amplification step, a DNA copy (cDNA) of the RNA transcript of interest may be synthesized. A cDNA can be synthesized by reverse transcription, which can be carried out as a separate step, or in a homogeneous reverse transcription-polymerase chain reaction (RT-PCR), a modification of the polymerase chain reaction for amplifying RNA. Methods suitable for PCR amplification of ribonucleic acids are described by Romero and Rotbart in Diagnostic Molecular Biology: Principles and Applications pp. 401-406; Persing et al., eds., Mayo Foundation, Rochester, Minn., 1993; Egger et al., J. Clin. Microbiol. 33:1442-1447, 1995; and U.S. Pat. No. 5,075,212. Branched-DNA technology may be used to amplify the signal of RNA markers in maternal blood. For a review of branched-DNA (bDNA) signal amplification for direct quantification of nucleic acid sequences in clinical samples, see Nolte, Adv. Clin. Chem. 33:201-235, 1998.

Amplification also can be accomplished using digital PCR, in certain embodiments (e.g., Kalinina and colleagues (Kalinina et al., “Nanoliter scale PCR with TaqMan detection.” Nucleic Acids Research. 25; 1999-2004, (1997); Vogelstein and Kinzler (Digital PCR. Proc Natl Acad Sci USA. 96; 9236-41, (1999); PCT Patent Publication No. WO05023091A2; US Patent Publication No. US 20070202525). Digital PCR takes advantage of nucleic acid (DNA, cDNA or RNA) amplification on a single molecule level, and offers a highly sensitive method for quantifying low copy number nucleic acid. Systems for digital amplification and analysis of nucleic acids are available (e.g., Fluidigm® Corporation). Digital PCR is useful for studying variations in gene sequences (e.g., copy number variants, point mutations, and the like). In general, samples being analyzed by digital PCR are partitioned (e.g., captured, isolated) into reaction vessels or chambers such that a single nucleic acid is contained in each reaction, in some embodiments. Samples can be partitioned using any method known in the art, non-limiting examples of which include the use of micro well plates (e.g., microtiter plates) capillaries, the dispersed phase of an emulsion, microfluidic devices, solid supports, the like or combinations of the foregoing. Partitioning of the sample allows estimation of the number of molecules according to Poisson distribution. Generally, each reaction vessel will contain 0 or 1 starting nucleic acid molecules from which amplification occurs. Reactions with 0 nucleic acid molecules do no generate an amplified product, whereas reactions with 1 nucleic acid generate an amplified product. After amplification, nucleic acids may be quantified by counting the reactions that generate a PCR product. Digital PCR generally does not rely on the number of amplification cycles performed to determine the number of copies of a nucleic acid of interest in a sample. Thus, digital PCR reduces or eliminates reliance on data from procedures that use exponential amplification, which sometimes can introduce amplification artifacts. Digital PCR generally provides a more robust method of quantification than conventional PCR.

In some embodiments, digital PCR is performed with primer sets that include one or more primers that anneal to nucleic acid sequences located within a multiplied region (e.g., a multiplied CFH allele or CFHR allele). In certain embodiments, digital PCR is performed with primer sets that include one or more primers that anneal to nucleic acid sequences located within a multiplied region and/or one or more primers that anneal to nucleic acid sequences located outside of a multiplied region. In some embodiments, a primer set includes one or more primers that amplify a control region, which control region does not include a multiplied region. In some embodiments, one or more primers utilized in a digital PCR assay described herein includes a polymorphic nucleotide position, and in certain embodiments, the polymorphic nucleotide position is determinative of the presence or absence of a haplotype associated with a disease condition. In some embodiments, a haplotype is associated with a polymorphic nucleotide, a multiplied region or a polymorphic nucleotide and a multiplied region. In some embodiments, the disease condition is AMD.

Use of a primer extension reaction also can be applied in methods of the technology. A primer extension reaction operates, for example, by discriminating nucleic acid sequences at a single nucleotide mismatch, in some embodiments. The mismatch is detected by the incorporation of one or more deoxynucleotides and/or dideoxynucleotides to an extension oligonucleotide, which hybridizes to a region adjacent to the mismatch site. The extension oligonucleotide generally is extended with a polymerase. In some embodiments, a detectable tag or detectable label is incorporated into the extension oligonucleotide or into the nucleotides added on to the extension oligonucleotide (e.g., biotin or streptavidin). The extended oligonucleotide can be detected by any known suitable detection process (e.g., mass spectrometry; sequencing processes). In some embodiments, the mismatch site is extended only by one or two complementary deoxynucleotides or dideoxynucleotides that are tagged by a specific label or generate a primer extension product with a specific mass, and the mismatch can be discriminated and quantified.

In some embodiments, amplification may be performed on a solid support. In some embodiments, primers may be associated with a solid support. In certain embodiments, target nucleic acid (e.g., template nucleic acid) may be associated with a solid support. A nucleic acid (primer or target) in association with a solid support often is referred to as a solid phase nucleic acid.

In some embodiments, nucleic acid molecules provided for amplification and in a “microreactor”. As used herein, the term “microreactor” refers to a partitioned space in which a nucleic acid molecule can hybridize to a solid support nucleic acid molecule. Examples of microreactors include, without limitation, an emulsion globule (described hereafter) and a void in a substrate. A void in a substrate can be a pit, a pore or a well (e.g., microwell, nanowell, picowell, micropore, or nanopore) in a substrate constructed from a solid material useful for containing fluids (e.g., plastic (e.g., polypropylene, polyethylene, polystyrene) or silicon) in certain embodiments. Emulsion globules are partitioned by an immiscible phase as described in greater detail hereafter. In some embodiments, the microreactor volume is large enough to accommodate one solid support (e.g., bead) in the microreactor and small enough to exclude the presence of two or more solid supports in the microreactor.

The term “emulsion” as used herein refers to a mixture of two immiscible and unblendable substances, in which one substance (the dispersed phase) often is dispersed in the other substance (the continuous phase). The dispersed phase can be an aqueous solution (i.e., a solution comprising water) in certain embodiments. In some embodiments, the dispersed phase is composed predominantly of water (e.g., greater than 70%, greater than 75%, greater than 80%, greater than 85%, greater than 90%, greater than 95%, greater than 97%, greater than 98% and greater than 99% water (by weight)). Each discrete portion of a dispersed phase, such as an aqueous dispersed phase, is referred to herein as a “globule” or “microreactor.” A globule sometimes may be spheroidal, substantially spheroidal or semi-spheroidal in shape, in certain embodiments.

The terms “emulsion apparatus” and “emulsion component(s)” as used herein refer to apparatus and components that can be used to prepare an emulsion. Non-limiting examples of emulsion apparatus include without limitation counter-flow, cross-current, rotating drum and membrane apparatus suitable for use to prepare an emulsion. An emulsion component forms the continuous phase of an emulsion in certain embodiments, and includes without limitation a substance immiscible with water, such as a component comprising or consisting essentially of an oil (e.g., a heat-stable, biocompatible oil (e.g., light mineral oil)). A biocompatible emulsion stabilizer can be utilized as an emulsion component. Emulsion stabilizers include without limitation Atlox 4912, Span 80 and other biocompatible surfactants.

In some embodiments, components useful for biological reactions can be included in the dispersed phase. Globules of the emulsion can include (i) a solid support unit (e.g., one bead or one particle); (ii) sample nucleic acid molecule; and (iii) a sufficient amount of extension agents to elongate solid phase nucleic acid and amplify the elongated solid phase nucleic acid (e.g., extension nucleotides, polymerase, primer). Inactive globules in the emulsion may include a subset of these components (e.g., solid support and extension reagents and no sample nucleic acid) and some can be empty (i.e., some globules will include no solid support, no sample nucleic acid and no extension agents).

Emulsions may be prepared using known suitable methods (e.g., Nakano et al. “Single-molecule PCR using water-in-oil emulsion;” Journal of Biotechnology 102 (2003) 117-124). Emulsification methods include without limitation adjuvant methods, counter-flow methods, cross-current methods, rotating drum methods, membrane methods, and the like. In certain embodiments, an aqueous reaction mixture containing a solid support (hereafter the “reaction mixture”) is prepared and then added to a biocompatible oil. In certain embodiments, the reaction mixture may be added dropwise into a spinning mixture of biocompatible oil (e.g., light mineral oil (Sigma)) and allowed to emulsify. In some embodiments, the reaction mixture may be added dropwise into a cross-flow of biocompatible oil. The size of aqueous globules in the emulsion can be adjusted, such as by varying the flow rate and speed at which the components are added to one another, for example.

The size of emulsion globules can be selected in certain embodiments based on two competing factors: (i) globules are sufficiently large to encompass one solid support molecule, one sample nucleic acid molecule, and sufficient extension agents for the degree of elongation and amplification required; and (ii) globules are sufficiently small so that a population of globules can be amplified by conventional laboratory equipment (e.g., thermocycling equipment, test tubes, incubators and the like). Globules in the emulsion can have a nominal, mean or average diameter of about 5 microns to about 500 microns, about 10 microns to about 350 microns, about 50 to 250 microns, about 100 microns to about 200 microns, or about 5, 10, 15, 20, 25, 30, 35, 40, 45, 50, 55, 60, 65, 70, 75, 80, 85, 90, 95, 100, 200, 300, 400 or 500 microns in certain embodiments.

In certain embodiments, amplified nucleic acid in a set are of identical length, and sometimes the amplified nucleic acid in a set are of a different length. For example, one amplified nucleic acid may be longer than one or more other amplified nucleic acid in the set by about 1 to about 100 nucleotides (e.g., about 2, 3, 4, 5, 6, 7, 8, 9, 10, 11, 12, 13, 14, 15, 16, 17, 18, 19, 20, 30, 40, 50, 60, 70, 80 or 90 nucleotides longer).

In some embodiments, a ratio can be determined for the amount of one amplified nucleic acid in a set to the amount of another amplified nucleic acid in the set (hereafter a “set ratio”). In some embodiments, the amount of one amplified nucleic acid in a set is about equal to the amount of another amplified nucleic acid in the set (i.e., amounts of amplified nucleic acid in a set are about 1:1), which generally is the case when the number of chromosomes in a sample bearing each nucleic acid amplified is about equal. The term “amount” as used herein with respect to amplified nucleic acid refers to any suitable measurement, including, but not limited to, copy number, weight (e.g., grams) and concentration (e.g., grams per unit volume (e.g., milliliter); molar units). In certain embodiments, the amount of one amplified nucleic acid in a set can differ from the amount of another amplified nucleic acid in a set, even when the number of chromosomes in a sample bearing each nucleic acid amplified is about equal. In some embodiments, amounts of amplified nucleic acid within a set may vary up to a threshold level at which a chromosome abnormality can be detected with a confidence level of about 95% (e.g., about 90, 91, 92, 93, 94, 95, 96, 97, 98, 99, or greater than 99%). In certain embodiments, the amounts of the amplified nucleic acid in a set vary by about 50% or less (e.g., about 45, 40, 35, 30, 25, 20, 15, 10, 5, 4, 3, 2 or 1%, or less than 1%). Thus, in certain embodiments amounts of amplified nucleic acid in a set may vary from about 1:1 to about 1:1.5. Without being limited by theory, certain factors can lead to the observation that the amount of one amplified nucleic acid in a set can differ from the amount of another amplified nucleic acid in a set, even when the number of chromosomes in a sample bearing each nucleic acid amplified is about equal. Such factors may include different amplification efficiency rates and/or amplification from a chromosome not intended in the assay design.

Each amplified nucleic acid in a set generally is amplified under conditions that amplify that species at a substantially reproducible level. The term “substantially reproducible level” as used herein refers to consistency of amplification levels for a particular amplified nucleic acid per unit template nucleic acid (e.g., per unit template nucleic acid that contains the particular nucleic acid amplified). A substantially reproducible level varies by about 1% or less in certain embodiments, after factoring the amount of template nucleic acid giving rise to a particular amplification nucleic acid species (e.g., normalized for the amount of template nucleic acid). In some embodiments, a substantially reproducible level varies by 10%, 5%, 4%, 3%, 2%, 1.5%, 1%, 0.5%, 0.1%, 0.05%, 0.01%, 0.005% or 0.001% after factoring the amount of template nucleic acid giving rise to a particular amplification nucleic acid species. Alternatively, substantially reproducible means that any two or more measurements of an amplification level are within a particular coefficient of variation (“CV”) from a given mean. Such CV may be 20% or less, sometimes 10% or less and at times 5% or less. The two or more measurements of an amplification level may be determined between two or more reactions and/or two or more of the same sample types (for example, two normal samples or two trisomy samples)

### Primers

Primers useful for detection, quantification, amplification, sequencing and analysis of nucleic acid are provided. In some embodiments primers are used in sets, where a set contains at least a pair. In some embodiments a set of primers may include a third or a fourth nucleic acid (e.g., two pairs of primers or nested sets of primers, for example). A plurality of primer pairs may constitute a primer set in certain embodiments (e.g., about 2, 3, 4, 5, 6, 7, 8, 9, 10, 15, 20, 25, 30, 35, 40, 45, 50, 55, 60, 65, 70, 75, 80, 85, 90, 95 or 100 pairs). In some embodiments a plurality of primer sets, each set comprising pair(s) of primers, may be used. The term “primer” as used herein refers to a nucleic acid that comprises a nucleotide sequence capable of hybridizing or annealing to a target nucleic acid, at or near (e.g., adjacent to) a specific region of interest. Primers can allow for specific determination of a target nucleic acid nucleotide sequence or detection of the target nucleic acid (e.g., presence or absence of a sequence or copy number of a sequence), or feature thereof, for example. A primer may be naturally occurring or synthetic. The term “specific” or “specificity”, as used herein, refers to the binding or hybridization of one molecule to another molecule, such as a primer for a target polynucleotide. That is, “specific” or “specificity” refers to the recognition, contact, and formation of a stable complex between two molecules, as compared to substantially less recognition, contact, or complex formation of either of those two molecules with other molecules. As used herein, the term “anneal” refers to the formation of a stable complex between two molecules. The terms “primer”, “oligo”, or “oligonucleotide” may be used interchangeably throughout the document, when referring to primers.

A primer nucleic acid can be designed and synthesized using suitable processes, and may be of any length suitable for hybridizing to a nucleotide sequence of interest (e.g., where the nucleic acid is in liquid phase or bound to a solid support) and performing analysis processes described herein. Primers may be designed based upon a target nucleotide sequence. A primer in some embodiments may be about 10 to about 100 nucleotides, about 10 to about 70 nucleotides, about 10 to about 50 nucleotides, about 15 to about 30 nucleotides, or about 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100 nucleotides in length. A primer may be composed of naturally occurring and/or non-naturally occurring nucleotides (e.g., labeled nucleotides), or a mixture thereof. Primers suitable for use with embodiments described herein, may be synthesized and labeled using known techniques. Oligonucleotides (e.g., primers) may be chemically synthesized according to the solid phase phosphoramidite triester method first described by Beaucage and Caruthers, Tetrahedron Letts., 22:1859-1862, 1981, using an automated synthesizer, as described in Needham-VanDevanter et al., Nucleic Acids Res. 12:6159-6168, 1984. Purification of oligonucleotides can be effected by native acrylamide gel electrophoresis or by anion-exchange high-performance liquid chromatography (HPLC), for example, as described in Pearson and Regnier, J. Chrom., 255:137-149, 1983.

All or a portion of a primer nucleic acid sequence (naturally occurring or synthetic) may be substantially complementary to a target nucleic acid, in some embodiments. As referred to herein, “substantially complementary” with respect to sequences refers to nucleotide sequences that will hybridize with each other. The stringency of the hybridization conditions can be altered to tolerate varying amounts of sequence mismatch. Included are regions of counterpart, target and capture nucleotide sequences 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other.

Primers that are substantially complimentary to a target nucleic acid sequence are also substantially identical to the compliment of the target nucleic acid sequence. That is, primers are substantially identical to the anti-sense strand of the nucleic acid. As referred to herein, “substantially identical” with respect to sequences refers to nucleotide sequences that are 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more identical to each other. One test for determining whether two nucleotide sequences are substantially identical is to determine the percent of identical nucleotide sequences shared.

Primer sequences and length may affect hybridization to target nucleic acid sequences. Depending on the degree of mismatch between the primer and target nucleic acid, low, medium or high stringency conditions may be used to effect primer/target annealing. As used herein, the term “stringent conditions” refers to conditions for hybridization and washing. Methods for hybridization reaction temperature condition optimization are known to those of skill in the art, and may be found in Current Protocols in Molecular Biology, John Wiley & Sons, N.Y., 6.3.1-6.3.6 (1989). Aqueous and non-aqueous methods are described in that reference and either can be used. Non-limiting examples of stringent hybridization conditions are hybridization in 6× sodium chloride/sodium citrate (SSC) at about 45° C., followed by one or more washes in 0.2×SSC, 0.1% SDS at 50° C. Another example of stringent hybridization conditions are hybridization in 6× sodium chloride/sodium citrate (SSC) at about 45° C., followed by one or more washes in 0.2×SSC, 0.1% SDS at 55° C. A further example of stringent hybridization conditions is hybridization in 6× sodium chloride/sodium citrate (SSC) at about 45° C., followed by one or more washes in 0.2×SSC, 0.1% SDS at 60° C. Often, stringent hybridization conditions are hybridization in 6× sodium chloride/sodium citrate (SSC) at about 45° C., followed by one or more washes in 0.2×SSC, 0.1% SDS at 65° C. More often, stringency conditions are 0.5M sodium phosphate, 7% SDS at 65° C., followed by one or more washes at 0.2×SSC, 1% SDS at 65° C. Stringent hybridization temperatures can also be altered (i.e. lowered) with the addition of certain organic solvents, formamide for example. Organic solvents, like formamide, reduce the thermal stability of double-stranded polynucleotides, so that hybridization can be performed at lower temperatures, while still maintaining stringent conditions and extending the useful life of nucleic acids that may be heat labile.

As used herein, the phrase “hybridizing” or grammatical variations thereof, refers to binding of a first nucleic acid molecule to a second nucleic acid molecule under low, medium or high stringency conditions, or under nucleic acid synthesis conditions. Hybridizing can include instances where a first nucleic acid molecule binds to a second nucleic acid molecule, where the first and second nucleic acid molecules are complementary. As used herein, “specifically hybridizes” refers to preferential hybridization under nucleic acid synthesis conditions of a primer, to a nucleic acid molecule having a sequence complementary to the primer compared to hybridization to a nucleic acid molecule not having a complementary sequence. For example, specific hybridization includes the hybridization of a primer to a target nucleic acid sequence that is complementary to the primer.

In some embodiments primers can include a nucleotide subsequence that may be complementary to a solid phase nucleic acid primer hybridization sequence or substantially complementary to a solid phase nucleic acid primer hybridization sequence (e.g., about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater than 99% identical to the primer hybridization sequence complement when aligned). A primer may contain a nucleotide subsequence not complementary to or not substantially complementary to a solid phase nucleic acid primer hybridization sequence (e.g., at the 3′ or 5′ end of the nucleotide subsequence in the primer complementary to or substantially complementary to the solid phase primer hybridization sequence).

A primer, in certain embodiments, may contain a modification such as inosines, abasic sites, locked nucleic acids, minor groove binders, duplex stabilizers (e.g., acridine, spermidine), Tm modifiers or any modifier that changes the binding properties of the primers or probes.

A primer, in certain embodiments, may contain a detectable molecule or entity (e.g., a fluorophore, radioisotope, colorimetric agent, particle, enzyme and the like). When desired, the nucleic acid can be modified to include a detectable label using any method known to one of skill in the art. The label may be incorporated as part of the synthesis, or added on prior to using the primer in any of the processes described herein. Incorporation of label may be performed either in liquid phase or on solid phase. In some embodiments the detectable label may be useful for detection of targets. In some embodiments the detectable label may be useful for the quantification target nucleic acids (e.g., determining copy number of a particular sequence or species of nucleic acid). Any detectable label suitable for detection of an interaction or biological activity in a system can be appropriately selected and utilized by the artisan. Examples of detectable labels are fluorescent labels such as fluorescein, rhodamine, and others (e.g., Anantha, et al., Biochemistry (1998) 37:2709 2714; and Qu & Chaires, Methods Enzymol. (2000) 321:353 369); radioactive isotopes (e.g., 125I, 131I, 35S, 31P, 32P, 33P, 14C, 3H, 7Be, 28Mg, 57Co, 65Zn, 67Cu, 68Ge, 82Sr, 83Rb, 95Tc, 96Tc, 103Pd, 109Cd, and 127Xe); light scattering labels (e.g., U.S. Pat. No. 6,214,560, and commercially available from Genicon Sciences Corporation, CA); chemiluminescent labels and enzyme substrates (e.g., dioxetanes and acridinium esters), enzymic or protein labels (e.g., green fluorescence protein (GFP) or color variant thereof, luciferase, peroxidase); other chromogenic labels or dyes (e.g., cyanine), and other cofactors or biomolecules such as digoxigenin, strepdavidin, biotin (e.g., members of a binding pair such as biotin and avidin for example), affinity capture moieties and the like. In some embodiments a primer may be labeled with an affinity capture moiety. Also included in detectable labels are those labels useful for mass modification for detection with mass spectrometry (e.g., matrix-assisted laser desorption ionization (MALDI) mass spectrometry and electrospray (ES) mass spectrometry).

A primer also may refer to a polynucleotide sequence that hybridizes to a subsequence of a target nucleic acid or another primer and facilitates the detection of a primer, a target nucleic acid or both, as with molecular beacons, for example. The term “molecular beacon” as used herein refers to detectable molecule, where the detectable property of the molecule is detectable only under certain specific conditions, thereby enabling it to function as a specific and informative signal. Non-limiting examples of detectable properties are, optical properties, electrical properties, magnetic properties, chemical properties and time or speed through an opening of known size.

In some embodiments a molecular beacon can be a single-stranded oligonucleotide capable of forming a stem-loop structure, where the loop sequence may be complementary to a target nucleic acid sequence of interest and is flanked by short complementary arms that can form a stem. The oligonucleotide may be labeled at one end with a fluorophore and at the other end with a quencher molecule. In the stem-loop conformation, energy from the excited fluorophore is transferred to the quencher, through long-range dipole-dipole coupling similar to that seen in fluorescence resonance energy transfer, or FRET, and released as heat instead of light. When the loop sequence is hybridized to a specific target sequence, the two ends of the molecule are separated and the energy from the excited fluorophore is emitted as light, generating a detectable signal. Molecular beacons offer the added advantage that removal of excess probe is unnecessary due to the self-quenching nature of the unhybridized probe. In some embodiments molecular beacon probes can be designed to either discriminate or tolerate mismatches between the loop and target sequences by modulating the relative strengths of the loop-target hybridization and stem formation. As referred to herein, the term “mismatched nucleotide” or a “mismatch” refers to a nucleotide that is not complementary to the target sequence at that position or positions. A probe may have at least one mismatch, but can also have 2, 3, 4, 5, 6 or 7 or more mismatched nucleotides.

### Detection

Nucleic acid, or amplified nucleic acid, or detectable products prepared from the foregoing, can be detected by a suitable detection process. Non-limiting examples of methods of detection, quantification, sequencing and the like include mass detection of mass modified amplicons (e.g., matrix-assisted laser desorption ionization (MALD I) mass spectrometry and electrospray (ES) mass spectrometry), a primer extension method (e.g., iPLEX®; Sequenom, Inc.), direct DNA sequencing, Molecular Inversion Probe (MIP) technology from Affymetrix, restriction fragment length polymorphism (RFLP analysis), allele specific oligonucleotide (ASO) analysis, methylation-specific PCR (MSPCR), pyrosequencing analysis, acycloprime analysis, Reverse dot blot, GeneChip microarrays, Dynamic allele-specific hybridization (DASH), Peptide nucleic acid (PNA) and locked nucleic acids (LNA) probes, TaqMan, Molecular Beacons, Intercalating dye, FRET primers, AlphaScreen, SNPstream, genetic bit analysis (GBA), Multiplex minisequencing, SNaPshot, GOOD assay, Microarray miniseq, arrayed primer extension (APEX), Microarray primer extension, Tag arrays, Coded microspheres, Template-directed incorporation (TDI), fluorescence polarization, Colorimetric oligonucleotide ligation assay (OLA), Sequence-coded OLA, Microarray ligation, Ligase chain reaction, Padlock probes, Invader assay, hybridization using at least one probe, hybridization using at least one fluorescently labeled probe, in situ hybridization techniques (e.g., fluorescence in situ hybridization (FISH), including fiber FISH), cloning and sequencing, electrophoresis, the use of hybridization probes and quantitative real time polymerase chain reaction (QRT-PCR), digital PCR, nanopore sequencing, chips and combinations thereof. The detection and quantification of alleles or paralogs can be carried out using the “closed-tube” methods described in U.S. patent application Ser. No. 11/950,395, which was filed Dec. 4, 2007. In some embodiments the amount of each amplified nucleic acid is determined by mass spectrometry, primer extension, sequencing (e.g., any suitable method, for example nanopore or pyrosequencing), Quantitative PCR (Q-PCR or QRT-PCR), digital PCR, combinations thereof, and the like.

A target nucleic acid can be detected by detecting a detectable label or “signal-generating moiety” in some embodiments. The term “signal-generating” as used herein refers to any atom or molecule that can provide a detectable or quantifiable effect, and that can be attached to a nucleic acid. In certain embodiments, a detectable label generates a unique light signal, a fluorescent signal, a luminescent signal, an electrical property, a chemical property, a magnetic property and the like.

Detectable labels include, but are not limited to, nucleotides (labeled or unlabelled), compomers, sugars, peptides, proteins, antibodies, chemical compounds, conducting polymers, binding moieties such as biotin, mass tags, colorimetric agents, light emitting agents, chemiluminescent agents, light scattering agents, fluorescent tags, radioactive tags, charge tags (electrical or magnetic charge), volatile tags and hydrophobic tags, biomolecules (e.g., members of a binding pair antibody/antigen, antibody/antibody, antibody/antibody fragment, antibody/antibody receptor, antibody/protein A or protein G, hapten/anti-hapten, biotin/avidin, biotin/streptavidin, folic acid/folate binding protein, vitamin B12/intrinsic factor, chemical reactive group/complementary chemical reactive group (e.g., sulfhydryl/maleimide, sulfhydryl/haloacetyl derivative, amine/isotriocyanate, amine/succinimidyl ester, and amine/sulfonyl halides) and the like, some of which are further described below. In some embodiments a probe may contain a signal-generating moiety that hybridizes to a target and alters the passage of the target nucleic acid through a nanopore, and can generate a signal when released from the target nucleic acid when it passes through the nanopore (e.g., alters the speed or time through a pore of known size).

In certain embodiments, sample tags are introduced to distinguish between samples (e.g., from different patients), thereby allowing for the simultaneous testing of multiple samples. For example, sample tags may introduced as part of the extend primers such that extended primers can be associated with a particular sample.

A solution containing amplicons produced by an amplification process, or a solution containing extension products produced by an extension process, can be subjected to further processing. For example, a solution can be contacted with an agent that removes phosphate moieties from free nucleotides that have not been incorporated into an amplicon or extension product. An example of such an agent is a phosphatase (e.g., alkaline phosphatase). Amplicons and extension products also may be associated with a solid phase, may be washed, may be contacted with an agent that removes a terminal phosphate (e.g., exposure to a phosphatase), may be contacted with an agent that removes a terminal nucleotide (e.g., exonuclease), may be contacted with an agent that cleaves (e.g., endonuclease, ribonuclease), and the like.

The term “solid support” or “solid phase” as used herein refers to an insoluble material with which nucleic acid can be associated. Examples of solid supports for use with processes described herein include, without limitation, arrays, beads (e.g., paramagnetic beads, magnetic beads, microbeads, nanobeads) and particles (e.g., microparticles, nanoparticles). Particles or beads having a nominal, average or mean diameter of about 1 nanometer to about 500 micrometers can be utilized, such as those having a nominal, mean or average diameter, for example, of about 10 nanometers to about 100 micrometers; about 100 nanometers to about 100 micrometers; about 1 micrometer to about 100 micrometers; about 10 micrometers to about 50 micrometers; about 1, 5, 10, 15, 20, 25, 30, 35, 40, 45, 50, 55, 60, 65, 70, 75, 80, 85, 90, 95, 100, 200, 300, 400, 500, 600, 700, 800 or 900 nanometers; or about 1, 5, 10, 15, 20, 25, 30, 35, 40, 45, 50, 55, 60, 65, 70, 75, 80, 85, 90, 95, 100, 200, 300, 400, 500 micrometers.

A solid support can comprise virtually any insoluble or solid material, and often a solid support composition is selected that is insoluble in water. For example, a solid support can comprise or consist essentially of silica gel, glass (e.g. controlled-pore glass (CPG)), nylon, Sephadex®, Sepharose®, cellulose, a metal surface (e.g. steel, gold, silver, aluminum, silicon and copper), a magnetic material, a plastic material (e.g., polyethylene, polypropylene, polyamide, polyester, polyvinylidenedifluoride (PVDF)) and the like. Beads or particles may be swellable (e.g., polymeric beads such as Wang resin) or non-swellable (e.g., CPG). Commercially available examples of beads include without limitation Wang resin, Merrifield resin and Dynabeads® and SoluLink.

A solid support may be provided in a collection of solid supports. A solid support collection comprises two or more different solid support species. The term “solid support species” as used herein refers to a solid support in association with one particular solid phase nucleic acid species or a particular combination of different solid phase nucleic acid species. In certain embodiments, a solid support collection comprises 2 to 10,000 solid support species, 10 to 1,000 solid support species or about 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000, 9000 or 10000 unique solid support species. The solid supports (e.g., beads) in the collection of solid supports may be homogeneous (e.g., all are Wang resin beads) or heterogeneous (e.g., some are Wang resin beads and some are magnetic beads). Each solid support species in a collection of solid supports sometimes is labeled with a specific identification tag. An identification tag for a particular solid support species sometimes is a nucleic acid (e.g., “solid phase nucleic acid”) having a unique sequence in certain embodiments. An identification tag can be any molecule that is detectable and distinguishable from identification tags on other solid support species.

Nucleic acid, amplified nucleic acid, or detectable products generated from the foregoing may be subject to sequence analysis. The term “sequence analysis” as used herein refers to determining a nucleotide sequence of an amplification product. The entire sequence or a partial sequence of an amplification product can be determined, and the determined nucleotide sequence is referred to herein as a “read.” For example, linear amplification products may be analyzed directly without further amplification in some embodiments (e.g., by using single-molecule sequencing methodology (described in greater detail hereafter)). In certain embodiments, linear amplification products may be subject to further amplification and then analyzed (e.g., using sequencing by ligation or pyrosequencing methodology (described in greater detail hereafter)). Reads may be subject to different types of sequence analysis. Any suitable sequencing method can be utilized to detect, and determine the amount of, nucleic acid, amplified nucleic acid, or detectable products generated from the foregoing. In one embodiment, a heterogeneous sample is subjected to targeted sequencing (or partial targeted sequencing) where one or more sets of nucleic acid species are sequenced, and the amount of each sequenced nucleic acid species in the set is determined, whereby the presence or absence of a chromosome abnormality is identified based on the amount of the sequenced nucleic acid species. Examples of certain sequencing methods are described hereafter.

The terms “sequence analysis apparatus” and “sequence analysis component(s)” used herein refer to apparatus, and one or more components used in conjunction with such apparatus, that can be used to determine a nucleotide sequence from amplification products resulting from processes described herein (e.g., linear and/or exponential amplification products). Examples of sequencing platforms include, without limitation, the 454 platform (Roche) (Margulies, M. et al. 2005 Nature 437, 376-380), IIlumina Genomic Analyzer (or Solexa platform) or SOLID System (Applied Biosystems) or the Helicos True Single Molecule DNA sequencing technology (Harris T D et al. 2008 Science, 320, 106-109), the single molecule, real-time (SMRT™) technology of Pacific Biosciences, and nanopore sequencing (Soni GV and Meller A. 2007 Clin Chem 53: 1996-2001). Such platforms allow sequencing of many nucleic acid molecules isolated from a specimen at high orders of multiplexing in a parallel manner (Dear Brief Funct Genomic Proteomic 2003; 1: 397-416). Each of these platforms allow sequencing of clonally expanded or non-amplified single molecules of nucleic acid fragments. Certain platforms involve, for example, (i) sequencing by ligation of dye-modified probes (including cyclic ligation and cleavage), (ii) pyrosequencing, and (iii) single-molecule sequencing. Nucleic acid, amplified nucleic acid and detectable products generated there from can be considered a “study nucleic acid” for purposes of analyzing a nucleotide sequence by such sequence analysis platforms.

Sequencing by ligation is a nucleic acid sequencing method that relies on the sensitivity of DNA ligase to base-pairing mismatch. DNA ligase joins together ends of DNA that are correctly base paired. Combining the ability of DNA ligase to join together only correctly base paired DNA ends, with mixed pools of fluorescently labeled oligonucleotides or primers, enables sequence determination by fluorescence detection. Longer sequence reads may be obtained by including primers containing cleavable linkages that can be cleaved after label identification. Cleavage at the linker removes the label and regenerates the 5′ phosphate on the end of the ligated primer, preparing the primer for another round of ligation. In some embodiments primers may be labeled with more than one fluorescent label (e.g., 1 fluorescent label, 2,3, or 4 fluorescent labels).

An example of a system that can be used based on sequencing by ligation generally involves the following steps. Clonal bead populations can be prepared in emulsion microreactors containing study nucleic acid (“template”), amplification reaction components, beads and primers. After amplification, templates are denatured and bead enrichment is performed to separate beads with extended templates from undesired beads (e.g., beads with no extended templates). The template on the selected beads undergoes a 3′ modification to allow covalent bonding to the slide, and modified beads can be deposited onto a glass slide. Deposition chambers offer the ability to segment a slide into one, four or eight chambers during the bead loading process. For sequence analysis, primers hybridize to the adapter sequence. A set of four color dye-labeled probes competes for ligation to the sequencing primer. Specificity of probe ligation is achieved by interrogating every 4th and 5th base during the ligation series. Five to seven rounds of ligation, detection and cleavage record the color at every 5th position with the number of rounds determined by the type of library used. Following each round of ligation, a new complimentary primer offset by one base in the 5′ direction is laid down for another series of ligations. Primer reset and ligation rounds (5-7 ligation cycles per round) are repeated sequentially five times to generate 25-35 base pairs of sequence for a single tag. With mate-paired sequencing, this process is repeated for a second tag. Such a system can be used to exponentially amplify amplification products generated by a process described herein, e.g., by ligating a heterologous nucleic acid to the first amplification product generated by a process described herein and performing emulsion amplification using the same or a different solid support originally used to generate the first amplification product. Such a system also may be used to analyze amplification products directly generated by a process described herein by bypassing an exponential amplification process and directly sorting the solid supports described herein on the glass slide.

Pyrosequencing is a nucleic acid sequencing method based on sequencing by synthesis, which relies on detection of a pyrophosphate released on nucleotide incorporation. Generally, sequencing by synthesis involves synthesizing, one nucleotide at a time, a DNA strand complimentary to the strand whose sequence is being sought. Study nucleic acids may be immobilized to a solid support, hybridized with a sequencing primer, incubated with DNA polymerase, ATP sulfurylase, luciferase, apyrase, adenosine 5′ phosphsulfate and luciferin. Nucleotide solutions are sequentially added and removed. Correct incorporation of a nucleotide releases a pyrophosphate, which interacts with ATP sulfurylase and produces ATP in the presence of adenosine 5′ phosphsulfate, fueling the luciferin reaction, which produces a chemiluminescent signal allowing sequence determination.

An example of a system that can be used based on pyrosequencing generally involves the following steps: ligating an adaptor nucleic acid to a study nucleic acid and hybridizing the study nucleic acid to a bead; amplifying a nucleotide sequence in the study nucleic acid in an emulsion; sorting beads using a picoliter multiwell solid support; and sequencing amplified nucleotide sequences by pyrosequencing methodology (e.g., Nakano et al., “Single-molecule PCR using water-in-oil emulsion;” Journal of Biotechnology 102: 117-124 (2003)). Such a system can be used to exponentially amplify amplification products generated by a process described herein, e.g., by ligating a heterologous nucleic acid to the first amplification product generated by a process described herein.

Certain single-molecule sequencing embodiments are based on the principal of sequencing by synthesis, and utilize single-pair Fluorescence Resonance Energy Transfer (single pair FRET) as a mechanism by which photons are emitted as a result of successful nucleotide incorporation. The emitted photons often are detected using intensified or high sensitivity cooled charge-couple-devices in conjunction with total internal reflection microscopy (TIRM). Photons are only emitted when the introduced reaction solution contains the correct nucleotide for incorporation into the growing nucleic acid chain that is synthesized as a result of the sequencing process. In FRET based single-molecule sequencing, energy is transferred between two fluorescent dyes, sometimes polymethine cyanine dyes Cy3 and Cy5, through long-range dipole interactions. The donor is excited at its specific excitation wavelength and the excited state energy is transferred, non-radiatively to the acceptor dye, which in turn becomes excited. The acceptor dye eventually returns to the ground state by radiative emission of a photon. The two dyes used in the energy transfer process represent the “single pair”, in single pair FRET. Cy3 often is used as the donor fluorophore and often is incorporated as the first labeled nucleotide. Cy5 often is used as the acceptor fluorophore and is used as the nucleotide label for successive nucleotide additions after incorporation of a first Cy3 labeled nucleotide. The fluorophores generally are within 10 nanometers of each for energy transfer to occur successfully.

An example of a system that can be used based on single-molecule sequencing generally involves hybridizing a primer to a study nucleic acid to generate a complex; associating the complex with a solid phase; iteratively extending the primer by a nucleotide tagged with a fluorescent molecule; and capturing an image of fluorescence resonance energy transfer signals after each iteration (e.g., U.S. Pat. No. 7,169,314; Braslaysky et al., PNAS 100(7): 3960-3964 (2003)). Such a system can be used to directly sequence amplification products generated by processes described herein. In some embodiments the released linear amplification product can be hybridized to a primer that contains sequences complementary to immobilized capture sequences present on a solid support, a bead or glass slide for example. Hybridization of the primer—released linear amplification product complexes with the immobilized capture sequences, immobilizes released linear amplification products to solid supports for single pair FRET based sequencing by synthesis. The primer often is fluorescent, so that an initial reference image of the surface of the slide with immobilized nucleic acids can be generated. The initial reference image is useful for determining locations at which true nucleotide incorporation is occurring. Fluorescence signals detected in array locations not initially identified in the “primer only” reference image are discarded as non-specific fluorescence. Following immobilization of the primer—released linear amplification product complexes, the bound nucleic acids often are sequenced in parallel by the iterative steps of, a) polymerase extension in the presence of one fluorescently labeled nucleotide, b) detection of fluorescence using appropriate microscopy, TIRM for example, c) removal of fluorescent nucleotide, and d) return to step a with a different fluorescently labeled nucleotide.

In some embodiments, nucleotide sequencing may be by solid phase single nucleotide sequencing methods and processes. Solid phase single nucleotide sequencing methods involve contacting sample nucleic acid and solid support under conditions in which a single molecule of sample nucleic acid hybridizes to a single molecule of a solid support. Such conditions can include providing the solid support molecules and a single molecule of sample nucleic acid in a “microreactor.” Such conditions also can include providing a mixture in which the sample nucleic acid molecule can hybridize to solid phase nucleic acid on the solid support. Single nucleotide sequencing methods useful in the embodiments described herein are described in U.S. Provisional Patent Application Ser. No. 61/021,871 filed Jan. 17, 2008.

In certain embodiments, nanopore sequencing detection methods include (a) contacting a nucleic acid for sequencing (“base nucleic acid,” e.g., linked probe molecule) with sequence-specific detectors, under conditions in which the detectors specifically hybridize to substantially complementary subsequences of the base nucleic acid; (b) detecting signals from the detectors and (c) determining the sequence of the base nucleic acid according to the signals detected. In certain embodiments, the detectors hybridized to the base nucleic acid are disassociated from the base nucleic acid (e.g., sequentially dissociated) when the detectors interfere with a nanopore structure as the base nucleic acid passes through a pore, and the detectors disassociated from the base sequence are detected. In some embodiments, a detector disassociated from a base nucleic acid emits a detectable signal, and the detector hybridized to the base nucleic acid emits a different detectable signal or no detectable signal. In certain embodiments, nucleotides in a nucleic acid (e.g., linked probe molecule) are substituted with specific nucleotide sequences corresponding to specific nucleotides (“nucleotide representatives”), thereby giving rise to an expanded nucleic acid (e.g., U.S. Pat. No. 6,723,513), and the detectors hybridize to the nucleotide representatives in the expanded nucleic acid, which serves as a base nucleic acid. In such embodiments, nucleotide representatives may be arranged in a binary or higher order arrangement (e.g., Soni and Meller, Clinical Chemistry 53(11): 1996-2001 (2007)). In some embodiments, a nucleic acid is not expanded, does not give rise to an expanded nucleic acid, and directly serves a base nucleic acid (e.g., a linked probe molecule serves as a non-expanded base nucleic acid), and detectors are directly contacted with the base nucleic acid. For example, a first detector may hybridize to a first subsequence and a second detector may hybridize to a second subsequence, where the first detector and second detector each have detectable labels that can be distinguished from one another, and where the signals from the first detector and second detector can be distinguished from one another when the detectors are disassociated from the base nucleic acid. In certain embodiments, detectors include a region that hybridizes to the base nucleic acid (e.g., two regions), which can be about 3 to about 100 nucleotides in length (e.g., about 4, 5, 6, 7, 8, 9, 10, 11, 12, 13, 14, 15, 16, 17, 18, 19, 20, 25, 30, 35, 40, 50, 55, 60, 65, 70, 75, 80, 85, 90, or 95 nucleotides in length). A detector also may include one or more regions of nucleotides that do not hybridize to the base nucleic acid. In some embodiments, a detector is a molecular beacon. A detector often comprises one or more detectable labels independently selected from those described herein. Each detectable label can be detected by any convenient detection process capable of detecting a signal generated by each label (e.g., magnetic, electric, chemical, optical and the like). For example, a CD camera can be used to detect signals from one or more distinguishable quantum dots linked to a detector.

In some embodiments, detection of the presence or absence of a multiplied chromosomal region can be performed using fluorescence in situ hybridization (e.g., FISH), and in certain embodiments detection of the presence or absence of a multiplied chromosomal region can be performed using a method referred to as Fiber FISH. FISH is a cytogenetic technique often used to detect and localize the presence or absence of specific DNA sequences on chromosomes. FISH methodology generally makes use of fluorescent probes that bind to only those parts of the chromosome with which they show a high degree of sequence complimentarity. The fluorescent signal typically is visualized utilizing fluorescence microscopy. Fiber FISH is a specialized FISH methodology that makes use of chromatin spreads in which the chromosomes have been mechanically stretched, thereby allowing a higher resolution analysis than conventional FISH. Generally Fiber FISH provides more precise information as to the localization of a specific DNA probe on a chromosome.

In certain sequence analysis embodiments, reads may be used to construct a larger nucleotide sequence, which can be facilitated by identifying overlapping sequences in different reads and by using identification sequences in the reads. Such sequence analysis methods and software for constructing larger sequences from reads are known in the art (e.g., Venter et al., Science 291: 1304-1351 (2001)). Specific reads, partial nucleotide sequence constructs, and full nucleotide sequence constructs may be compared between nucleotide sequences within a sample nucleic acid (i.e., internal comparison) or may be compared with a reference sequence (i.e., reference comparison) in certain sequence analysis embodiments. Internal comparisons sometimes are performed in situations where a sample nucleic acid is prepared from multiple samples or from a single sample source that contains sequence variations. Reference comparisons sometimes are performed when a reference nucleotide sequence is known and an objective is to determine whether a sample nucleic acid contains a nucleotide sequence that is substantially similar or the same, or different, than a reference nucleotide sequence. Sequence analysis is facilitated by sequence analysis apparatus and components known in the art.

Mass spectrometry is a particularly effective method for the detection of a nucleic acids (e.g., PCR amplicon, primer extension product, detector probe cleaved from a target nucleic acid). Presence of a target nucleic acid is verified by comparing the mass of the detected signal with the expected mass of the target nucleic acid. The relative signal strength, e.g., mass peak on a spectra, for a particular target nucleic acid indicates the relative population of the target nucleic acid amongst other nucleic acids, thus enabling calculation of a ratio of target to other nucleic acid or sequence copy number directly from the data. For a review of genotyping methods using Sequenom® standard iPLEX® assay and MassARRAY® technology, see Jurinke, C., Oeth, P., van den Boom, D., “MALDI-TOF mass spectrometry: a versatile tool for high-performance DNA analysis.” Mol. Biotechnol. 26, 147-164 (2004). For a review of detecting and quantifying target nucleic using cleavable detector probes that are cleaved during the amplification process and detected by mass spectrometry, see U.S. patent application Ser. No. 11/950,395, which was filed Dec. 4, 2007, and is hereby incorporated by reference. Such approaches may be adapted to detection of chromosome abnormalities by methods described herein.

In some embodiments, a MassARRAY® system (Sequenom, Inc.) can be utilized to perform SNP genotyping in a high-throughput fashion. The MassARRAY® genotyping platform often is complemented by a homogeneous, single-tube assay method (hME or homogeneous MassEXTEND® (Sequenom, Inc.)) in which two genotyping primers anneal to and amplify a genomic target surrounding a polymorphic site of interest. A third primer (the MassEXTEND® primer), which is complementary to the amplified target up to but not including the polymorphism, is enzymatically extended one or a few bases through the polymorphic site and then terminated.

For each polymorphism, a primer set is generated (e.g., a set of PCR primers and a MassEXTEND® primer) to genotype the polymorphism. Primer sets can be generated using any method known in the art. In some embodiments, SpectroDESIGNER™ software (Sequenom, Inc.) is used to design a primer set. Examples of primers that can be used in a MassARRAY® assay are provided in Example 2. A non-limiting example of a PCR amplification scheme suitable for use with a MassARRAY® assay includes a 5 μl total volume containing 1×PCR buffer with 1.5 mM MgCl₂ (Qiagen), 200 μM each of dATP, dGTP, dCTP, dTTP (Gibco-BRL), 2.5 ng of genomic DNA, 0.1 units of HotStar DNA polymerase (Qiagen), and 200 nM each of forward and reverse PCR primers specific for the polymorphic region of interest and inclubation at 95° C. for 15 minutes, followed by 45 cycles of 95° C. for 20 seconds, 56° C. for 30 seconds, and 72° C. for 1 minute, finishing with a 3 minute final extension at 72° C. Following amplification, shrimp alkaline phosphatase (SAP) (0.3 units in a 2 μl volume) (Amersham Pharmacia) can be added to each reaction (total reaction volume was 7 μl) to remove any residual dNTPs that were not consumed in the PCR step, in some embodiments. Reactions are incubated for 20 minutes at 37° C., followed by 5 minutes at 85° C. to denature the SAP.

After SAP treatment, a primer extension reaction is initiated by adding a polymorphism-specific MassEXTEND® primer cocktail to each sample, in certain embodiments. Each MassEXTEND® cocktail often includes a specific combination of dideoxynucleotides (ddNTPs) and deoxynucleotides (dNTPs) used to distinguish polymorphic alleles from one another. The MassEXTEND® reaction is performed in a total volume of 9 μl, with the addition of 1× ThermoSequenase buffer, 0.576 units of ThermoSequenase (Amersham Pharmacia), 600 nM MassEXTEND® primer, 2 mM of ddATP and/or ddCTP and/or ddGTP and/or ddTTP, and 2 mM of dATP or dCTP or dGTP or dTTP, in some embodiments. The deoxy nucleotide (dNTP) used in the assay generally is complementary to the nucleotide at the polymorphic site in the amplicon. A non-limiting example of reaction conditions for primer extension reactions include incubating reactions at 94° C. for 2 minutes, followed by 55 cycles of 5 seconds at 94° C., 5 seconds at 52° C., and 5 seconds at 72° C.

Following incubation, samples are desalted by adding 16 μl of water (total reaction volume was 25 μl), 3 mg of SpectroCLEAN™ sample cleaning beads (Sequenom, Inc.) and incubating for 3 minutes with rotation, in some embodiments. For MALDI-TOF analysis, samples are dispensed onto either 96-spot or 384-spot silicon chips containing a matrix that crystallized each sample (SpectroCHIP® (Sequenom, Inc.)), in certain embodiments. In some embodiments, MALDI-TOF mass spectrometry (Biflex and Autoflex MALDI-TOF mass spectrometers (Bruker Daltonics) can be used) and SpectroTYPER RT™ software (Sequenom, Inc.) were used to analyze and interpret the SNP genotype for each sample.

In some embodiments, amplified nucleic acid may be detected by (a) contacting the amplified nucleic acid (e.g., amplicons) with extension primers (e.g., detection or detector primers), (b) preparing extended extension primers, and (c) determining the relative amount of the one or more mismatch nucleotides (e.g., SNP that exist between paralogous sequences) by analyzing the extended detection primers (e.g., extension primers). In certain embodiments one or more mismatch nucleotides may be analyzed by mass spectrometry. In some embodiments amplification, using methods described herein, may generate between about 1 to about 100 amplicon sets, about 2 to about 80 amplicon sets, about 4 to about 60 amplicon sets, about 6 to about 40 amplicon sets, and about 8 to about 20 amplicon sets (e.g., about 1, 2, 3, 4, 5, 6, 7, 8, 9, 10, 15, 20, 25, 30, 35, 40, 45, 50, 55, 60, 65, 70, 75, 80, 85, 90, 95, or about 100 amplicon sets).

An example using mass spectrometry for detection of amplicon sets is presented herein. Amplicons may be contacted (in solution or on solid phase) with a set of oligonucleotides (the same primers used for amplification or different primers representative of subsequences in the primer or target nucleic acid) under hybridization conditions, where: (1) each oligonucleotide in the set comprises a hybridization sequence capable of specifically hybridizing to one amplicon under the hybridization conditions when the amplicon is present in the solution, (2) each oligonucleotide in the set comprises a distinguishable tag located 5′ of the hybridization sequence, (3) a feature of the distinguishable tag of one oligonucleotide detectably differs from the features of distinguishable tags of other oligonucleotides in the set; and (4) each distinguishable tag specifically corresponds to a specific amplicon and thereby specifically corresponds to a specific target nucleic acid. The hybridized amplicon and “detection” primer are subjected to nucleotide synthesis conditions that allow extension of the detection primer by one or more nucleotides (labeled with a detectable entity or moiety, or unlabeled), where one of the one of more nucleotides can be a terminating nucleotide. In some embodiments one or more of the nucleotides added to the primer may comprises a capture agent. In embodiments where hybridization occurred in solution, capture of the primer/amplicon to solid support may be desirable. The detectable moieties or entities can be released from the extended detection primer, and detection of the moiety determines the presence, absence or copy number of the nucleotide sequence of interest. In certain embodiments, the extension may be performed once yielding one extended oligonucleotide. In some embodiments, the extension may be performed multiple times (e.g., under amplification conditions) yielding multiple copies of the extended oligonucleotide. In some embodiments performing the extension multiple times can produce a sufficient number of copies such that interpretation of signals, representing copy number of a particular sequence, can be made with a confidence level of 95% or more (e.g., confidence level of 95% or more, 96% or more, 97% or more, 98% or more, 99% or more, or a confidence level of 99.5% or more).

Methods provided herein allow for high-throughput detection of nucleic acid in a plurality of nucleic acids (e.g., nucleic acid, amplified nucleic acid and detectable products generated from the foregoing). Multiplexing refers to the simultaneous detection of more than one nucleic acid. General methods for performing multiplexed reactions in conjunction with mass spectrometry, are known (see, e.g., U.S. Pat. Nos. 6,043,031; 5,547,835 and International PCT Application No. WO 97/37041). Multiplexing provides an advantage that a plurality of nucleic acid species (e.g., some having different sequence variations) can be identified in as few as a single mass spectrum, as compared to having to perform a separate mass spectrometry analysis for each individual target nucleic acid species. Methods provided herein lend themselves to high-throughput, highly-automated processes for analyzing sequence variations with high speed and accuracy, in some embodiments. In some embodiments, methods herein may be multiplexed at high levels in a single reaction.

In certain embodiments, the number of nucleic acid species multiplexed include, without limitation, about 1 to about 500 (e.g., about 1-3, 3-5, 5-7, 7-9, 9-11, 11-13, 13-15, 15-17, 17-19, 19-21, 21-23, 23-25, 25-27, 27-29, 29-31, 31-33, 33-35, 35-37, 37-39, 39-41, 41-43, 43-45, 45-47, 47-49, 49-51, 51-53, 53-55, 55-57, 57-59, 59-61, 61-63, 63-65, 65-67, 67-69, 69-71, 71-73, 73-75, 75-77, 77-79, 79-81, 81-83, 83-85, 85-87, 87-89, 89-91, 91-93, 93-95, 95-97, 97-101, 101-103, 103-105, 105-107, 107-109, 109-111, 111-113, 113-115, 115-117, 117-119, 121-123, 123-125, 125-127, 127-129, 129-131, 131-133, 133-135, 135-137, 137-139, 139-141, 141-143, 143-145, 145-147, 147-149, 149-151, 151-153, 153-155, 155-157, 157-159, 159-161, 161-163, 163-165, 165-167, 167-169, 169-171, 171-173, 173-175, 175-177, 177-179, 179-181, 181-183, 183-185, 185-187, 187-189, 189-191, 191-193, 193-195, 195-197, 197-199, 199-201, 201-203, 203-205, 205-207, 207-209, 209-211, 211-213, 213-215, 215-217, 217-219, 219-221, 221-223, 223-225, 225-227, 227-229, 229-231, 231-233, 233-235, 235-237, 237-239, 239-241, 241-243, 243-245, 245-247, 247-249, 249-251, 251-253, 253-255, 255-257, 257-259, 259-261, 261-263, 263-265, 265-267, 267-269, 269-271, 271-273, 273-275, 275-277, 277-279, 279-281, 281-283, 283-285, 285-287, 287-289, 289-291, 291-293, 293-295, 295-297, 297-299, 299-301, 301-303, 303-305, 305-307, 307-309, 309-311, 311-313, 313-315, 315-317, 317-319, 319-321, 321-323, 323-325, 325-327, 327-329, 329-331, 331-333, 333-335, 335-337, 337-339, 339-341, 341-343, 343-345, 345-347, 347-349, 349-351, 351-353, 353-355, 355-357, 357-359, 359-361, 361-363, 363-365, 365-367, 367-369, 369-371, 371-373, 373-375, 375-377, 377-379, 379-381, 381-383, 383-385, 385-387, 387-389, 389-391, 391-393, 393-395, 395-397, 397-401, 401-403, 403-405, 405-407, 407-409, 409-411, 411-413, 413-415, 415-417, 417-419, 419-421, 421-423, 423-425, 425-427, 427-429, 429-431, 431-433, 433-435, 435-437, 437-439, 439-441, 441-443, 443-445, 445-447, 447-449, 449-451, 451-453, 453-455, 455-457, 457-459, 459-461, 461-463, 463-465, 465-467, 467-469, 469-471, 471-473, 473-475, 475-477, 477-479, 479-481, 481-483, 483-485, 485-487, 487-489, 489-491, 491-493, 493-495, 495-497, 497-501).

Design methods for achieving resolved mass spectra with multiplexed assays can include primer and oligonucleotide design methods and reaction design methods. For primer and oligonucleotide design in multiplexed assays, the same general guidelines for primer design applies for uniplexed reactions, such as avoiding false priming and primer dimers, only more primers are involved for multiplex reactions. For mass spectrometry applications, analyte peaks in the mass spectra for one assay are sufficiently resolved from a product of any assay with which that assay is multiplexed, including pausing peaks and any other by-product peaks. Also, analyte peaks optimally fall within a user-specified mass window, for example, within a range of 5,000-8,500 Da.

In some embodiments multiplex analysis may be adapted to mass spectrometric detection of chromosome abnormalities, for example. In certain embodiments multiplex analysis may be adapted to various single nucleotide or nanopore based sequencing methods described herein. Commercially produced micro-reaction chambers or devices or arrays or chips may be used to facilitate multiplex analysis, and are commercially available.

### EXAMPLES

The following examples illustrate but do not limit the technology.

### Example 1

### Evaluation of Genetic Structure in CEU HapMap Samples across RCA Region-Identification of Novel RCA Haplotypes

Using Phased HapMap data from the CEU sample collection, it was possible to identify CFH haplotype specific SNP blocks or variant motifs that are maintained across the RCA region (gene region containing CFH through CFHR5). See Table 1 below. Table 1 shows that wild-type alleles contain haplotype-specific motifs/sequence blocks that can be used to monitor recombination/structural changes across loci. Tables 2-5 (see below) show alignment of genotyping phased data for CEU Hap Map sample collection across the CFH-CFHR5 region defined by six (6) of the eight (8) SNPs Hageman et al. used to differentiate and assign the four (4) most prevalent CFH haplotypes (Hageman et al. PNAS 2005). See Tables 2-5 below. The most prevalent haplotypes reported in the literature are CFH H1-H4 and have been reported to extend beyond CFH across the CFHR genes. Haplotypes observed in the HapMap sample collection were consistent with expected combinations and at frequencies consistent with those reported in the literature. Examples showing the most prevalent haplotype combinations found in the CEU HapMap database are shown in Table 6. Frequencies associated with these combinations are shown in Table 7. Additional haplotypes observed in the HapMap sample collection reveal motifs/structures suggestive of recombination between H1-H4 haplotypes. See Table 8. The four most prevalent haplotypes observed in Caucasian individuals have been reported with the following disease associations:
a. H1=the most prevalent AMD risk haplotype (associated with rs1061170 (SEQ ID NO: 16) “C” variant)
b. H2=the most prevalent protective AMD haplotype (associated with rs800292 “A” variant)
c. H3=reported as either risk or neutral for susceptibility/protection from AMD
d. H4=has similar prevalence of H2, shown to be highly protective against AMD (associated with rs12144939 “T” variant). This haplotype tags the CFHR3/CFHR1 deletion associated with protection from AMD and susceptibility to aHUS.

By observing the exchange of the haplotype specific blocks or motifs, novel haplotypes were identified that appear to result from homologous recombination of the most prevalent wild type CFH haplotypes (H1, H2, H3, and H4). The CFH gene located in the Regulator of Complement Activation (RCA) gene cluster on chromosome 1. Sequence analysis of the RCA gene cluster at chromosome position 1q32 shows evidence of several large segmental copy number variants (Venables et al 2006). These copy number variants have resulted in a high degree of sequence identity between the gene for factor H(CFH) and the genes for the five factor H-related proteins (CFHR1-5). Genomic copy number variants including the different exons of the six genes have been described by Venables et al (2006).

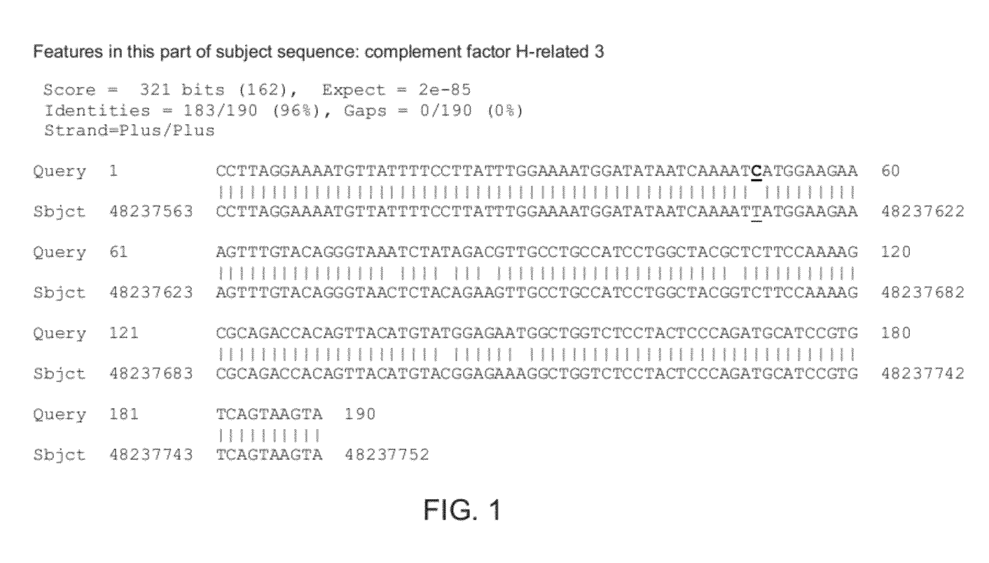

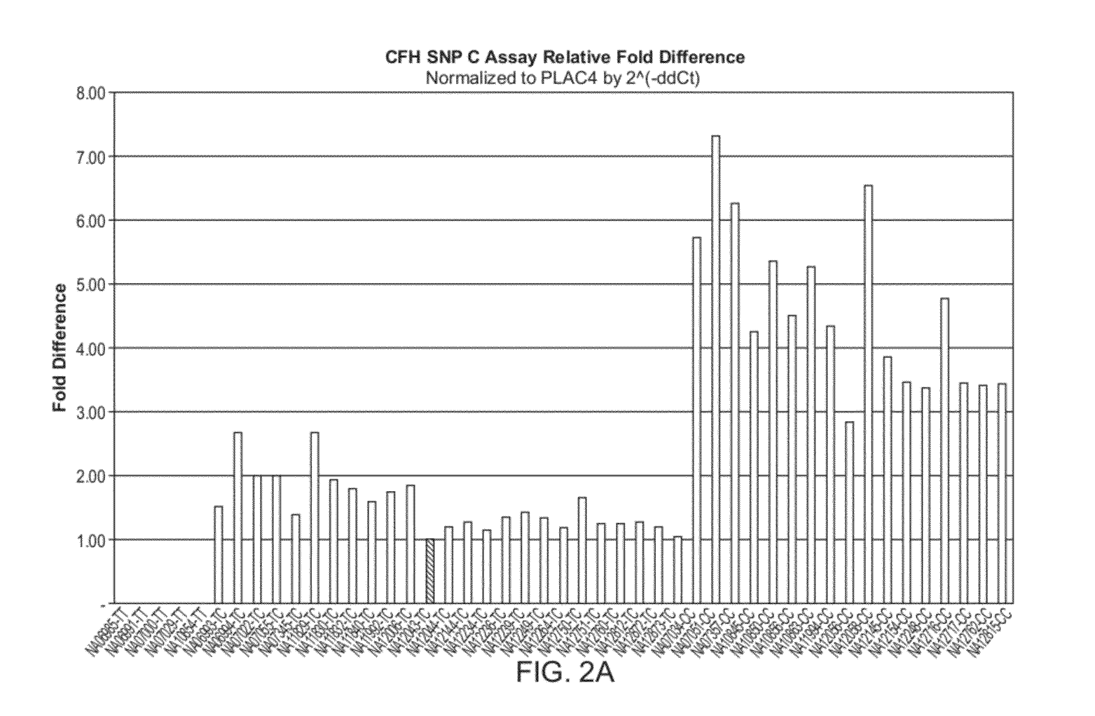

Allelic recombination was observed in a collection of HapMap samples at several “hot-spot” regions in CFH and the CFH-related genes presumably due to the high sequence identity reported in these closely related genes (See Table 9). Identified was a highly-specific, novel copy number variant that requires a remodeling of what was originally described by Venable as the likely genetic architecture across the RCA region. Close inspection of the region flanking the disease associated SNP rs1061170 (SEQ ID NO: 16) in CFH exon 9 compared to the homologous region identified by Venables in CFHR3 and in the intronic region upstream of CFHR4 revealed very high sequence identity. The sequence identity of the region flanking the Y402H CFH SNP, showed 96% identity to the region in CFHR3 (See FIG. 1) and somewhat lower identity (90%) to the intronic region upstream of CFHR4. In both regions, however, the variant base associated with the corresponding position in CFH Y402 (rs1061170 (SEQ ID NO: 16)) was reported as a “T” whereas in CFH gene, this variant position was observed as a “C” or “T” depending on the combination of haplotypes present in an individual. The key H1 AMD risk haplotype (most highly cited as having association with AMD) is specifically tagged by the “C” variant at SNP rs1061170 (SEQ ID NO: 16). This observation confirms that the homologous regions reported by Venables are not copy number variants of the CFH rs 1061170 (SEQ ID NO: 16) C variant region, rather these sequences represented DNA segments that are close homologs to the CFH exon 9 structure.

Regions associated with recombination spanned intron 9 of CFH surrounding chromosomal position 196673802 (build 37.1) 194940425 (build 36) in the region associated with SNP rs9970784 (SEQ ID NO: 35) and at downstream locations in the CFHR genes including CFHR3, CFHR1 and CFHR4. In addition to the four most prevalent haplotypes described by Hageman et al in 2005, there were eight (8) novel haplotypes identified in the HapMap CEU sample collection, each of which was observed in at least 2 chromosomes with frequencies ranging from 2-8% of the chromosomes surveyed. Analysis of the phased chromosomes of the HapMap sample collection revealed the CFH intron 9 region appeared to be a hot spot associated with the generation of structural chromosomal rearrangements via non-allelic homologous recombination as evidenced in the observation of the novel haplotypes with shared sequence motifs otherwise found exclusively in the most prevalent CFH haplotypes. This suggests this region might be subject to the generation of larger CNVs and/or gross structural rearrangements due to the genomic instability associated with this region.

TABLE 1
Haplotype Specific Motifs.

| CFH5′ | CFH3′ | R3 | R1 | R4 | R2 | R5 |
|---|---|---|---|---|---|---|
| H1 | H1 | H1 | H1 | H1 | H1 | H1 |
| H2 | H2 | H2 | H2 | H2 | H2 | H2 |
| H3 | H3 | H3 | H3 | H3 | H3 | H3 |
| H4 | H4 | H4 | H4 | H4 | H4 | H4 |

The four most prevalent haplotypes described by Hageman et al. PNAS 2005 based on 8 CFH SNPs are observed to extend beyond the CFH gene to include downstream genes CFHR3, CFHR1, CFHR4, and CFHR5 in the CEU HapMap sample collection. For Tables 2-5 and 8-9 below (Phased HapMap chromosome data across RCA region), the following legend applies:
1. HapMap Sample Ids listed in column B.
2. Chromosomal Coordinates of individual SNPs surveyed across RCA region provided in row A (build 36).
3. SNP IDs provided in row B.
4. The six SNPs used to define and differentiate the four most prevalent CFH haplotypes (H1-H4) described by Hageman et al 2005 highlighted in bold box (row B).
5. Double vertical line delineates last SNP in CFH. All SNPs to the right of this line reflect variant positions in located in CFHR3, CFHR1, CFHR2, CFHR4, CFHR5.
6. Consensus sequence defined as sequence associated with H1 AMD risk allele=white background
7. Variant base to consensus sequence=grey background and bold bases.
8. Haplotype tagging SNPs (SNPs that specifically tag a specific H1-H4 haplotype)=black background and white bases.

TABLE 2
Phased data of HapMap Caucasian (CEU) chromosomes identified as CFH H1 using 6 defining SNPs described by Hageman et al. 2005.
Chromosome position provided in row 1 are from NCBI Build 36.

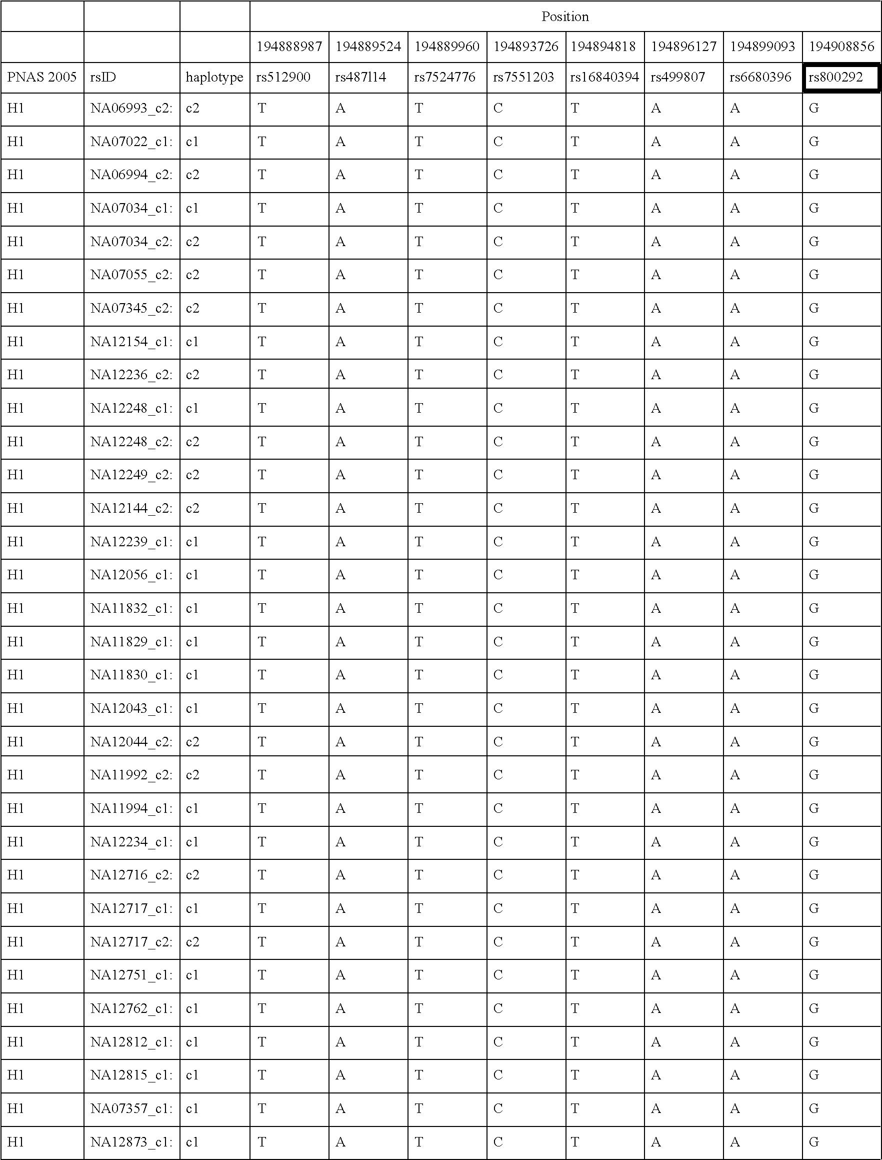

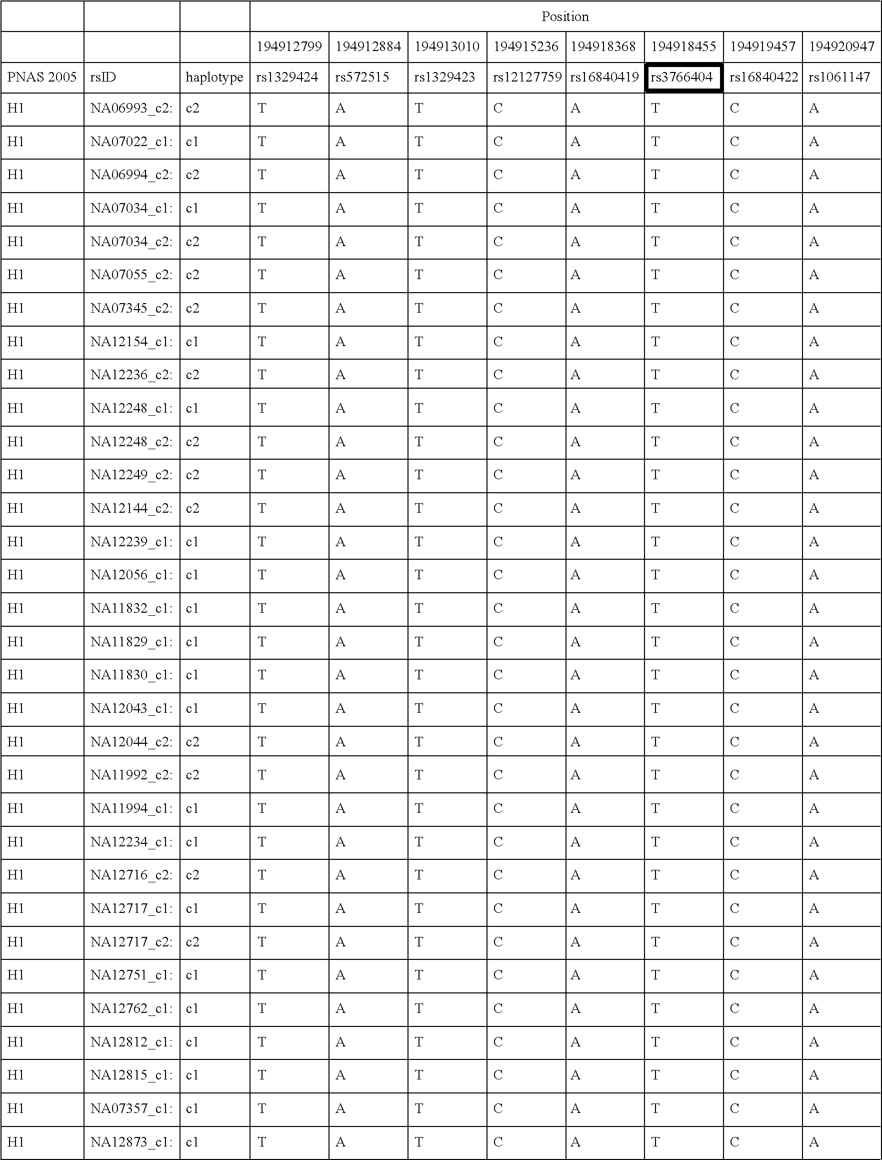

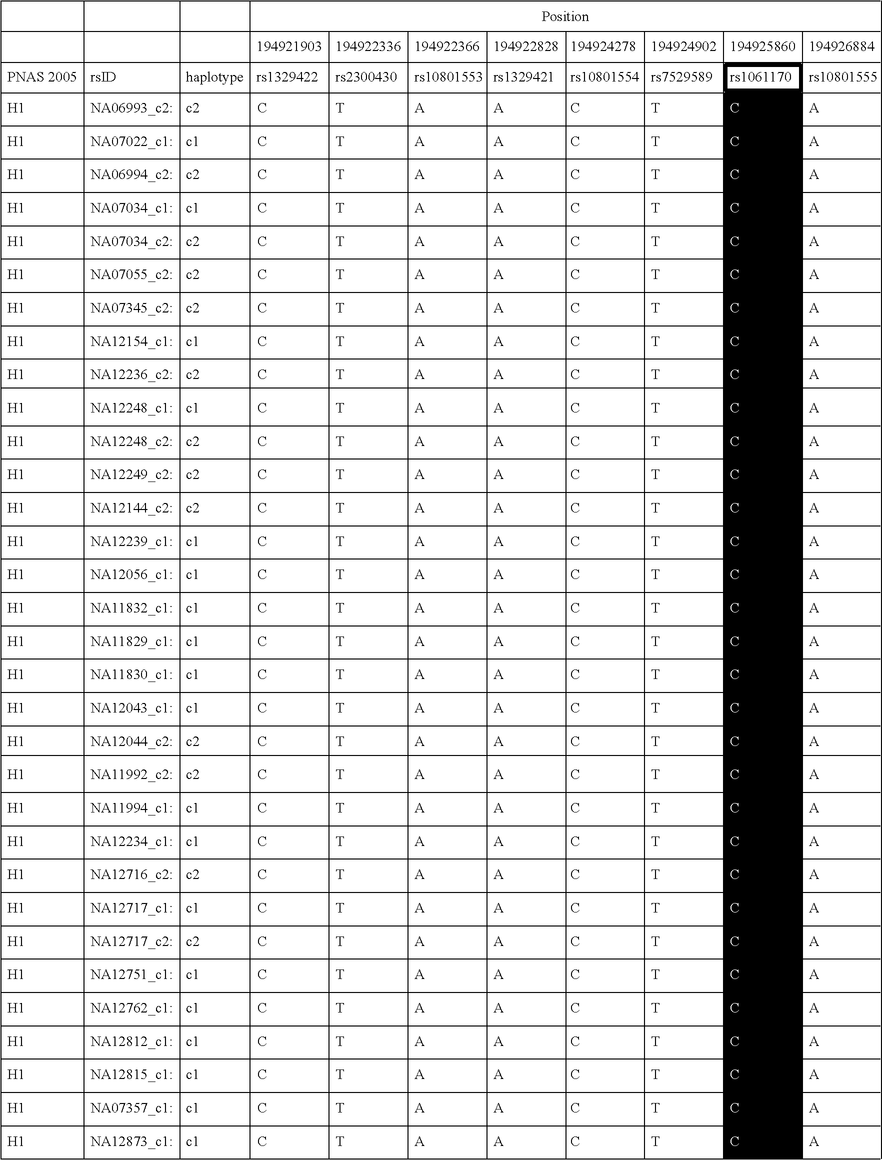

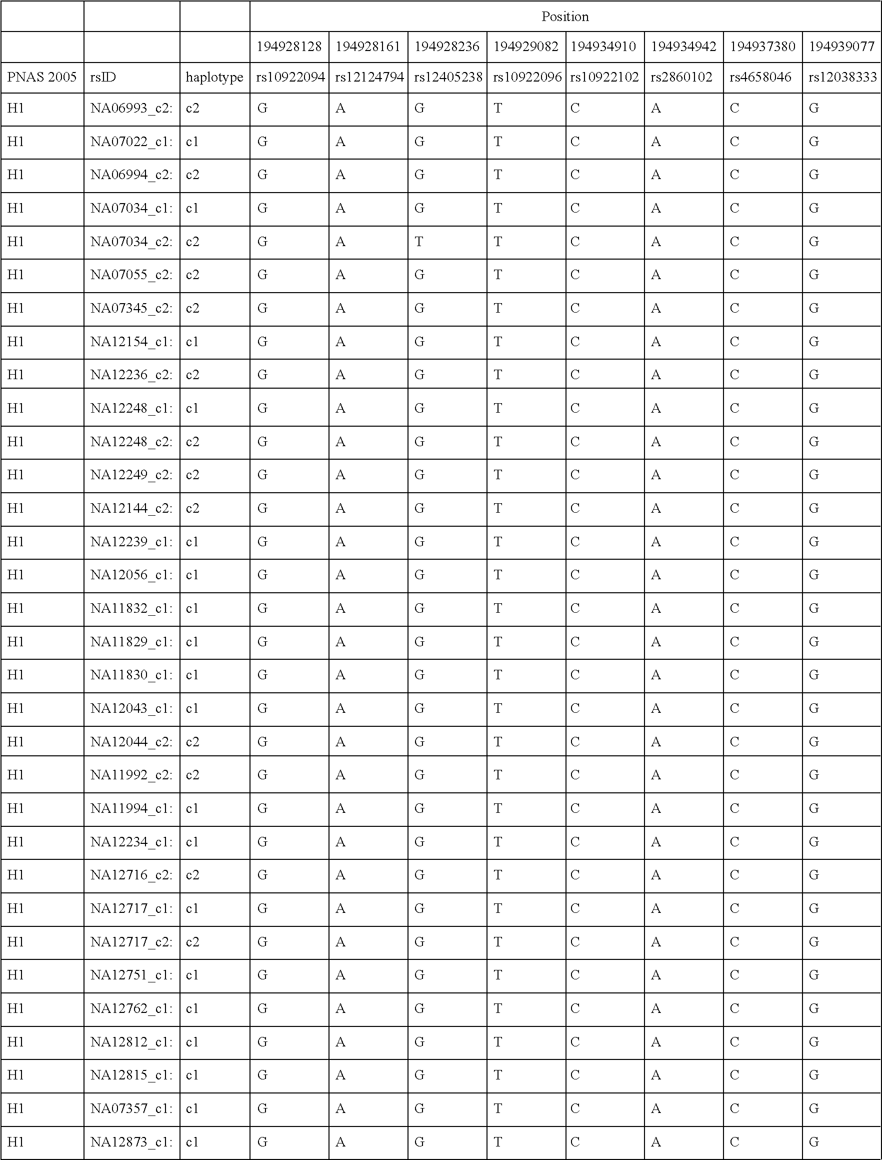

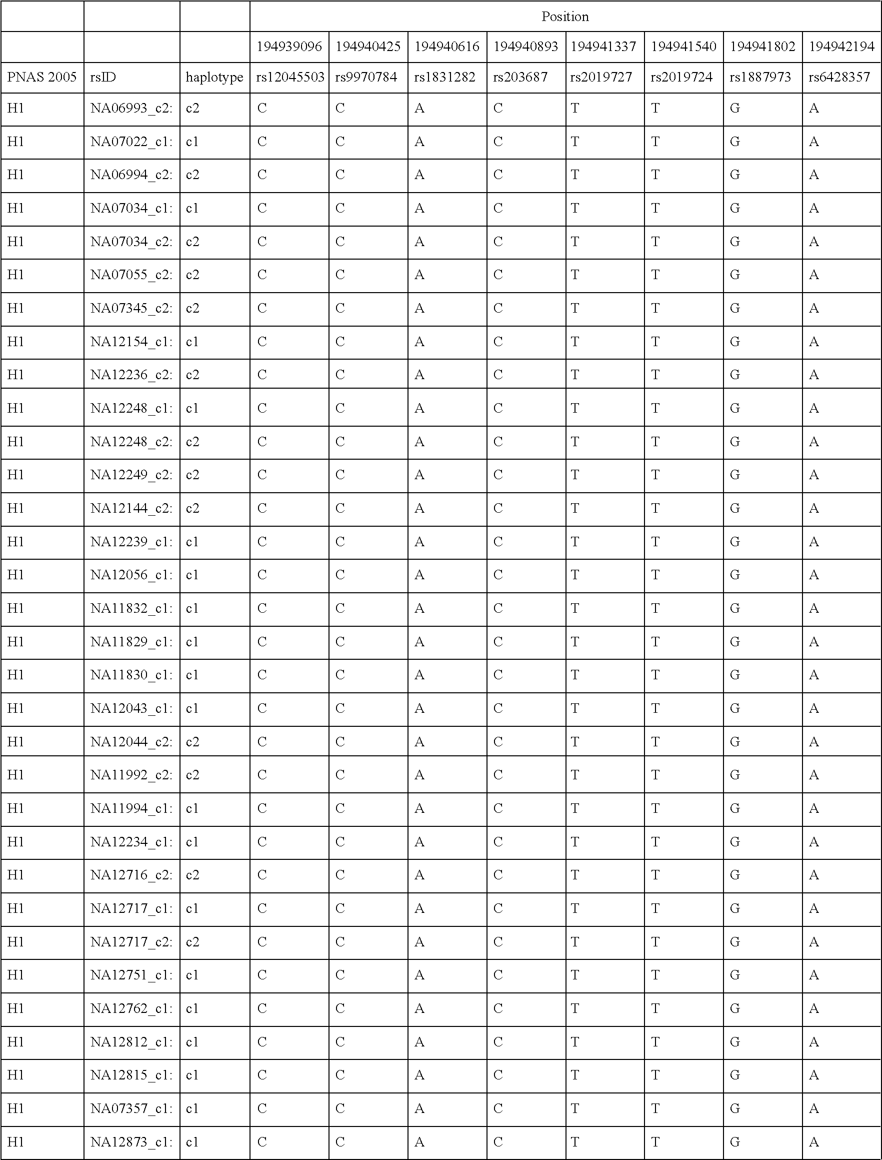

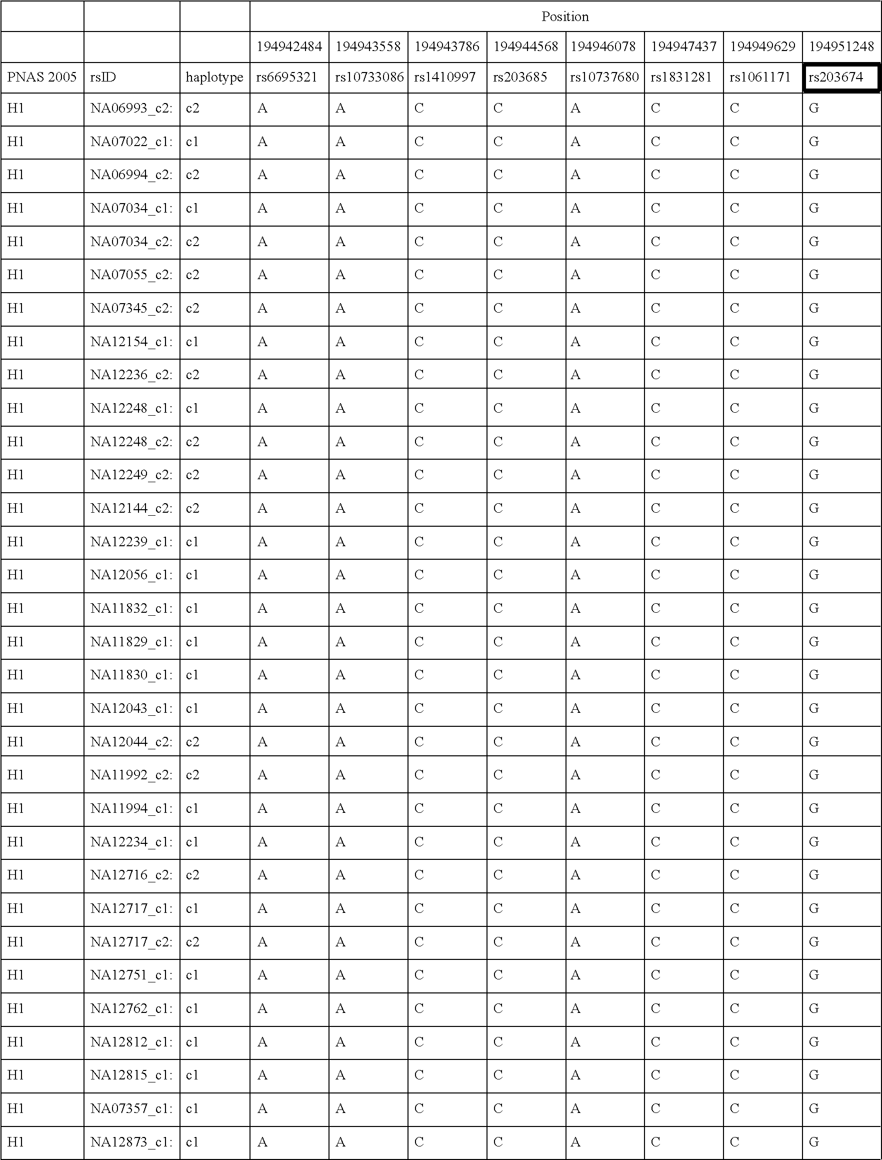

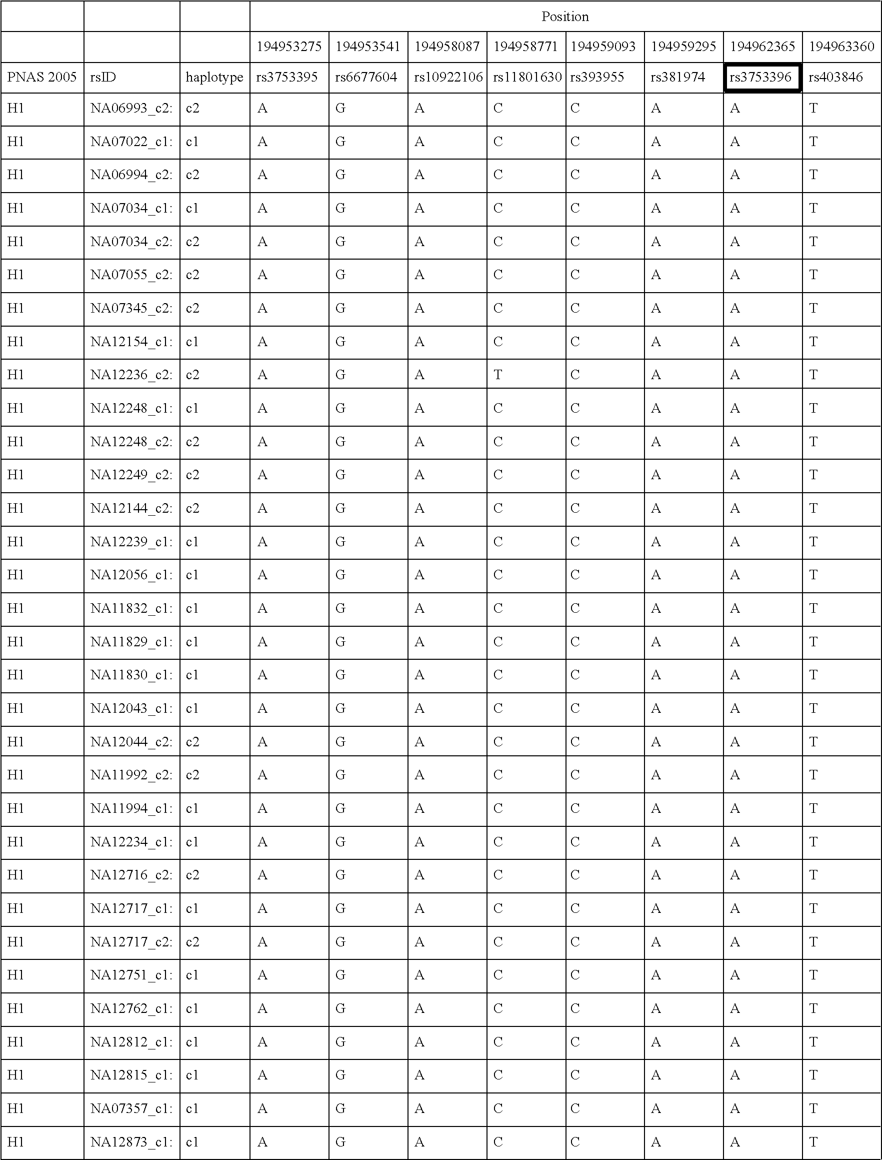

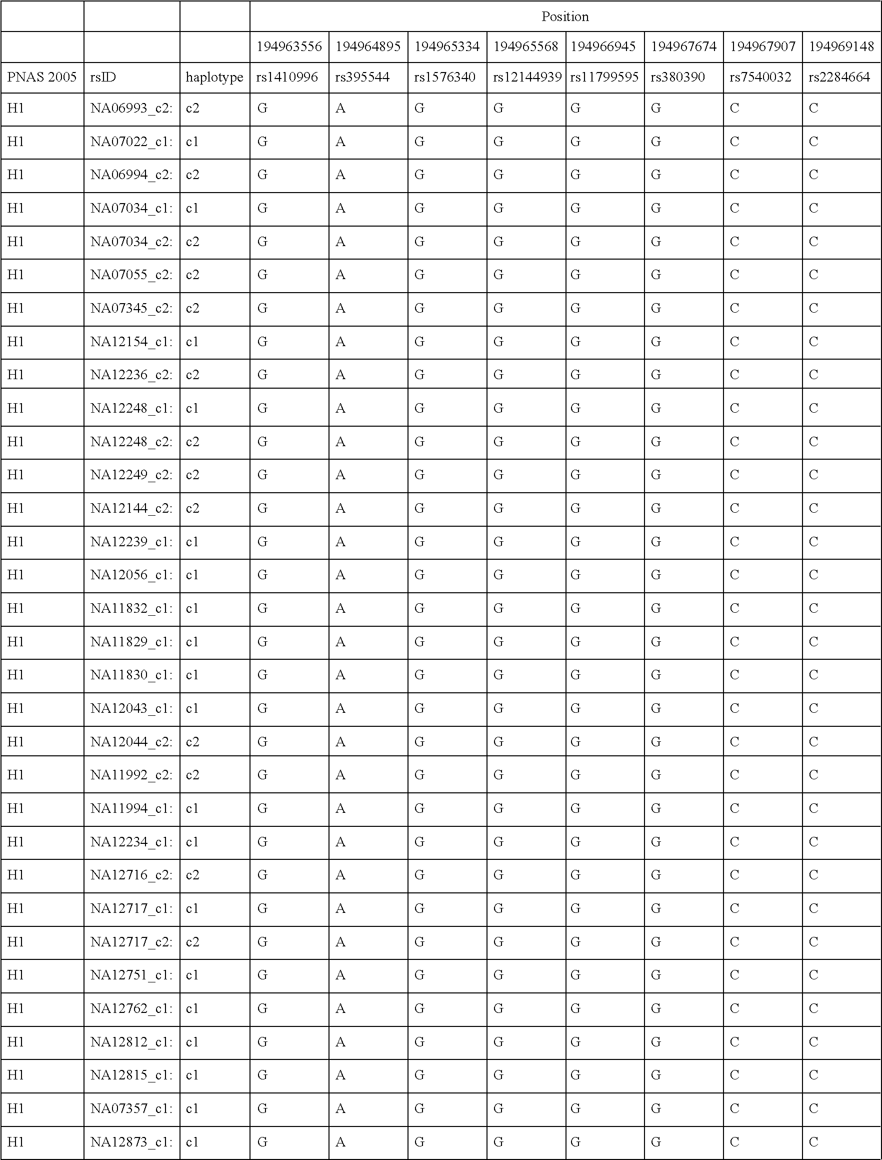

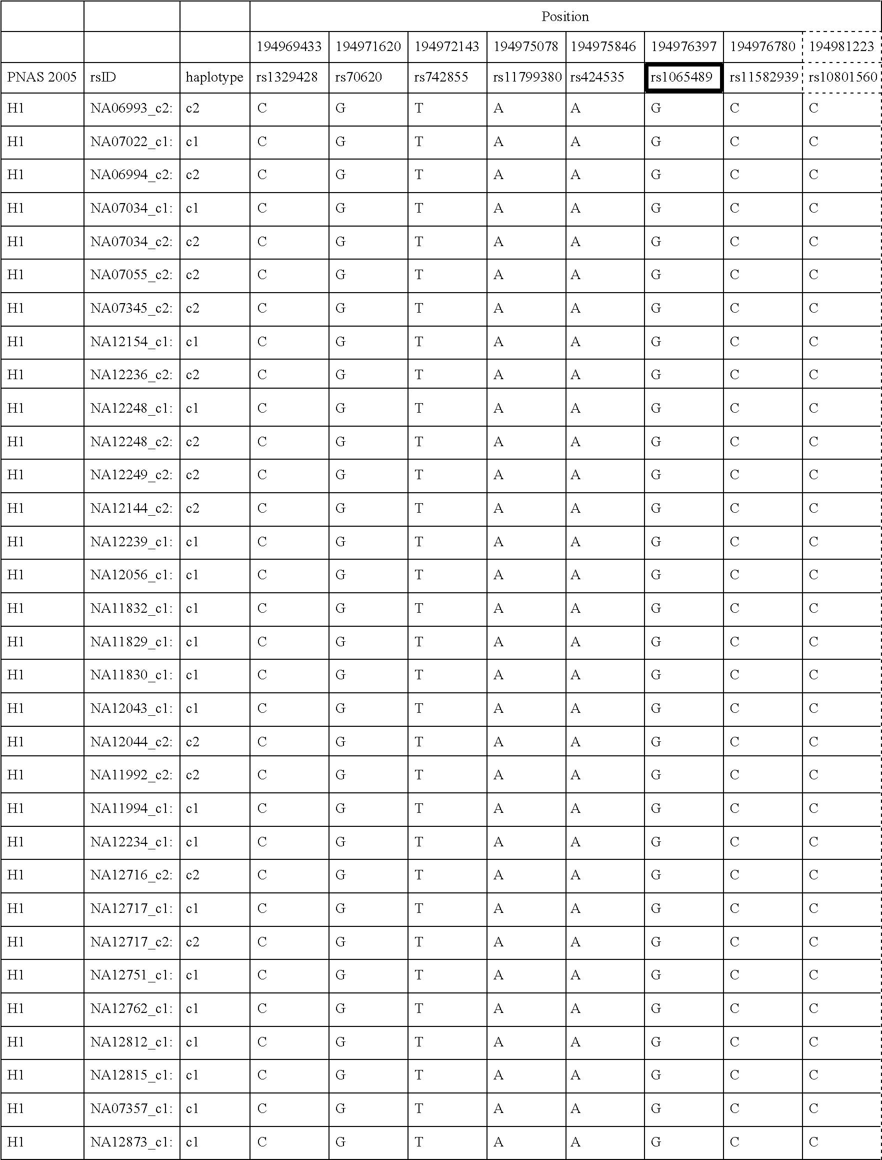

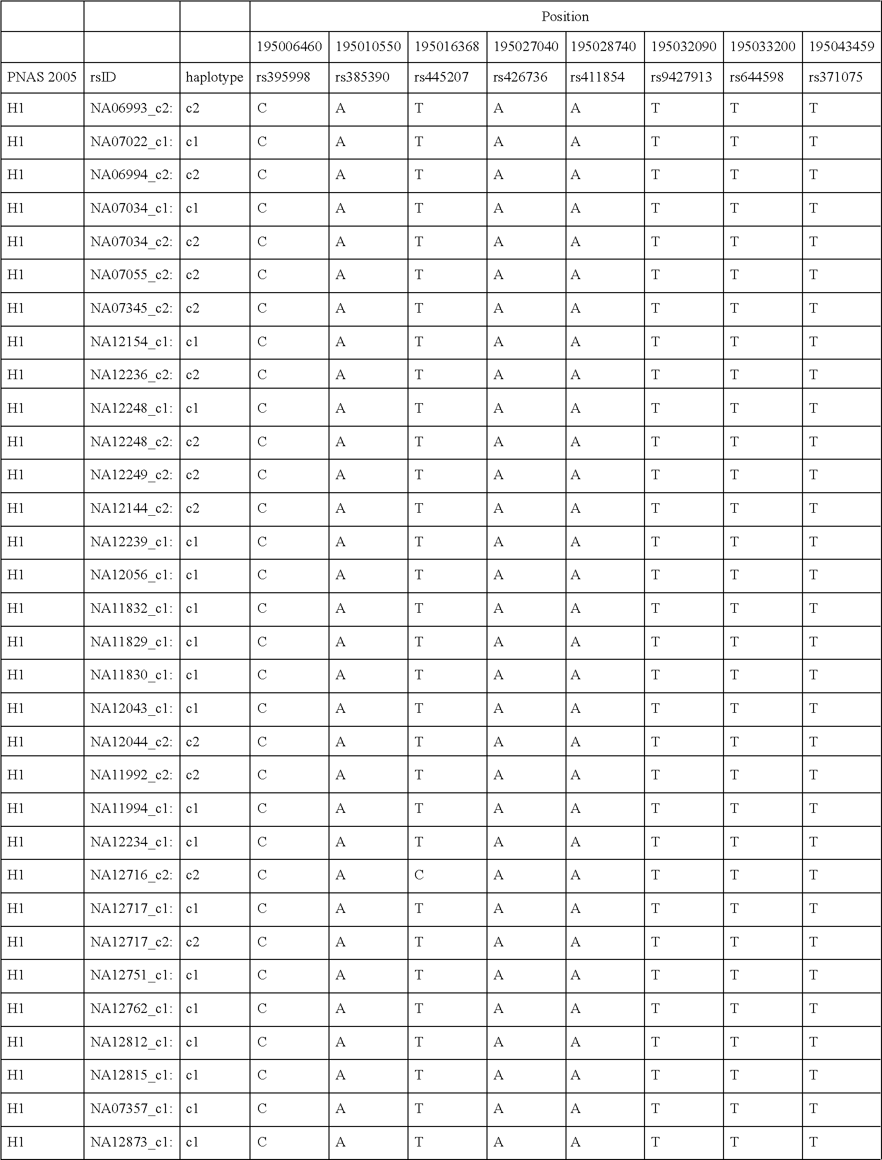

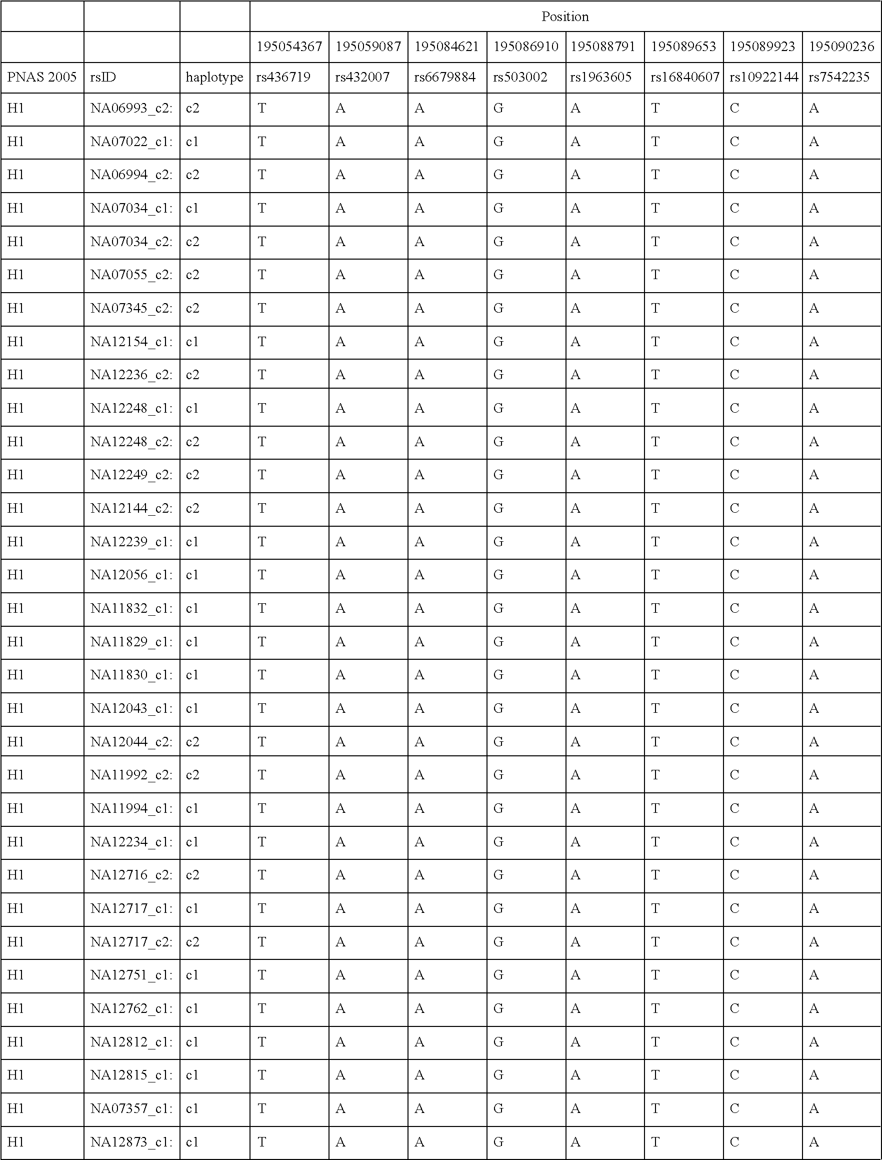

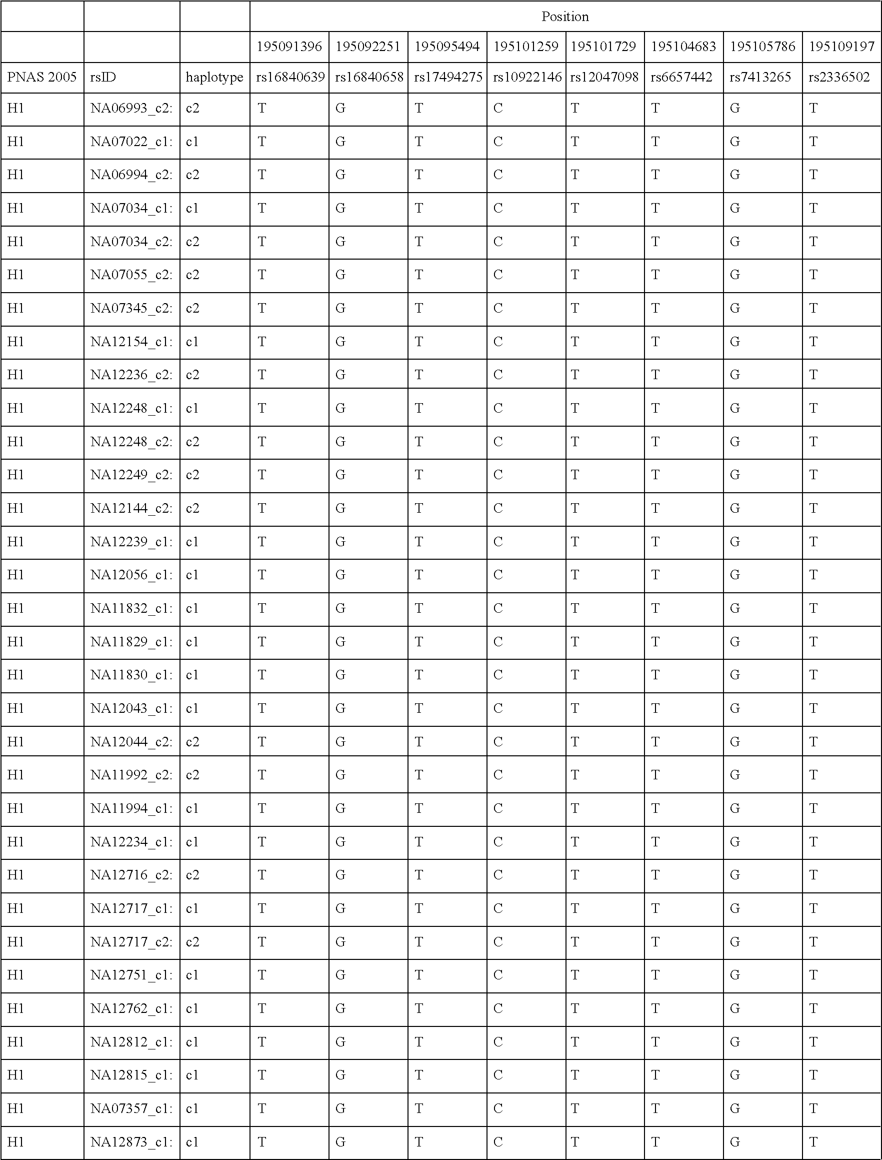

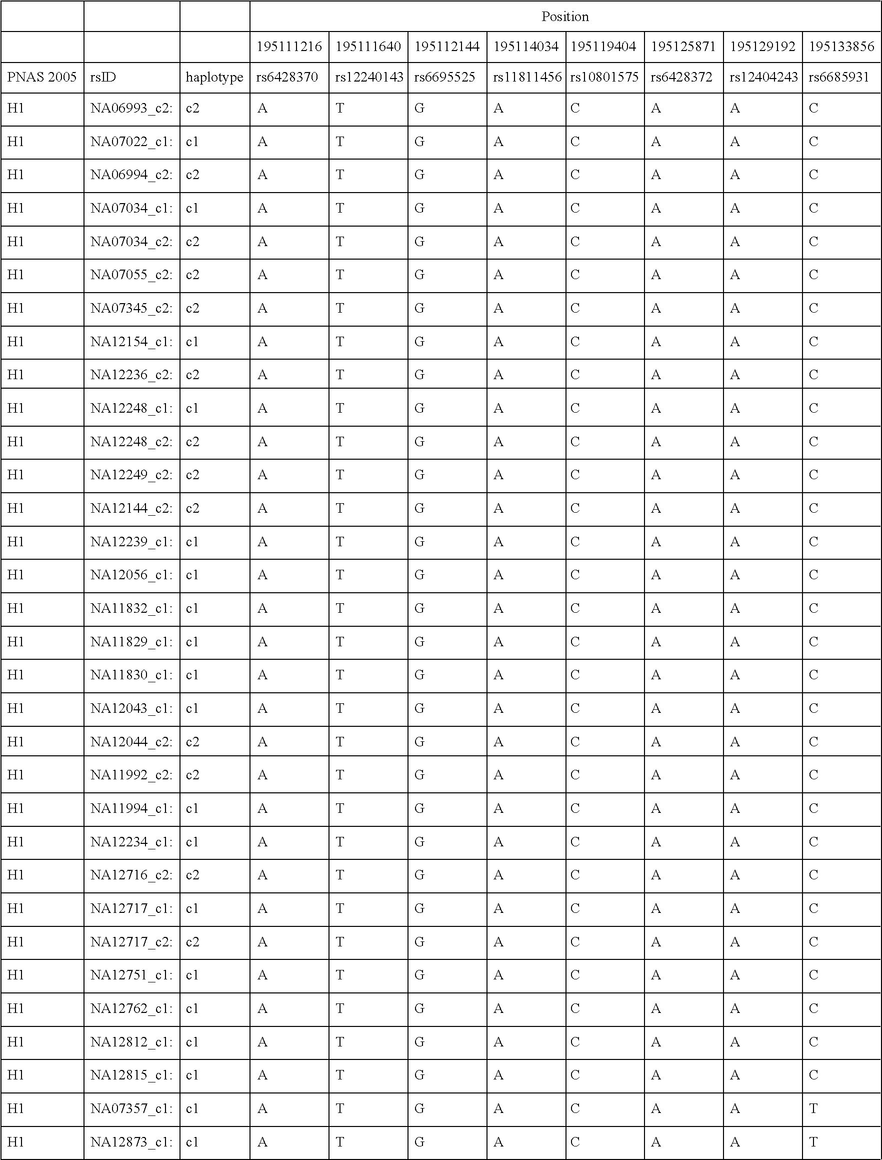

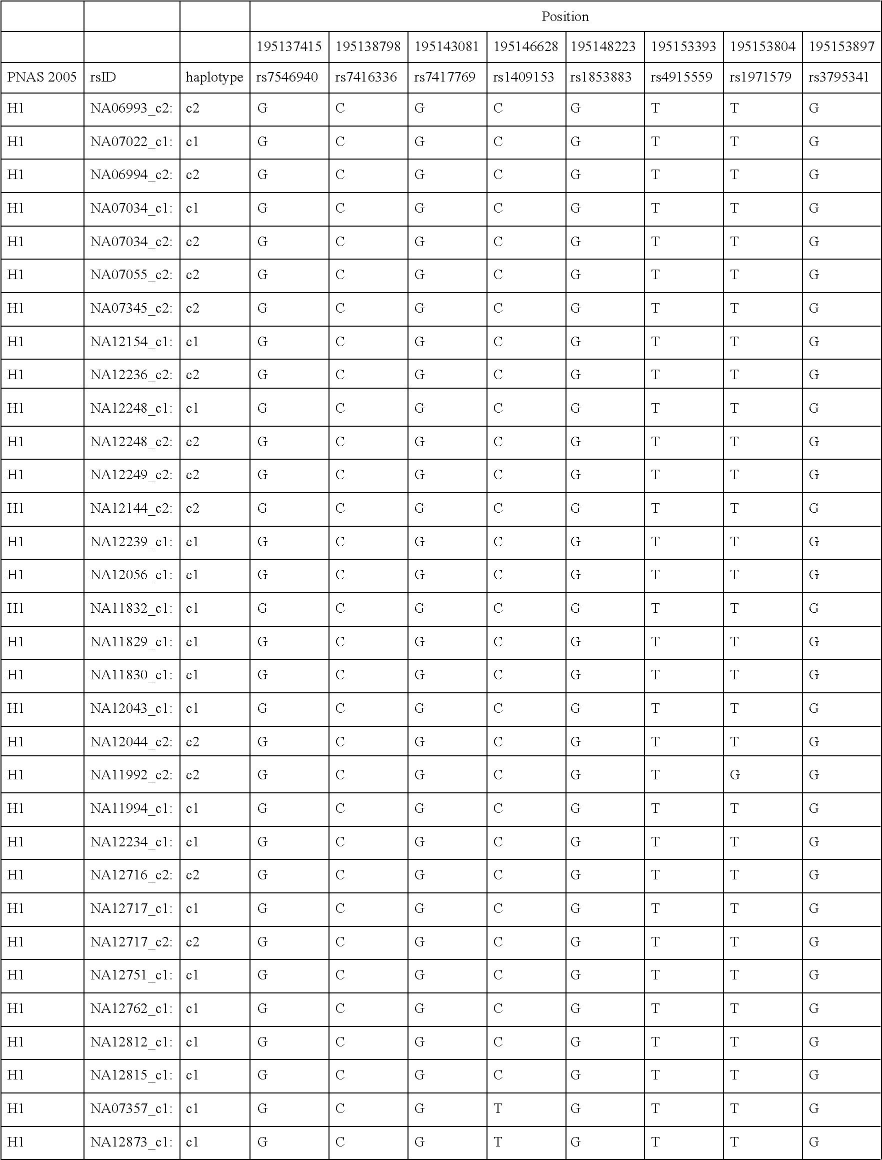

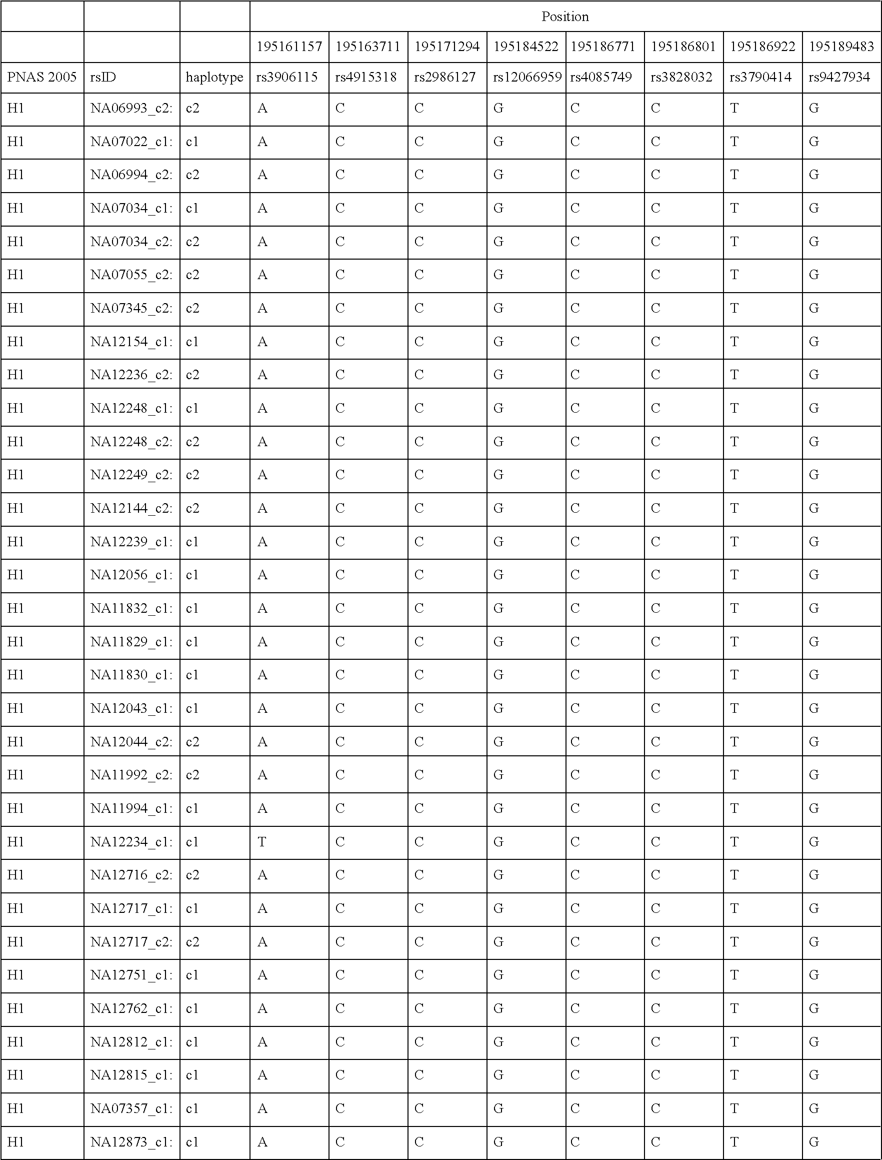

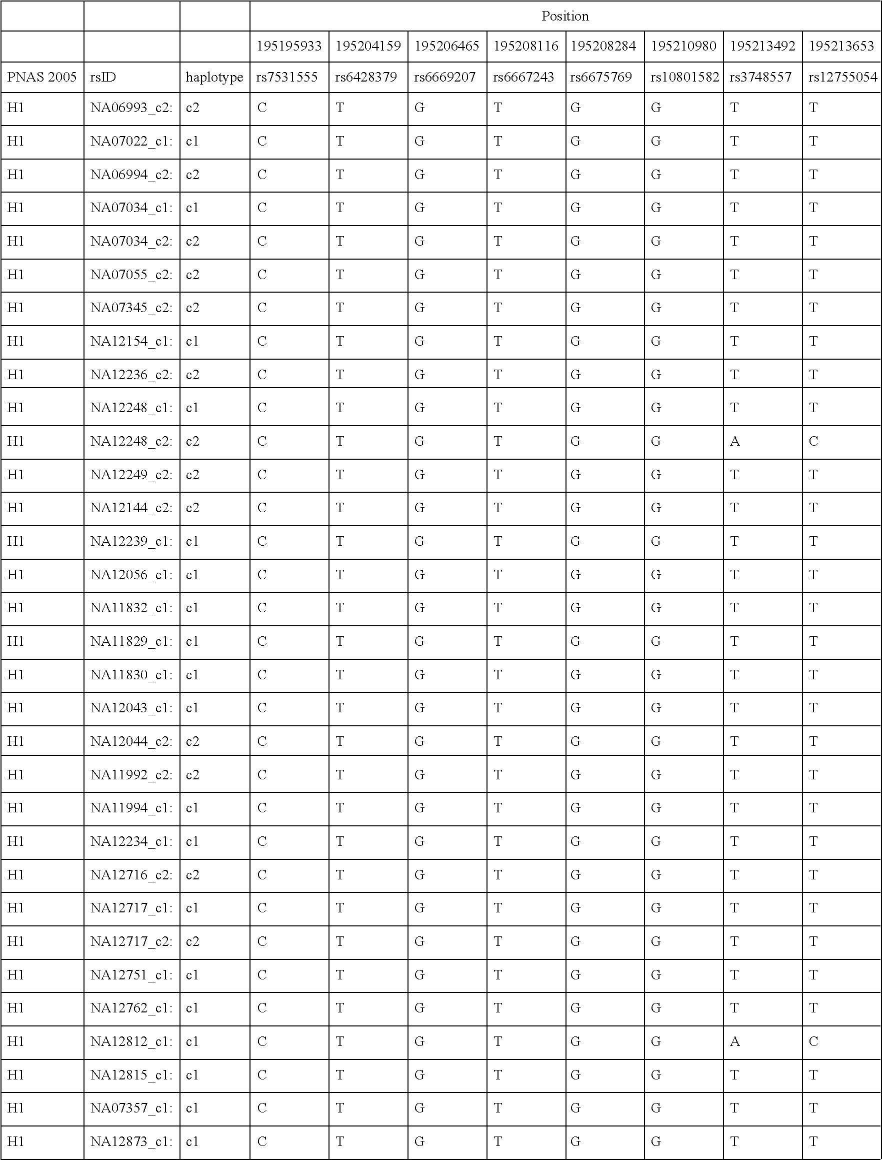

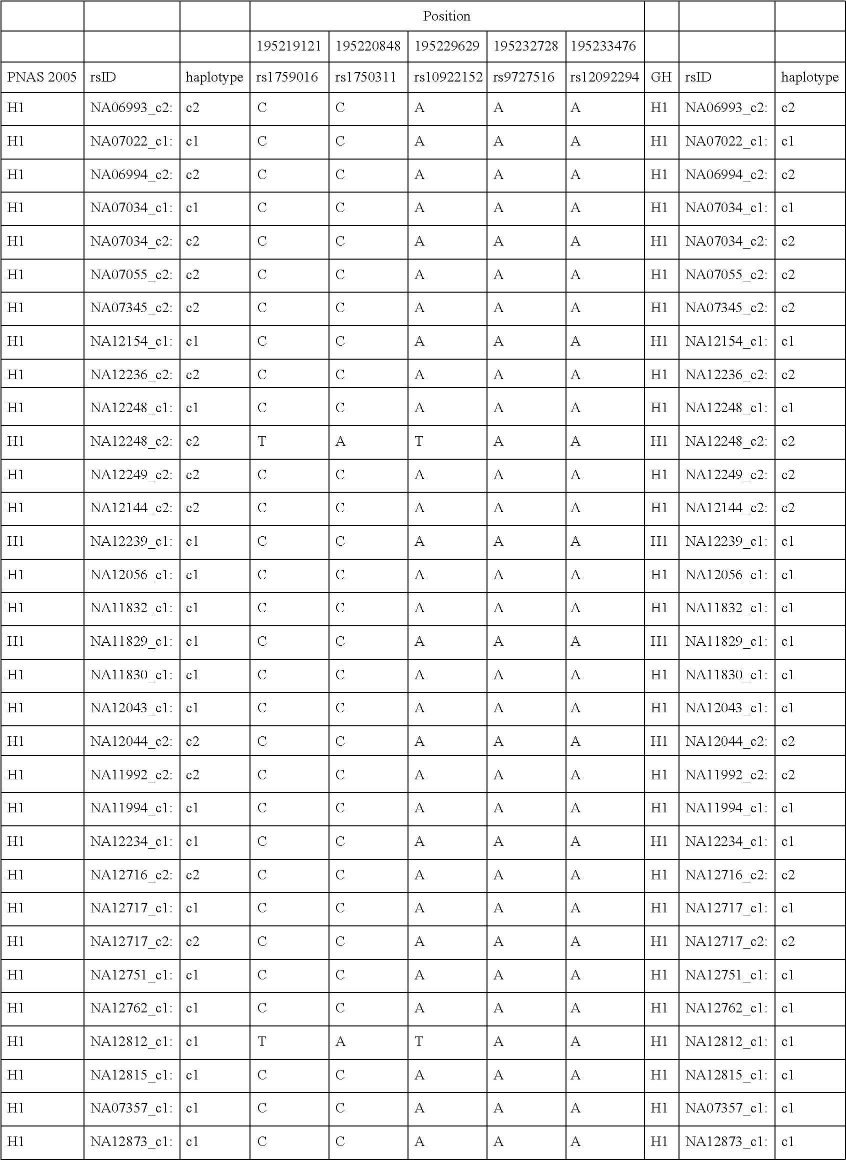

In Table 2 “rs1061170” is disclosed as SEQ ID NO: 16, “rs10922094” is disclosed as SEQ ID NO: 21, “rs12124794” is disclosed as SEQ ID NO: 22, “rs12405238” is disclosed as SEQ ID NO: 23, “rs10922102” is disclosed as SEQ ID NO: 28, “rs2860102” is disclosed as SEQ ID NO: 29, “rs4658046” is disclosed as SEQ ID NO: 30, “rs12038333” is disclosed as SEQ ID NO: 33, “rs12045503” is disclosed as SEQ ID NO: 34, “rs9970784” is disclosed as SEQ ID NO: 35, “rs1831282” is disclosed as SEQ ID NO: 36, “rs203687” is disclosed as SEQ ID NO: 37, “rs2016427” is disclosed as SEQ ID NO: 38, “rs2019727” is disclosed as SEQ ID NO: 39, “rs1887973” is disclosed as SEQ ID NO: 40, “rs6428357” is disclosed as SEQ ID NO: 41, “rs6695321” is disclosed as SEQ ID NO: 43, “rs10733086” is disclosed as SEQ ID NO: 44, “rs1410997” is disclosed as SEQ ID NO: 45, “rs203685” is disclosed as SEQ ID NO: 46, “rs10737680” is disclosed as SEQ ID NO: 48, “rs403846” is disclosed as SEQ ID NO: 17, “rs1409153” is disclosed as SEQ ID NO: 18, “rs1750311” is disclosed as SEQ ID NO: 20.

TABLE 3
Phased data of Hap Map Caucasian (CEU) chromosomes identified as CFH H2 using 6 defining SNPs described by
Hageman et al. 2005. Chromosome position provided in row 1 are from NCBI Build 36.

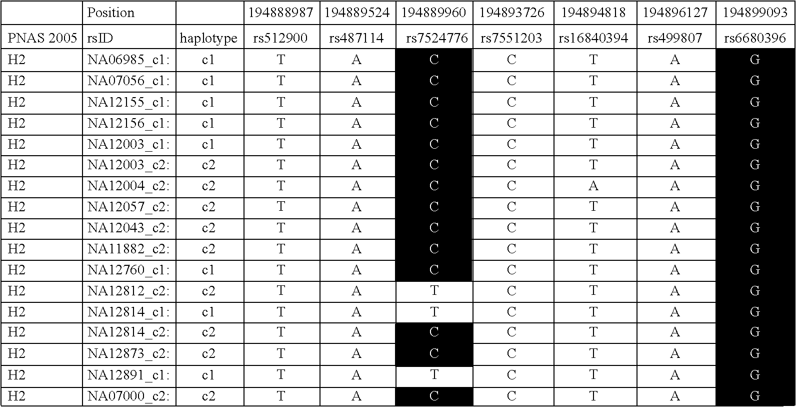

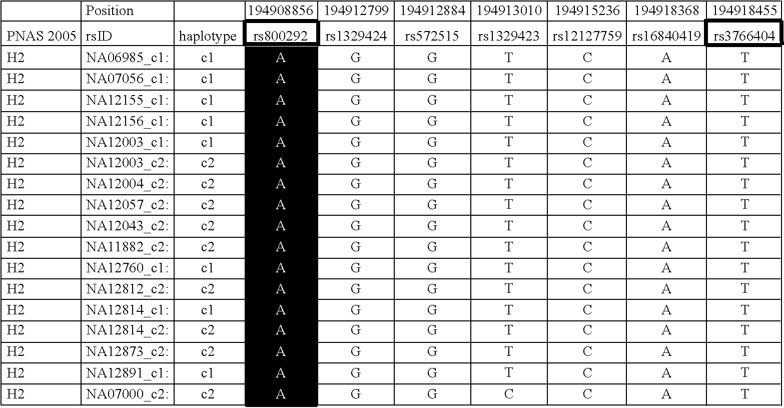

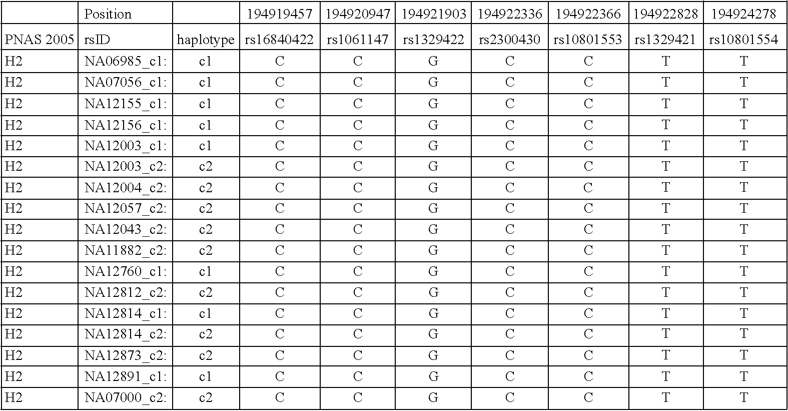

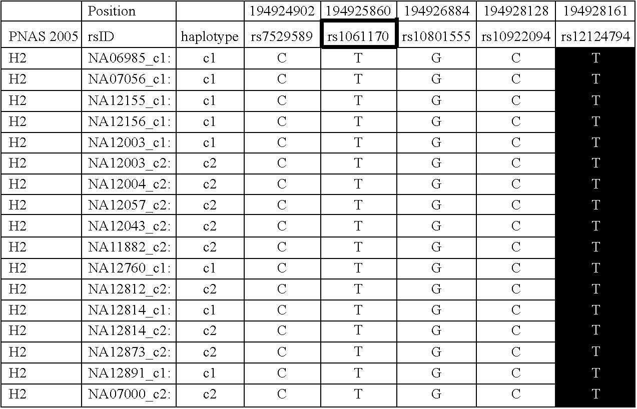

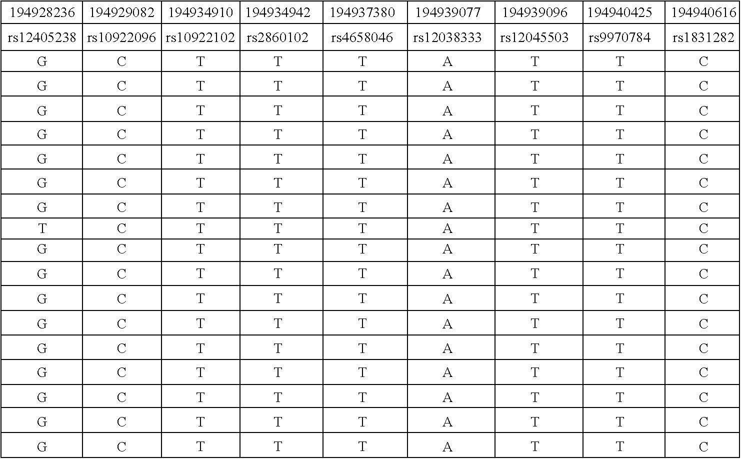

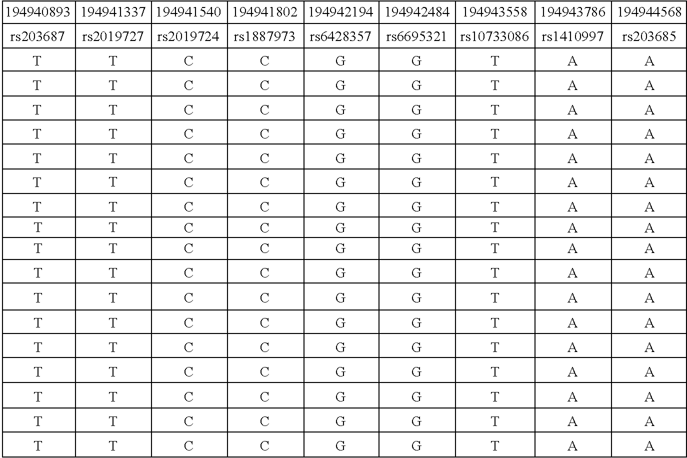

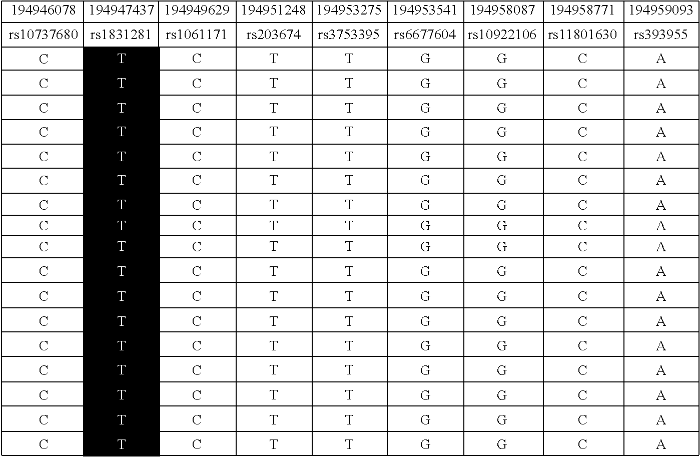

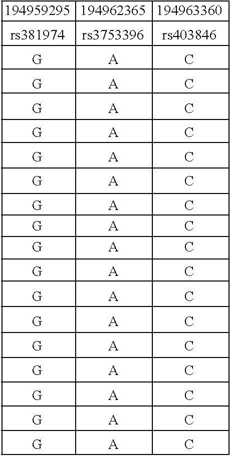

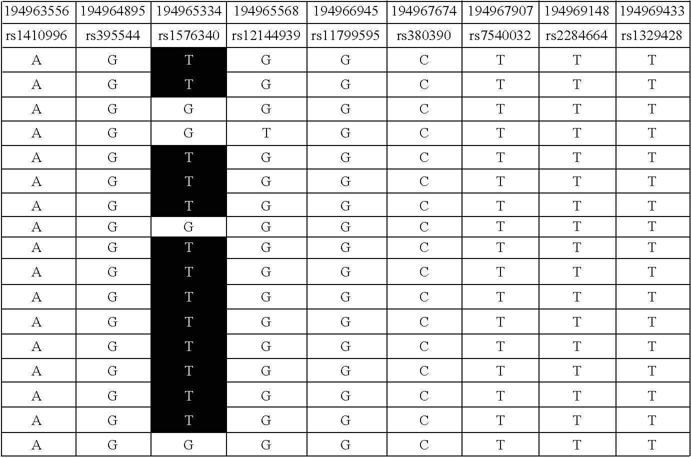

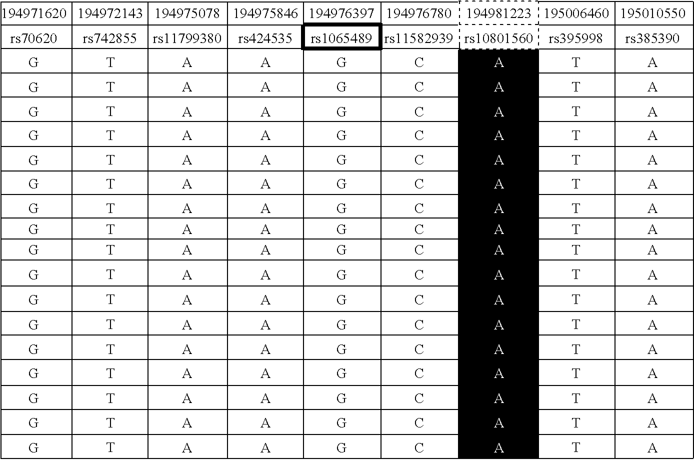

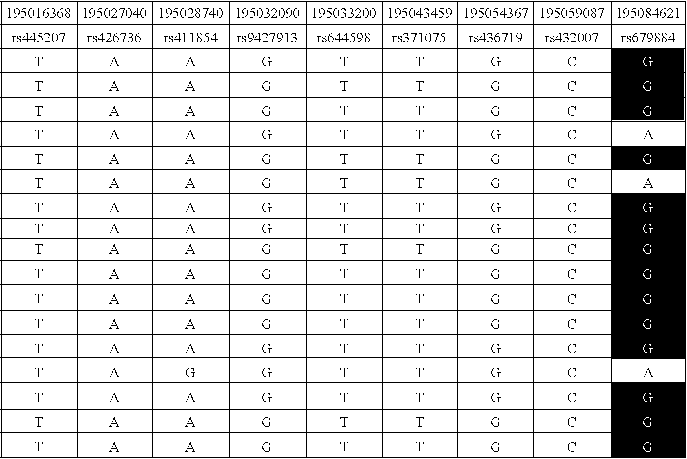

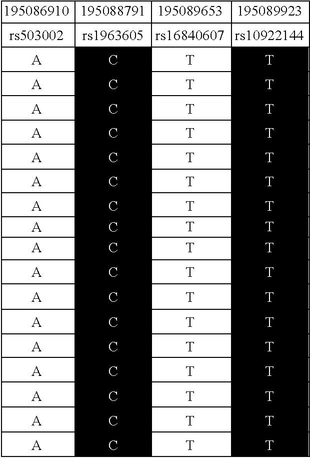

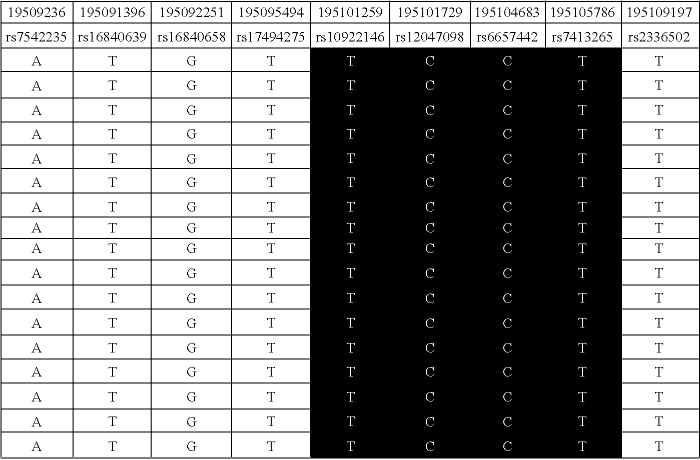

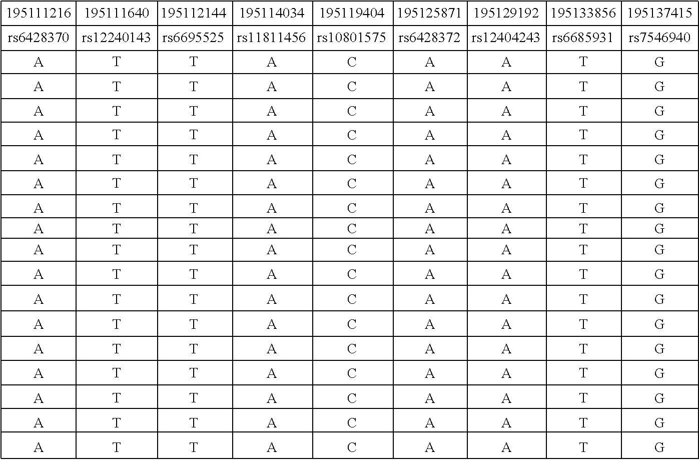

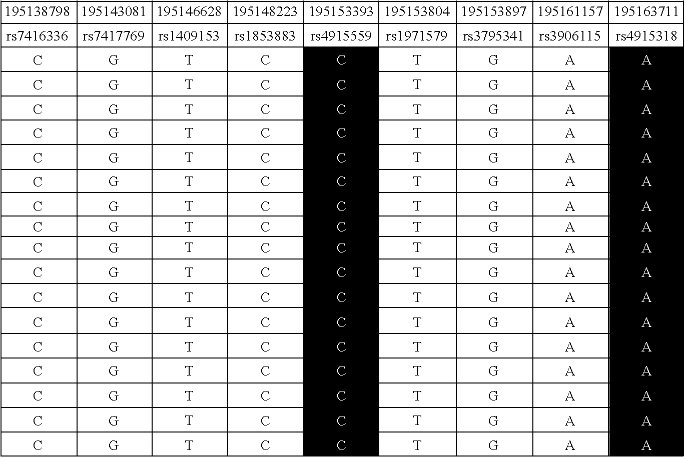

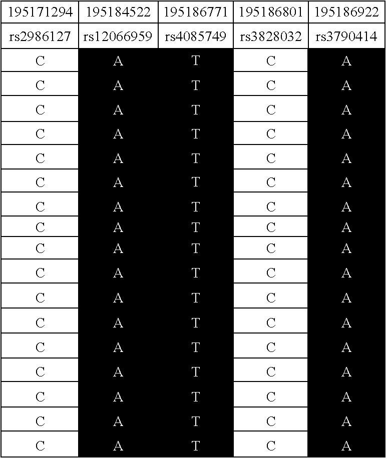

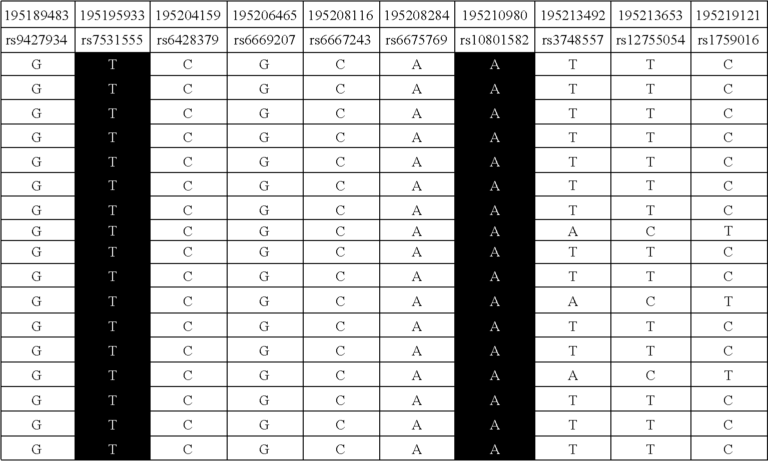

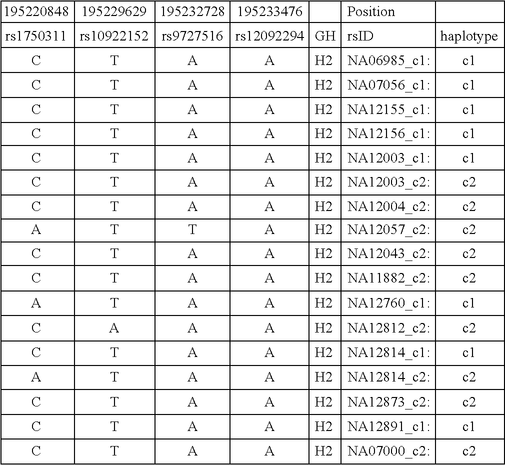

In Table 3 “rs1061170” is disclosed as SEQ ID NO: 16, “rs10922094” is disclosed as SEQ ID NO: 21, “rs12124794” is disclosed as SEQ ID NO: 22, “rs12405238” is disclosed as SEQ ID NO: 23, “rs10922096” is disclosed as SEQ ID NO: 24, “rs10922102” is disclosed as SEQ ID NO: 28, “rs2860102” is disclosed as SEQ ID NO: 29, “rs4658046” is disclosed as SEQ ID NO: 30, “rs12038333” is disclosed as SEQ ID NO: 33, “rs12045503” is disclosed as SEQ ID NO: 34, “rs9970784” is disclosed as SEQ ID NO: 35, “rs1831282” is disclosed as SEQ ID NO: 36, “rs203687” is disclosed as SEQ ID NO: 37, “rs2019727” is disclosed as SEQ ID NO: 38, “rs2019724” is disclosed as SEQ ID NO: 39, “rs1887973” is disclosed as SEQ ID NO: 40, “r56428357” is disclosed as SEQ ID NO: 41, “rs6695321” is disclosed as SEQ ID NO: 43, “rs10733086” is disclosed as SEQ ID NO: 44, “rs1410997” is disclosed as SEQ ID NO: 45, “rs203685” is disclosed as SEQ ID NO: 46, “rs10737680” is disclosed as SEQ ID NO: 48, “rs403846” is disclosed as SEQ ID NO: 17, “rs1409153” is disclosed as SEQ ID NO: 18 and “rs1750311” is disclosed as SEQ ID NO: 20.

TABLE 4
Phased data of HapMap Caucasian (CEU) chromosomes identified as CFH H3 using 6 defining SNPs described by
Hageman et al. 2005. Chromosome position provided in row 1 are from NCBI Build 36.

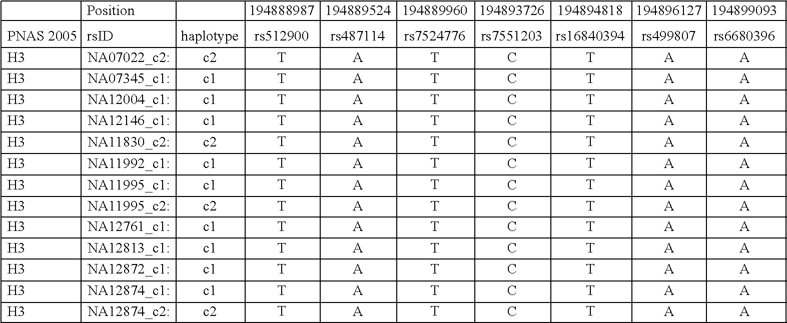

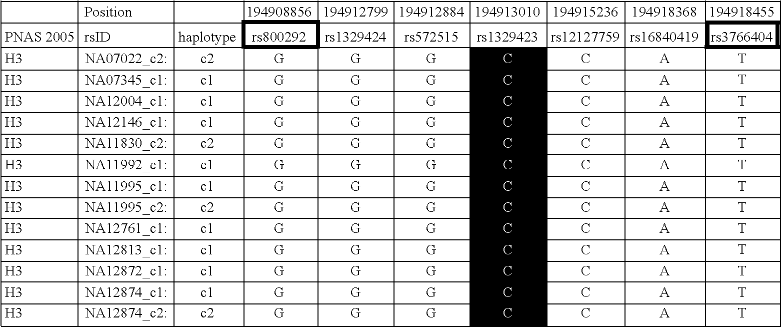

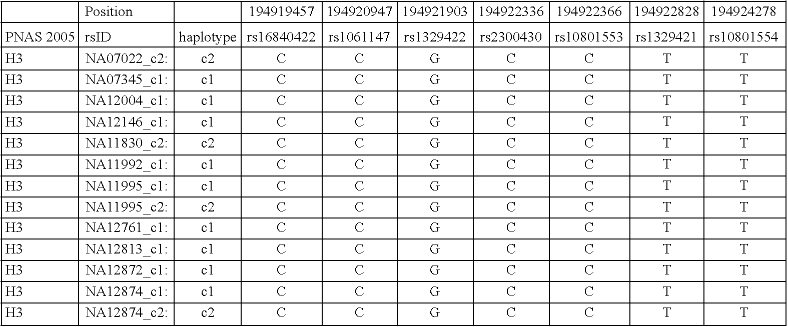

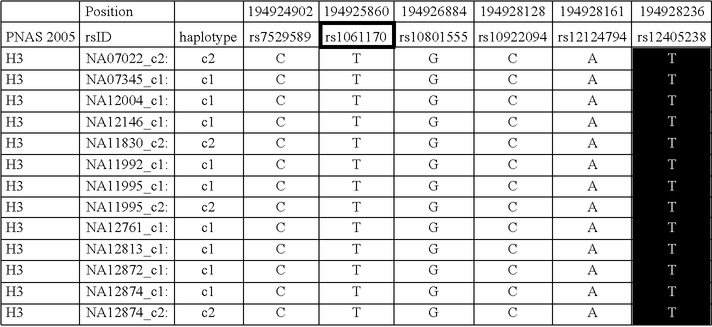

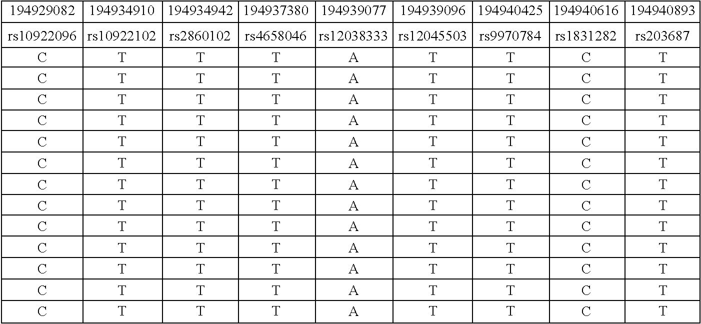

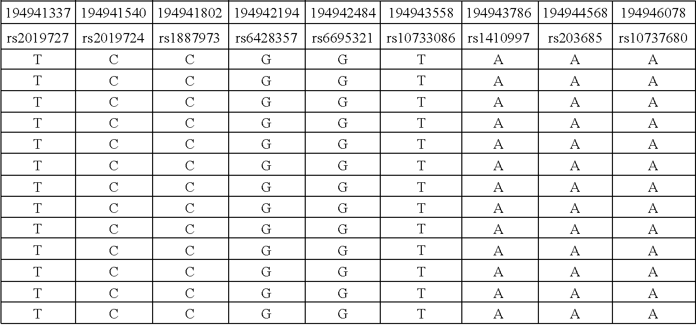

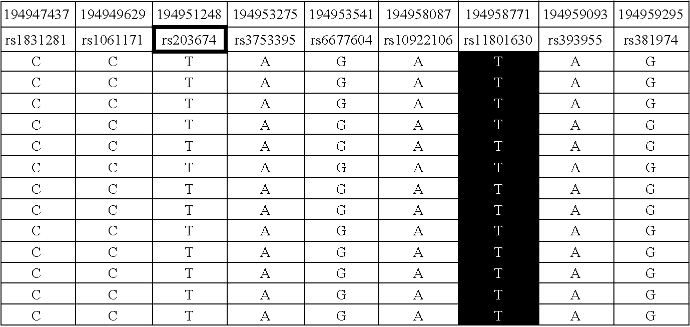

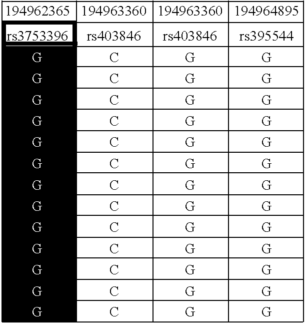

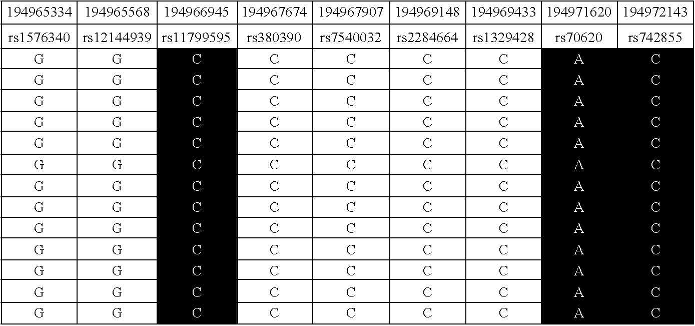

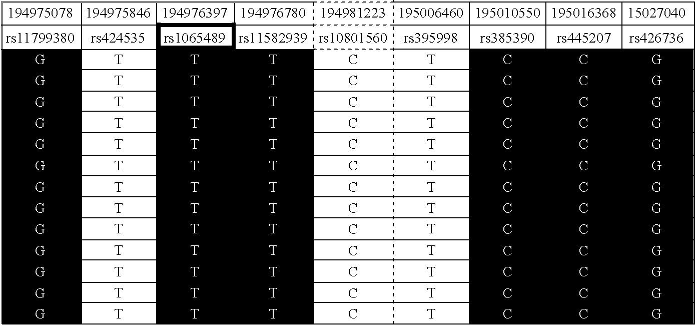

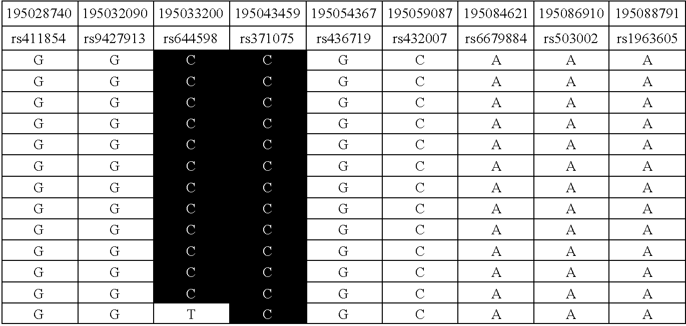

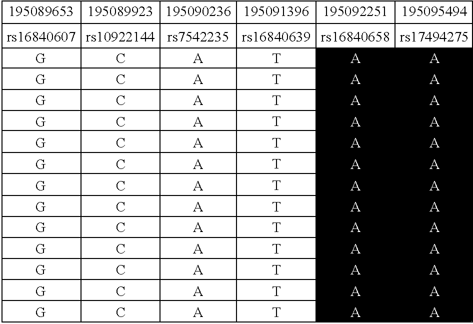

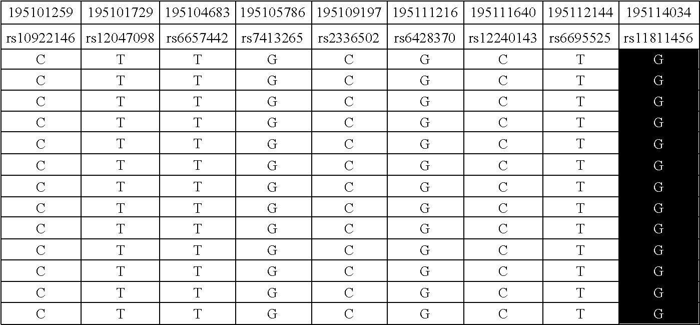

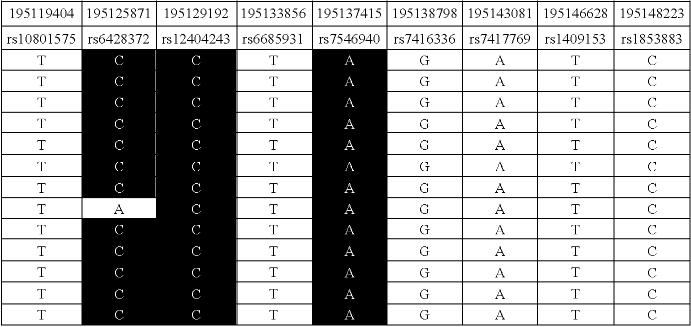

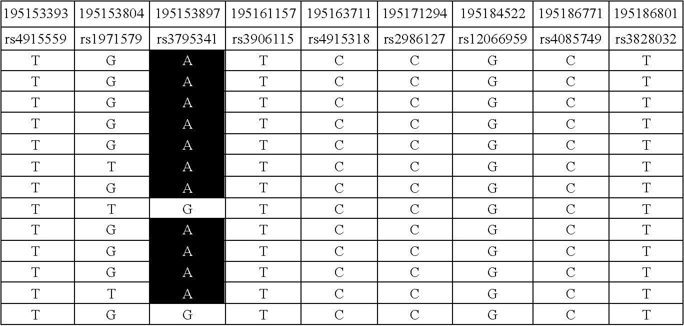

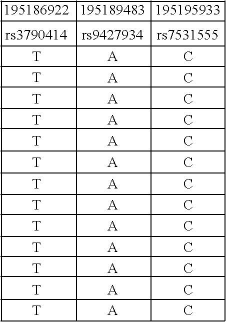

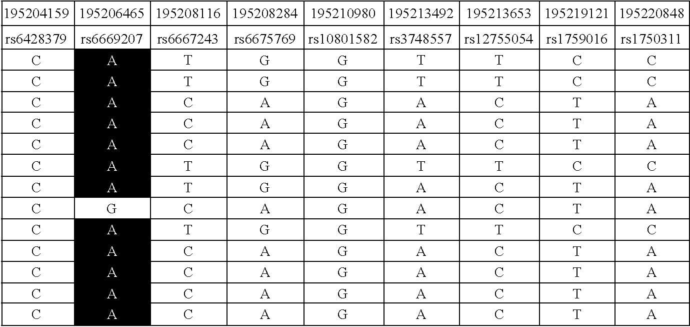

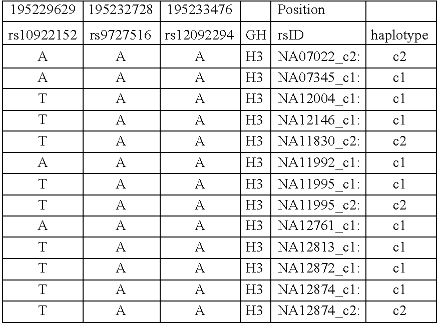

In Table 4 “rs1061170” is disclosed as SEQ ID NO: 16, “rs10922094” is disclosed as SEQ ID NO: 21, “rs12124794” is disclosed as SEQ ID NO: 22, “rs12405238” is disclosed as SEQ ID NO: 23, “rs10922096” is disclosed as SEQ ID NO: 24, “rs10922102” is disclosed as SEQ ID NO: 28, “rs2860102” is disclosed as SEQ ID NO: 29, “rs4658046” is disclosed as SEQ ID NO: 30, “rs12038333” is disclosed as SEQ ID NO: 33, “rs12045503” is disclosed as SEQ ID NO: 34, “rs9970784” is disclosed as SEQ ID NO: 35, “rs1831282” is disclosed as SEQ ID NO: 36, “rs203687” is disclosed as SEQ ID NO: 37, “rs2019727” is disclosed as SEQ ID NO: 38, “rs2019724” is disclosed as SEQ ID NO: 39, “rs1887973” is disclosed as SEQ ID NO: 40, “r56428357” is disclosed as SEQ ID NO: 41, “rs6695321” is disclosed as SEQ ID NO: 43, “rs10733086” is disclosed as SEQ ID NO: 44, “rs1410997” is disclosed as SEQ ID NO: 45, “rs203685” is disclosed as SEQ ID NO: 46, “rs10737680” is disclosed as SEQ ID NO: 48, “rs403846” is disclosed as SEQ ID NO: 17, “rs1409153” is disclosed as SEQ ID NO: 18 and “rs1750311” is disclosed as SEQ ID NO: 20.

TABLE 5
Phased data of HapMap Caucasian (CEU) chromosomes identified as CFH H4 using 6 defining SNPs described by
Hageman et al. 2005 Chromosome position provided in row 1 are from NCBI Build 36.

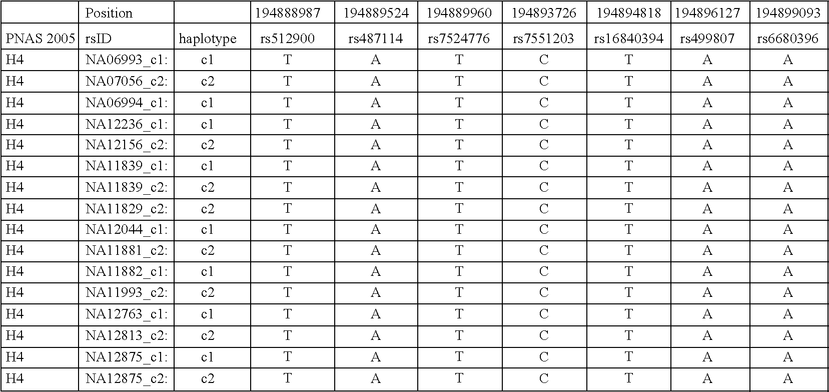

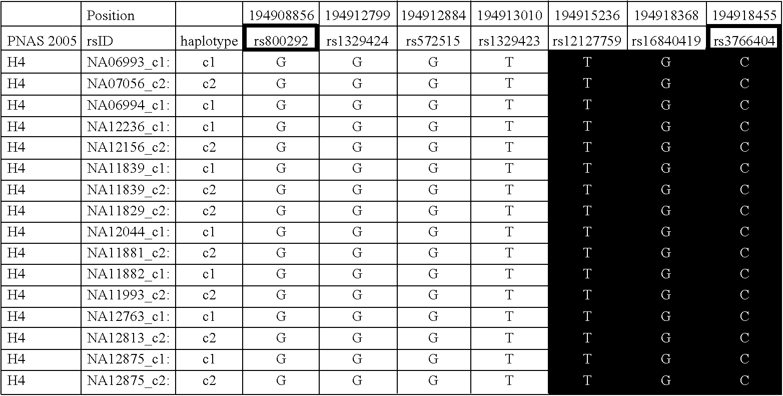

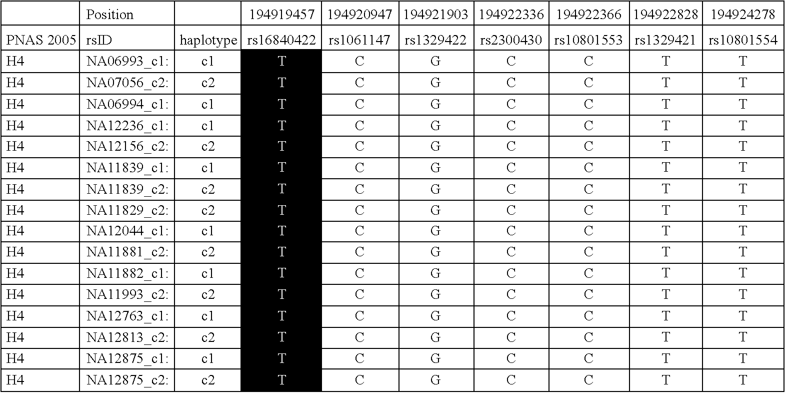

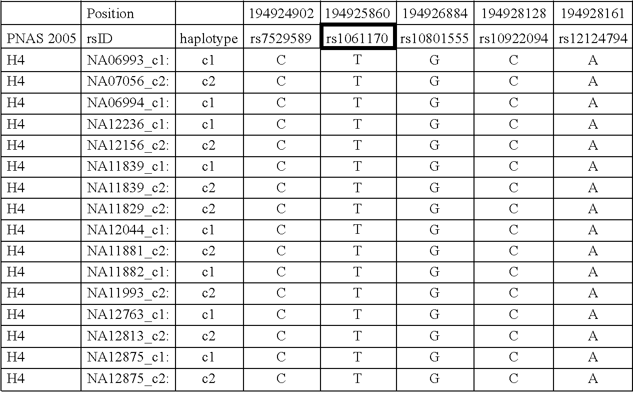

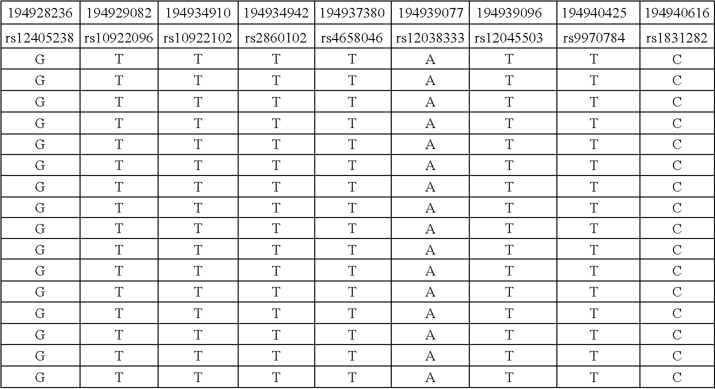

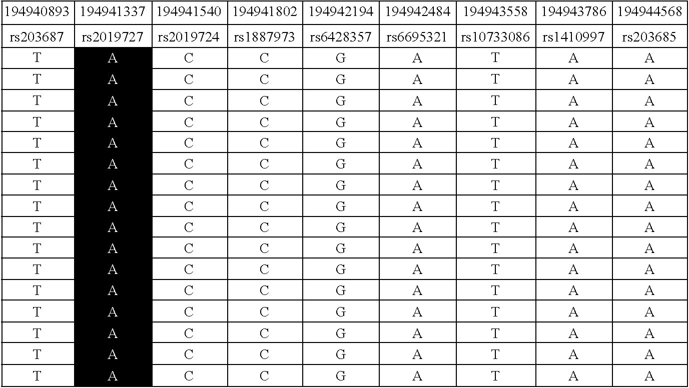

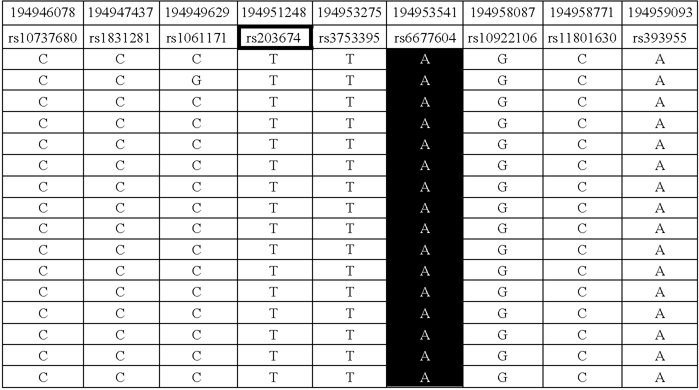

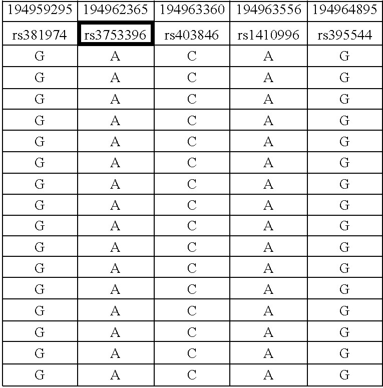

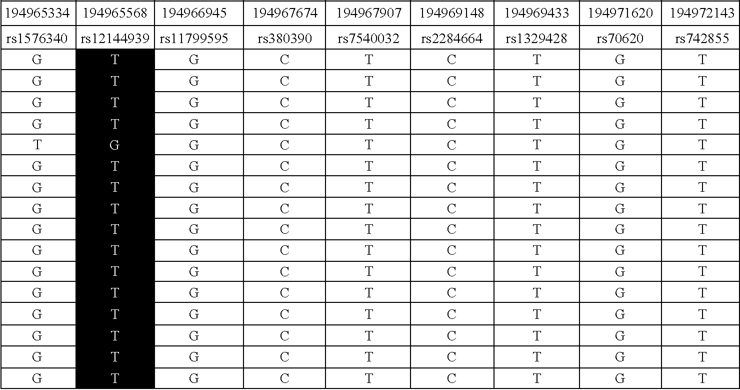

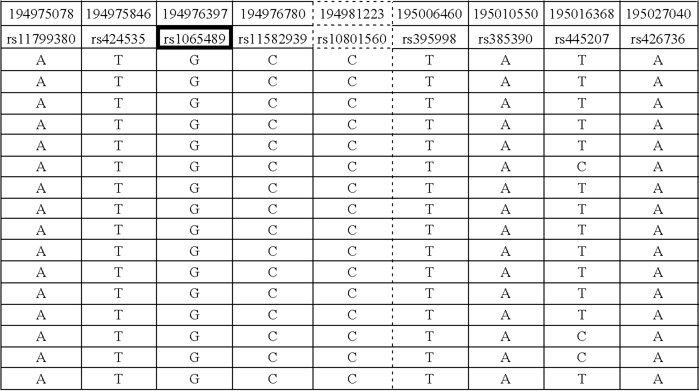

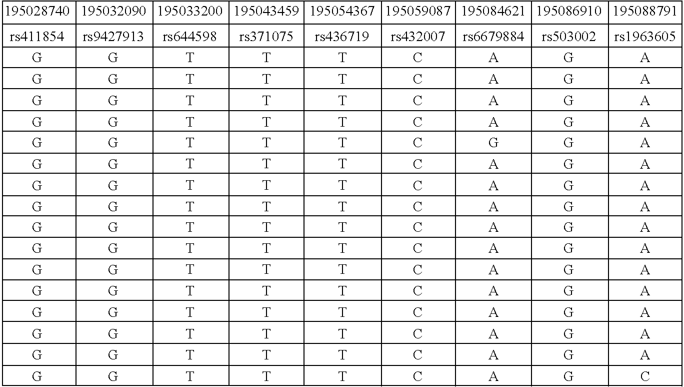

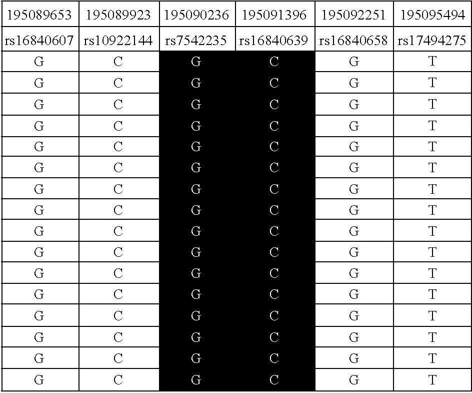

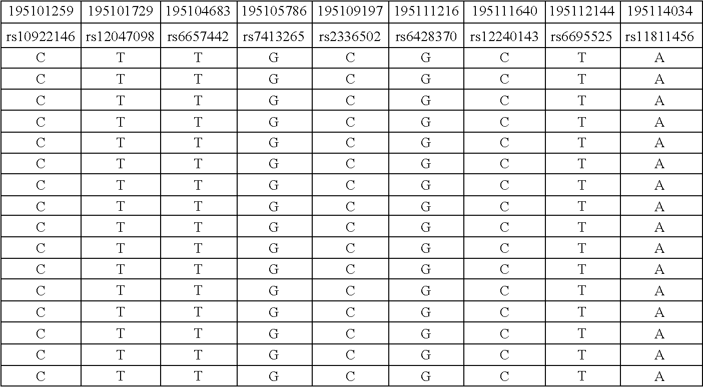

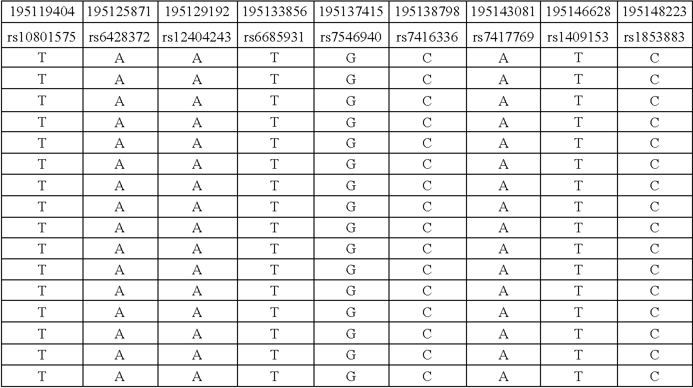

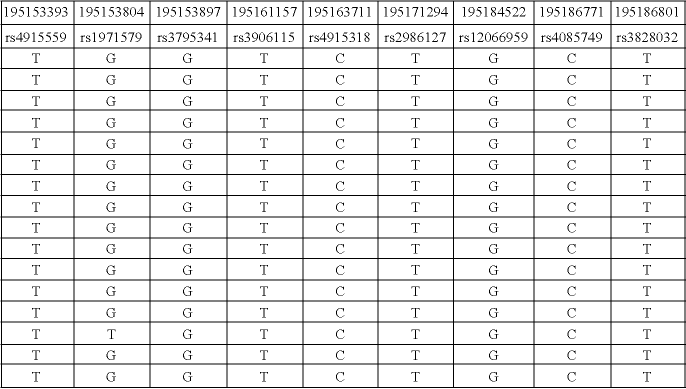

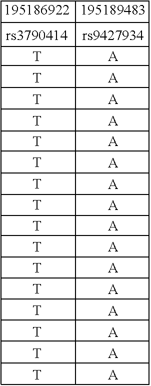

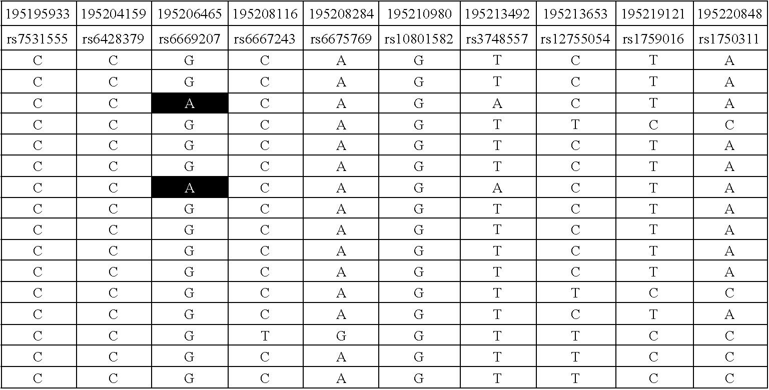

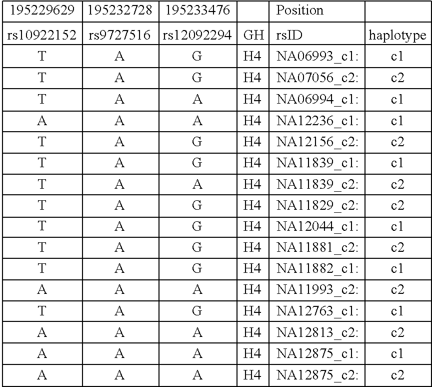

In Table 5 “rs1061170” is disclosed as SEQ ID NO: 16, “rs10922094” is disclosed as SEQ ID NO: 21, “rs12124794” is disclosed as SEQ ID NO: 22, “rs12405238” is disclosed as SEQ ID NO: 23, “rs10922096” is disclosed as SEQ ID NO: 24, “rs10922102” is disclosed as SEQ ID NO: 28, “rs2860102” is disclosed as SEQ ID NO: 29, “rs4658046” is disclosed as SEQ ID NO: 30, “rs12038333” is disclosed as SEQ ID NO: 33, “rs12045503” is disclosed as SEQ ID NO: 34, “rs9970784” is disclosed as SEQ ID NO: 35, “rs1831282” is disclosed as SEQ ID NO: 36, “rs203687” is disclosed as SEQ ID NO: 37, “rs2019727” is disclosed as SEQ ID NO: 38, “rs2019724” is disclosed as SEQ ID NO: 39, “rs1887973” is disclosed as SEQ ID NO: 40, “r6428357” is disclosed as SEQ ID NO: 41, “rs6695321” is disclosed as SEQ ID NO: 43, “rs10733086” is disclosed as SEQ ID NO: 44, “rs1410997” is disclosed as SEQ ID NO: 45, “rs203685” is disclosed as SEQ ID NO: 46, “rs10737680” is disclosed as SEQ ID NO: 48, “rs403846” is disclosed as SEQ ID NO: 17, “rs1409153” is disclosed as SEQ ID NO: 18 and “rs1750311” is disclosed as SEQ ID NO: 20.

TABLE 6

| CFH5′ | CFH3′ | R3 | R1 | R4 | R2 | R5 | | |
|---|---|---|---|---|---|---|---|---|
| H1 | H1 | H1 | H1 | H1 | H1 | H1 | H1/H1 | NA07034_c1: |
| H1 | H1 | H1 | H1 | H1 | H1 | H1 | | NA12248_c1: |
| | | | | | | | | NA12717_c1: |
| | | | | | | | | NA07357_c1: |
| | | | | | | | | NA12056_c1: |
| | | | | | | | | NA12716_c1: |
| | | | | | | | | NA12762_c1: |
| | | | | | | | | NA12815_c1: |
| H1 | H1 | H1 | H1 | H1 | H1 | H1 | H1/H2 | NA12043_c1: |
| H2 | H2 | H2 | H2 | H2 | H2 | H2 | | NA12812_c1: |
| | | | | | | | | NA12873_c1: |
| H1 | H1 | H1 | H1 | H1 | H1 | H1 | H1/H3 | NA07022_c1: |
| H3 | H3 | H3 | H3 | H3 | H3 | H3 | | NA07055_c1: |
| | | | | | | | | NA07345_c1: |
| | | | | | | | | NA11830_c1: |
| | | | | | | | | NA11992_c1: |
| | | | | | | | | NA12239_c1: |
| H1 | H1 | H1 | H1 | H1 | H1 | H1 | H1/H4 | NA06993_c1: |
| H4 | H4 | H4 | H4 | H4 | H4 | H4 | | NA06994_c1: |
| | | | | | | | | NA11829_c1: |
| | | | | | | | | NA12044_c1: |
| | | | | | | | | NA12236_c1: |

HapMap Allele Combinations: Examples of the most commonly observed CEU HapMap sample haplotype combinations revealed by analysis of phased chromosomes across multiple genes (CFH-CFHR5) in the RCA region.

TABLE 7

| Allele Combination | Percentage HapMap samples |
|---|---|
| H1/H1 | 8% |
| H1/H2 | 3% |
| H1/H3 | 3% |
| H1/H4 | 4% |
| H2/H2 | 3% |
| H2/H3 | 1% |
| H2/H4 | 3% |
| H3/H3 | 3% |
| H3/H4 | 1% |
| H4/H4 | 3% |
| TOTAL | 29% |

BOLD = risk allele
Italics and underline = protective allele

Prevalence of CEU HapMap Alleles. Percentage of CEU HapMap samples observed across all possible allele combinations of the most prevalent CFH-defined haplotypes (H1, H2, H3, H4). Only 30% of the CEU HapMap sample collection contains combinations based on previously described CFH haplotypes. The balance of the sample collection reveals haplotype combinations that are comprised of at least 1 novel allele.

TABLE 8
Phased data of HapMap Caucasian (CEU) chromosomes identified as novel CFH halotypes using 6 defining SNPs
described by Hageman et al. 2005. Chromosome position provided in row 1 are from NCBI Build 36.

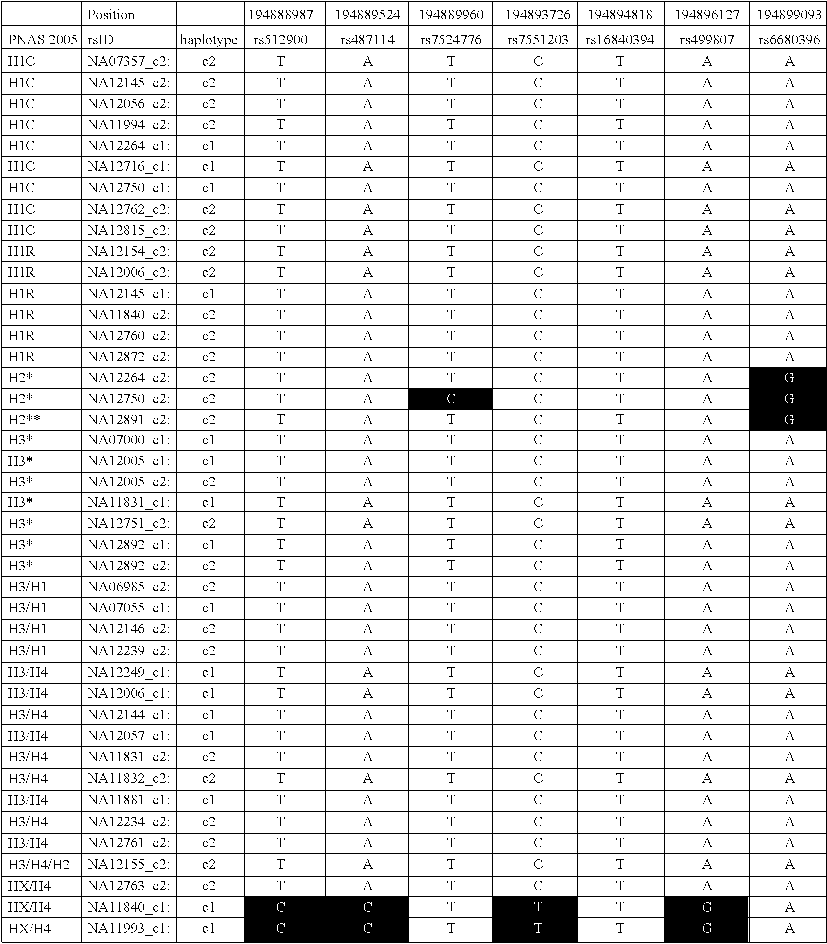

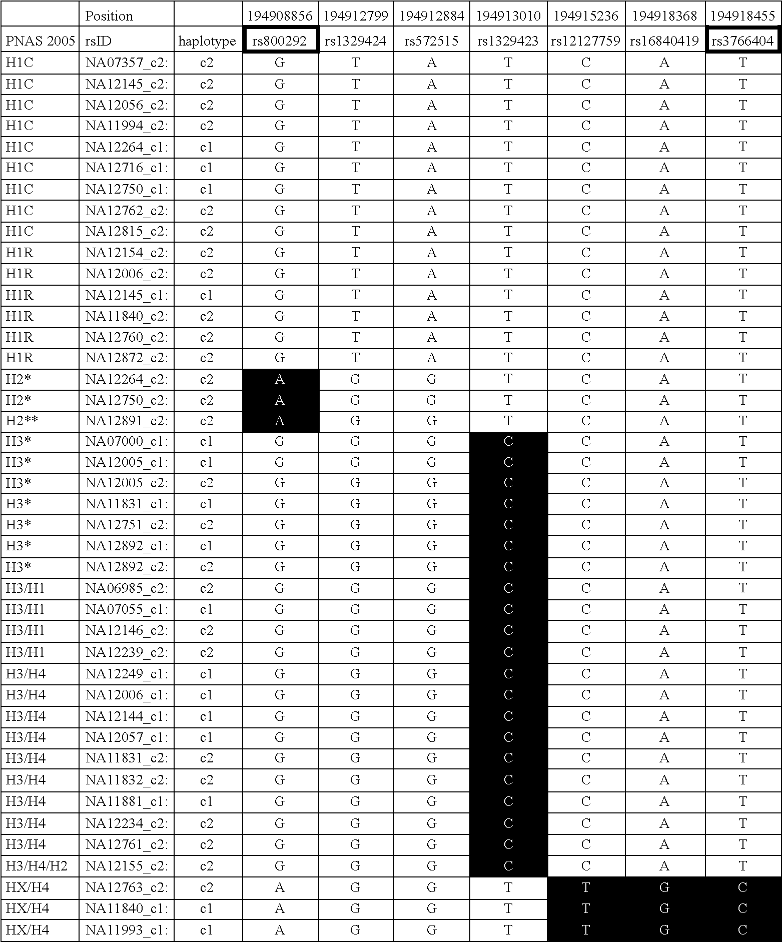

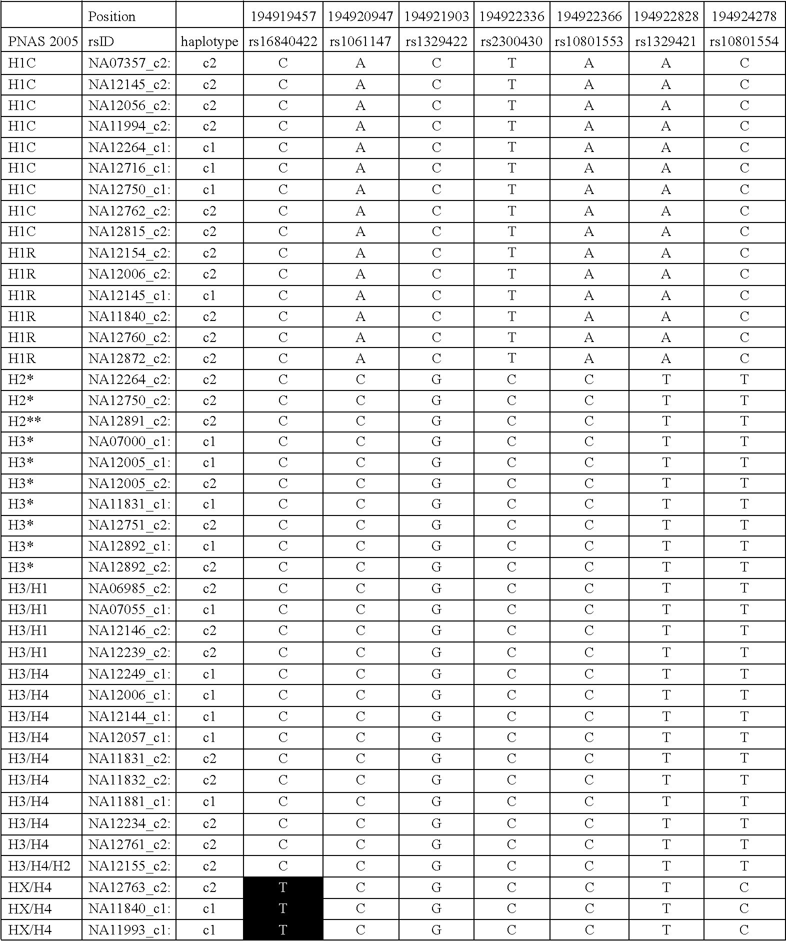

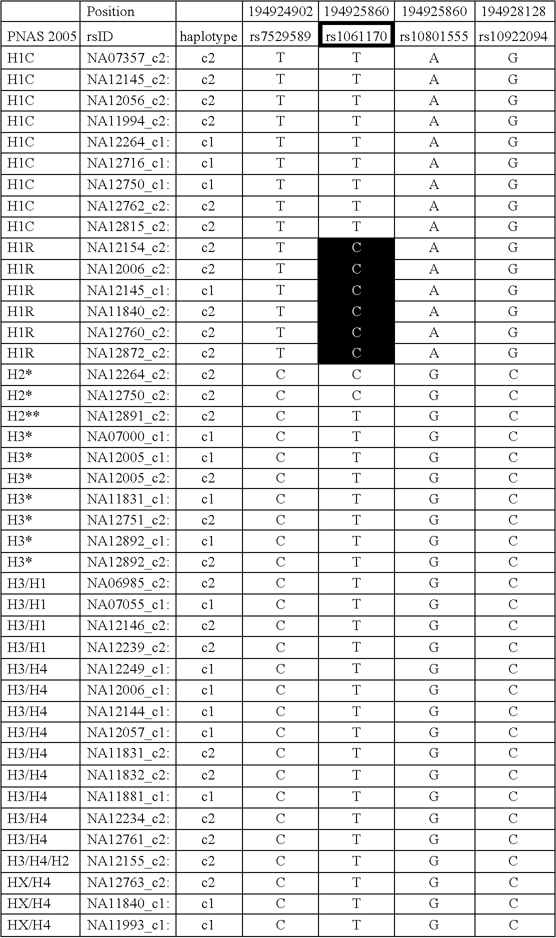

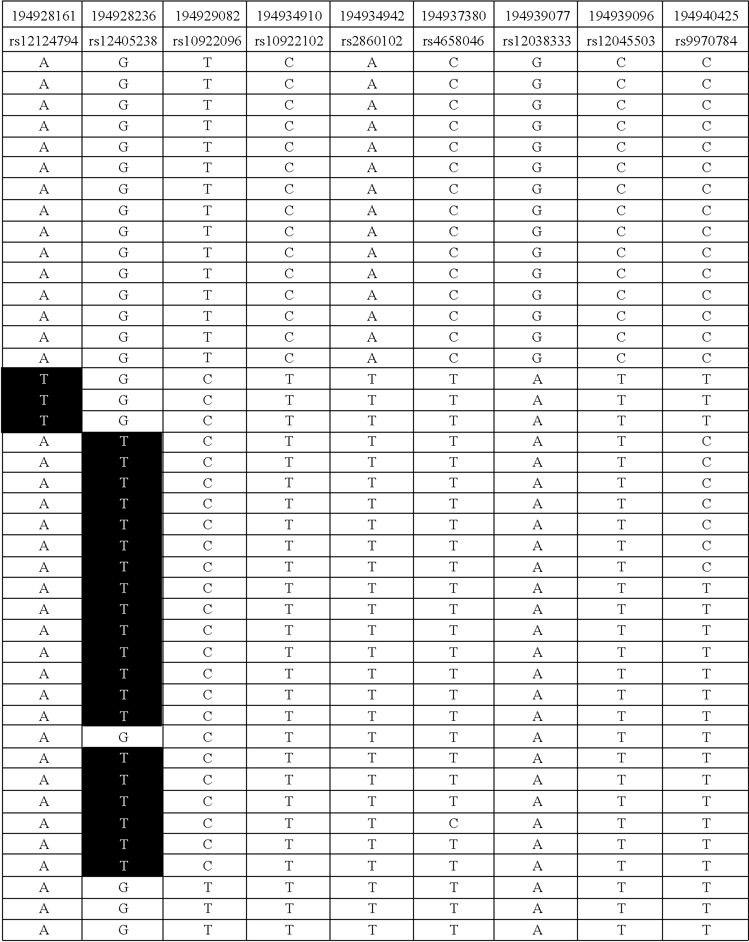

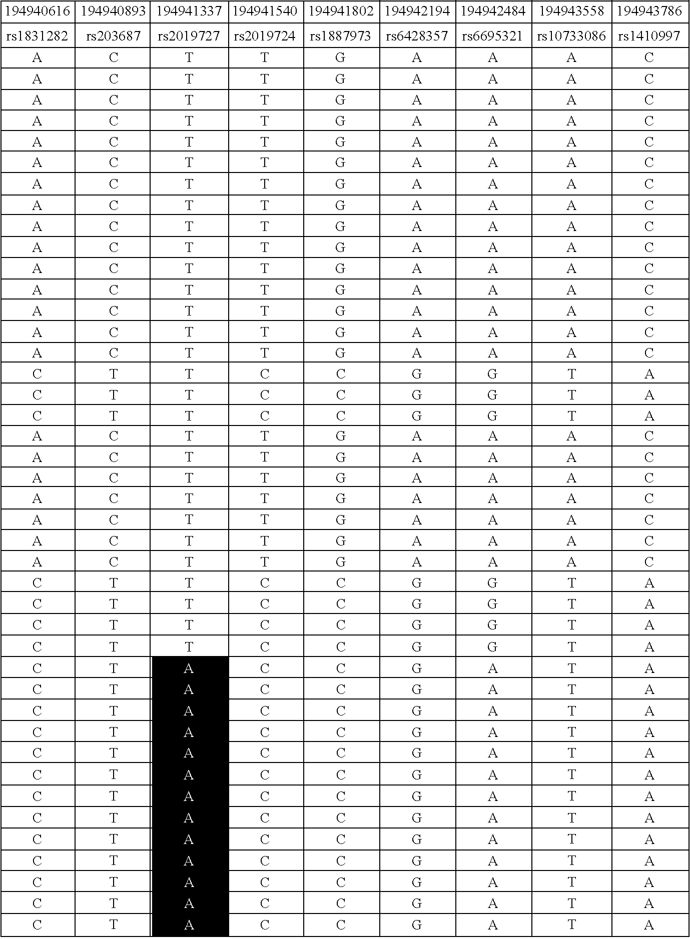

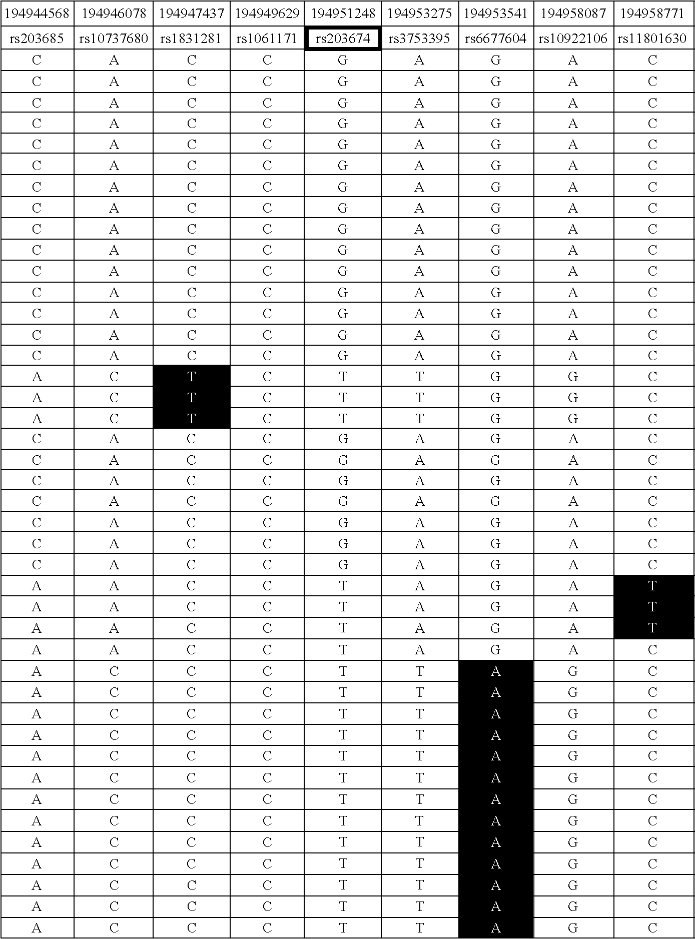

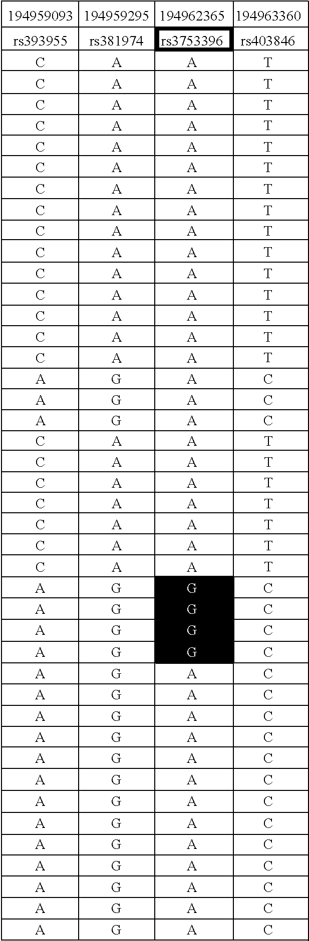

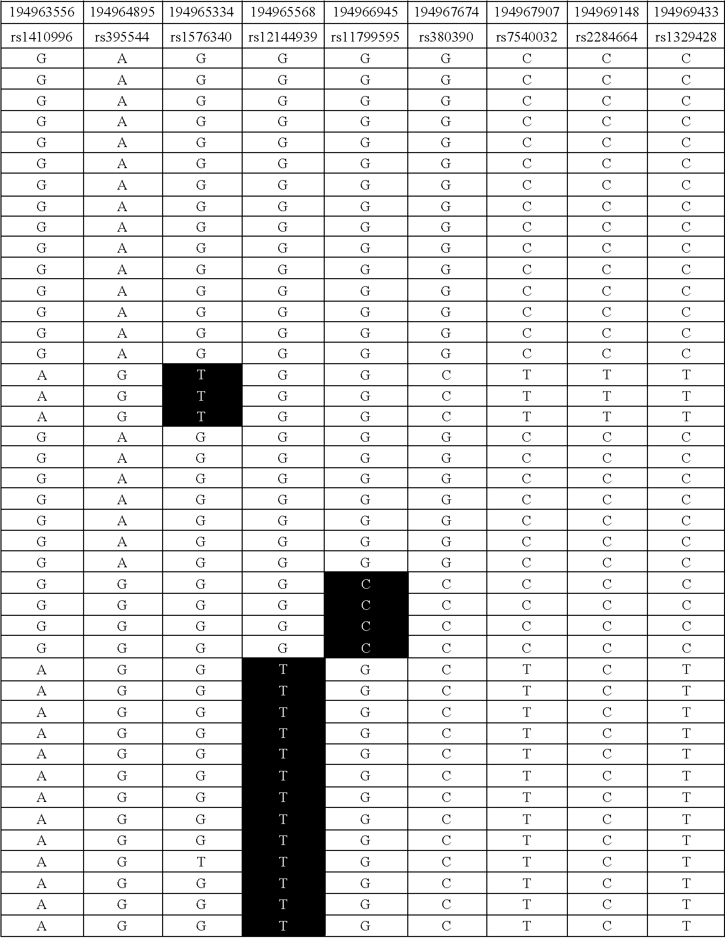

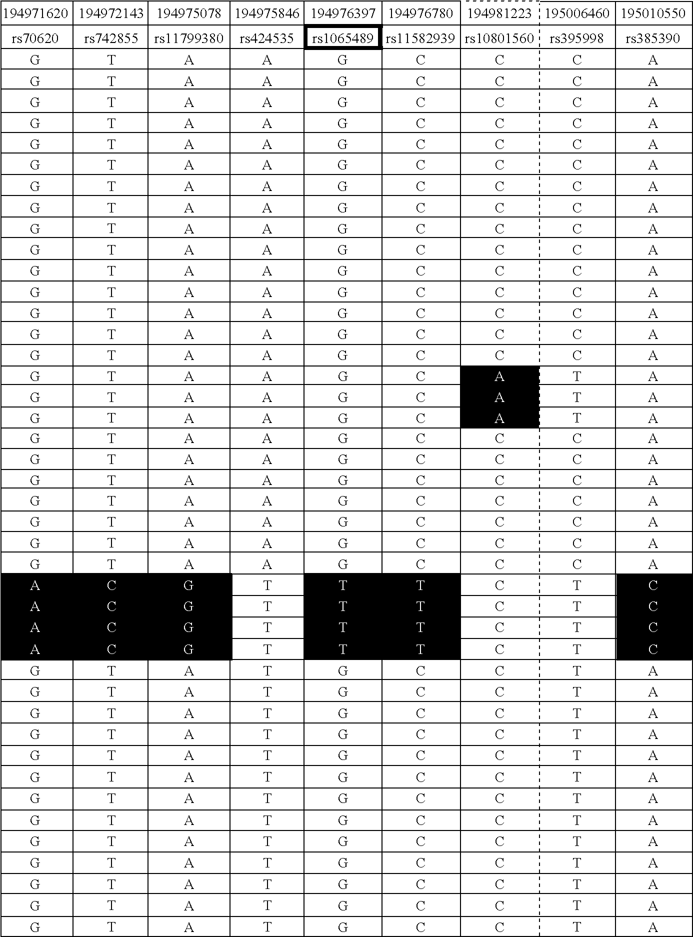

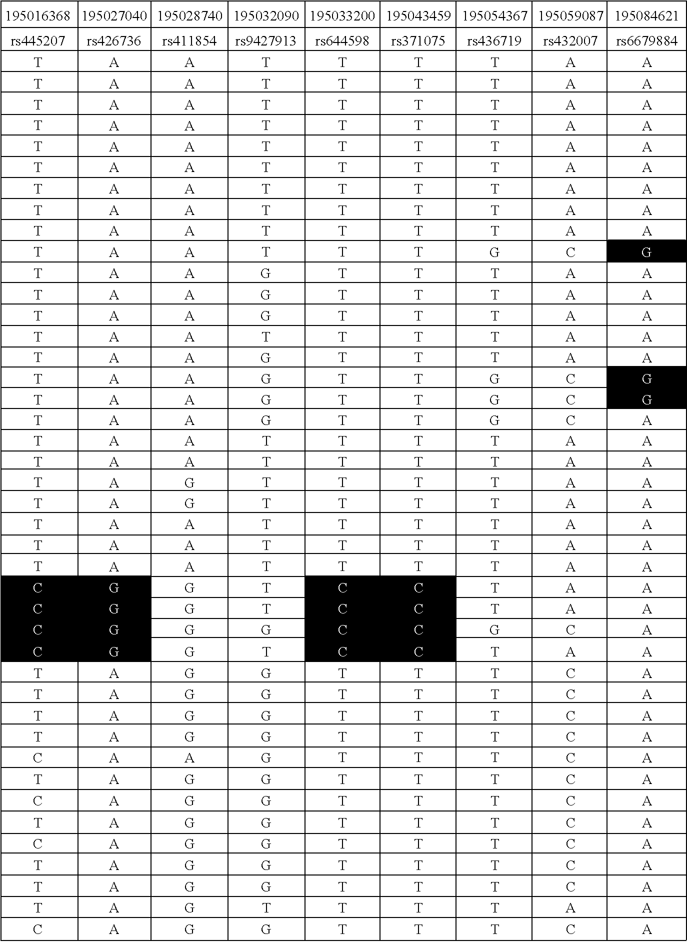

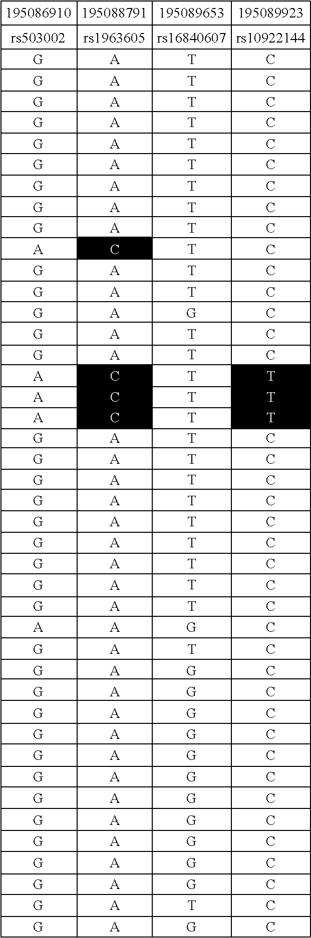

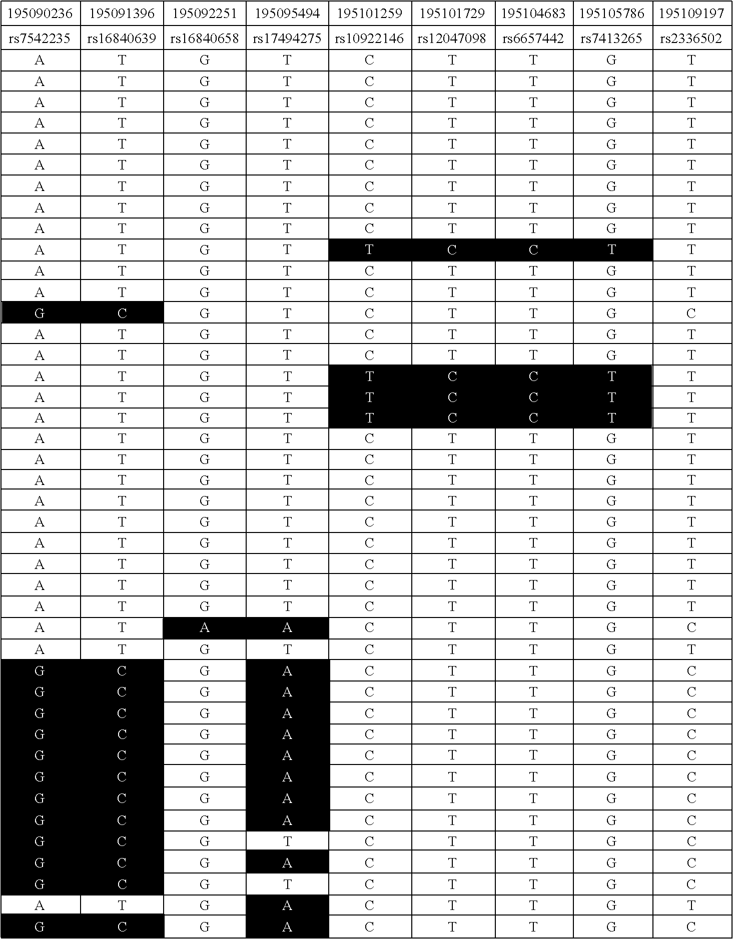

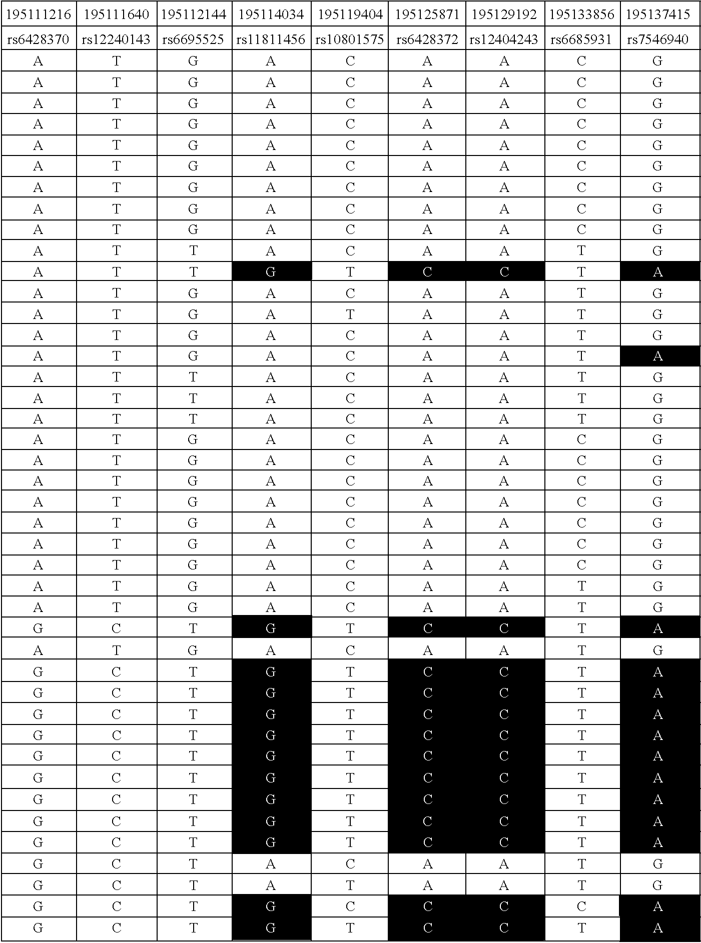

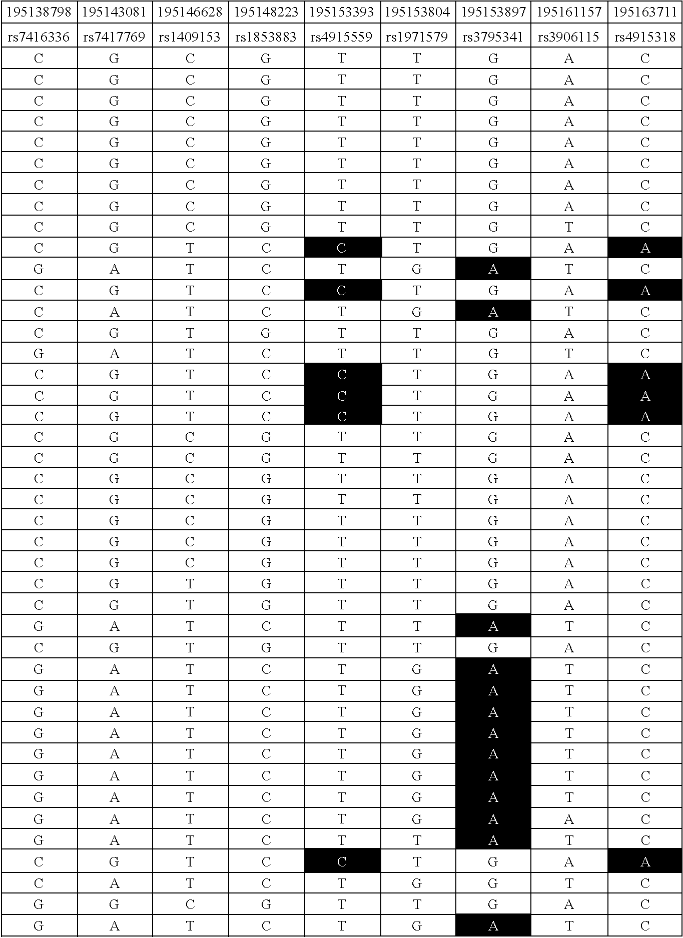

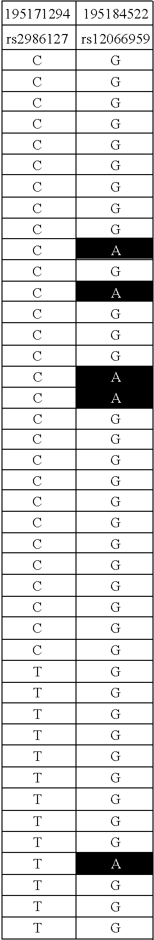

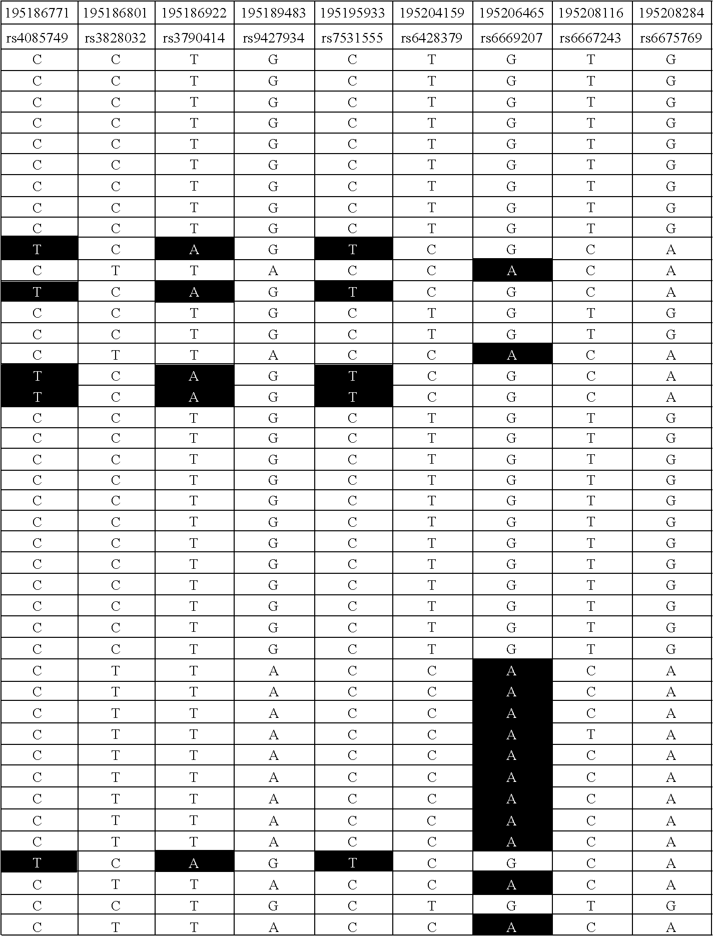

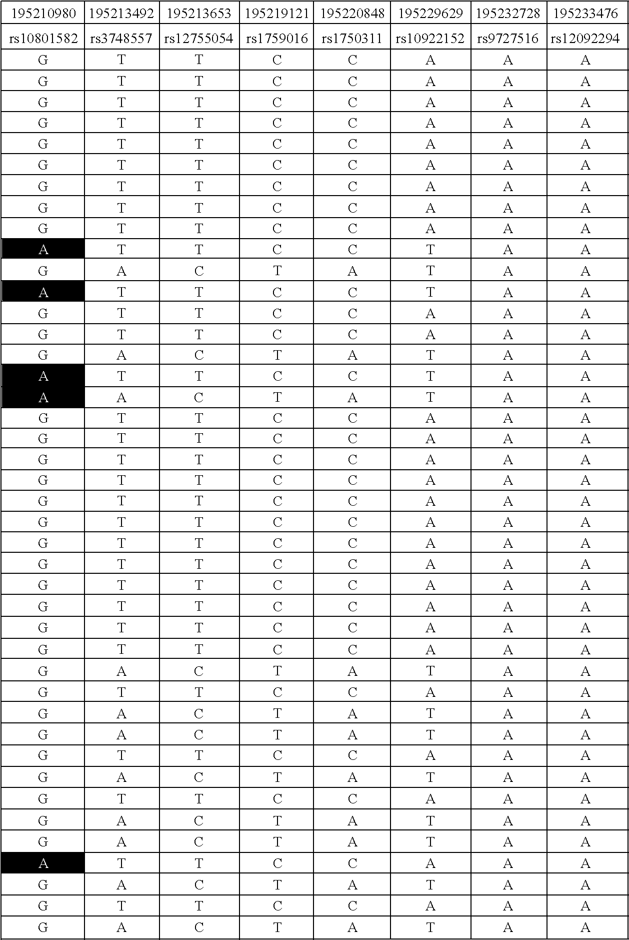

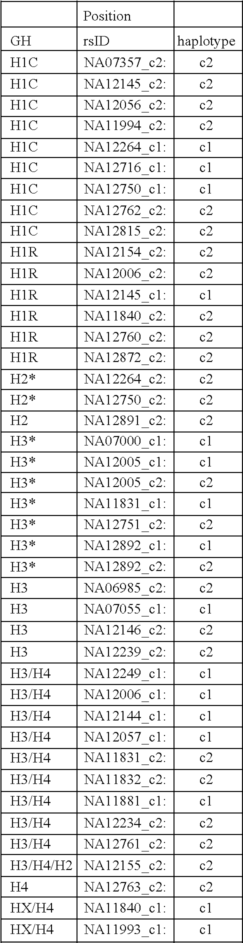

In Table 8 “rs1061170” is disclosed as SEQ ID NO: 16, “rs10922094” is disclosed as SEQ ID NO: 21, “rs12124794” is disclosed as SEQ ID NO: 22, “rs12405238” is disclosed as SEQ ID NO: 23, “rs10922096” is disclosed as SEQ ID NO: 24, “rs10922102” is disclosed as SEQ ID NO: 28, “rs2860102” is disclosed as SEQ ID NO: 29, “rs4658046” is disclosed as SEQ ID NO: 30, “rs12038333” is disclosed as SEQ ID NO: 33, “rs12045503” is disclosed as SEQ ID NO: 34, “rs9970784” is disclosed as SEQ ID NO: 35, “rs1831282” is disclosed as SEQ ID NO: 36, “rs203687” is disclosed as SEQ ID NO: 37, “rs2019727” is disclosed as SEQ ID NO: 38, “rs2019724” is disclosed as SEQ ID NO: 39, “rs1887973” is disclosed as SEQ ID NO: 40, “r6428357” is disclosed as SEQ ID NO: 41, “rs6695321” is disclosed as SEQ ID NO: 43, “rs10733086” is disclosed as SEQ ID NO: 44, “rs1410997” is disclosed as SEQ ID NO: 45, “rs203685” is disclosed as SEQ ID NO: 46, “rs10737680” is disclosed as SEQ ID NO: 48, “rs403846” is disclosed as SEQ ID NO: 17, “rs1409153” is disclosed as SEQ ID NO: 18 and “rs1750311” is disclosed as SEQ ID NO: 20.

TABLE 9
“Hot spot” region associated with recombination of haplotypes within CFH gene.
Region associated with recombination depicted with arrow.

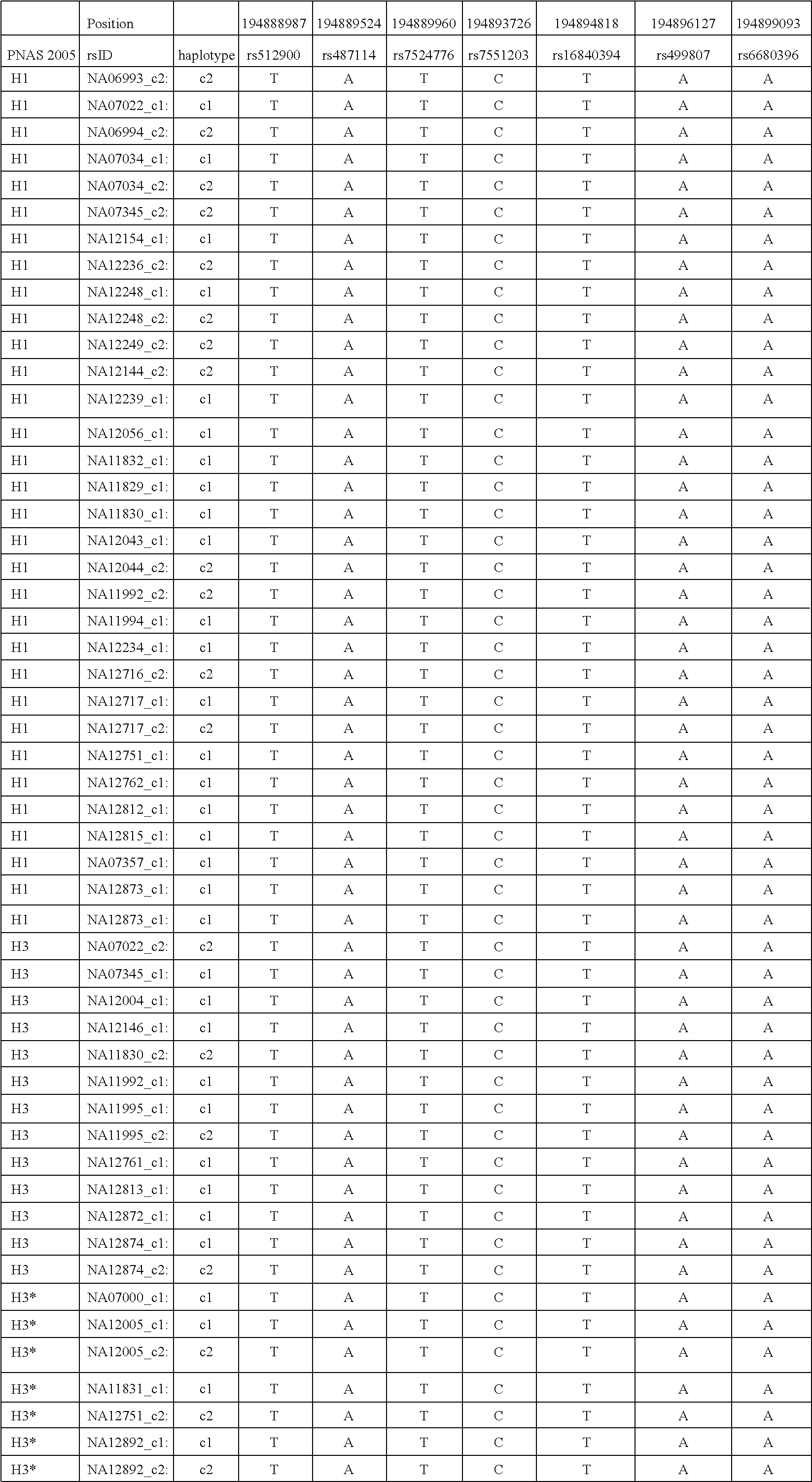

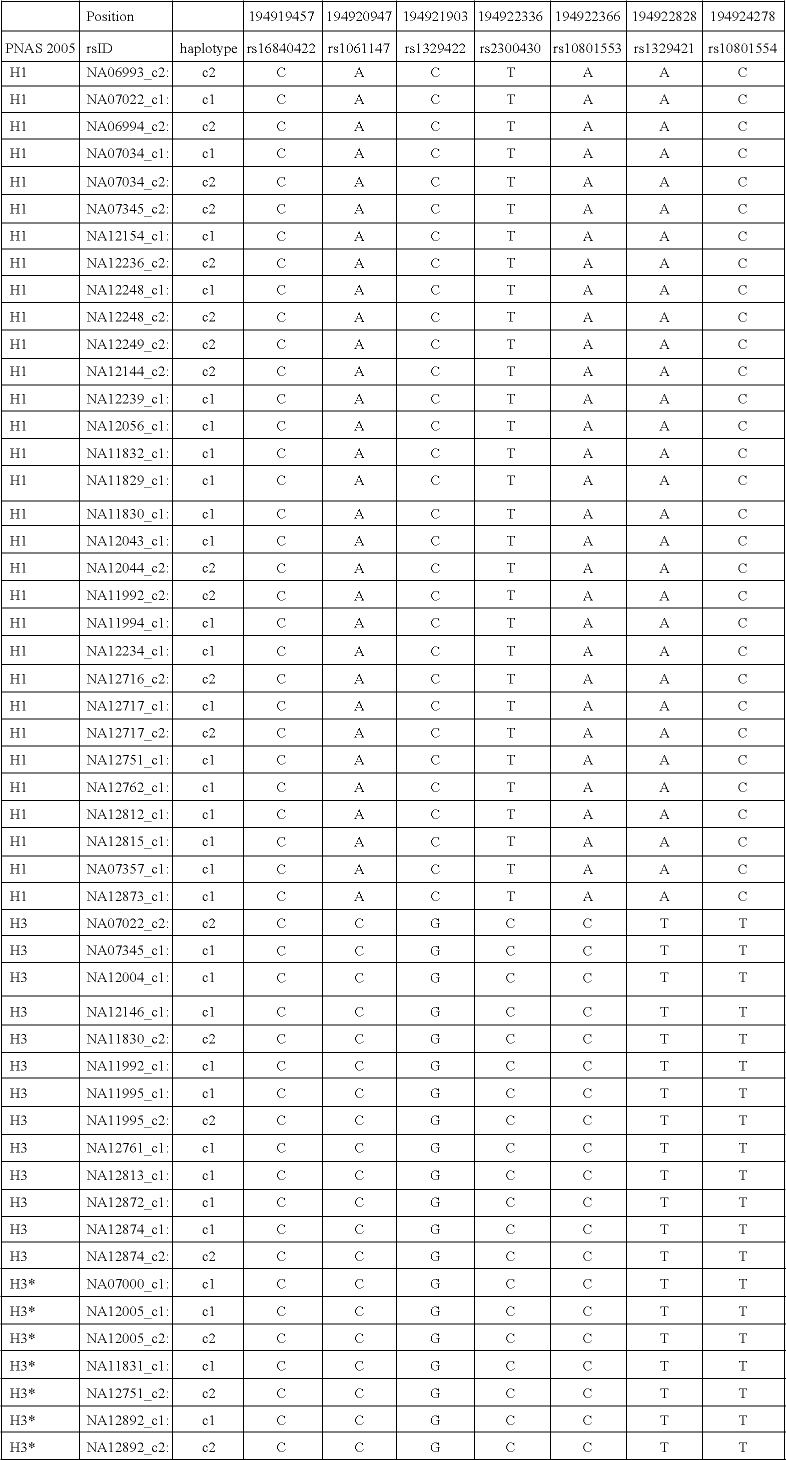

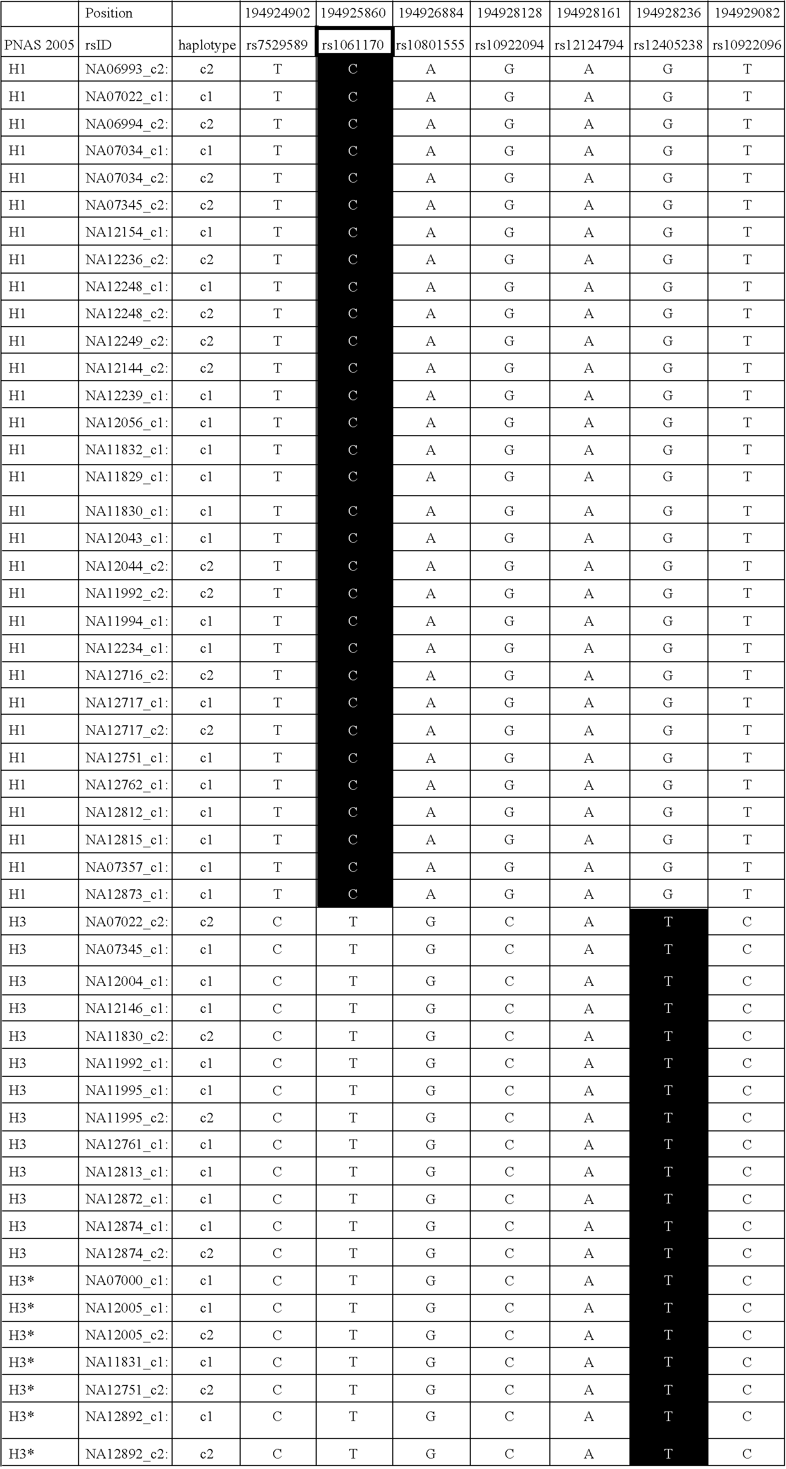

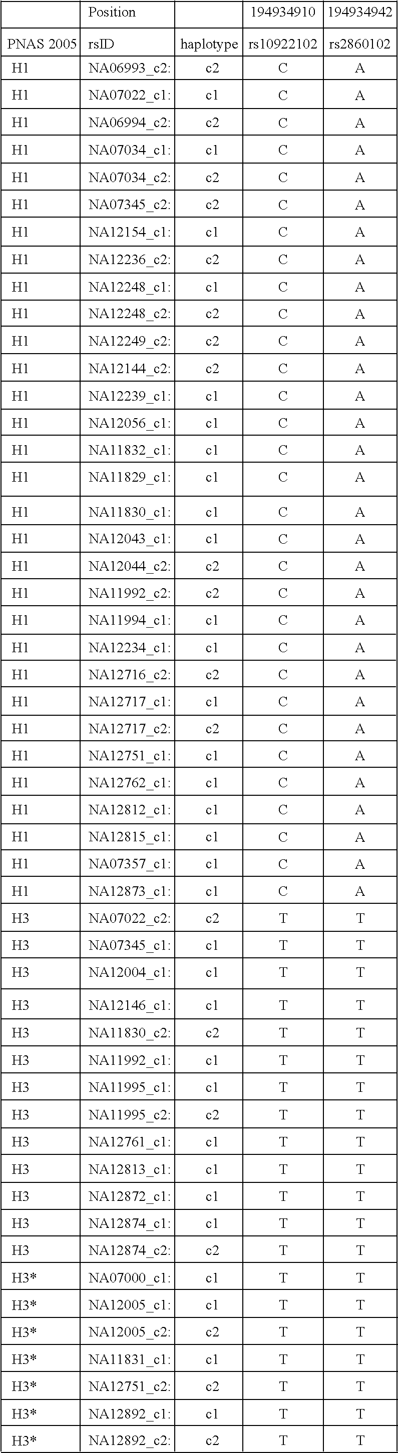

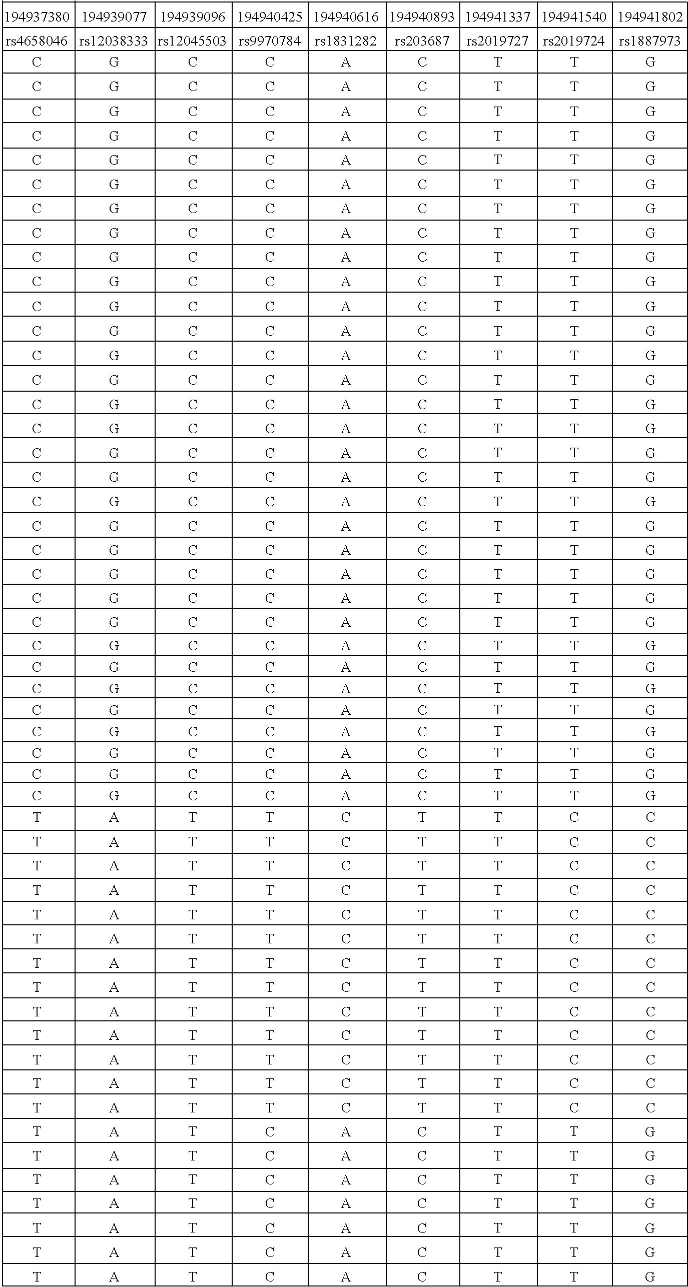

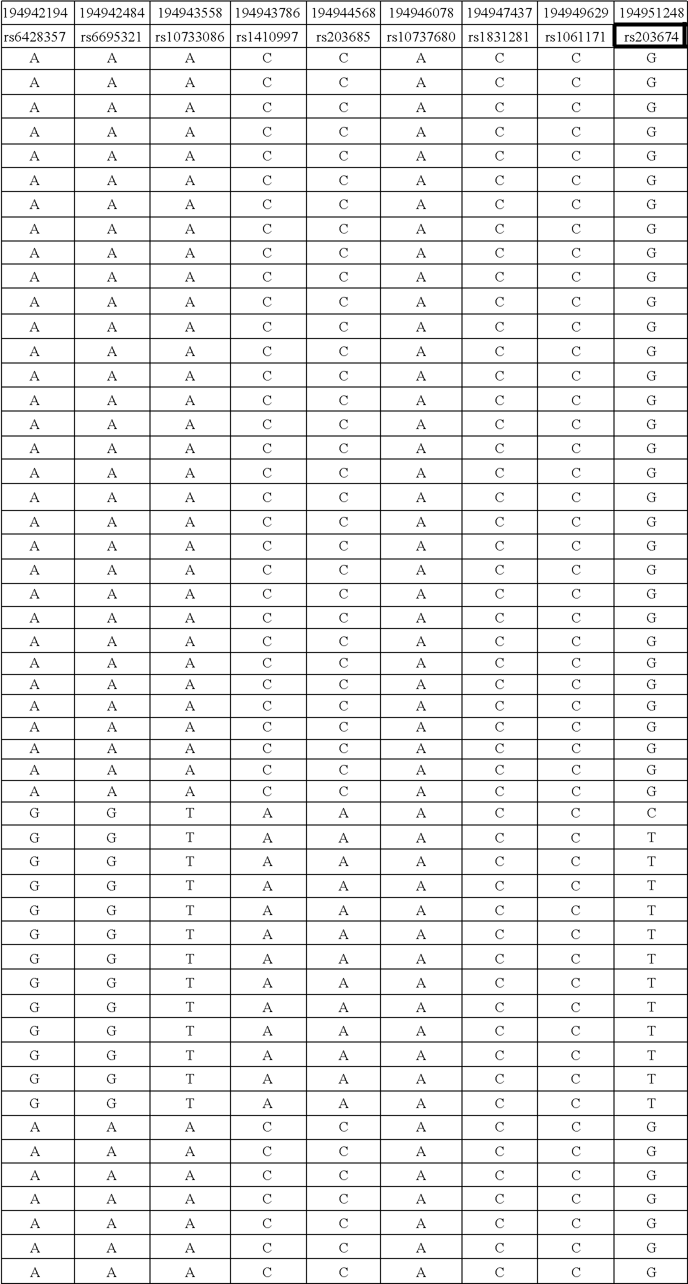

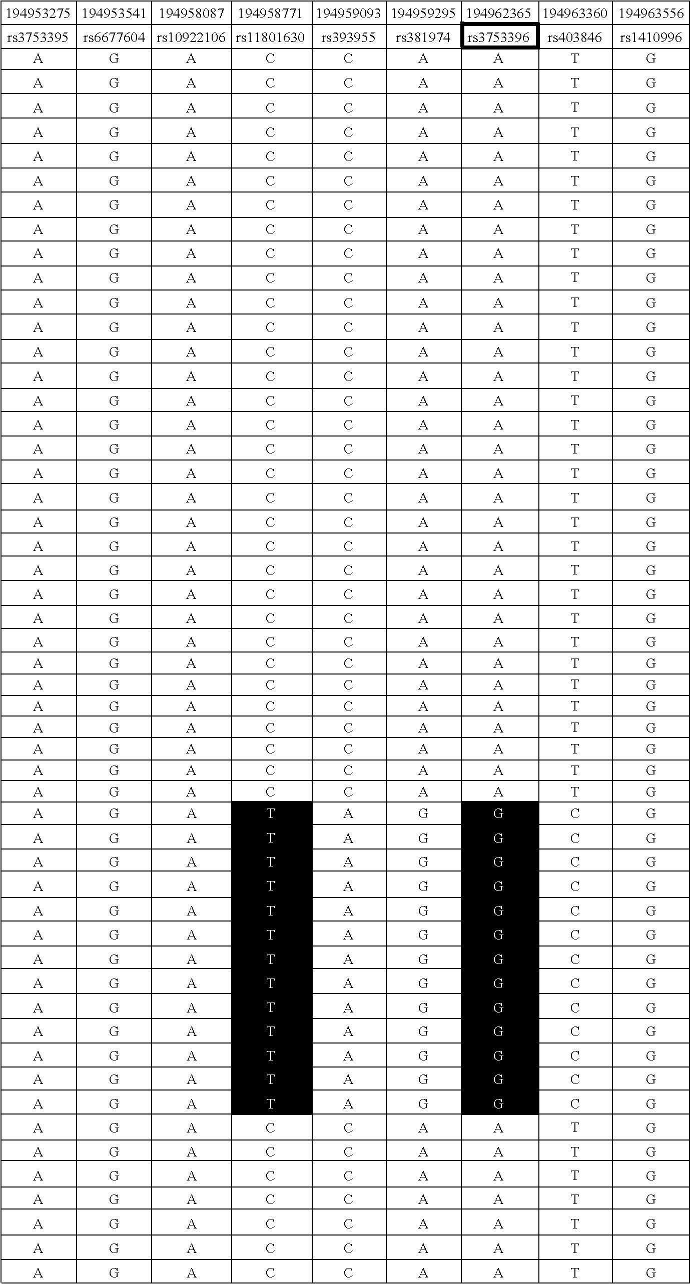

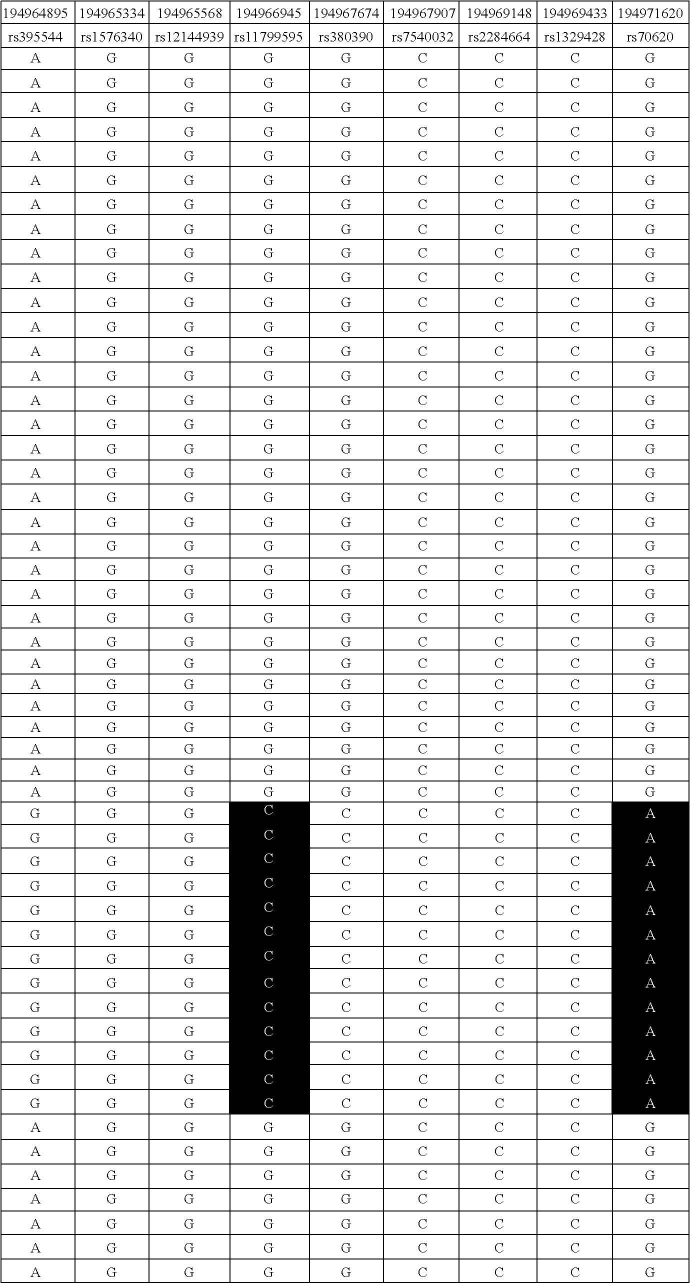

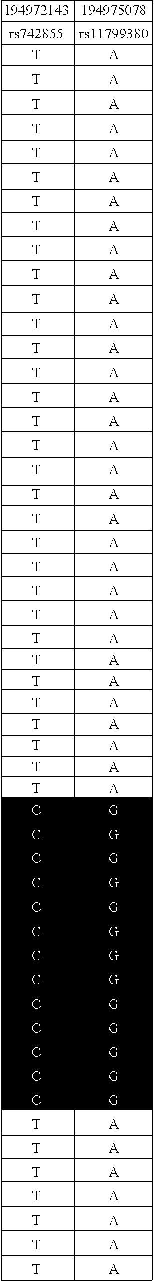

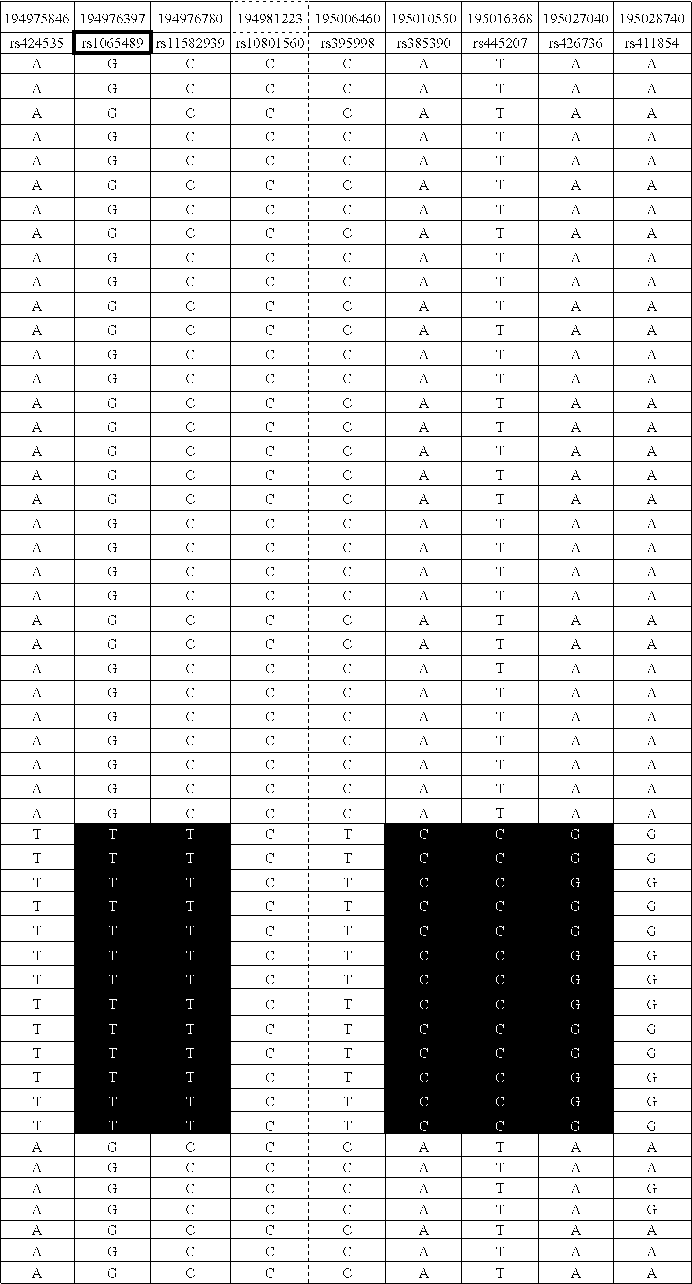

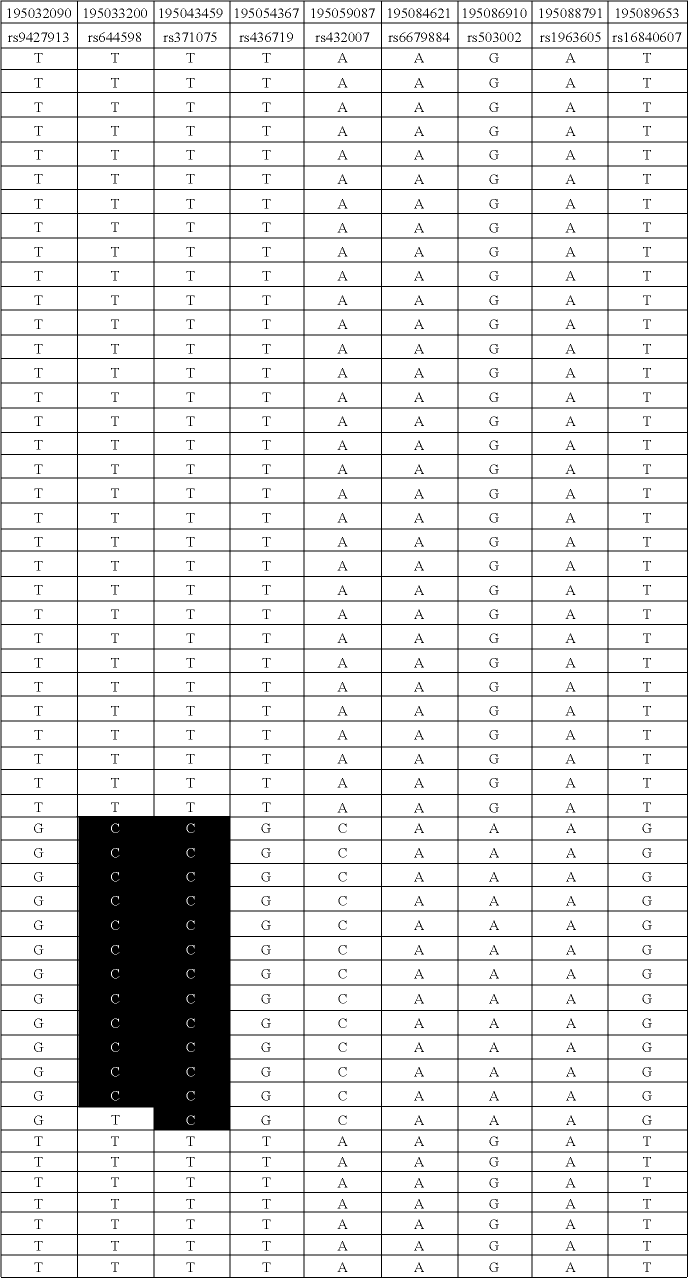

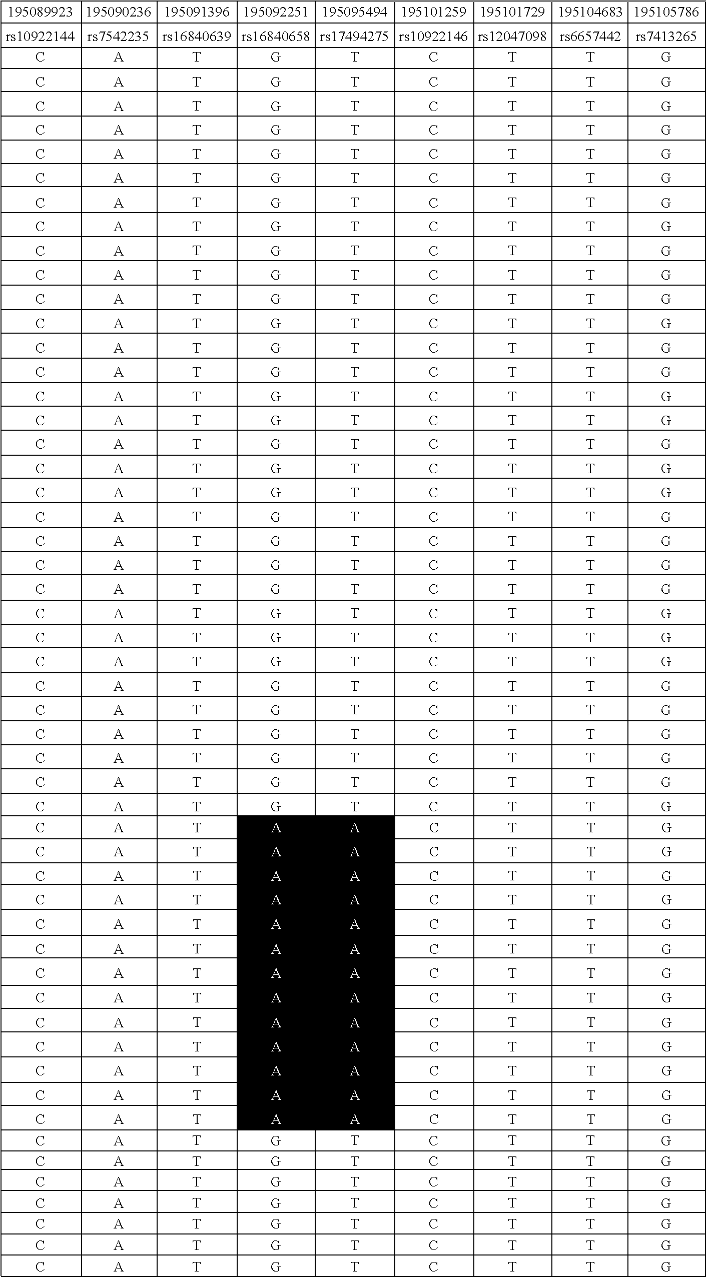

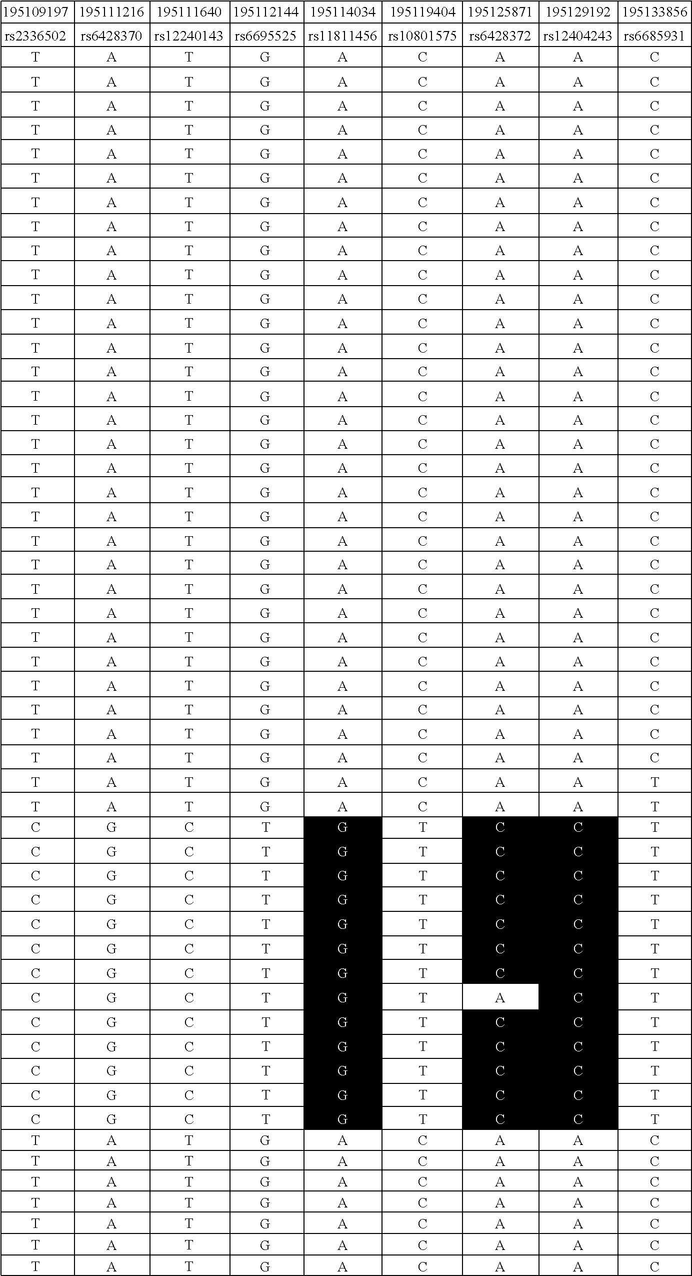

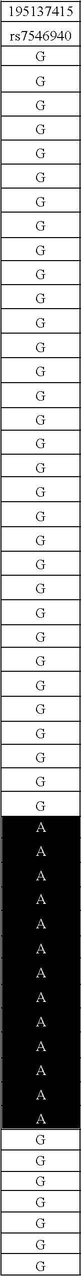

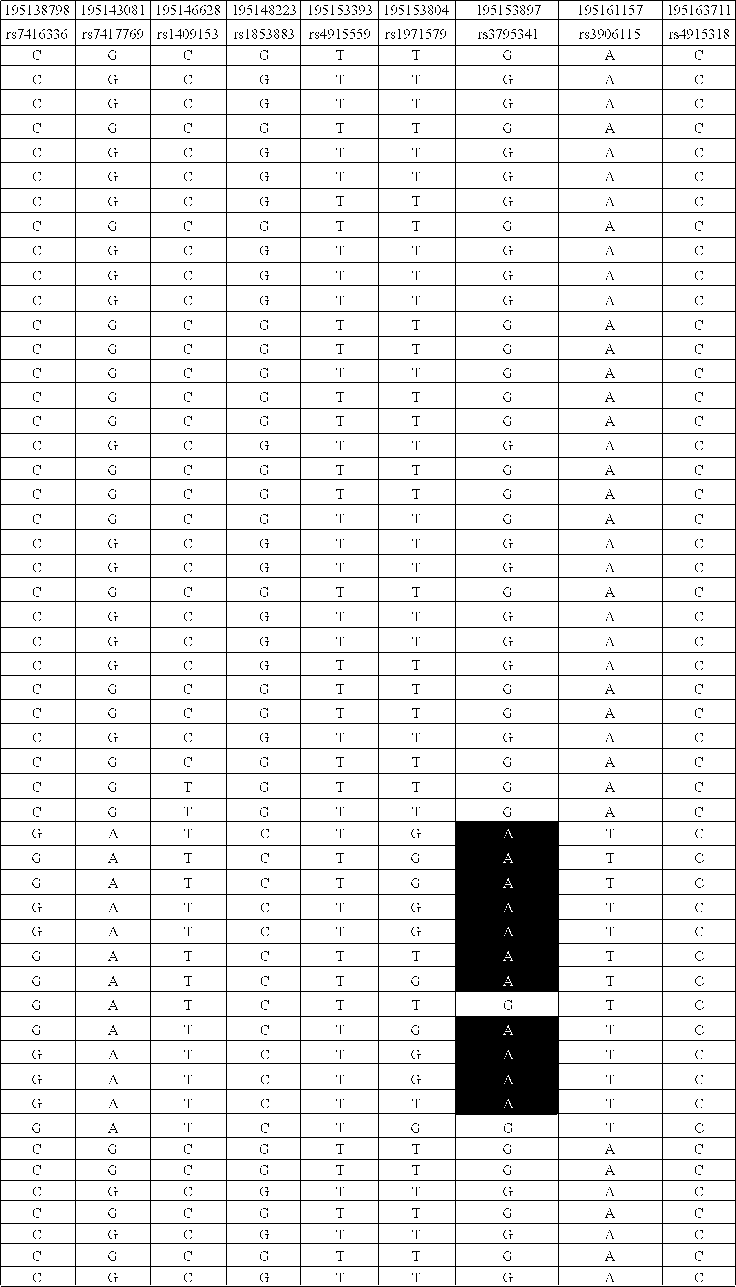

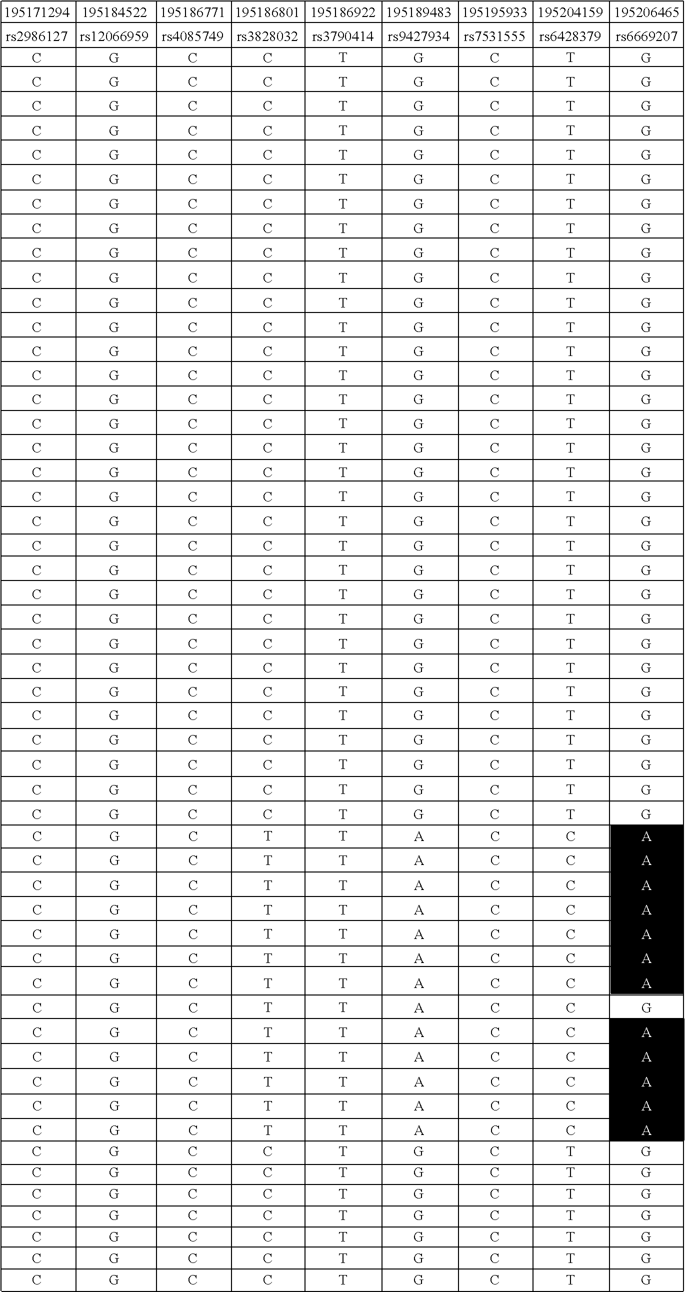

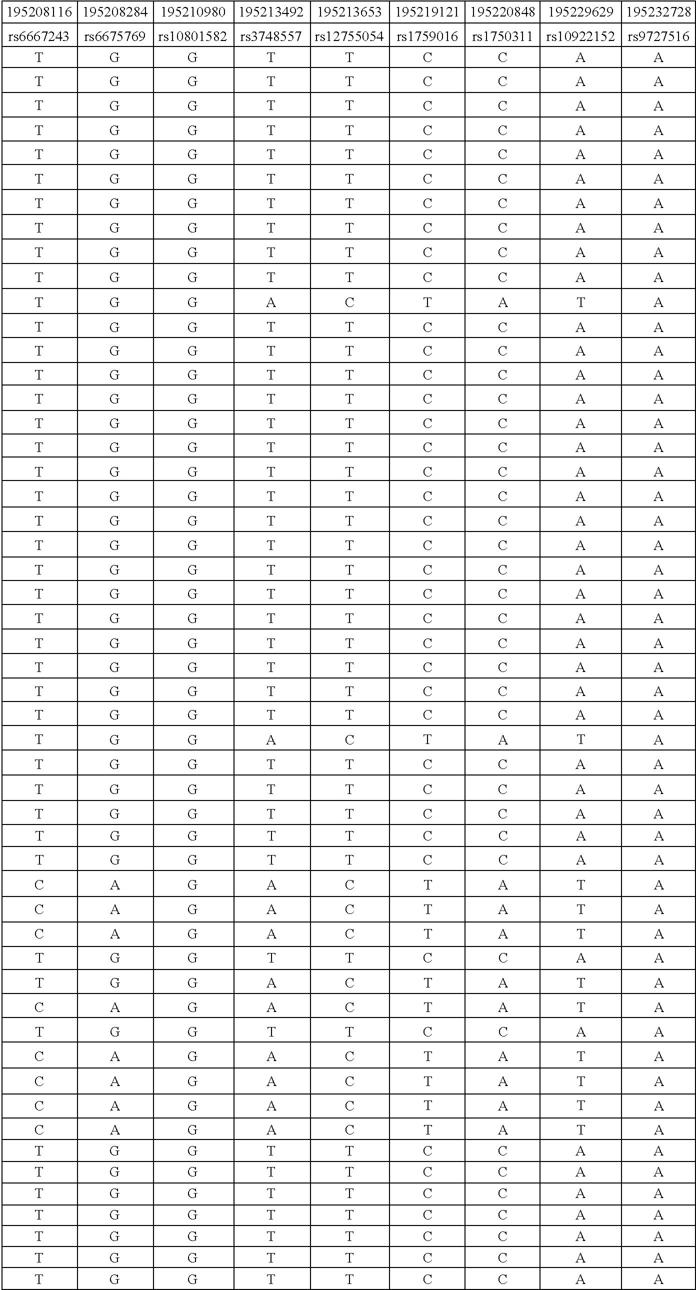

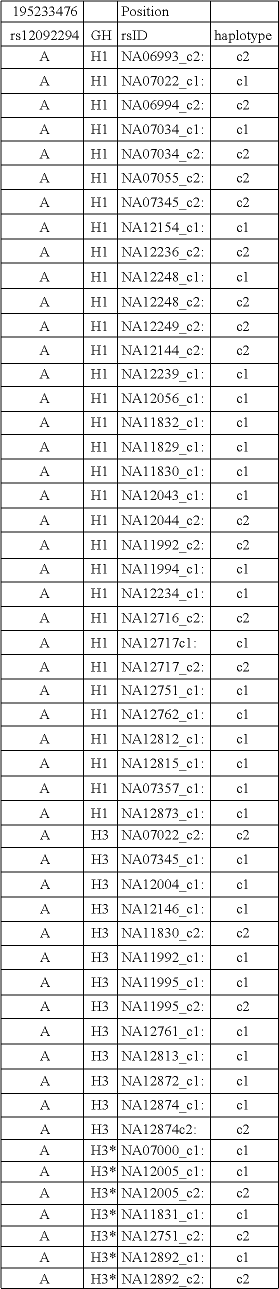

Table 9 shows a collection of HapMap H1 alleles and H3 alleles and collection of chromosomes in between reflecting a haplotype that reveals a shift from H3 at the 5′ end transitioning to an H1 motif at the hotspot location. Chromosome position provided in row 1 are from NCBI Build 36. In Table 9 “rs1061170” is disclosed as SEQ ID NO: 16, “rs10922094” is disclosed as SEQ ID NO: 21, “rs12124794” is disclosed as SEQ ID NO: 22, “rs12405238” is disclosed as SEQ ID NO: 23, “rs10922096” is disclosed as SEQ ID NO: 24, “rs10922102” is disclosed as SEQ ID NO: 28, “rs2860102” is disclosed as SEQ ID NO: 29, “rs4658046” is disclosed as SEQ ID NO: 30, “rs12038333” is disclosed as SEQ ID NO: 33, “rs12045503” is disclosed as SEQ ID NO: 34, “rs9970784” is disclosed as SEQ ID NO: 35, “rs1831282” is disclosed as SEQ ID NO: 36, “rs203687” is disclosed as SEQ ID NO: 37, “rs2019727” is disclosed as SEQ ID NO: 38, “rs2019724” is disclosed as SEQ ID NO: 39, “rs1887973” is disclosed as SEQ ID NO: 40, “r56428357” is disclosed as SEQ ID NO: 41, “rs6695321” is disclosed as SEQ ID NO: 43, “rs10733086” is disclosed as SEQ ID NO: 44, “rs1410997” is disclosed as SEQ ID NO: 45, “rs203685” is disclosed as SEQ ID NO: 46, “rs10737680” is disclosed as SEQ ID NO: 48, “rs403846” is disclosed as SEQ ID NO: 17, “rs1409153” is disclosed as SEQ ID NO: 18 and “rs1750311” is disclosed as SEQ ID NO: 20.

### Example 2

### Evaluation of Discordant HapMap Genotyping Results with Real-Time PCR

Comparison of genotyping results obtained from HapMap phased chromosomes revealed discordant genotyping results in nine samples at SNP rs1061170 (SEQ ID NO: 16) as compared to results obtained on the MassARRAY® Platform (Sequenom, Inc. San Diego Calif.) and by standard Sanger dideoxy Sequencing. MassARRAY assay designs are provided below. In all cases, the genotyping results obtained on MassARRAY and by Sequencing generated a CC result for each of the nine samples that were reported as CT in the HapMap database for rs1061170 (SEQ ID NO: 16). This SNP is in linkage disequilibrium with rs1061147 (see Table 10), and the expected genotype for these nine samples is CC (as rs1061147 genotypes as AA for these individuals), further confirming the genotyping results by MassARRAY and sequencing. The rs1061170 (SEQ ID NO: 16) SNP identifies the Y402H variant, which is significantly associated with AMD ((Klein, R. J. et al. Complement factor H polymorphism in age-related macular degeneration. Science (2005) 308, 385-389; Edwards, A. O. et al. Complement factor H polymorphism and age-related macular degeneration. Science (2005) 308, 421-424; Haines, J. L. et al. Complement factor H variant increases the risk of age-related macular degeneration. Science (2005) 308, 419-421; Zareparsi, S. et al. Strong association of the Y402H Variant in Complement Factor H at 1q32 with Susceptibility to Age-Related Macular Degeneration. Am. J. Hum. Genet. (2005) 77; Hageman, G. S. et al. A common haplotype in the complement regulatory gene factor H(HF1/CFH) predisposes individuals to age-related macular degeneration. Proc. Natl. Acad. Sci. (2005) U.S.A 102[20], 7227-7232)). The nine discordant samples along with other samples with other genotypes for control purposes were then subjected to a real-time qPCR assay to detect relative copy numbers of the C and T alleles present at rs1061170 (SEQ ID NO: 16).

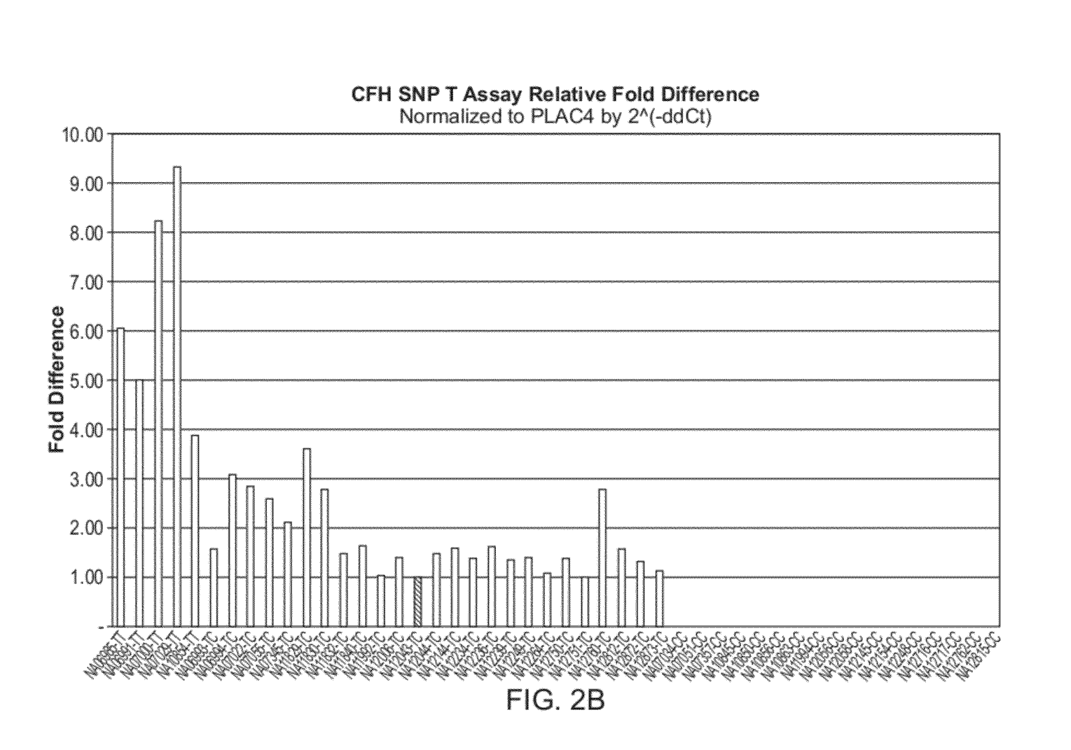

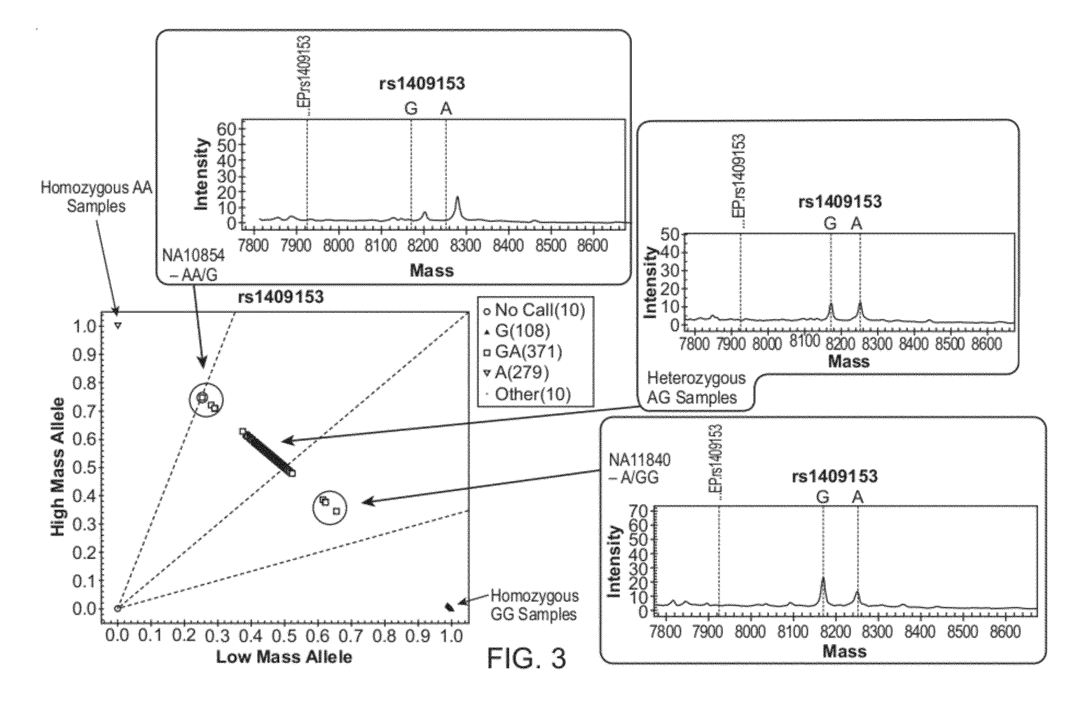

Real-time qPCR using Taqman probes for rs1061170 (SEQ ID NO: 16) was conducted based on the manufacturer's recommendations found in the manuals (Life Technologies (formerly Applied Biosystems), using the Viia7 Real-Time Cycler and softwre. The primers and conditions for this assay are described below. The real-time qPCR assay was designed to interrogate the variant C/T position at rs1061170 (SEQ ID NO: 16) using Taqman probes for each allele respectively. Each sample was also measured with a 2N reference assay in the PLAC4 gene (Chromosome 21) in order to normalize for inter-sample variations. A second level of normalization was applied using a 1N reference sample (NA12043) for the given rs1061170 (SEQ ID NO: 16) variant under study. The sample is heterozygous for the SNP (one copy of the C and T allele each) and had the highest C, Fold difference was calculated using the 44C, method (2001, Pfaffl). The 44C, data for the rs1061170 (SEQ ID NO: 16) qPCR assay are shown in FIG. 2A (C allele) and FIG. 2B (T allele). The data was generated from quadruplicate reactions per sample and the ΔΔCt shown represents the mean of those observations after normalization. The X-axis lists sample ID and genotype and the Y-axis the relative difference between samples based on normalization to PLAC4 then to NA12043 (note its value is 1). The samples segregate into two major groups based on genotype. The heterozygous samples (CT) all have ratio between 1-approximately 2.5 relative to NA12043; whereas homozygous samples (CC) all exhibit a ratio greater than three with a mean close to 5. Six homozygous samples (NA07034, NA07051, NA07357, NA10850, NA10863, and NA12058) in particular exhibited the highest fold difference when compared to the reference sample. The data clearly show that 1N heterozygous individuals and 2N (or 3N) homozygous individuals can be distinguished. It is also highly suggestive that NA07034 in particular may carry and extra C allele. The assay is clearly specific as TT homozygous samples did not produce a signal when only the C probe was used in the reaction. Additionally, seven of the nine samples that had the correct “discordant” CT genotyping revealed no signal in the T-variant assay. This suggests the discordant typing in the HapMap database was due to cross hybridization of highly homologous regions (e.g. CFHR3) due to a low stringency assay artifact present in the rs1061170 (SEQ ID NO: 16) IIlumina genotyping assay. Two discordant samples that were typed as H1/H2 haplotypes revealed the expected CT typing, thereby indicating that the C and T assignment at rs1061170 (SEQ ID NO: 16) across the two alleles was likely due to phase assignment errors. Similar results were obtained using the T allele probe in terms of clear identification of 1N heterozygous samples vs 2N (or 3N) homozygous samples (FIG. 2B). In particular, sample NA07029 appears to be an example of a 3N individual. The association between the discordant typing observed in H1/H1 homozygous HapMap samples and the presence of a copy number variant, however, seemed to reveal a lower association, although additional analysis was necessary to confirm the boundaries and the dimension of the copy number variant across the CFH-CFHR5 region.

An additional piece of data related to CNV across this collection of samples was obtained in samples NA11840 and NA10854 at SNP rs1409153 (SEQ ID NO: 18) in CFHR4. The MassARRAY platform is highly sensitive for the detection copy number variants when samples are in an unbalanced heterozygous status. Therefore it was used to investigate the rs1409153 (SEQ ID NO: 18) SNP is CFHR4. The results are shown in FIG. 3. It shows an extra allele detected for these two samples. The ability to detect a CNV in the region surrounding rs1409153 (SEQ ID NO: 18) in CFHR4 indicated there might be multiple copy number variants present across this region containing highly homologous genes.

| | | | | | | | | | | | | | | |
|---|---|---|---|---|---|---|---|---|---|---|---|---|---|---|
| H1C | NA07357_c2: | c2 | T | A | T | C | T | A | A | G | T | A | T | C |
| H1C | NA12145_c2: | c2 | T | A | T | C | T | A | A | G | T | A | T | C |
| H1C | NA12056_c2: | c2 | T | A | T | C | T | A | A | G | T | A | T | C |
| H1C | NA11994_c2: | c2 | T | A | T | C | T | A | A | G | T | A | T | C |
| H1C | NA12264_c1: | c1 | T | A | T | C | T | A | A | G | T | A | T | C |
| H1C | NA12716_c1: | c1 | T | A | T | C | T | A | A | G | T | A | T | C |
| H1C | NA12750_c1: | c1 | T | A | T | C | T | A | A | G | T | A | T | C |
| H1C | NA12762_c2: | c2 | T | A | T | C | T | A | A | G | T | A | T | C |
| H1C | NA12815_c2: | c2 | T | A | T | C | T | A | A | G | T | A | T | C |

| | | | | | | rs1061147 | | | | | | | rs1061170 |
|---|---|---|---|---|---|---|---|---|---|---|---|---|---|
| H1C | NA07357_c2: | c2 | A | T | C | A | C | T | A | A | C | T | T |
| H1C | NA12145_c2: | c2 | A | T | C | A | C | T | A | A | C | T | T |
| H1C | NA12056_c2: | c2 | A | T | C | A | C | T | A | A | C | T | T |
| H1C | NA11994_c2: | c2 | A | T | C | A | C | T | A | A | C | T | T |
| H1C | NA12264_c1: | c1 | A | T | C | A | C | T | A | A | C | T | T |
| H1C | NA12716_c1: | c1 | A | T | C | A | C | T | A | A | C | T | T |
| H1C | NA12750_c1: | c1 | A | T | C | A | C | T | A | A | C | T | T |
| H1C | NA12762_c2: | c2 | A | T | C | A | C | T | A | A | C | T | T |
| H1C | NA12815_c2: | c2 | A | T | C | A | C | C | A | A | C | T | T |

Table 10 provides genotyping results from a collection of 9 HapMap samples that reveal discordant genotyping at SNP rs1061170 (SEQ ID NO: 16). More specifically, it identifies 9 HapMap H1/H1 homozygotes with an artifact at CFH 1277 C showing “T” instead of “C” in otherwise identical H1 samples. Thus, there is a loss of LD between the two SNPs.

### MassARRAY Genotyping and CNV Analysis—Materials and Methods

MassARRAY genotyping for rs1061170 (SEQ ID NO: 16) and rs1409153 (SEQ ID NO: 18) was performed as previously described (2009, Oeth et al) with the exception that Thermosequenase DNA Polymerase (GE Healthcare) was substituted for iPLEX® enzyme. The primer sets for these two assays are shown in Figure X. Identification of samples carrying extra copies of either allele as found in the rs1409153 (SEQ ID NO: 18) assay were identified using cluster-based algorithm for MassARRAY data (2009, Oeth et al).

A. rs1061170 (SEQ ID NO: 16)—MassARRAY

Forward PCR:
(SEQ ID NO: 1)
5′-[ACGTTGGATG]GTTATGGTCCTTAGGAAAATG-3′
Reverse PCR:
(SEQ ID NO: 2)
5′-[ACGTTGGATG]ACGTCTATAGATTTACCCTG-3′
Extend:
(SEQ ID NO: 3)
5′-CTGTACAAACTTTCTTCCAT-3′

### Template:

(SEQ ID NO: 4)
CTTTGTTAGTAACTTTAGTTCGTCTTCAGTTATACATTATTTTTGGATGTTTATGCAATCTTAT
TTAAATATTGTAAAAATAATTGTAATATACTATTTTGAGCAAATTTATGTTTCTCATTTACTTTA
TTTATTTATCATTGTTATGGTCCTTAGGAAAATGTTATTTTCCTTATTTGGAAAATGGATATA
ATCAAAAT[C/T]ATGGAAGAAAGTTTGTACAGGGTAAATCTATAGACGTTGCCTGCCATCCT
GGCTACGCTCTTCCAAAAGCGCAGACCACAGTTACATGTATGGAGAATGGCTGGTCTCCTA
CTCCCAGATGCATCCGTGTCAGTAAGTACACTACTCTGAAATCCTAGCATGTTCATGTCTTT
CTAAGTAACATAGATGACATTCTAAG

B. rs1409153 (SEQ ID NO: 18)—MassARRAY

Forward PCR:
(SEQ ID NO: 5)
5′-[ACGTTGGATG]GACCATAAAATGATTAAAAGG-3′
Reverse PCR:
(SEQ ID NO: 6)
5′-[ACGTTGGATG]GTACTGATGCAGTCTTATTT-3′
Extend:
(SEQ ID NO: 7)
5′-TATACTATTTTGATCAAATTCATGTT-3′

### Template:

(SEQ ID NO: 8)
TTTACAGATTGACTCTGTAAAGATATTCCTTCATATTTTGTGTTATATCCATTCTCCAAATAAC
TGAGAATACATTGTCCTAAAGACCATAAAATGATTAAAAGGTAGATTAG[A/G]AACATGAAT
TTGATCAAAATAGTATATTAAAATAATTTTTTGAATATTTAAATAAGACTGCATCAGTACACA
AAAATGACGTATCACTGAAGGAAAACTAAAGCTACTACTAAATGTTTGTACAAAAAGGTCAG
TATTCAATGTTACTTATCTTTAGTTTTTATGATAAAATATGTTTAAATTATATAGGTATTCTCAT
AAGGTTCCTATATTTATTTCTCATGTGATTTTCATGAAGGTCTCATAACAGAAAAGATCTAGT
TTGGTGTTTTTGCATGAACAACTCTTCCTTTGGTACCATCTCTGTCATATAAGACAATGTAAT
CATTTGTTTGCTCTTCTCTCTCCATTCTTTGCAAGTTTTATGCACATATTGTTGTAAAGAGGT
TTGCTTACTGAGGCATGGGACTGTTGGCAACCACCCATCTTGTGTGCAGTGAATGTAATCC
CAGTAACTTCCTGAAGGAGTCACAAAATTTTGGTCACAGTAATAGGAGTAAGATTGTC

PCR primers and primer extension primers are depicted along with the target template for each assay respectively. Bold letters within the target sequence denote the PCR primers and the underlined sequence the extend primer. Primer sequence in brackets [ACGTTGGATG (SEQ ID NO: 9)] represents a universal tag sequence that improves multiplexing.

### TaqMan CNV Analysis—Materials and Methods

Real-time qPCR Primers for the rs1061170 (SEQ ID NO: 16) Copy Number Detection are provided below:

rs1061170 (SEQ ID NO: 16)—Taqman

Forward PCR:
(SEQ ID NO: 10)
5′- TTCCTTATTTGGAAAATGGATATAA -3′
Reverse PCR:
(SEQ ID NO: 11)
5′- GCAACGTCTATAGATTTACCCTGT -3′
C - Probe:
(SEQ ID NO: 12)
5′- FAM6-TTTCTTCCATGATTTTGA-MGBNFQ -3′
T - Probe:
(SEQ ID NO: 13)
5′- VIC-ACTTTCTTCCATAATTTTGA-MGBNFQ -3′

### C Allele:

(SEQ ID NO: 14)
CTTTGTTAGTAACTTTAGTTCGTCTTCAGTTATACATTATTTTTGGATGTTTATGCAATCTTAT
TTAAATATTGTAAAAATAATTGTAATATACTATTTTGAGCAAATTTATGTTTCTCATTTACTTTA
TTTATTTATCATTGTTATGGTCCTTAGGAAAATGTTATTTTCCTTATTTGGAAAATGGATATA
ATCAAAAT[C/T]ATGGAAGAAAGTTTGTACAGGGTAAATCTATAGACGTTGCCTGCCATCCT
GGCTACGCTCTTCCAAAAGCGCAGACCACAGTTACATGTATGGAGAATGGCTGGTCTCCTA
CTCCCAGATGCATCCGTGTCAGTAAGTACACTACTCTGAAATCCTAGCATGTTCATGTCTTT
CTAAGTAACATAGATGACATTCTAAGA

### T Allele:

(SEQ ID NO: 15)
CTTTGTTAGTAACTTTAGTTCGTCTTCAGTTATACATTATTTTTGGATGTTTATGCAATCTTAT
TTAAATATTGTAAAAATAATTGTAATATACTATTTTGAGCAAATTTATGTTTCTCATTTACTTTA
TTTATTTATCATTGTTATGGTCCTTAGGAAAATGTTATTTTCCTTATTTGGAAAATGGATATA
ATCAAAAT[C/T]ATGGAAGAAAGTTTGTACAGGGTAAATCTATAGACGTTGCCTGCCATCCT
GGCTACGCTCTTCCAAAAGCGCAGACCACAGTTACATGTATGGAGAATGGCTGGTCTCCTA
CTCCCAGATGCATCCGTGTCAGTAAGTACACTACTCTGAAATCCTAGCATGTTCATGTCTTT
CTAAGTAACATAGATGACATTCTAAGA

PCR primers and Taqman Probe primers are depicted along with the target template for each allele respectively. Bold letters within the target sequence denote the PCR primers and the underlined sequence the Taqman probe sequences. Assays were amplified for 45 cycles with a denaturation temperature of 95° C. and an annealing of 60° C. using Taqman Mastermix (Life Technologies) and 50 ng g DNA in a 25 ul reaction.

### Example 3

### Use of 1000 Genomes Project Next-Generation Sequencing Data to Detect CNVs

In order to confirm the presence of the copy number variant, a survey of short read aligned sequencing data extracted from the 1000 Genome Project database was performed on subjects tested with the TaqMan CNV assay and identified with the putative CFH copy number variant. The plotted aligned short read data for each subject was reviewed as a custom track in the UCSC genome browser and evaluated for gross deletions and copy number variants across the CFH-CFHR5 region. A deletion would be identified as a dip (or decrease) in the middle of the sequence read alignments, while a copy number variant would present as a peak (or increase) of additional reads. Next-generation sequencing technologies, such as the Illumina Solexa method (Bentley, et al 2008) have shown utility for CNV detection, based on variation in sequencing coverage, (depth of coverage (DOC) analysis), across a reference genome (Yoon et al 2009). CNV-calling algorithms are available which enable CNV-calling directly from next generation sequencing data files (Yoon et al 2009; Yie et al 2009); however, these tools require local availability of datafiles, which average around 5-10 Gb per subject and are impractical to download (A 5 Gb file takes ˜10 hrs to download from the 1000 Genomes FTP site). One practical alternative method for detection of putative CNVs across multiple subjects is to remotely access BAM format files using the UCSC custom track service. Confirmation of the CNVs detected can be confirmed using CNV calling algorithms.

BAM is the compressed binary version of the Sequence Alignment/Map (SAM) format, a compact and index-able representation of nucleotide sequence alignments. Many next-generation sequencing and analysis tools work with SAM/BAM. The UCSC genome browser allows custom track display of BAM files. As the files are indexed this allows limited transfer of the portions of the files that are needed to display a particular region. This makes it possible to display alignments from files that are so large that the connection to UCSC would time-out when attempting to upload the whole file to UCSC. Both the BAM file and its associated index file remain on the web-accessible server, not on the UCSC server. UCSC temporarily caches the accessed portions of the files to speed up interactive display allowing simultaneous viewing and comparison of 10s of subjects.

By reviewing the 1000 Genomes sequence read alignments, evidence of novel, large (˜20 kb) copy number variants present across the RCA region was identified.

### Genomic Characterization

Primary genomic characterisation of the CFH locus was carried out using the UCSC genome browser (http://genome.ucsc.edu/). Coordinates in the report are based on both NCBI36 and NCBI37 and are clearly indicated. Data from the 1000 Genomes project is reported using NCBI37 coordinates. The key regions for analysis were as follows:
1) RCA cluster, including CFH, CFHR3, CFHR1, CFHR4, CFHR2 and CFHR5 wider region spanning
   a. NCBI36: chr1:194852460-195233425
   b. NCBI37: chr1:196585837-196966802
2) CFH peak association, including rs1061170 (SEQ ID NO: 16), rs10737680 (SEQ ID NO: 48), Exon 9, Intron 9, Exon 10, Intron 10
   a. NCBI36: chr1:194896799-194954998
   b. NCBI37: chr1:196630176-196688375

### CNV Databases

3) The Database of Genomic Variation (DGV) (Universal resource locator (URL) projects.tcag.ca/variation/) was used as a reference for known CNVs across the CFH and wider RCA locus. The database is also available to view as a track at the UCSC genome browser.

### HapMap Data

4) HapMap data (Universal resource locator (URL) hapmap.org) across the CFH locus was reviewed and used to group subjects by genotype and haplotype. These groupings were used to select subjects for review in 1000 Genomes data, based on a review of phased data for the CFH-CFHR5 region sorted by the 6 of 8 CFH haplotype SNPs described by Hageman et al. (2005).

### 1000 Genomes Project Data

5) Data from the 1000 Genomes project is accessible at (Universal resource locator, (URL) world wide web.1000genomes.org/page.php.
6) BAM format sequence read alignment files for each individual subject are available at ftp://ftp-trace.NCBI.nih.gov/1000genomes/ftp/data/

Using DOC analysis of short read aligned sequencing data it is possible to identify copy number variants in the genome observed as increased depth of coverage across a given region. However, there is a high level of noise in the alignments which may obscure signal from CNV copy number variants. By their nature, a single copy number variant may be harder to detect as it would involve a 33% increase in signal from 2N to 3N, in comparison to a 50% signal decrease from 2N to 1N in a single deletion. It is also worth noting that known CNV boundaries are mostly defined by array cGH which may be inaccurate. The region of increased read depth identified with DOC analysis may present as a smaller CNV than reported with cGH, raising the possibility that the CNV is actually smaller than reported. Finally, some caution needs to be taken when interpreting increased depth of reads in regions with high GC ratios as there have been some reports of GC-bias among Solexa sequencing reads (Quail et al, 2008).

### Example 4

### Results of 1000 Genomes BAM Data Files and Formatting of UCSC Custom Tracks

In order to allow detailed analysis and comparison of each CFH haplotype, the 184 CEU HapMap subjects with phased data for the CFH-CFHR5 region sorted by the 6 of 8 SNPs described by Hageman et al. (2005), were searched for 1000 Genomes BAM file availability. 92 subjects had Illumina (Solexa) BAM file data available at various levels of sequence read coverage. Analysis-ready UCSC custom tracks were prepared for each subject and loaded to the UCSC genome browser. A file containing these custom tracks is available in Appendix A. BAM file-size is indicated for each subject, giving a relative measure of chromosome-wide read depth. Overall variability of read depth between subjects is due to variation in draft read depth. Two additional subjects with copy number variants in CFH reported in the DGV database are also included for reference (DGV9384, DGV9385).

Two possible duplicated regions (CNV1 & CNV2) are apparent in most of the subjects evaluated. The apparent boundary of CNV1 is located ˜2 Kb 3′ of RS1061170 (SEQ ID NO: 16), however precise boundaries of the putative copy number variant cannot be determined, therefore it is possible that RS1061170 (SEQ ID NO: 16) lies within CNV1. The copy number variants are also seen clearly in the Yoruba subject carrying DGV9385, this subject also appears to carry the protective CFHR3/CFHR1 deletion (DGV 38122). Table 13 below provides possible locations of CNV1 and CNV2 within the RCA locus.

TABLE 13

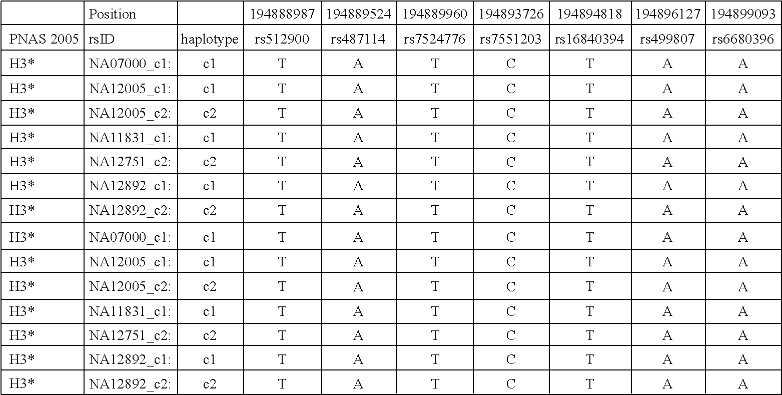

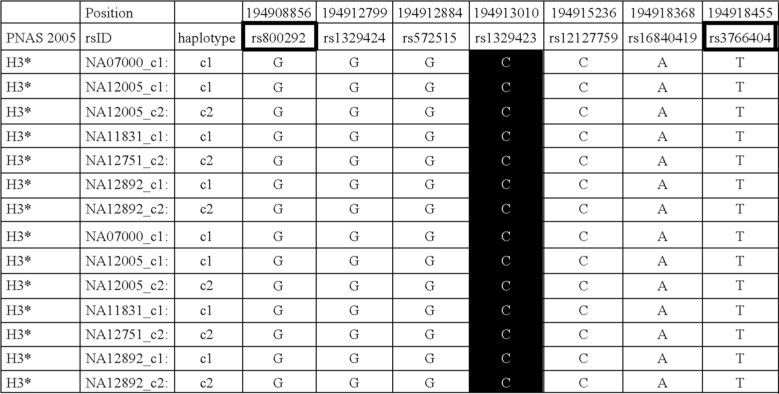

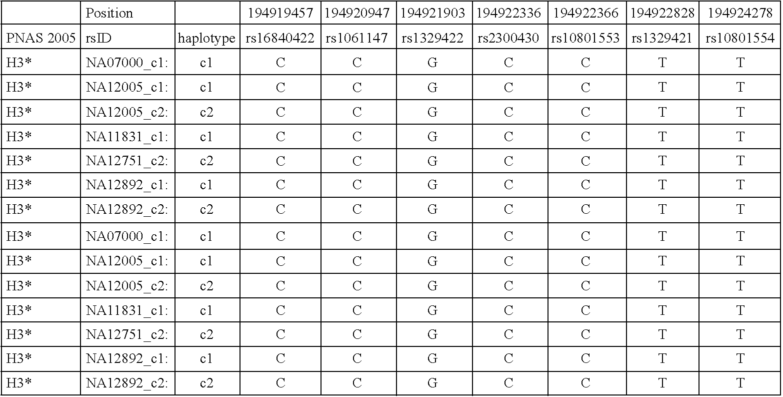

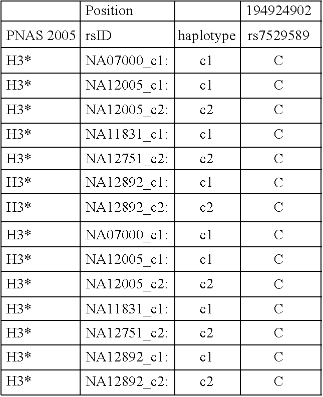

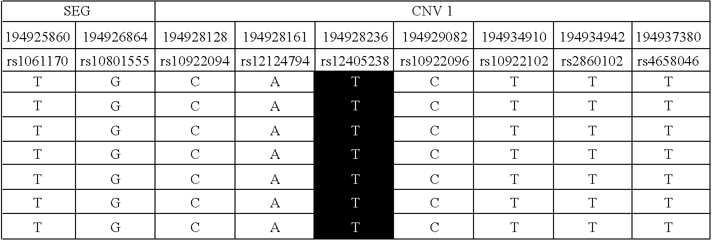

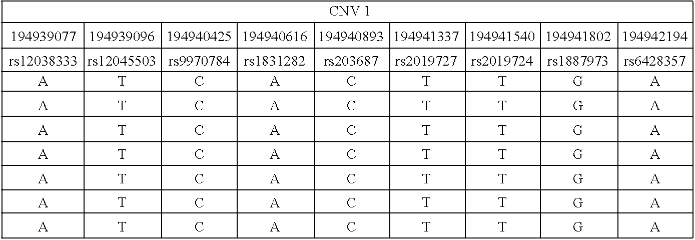

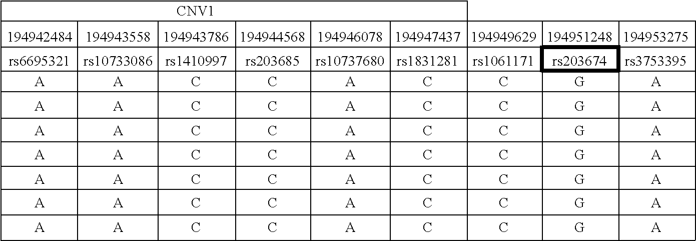

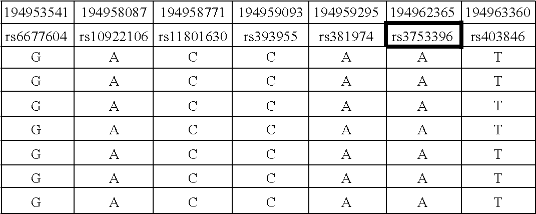

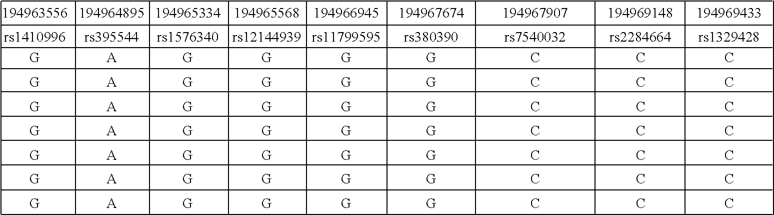

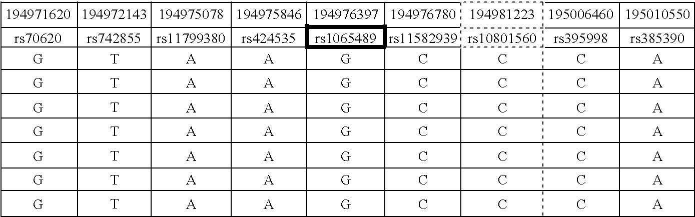

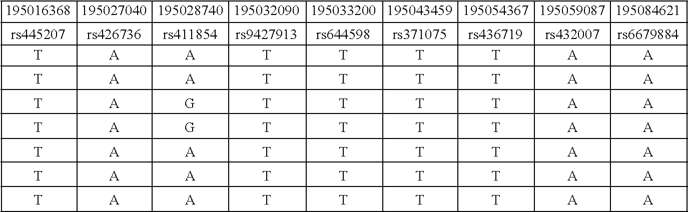

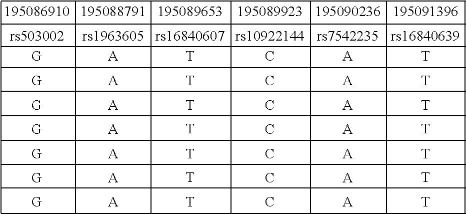

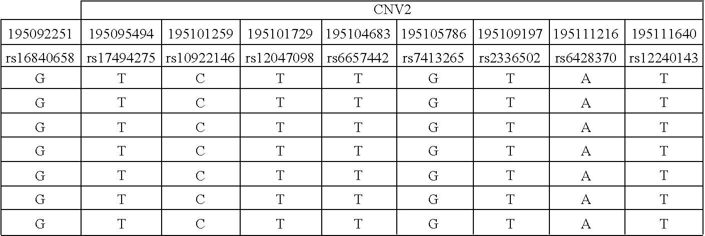

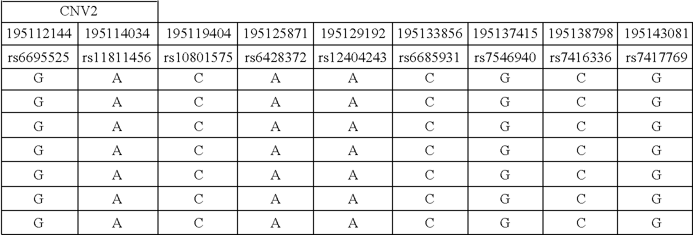

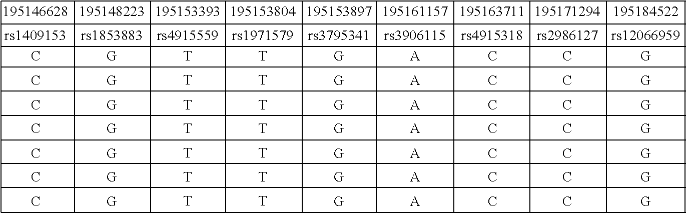

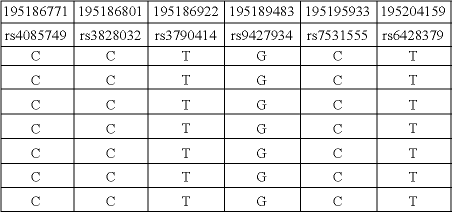

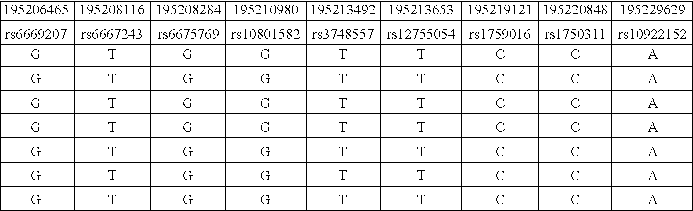

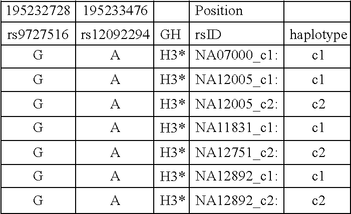

In Table 13 “rs1061170” is disclosed as SEQ ID NO: 16, “rs10922094” is disclosed as SEQ ID NO: 21, “rs12124794” is disclosed as SEQ ID NO: 22, “rs12405238” is disclosed as SEQ ID NO: 23, “rs10922096” is disclosed as SEQ ID NO: 24, “rs10922102” is disclosed as SEQ ID NO: 28, “rs2860102” is disclosed as SEQ ID NO: 29, “rs4658046” is disclosed as SEQ ID NO: 30, “rs12038333” is disclosed as SEQ ID NO: 33, “rs12045503” is disclosed as SEQ ID NO: 34, “rs9970784” is disclosed as SEQ ID NO: 35, “rs1831282” is disclosed as SEQ ID NO: 36, “rs203687” is disclosed as SEQ ID NO: 37, “rs2019727” is disclosed as SEQ ID NO: 38, “rs2019724” is disclosed as SEQ ID NO: 39, “rs1887973” is disclosed as SEQ ID NO: 40, “r56428357” is disclosed as SEQ ID NO: 41, “rs6695321” is disclosed as SEQ ID NO: 43, “rs10733086” is disclosed as SEQ ID NO: 44, “rs1410997” is disclosed as SEQ ID NO: 45, “rs203685” is disclosed as SEQ ID NO: 46, “rs10737680” is disclosed as SEQ ID NO: 48, “rs403846” is disclosed as SEQ ID NO: 17, “rs1409153” is disclosed as SEQ ID NO: 18 and “rs1750311” is disclosed as SEQ ID NO: 20.

### Estimated Loci for CNV1 and 2

CNV1 (NCBI37) chr1:196,660,832-196,680,665/(NCBI36) chr1:194927555-194947188 CNV2 (NCBI37) chr1:196,826,876-196,851,899/(NCBI36) chr1:195093499-195118522 Subjects revealing the highest fold difference in copy number using the qPCR assay were also reviewed for availability of 1000 Genomes BAM data. Four subjects were available in the C allele copy number variant group and two subjects in the T allele copy number variant group.

10 Subjects Showing Strongest Evidence of Copy Number Variant at the rs1061170 (SEQ ID NO: 16) Locus with qPCR

1) NA07034 (5.5 fold difference C)

2) NA07051 (7 fold difference C)*

3) NA07357 (6 fold difference C)*

4) NA10863 (5 fold difference C)

5) NA11994 (4.5 fold difference C)*

6) NA12058 (6.5 fold difference C)*

7) NA06985 (6 fold difference T)*

8) NA06991 (5 fold difference T)

9) NA07000 (8 fold difference T)*

10) NA07029 (9 fold difference T)
Subject with available 1000 Genomes data

Again the same two possible duplicated regions (CNV1 & CNV2) are apparent in most or all of the subjects evaluated. Relative depth of read may differ between subjects supporting the possibility of variable copy number between subjects.

Comparison of Subjects with High And Low Fold Changes by RS1061170 (SEQ ID NO: 16) Intensity Assay

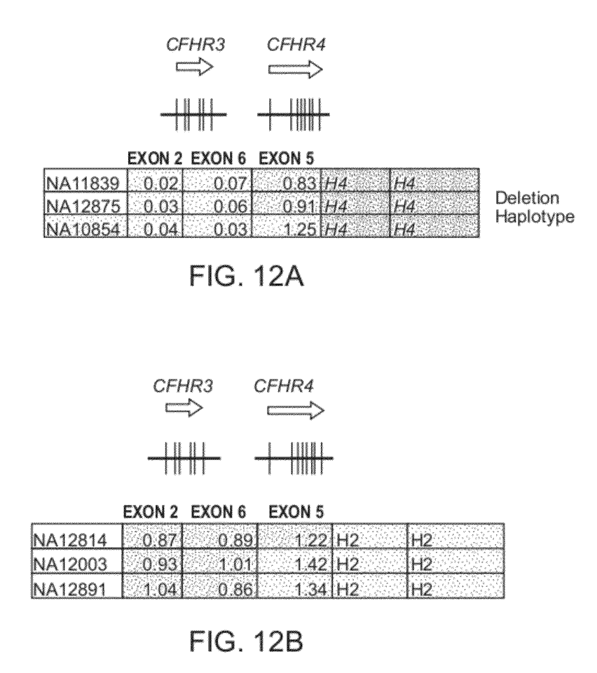

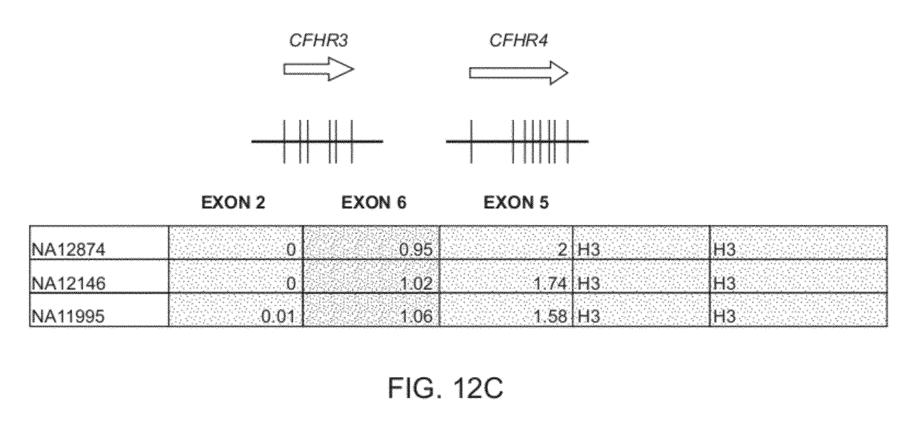

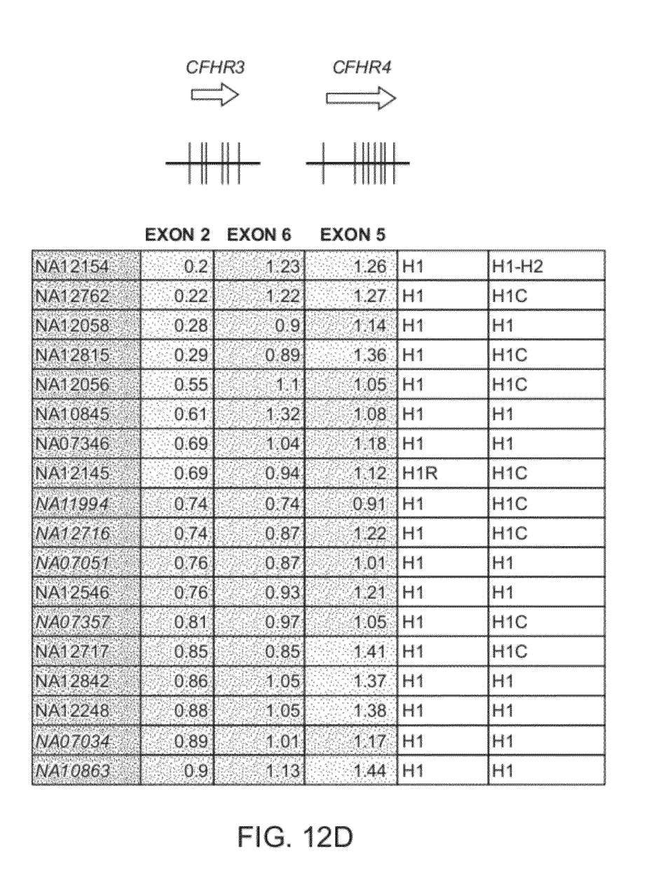

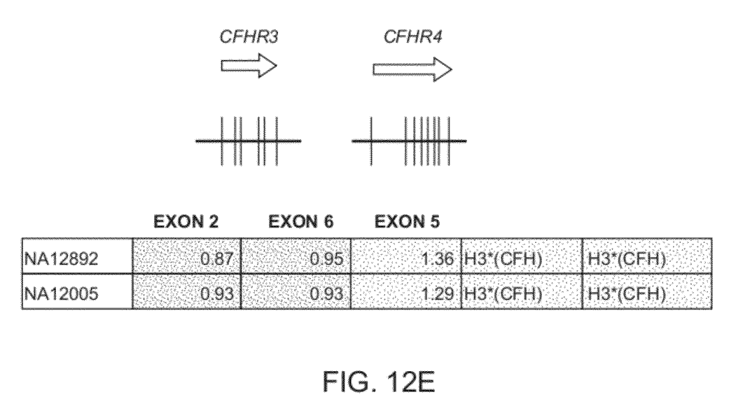

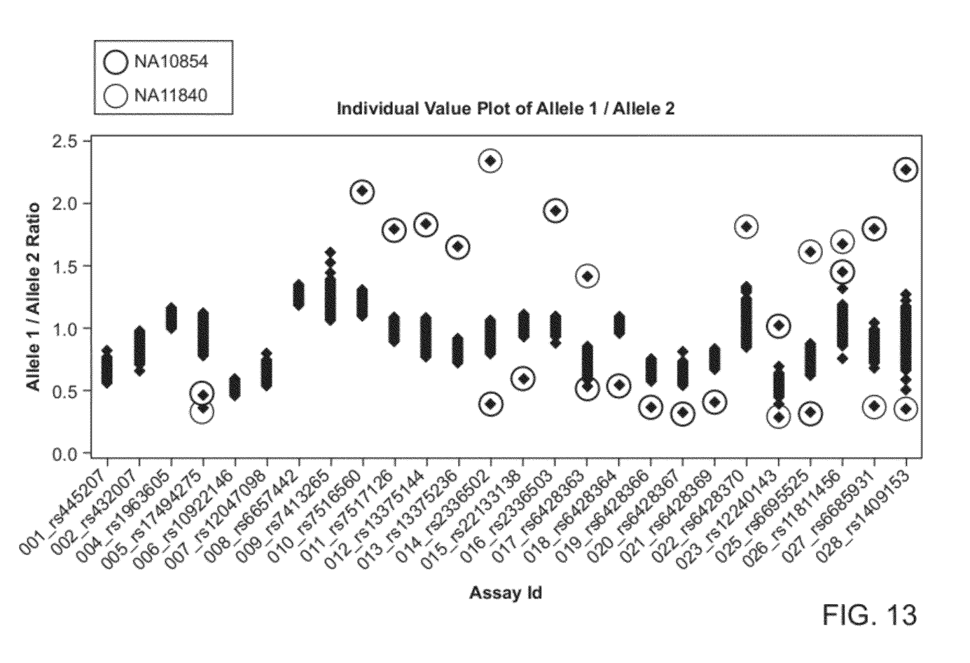

A selection of subjects were tested for copy number variant of the rs1061170 (SEQ ID NO: 16) C and T alleles (See FIGS. 12 and 13). Two groups were compared, group 1 contained subjects with >4fold intensity change, group 2 contained subjects with 1-2 fold change. Results are shown in Table 11 below. Subjects showing >4fold change for the C or T allele mostly show clear evidence for CNV1 and CNV2 where depth of reads are adequate. Notably subjects showing 1-2 fold change for the C or T allele, mostly show evidence for the known CFHR1/3 protective deletion, some also show possible, but generally weaker evidence for CNV1 and CNV2.

TABLE 11

| | Subject BAM | Subject BAM | | Subject BAM |
|---|---|---|---|---|
| Group | Assay fold | Assay fold | | Assay fold |
| 1 | NA11994 | 5.4 | gb | 4.5 |
| 1 | NA12716 | 3.8 | gb | 4.8 |
| 1 | NA07051 | 4.9 | gb | 7.3 |
| 1 | NA07357 | 1.60 | gb | 6.3 |
| 1 | NA12058 | 2.2 | gb | 6.5 |
| 2 | NA12234 | 1.0 | gb | 1.1 |
| 2 | NA11993 | 1.4 | gb | ND |
| 2 | NA12044 | 1.6 | gb | 1.1 |
| 2 | NA12043 | 0.9 | gb | 1.0 |
| 2 | NA12249 | 1.7 | gb | 1.3 |
| 2 | NA12144 | 1.5 | gb | 1.2 |
| 2 | NA12751 | 2.6 | gb | 1.2 |

Table 11 shows depth of read coverage for hapmap subjects showing >4 fold intensity change (group 1) and 1-2 fold intensity (group 2) for RS1061170 (SEQ ID NO: 16) C

TABLE 12

| | Subject BAM | Subject BAM | | Subject BAM |
|---|---|---|---|---|
| Group | Assay fold | Assay fold | | Assay fold |
| 1 | NA06985 | 0.62 | gb | 6.0 |
| 1 | NA07000 | 1.3 | gb | 8.2 |
| 2 | NA12234 | 1.0 | gb | 1.4 |
| 2 | NA12044 | 1.8 | gb | 1.5 |
| 2 | NA12043 | 0.9 | gb | 1.0 |
| 2 | NA12249 | 1.7 | gb | 1.3 |
| 2 | NA12144 | 1.5 | gb | 1.6 |
| 2 | NA12751 | 2.8 | gb | 1.0 |
| 2 | NA12006 | 1.0 | gb | 1.4 |
| 2 | NA11832 | 1.4 | gb | 1.5 |
| 2 | NA11992 | 2.8 | gb | 1.0 |

Table 12 shows depth of read coverage for hapmap subjects showing >4 fold intensity change (group 1) and 1-2 fold intensity (group 2) for RS1061170 (SEQ ID NO: 16) T

Comparison of subjects by HapMap “haplotype” across CNV1 region

HapMap subjects were sorted by markers described by Raychaudhuri et al (2010) that define the CFH risk haplotype, using only the 8 SNPs across the CNV1 locus. This sorted the subjects into 22 “haplotypes” across the CNV1 locus, including ˜10 common haplotypes. It was noted that 4/6 of the highly duplicated subjects were grouped in haplotype 21 (Excel FileCFH Genotypes). Most subjects in this grouping carried the H1/H1C risk haplotype.

Detailed characterization of CNV1 and CNV2

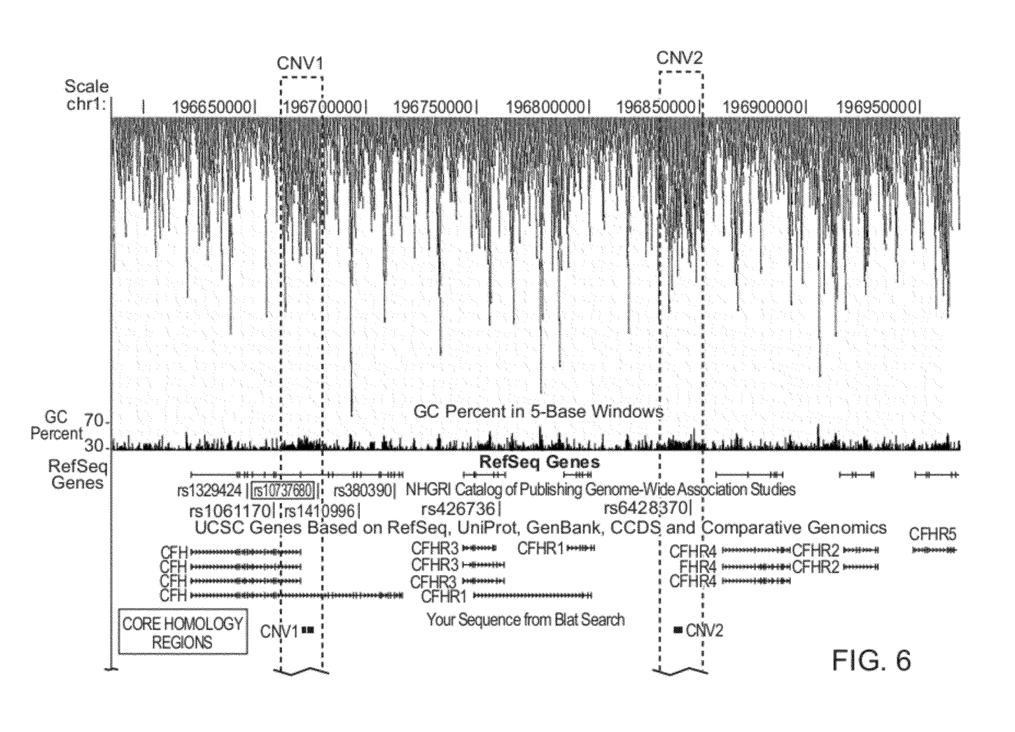

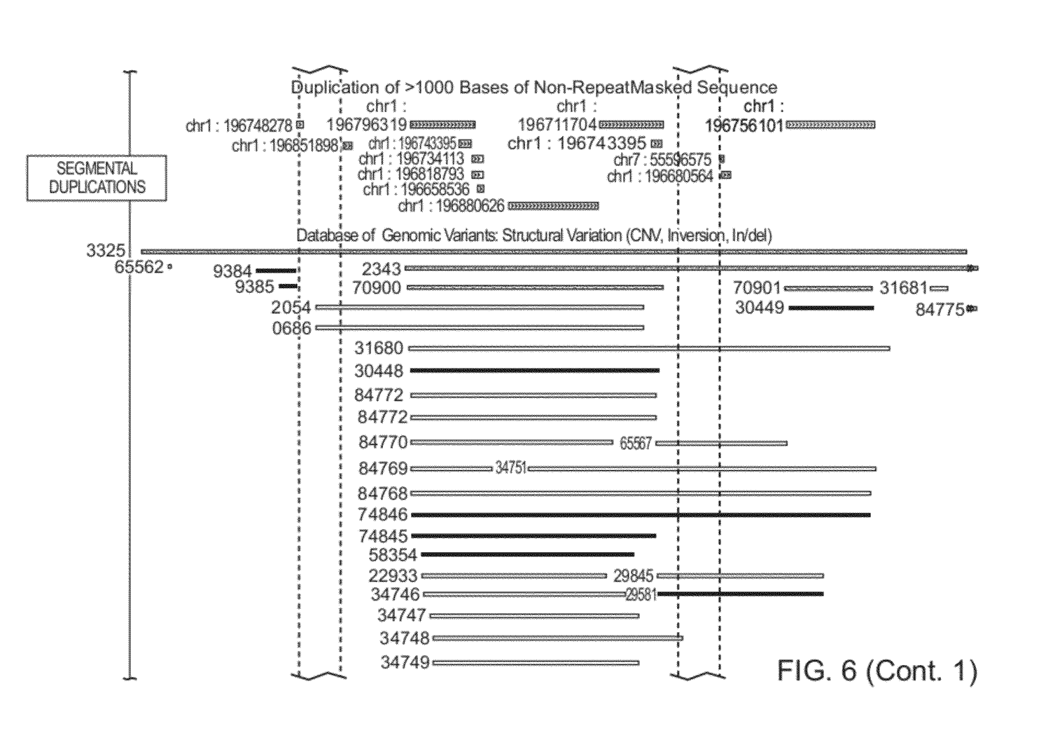

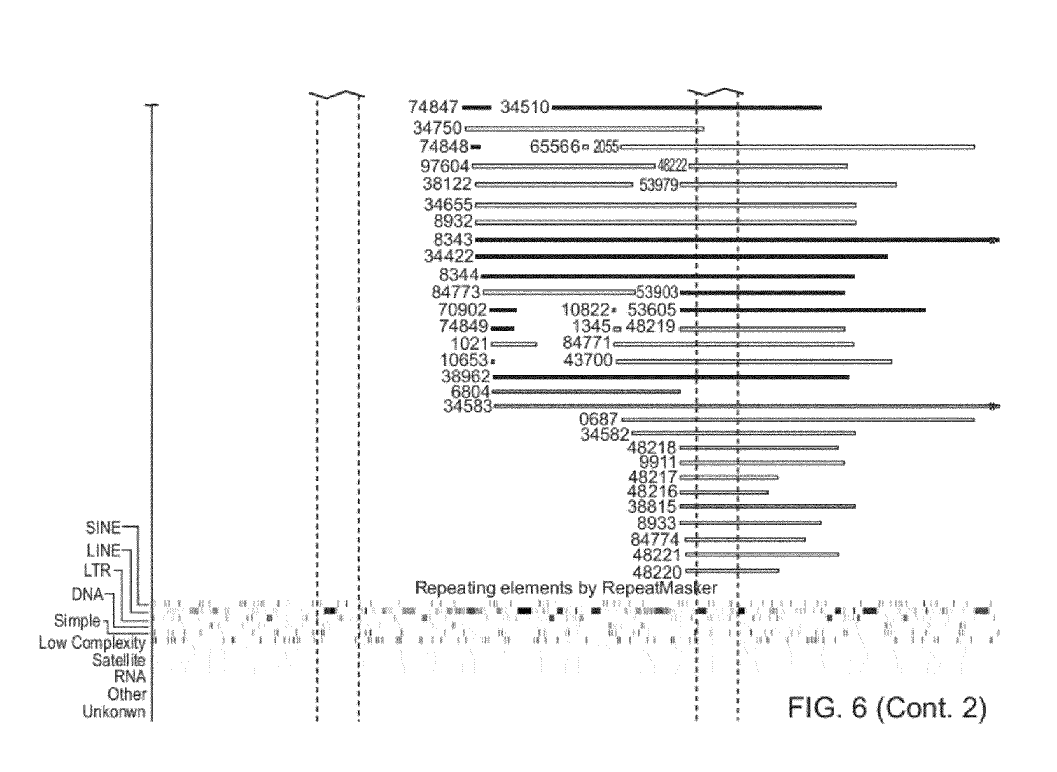

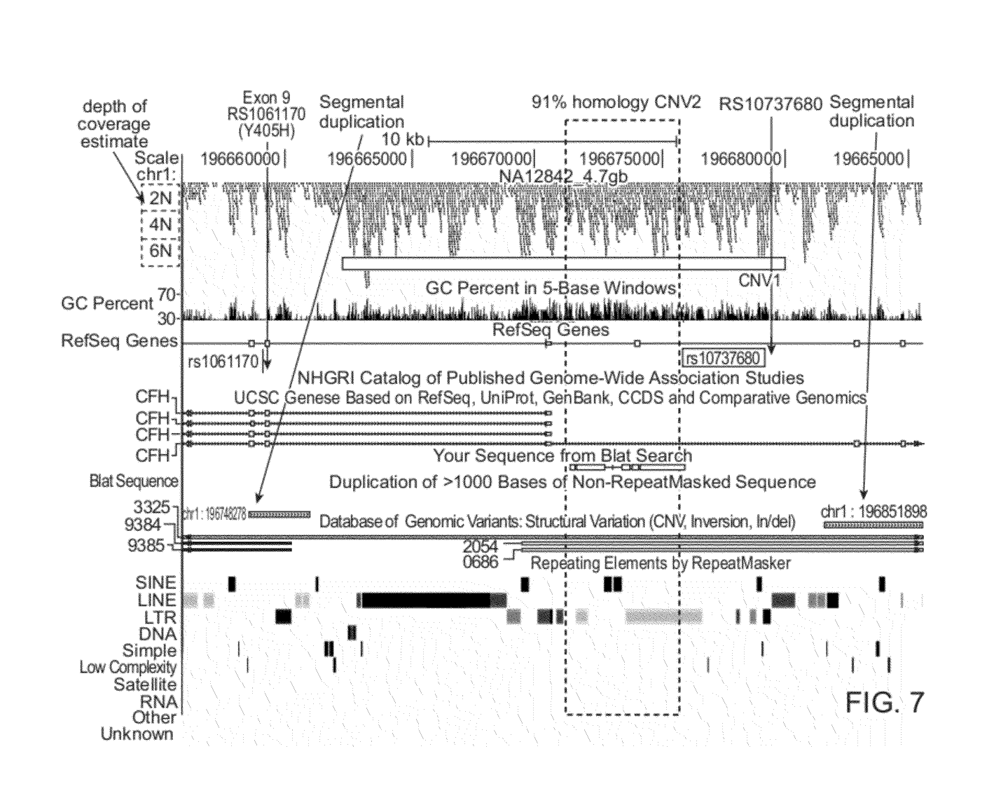

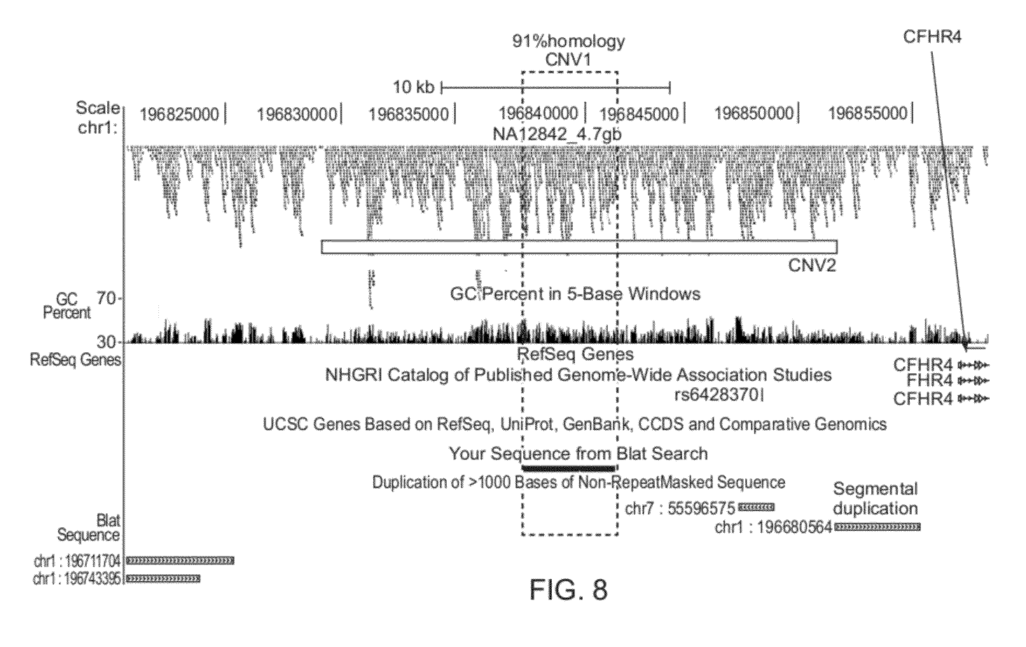

FIG. 6 shows a detailed view of subject NA12842 which shows the strongest evidence for CNV1 and CNV2 based on depth of read coverage. Detailed region views for CNV1 and CNV2 are shown in FIGS. 7 AND 8 respectively. It may be significant that CNV1 is closely flanked on both sides by segmental copy number variants—these are known to be a key mediator of CNV formation and are discussed further below. CNV1 and CNV2 seem to co-occur and it is also worth noting that both CNV1 and CNV2 share a core region of homology (CNV1: NCBI37: chr1:196671440-196676035; CNV2: NCBI37: chr1:196838070-196842074). It was noted that both CNV1 and CNV2 correlate with regions of high GC-ratio, this may lead to some bias in Solexa reads, however the CNVs are not seen in all subjects so this excludes the possibility that the putative CNVs are due to GC-ratio alone.

Determination of the boundaries of CNV1 and CNV2 at a sequence level

Custom track visualisation of BAM files using the UCSC browser allows sequence-review at the nucleotide level. Mis-matches to the genome reference sequence were identified. All available subjects were reviewed 2 kb either side of the putative CNV1 and CNV2 sequence boundaries, but no clear or consistent transition to duplicated coverage was observed.

### A Working Hypothesis: CNV1 and CNV2 are Cosmopolitan CNVs Mediated by Ancestral Segmental Copy Number Variants

A significant portion of CNVs have been identified in regions containing known segmental copy number variants Sharp et al. (2005). CNVs that are associated with segmental copy number variants may be susceptible to structural chromosomal rearrangements via non-allelic homologous recombination (NAHR) mechanisms (Lupski 1998). NAHR is a process whereby segmental copy number variants on the same chromosome can facilitate copy number changes of the segmental duplicated regions along with intervening sequences. In addition to the formation of CNVs in normal individuals, NAHR may also result in large structural polymorphisms and chromosomal rearrangements that directly lead to genomic instability or to early onset, highly penetrant disorders (Lupski 1998). CNVs mediated by segmental copy number variants have also been seen across multiple populations, including African populations, suggesting that these specific genomic imbalances may in some cases either predate the dispersal of modern humans out of Africa or recur independently in different populations. CNV1 and CNV2 are seen in the Yoruba subject carrying the known CFH copy number variant DGV9385, so this suggests that these CNVs may be ancient and highly dispersed among populations, although copy number may vary between populations.

### REFERENCES

Bentley D R, Balasubramanian S, Swerdlow H P, Smith G P, Milton J, Brown C G, Hall K P, Evers D J, Barnes C L, Bignell H R, et al. (2008) Accurate whole human genome sequencing using reversible terminator chemistry. Nature 456:53-59
Chen W, Stambolian D, Edwards A O, Branham K E, Othman M, Jakobsdottir J, Tosakulwong N, Pericak-Vance M A, Campochiaro P A, Klein M L, Tan P L, Conley Y P, Kanda A, Kopplin L, Li Y, Augustaitis K J, Karoukis A J, Scott W K, Agarwal A, Kovach J L, Schwartz S G, Postel E A, Brooks M, Baratz K H, Brown W L; Complications of Age-Related Macular Degeneration Prevention Trial Research Group, Brucker A J, Orlin A, Brown G, Ho A, Regillo C, Donoso L, Tian L, Kaderli B, Hadley D, Hagstrom S A, Peachey N S, Klein R, Klein B E, Gotoh N, Yamashiro K, Ferris Lii F, Fagerness J A, Reynolds R, Farrer L A, Kim I K, Miller J W, Cortón M, Carracedo A, Sanchez-Salorio M, Pugh E W, Doheny K F, Brion M, Deangelis M M, Weeks D E, Zack D J, Chew E Y, Heckenlively J R, Yoshimura N, lyengar S K, Francis P J, Katsanis N, Seddon J M, Haines J L, Gorin M B, Abecasis G R, Swaroop A. (2010) Genetic variants near TIMP3 and high-density lipoprotein-associated loci influence susceptibility to age-related macular degeneration. Proc Natl Acad Sci USA. 107(16):7401-6
Hageman G S, Anderson D H, Johnson L V, Hancox L S, Taiber A J, Hardisty L I, Hageman J L, Stockman H A, Borchardt J D, Gehrs K M, Smith R J, Silvestri G, Russell S R, Klayer C C, Barbazetto I, Chang S, Yannuzzi L A, Barile G R, Merriam J C, Smith R T, Olsh A K, Bergeron J, Zernant J, Merriam J E, Gold B, Dean M, Allikmets R. (2005) A common haplotype in the complement regulatory gene factor H(HF1/CFH) predisposes individuals to age-related macular degeneration. Proc Natl Acad Sci USA. 102(20):7227-32.
Hughes A E, Orr N, Esfandiary H, Diaz-Torres M, Goodship T, Chakravarthy U. (2006) A common CFH haplotype, with deletion of CFHR1 and CFHR3, is associated with lower risk of age-related macular degeneration. Nat. Genet. 2006 October; 38(10):1173-7
Lupski J R. (1998) Genomic disorders: structural features of the genome can lead to DNA rearrangements and human disease traits. Trends Genet. 1998 October; 14(10):417-22.
Oeth P, del Mistro G, Marnellos G, Shi T, van den Boom D. Qualitative and quantitative genotyping using single base primer extension coupled with matrix-assisted laser desorption/ionization time-of-flight mass spectrometry (MassARRAY). Methods Mol. Biol. 2009; 578:307-43.
Pfaffl Michael W, A new mathematical model for relative quantification in real-time RT-PCR. Nucleic Acids Res. 2001 29(9): E45
Quail M A, Kozarewa I, Smith F, Scally A, Stephens P J, Durbin R, Swerdlow H, Turner D J (2008) A large genome center's improvements to the Illumina sequencing system. Nat. Methods. 5(12):1005-1010.
Raychaudhuri S, Ripke S, Li M, Neale B M, Fagerness J, Reynolds R, Sobrin L, Swaroop A, Abecasis G, Seddon J M, Daly M J. (2010) Associations of CFHR1-CFHR3 deletion and a CFH SNP to age-related macular degeneration are not independent. Nat. Genet. 2010 July; 42(7):553-5;
Sharp A J, Locke D P, McGrath S D, Cheng Z, Bailey J A, Vallente R U, Pertz L M, Clark R A, Schwartz S, Segraves R, Oseroff V V, Albertson D G, Pinkel D, Eichler E E. (2005) Segmental copy number variants and copy-number variation in the human genome. Am J Hum Genet. 77(1):78-88
Xie C, Tammi M T. (2009) CNV-seq, a new method to detect copy number variation using high-throughput sequencing. BMC Bioinformatics. 10:80.
Fritsche et al. An imbalance of human complement regulatory proteins CFHR1, CFHR3 and factor H influences risk for age-related macular degeneration (AMD) Hum. WI. Genet. (2010) Sep. 30. [Epub ahead of print].
Venables J P, Strain L, Routledge D, Bourn D, Powell H M, Warwicker P, Diaz-Torres M L, Sampson A, Mead P, Webb M, Pirson Y, Jackson M S, Hughes A, Wood K M, Goodship J A, Goodship T H. Atypical haemolytic uraemic syndrome associated with a hybrid complement gene. PLoS Med. 2006 October; 3(10):e431.

### Example 5

### Evaluation of Copy Number Polymorphisms Observed Across the CFH-CFHR Region Using Digital PCR

Copy number polymorphisms in the CFH-CFHR region can be evaluated utilizing digital PCR, in some embodiments. Provided herein are the results of experiments performed, using digital PCR, to evaluate polymorphisms observed across the CFH-CFHR region of chromosome one (e.g., Chr 1). The results of the experiments provide additional evidence of the presence of copy number variation in well characterized HapMap samples and clinical samples derived from blood and/or buccal cells.

Digital PCR

Digital PCR was used to measure differences in copy number across multiple exons and introns of the CFH, CFRH3 and CFHR4 genes. Digital PCR can be used to amplify on or more segments of nucleic acid and compare the signal to a control amplification targeting a region on the same or different chromosomes (e.g., a region previously tested and confirmed for lack copy number variation), in some embodiments. Digital PCR reactions described herein were performed as multiplex reactions in a single tube along with the control amplifications. Resultant product signals were compared between tests and controls to detect differences reflective of duplications or deletions in the interrogated loci.

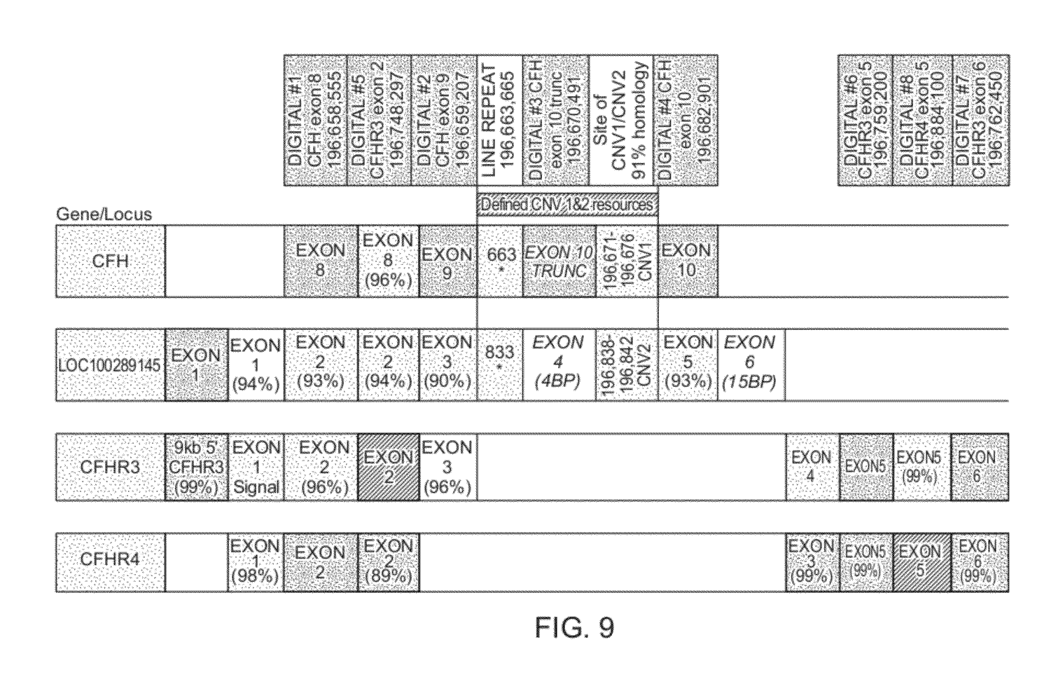

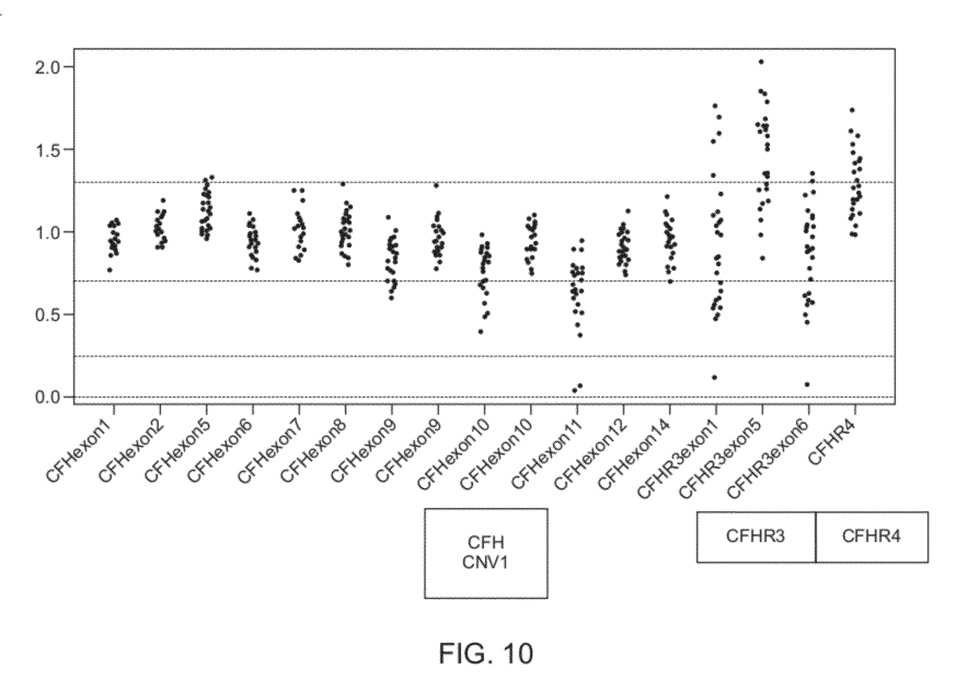

Sixteen digital PCR assays detecting sequences across the CFH-CFHR region were developed to detect differences in signal reflective of copy number variation. FIG. 9 provides evidence of the high sequence homology observed across CFH, LOC100289145, CFHR3 and CFHR4 regions contained in the RCA gene cluster. The eight assays listed in the top row (e.g., in dark gray) of FIG. 9 target exons in the CFH, CFHR3 and CFHR4 loci. Results from the digital PCR assays illustrate differences in signal reflective of copy number variation (e.g., deletions and duplications) are illustrated in FIG. 10. Differences in copy number across the CFH, CFHR3 and CFHR4 regions were established by comparison to well characterized control regions. Assays targeting regions in CFH (exon 9, 10 (truncated), and 11 (full length exon 10)) were most pronounced in observed variation. Additional polymorphism detected in CFHR3 revealed signal differences reflective of both deletions (consistent with the known CFHR3-CFHR184 kb deletion reported in this region by Hughes et al) but also novel duplications in selective samples.

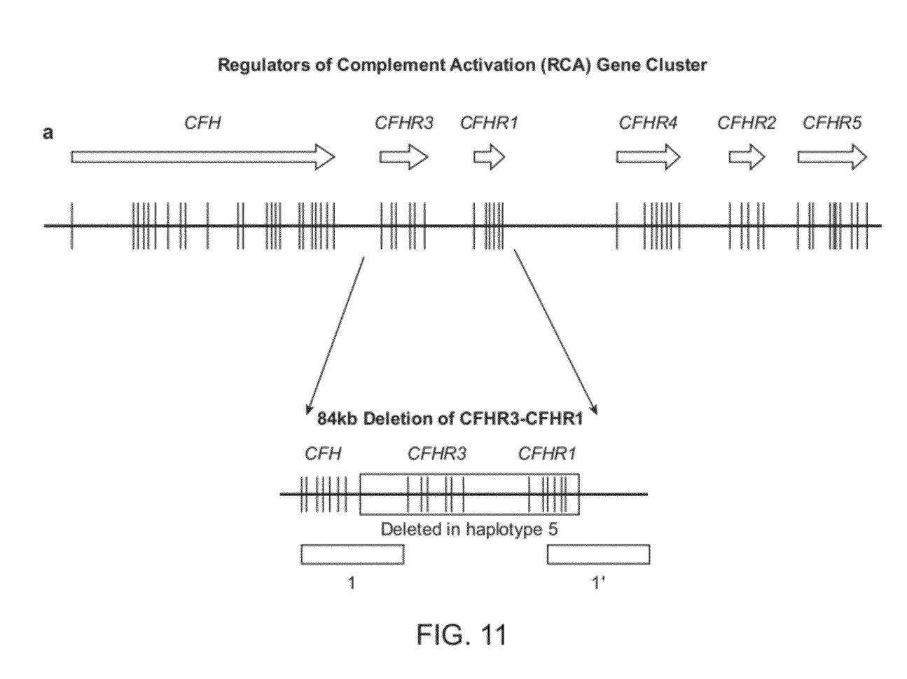

FIG. 11 schematically illustrates the 84 kb deletion of the CFHR3/CFHR1 region reported by Hughes et al. The deletion is reported to provide significant association with protection from AMD. Although the deletion in the CFHR3/CFHR1 region provides protection from AMD, it is believe that the same deletion may lead to increased susceptibility to aHUS. Without being limited by theory, it is believed that the absence of the CFHR3 gene product reduces competition for CFH binding and thereby increases the effectiveness of the key inhibitor of the alternative complement pathway. Thus, duplications of the CFHR3 gene product may shift the delicate balance of control away from inhibition and markedly increase susceptibility to AMD in the presence of a CFHR3 (or highly homologous protein) duplication.

Results from 3 informative digital PCR assays (e.g., performed on CFHR3 exon 2, CFHR3 exon 6 and CFHR4 exon 5) demonstrated CFH haplotype specific copy number differences. The differences were observed by testing known samples homozygous for the haplotypes of interest. Samples previously characterized as H4/H4, H3/H3, H2/H2 and H1/H1 were surveyed to identify copy number differences that would associate with disease haplotypes. Disease associated haplotypes include H1 and H3 while H2 and H4 are protective in nature. An additional sample homozygous for a haplotype identified as a hybrid (H3*) was also subject to evaluation.

Digital PCR assay results can be interpreted as follows; A result indicating no difference in copy number would be revealed in a value close to 1 (e.g., in the range of about 0.8 to about 1.2). A value of close to 0.5 (e.g., in the range of about 0.3 to about 0.7) would be reflective of 1 less copy number (n) compared to the expected (2n) copies. Values near 1.5 (e.g., in the range of about 1.3 to about 1.7) or near 2.0 (e.g., in the range of about 1.8 to about 2.2) may reflect 3-fold (e.g., 3n) and 4-fold (e.g., 4n), respectively.

SNP's probative for various CFH gene haplotype combinations were evaluated using a digital PCR assay. FIG. 12A illustrates the results of 3 samples that were previously identified as having an H4/H4 haplotype. As shown in FIG. 12A, no amplification signal is generated for exon 2 and exon 6, which is consistent with the H4/H4 haplotypes being homozygous for the CFHR3/CFHR1 deletion. The diploid (e.g., 2n) copy number observed in samples NA11839 and NA12875 for the assay detecting exon 5 in CFHR4 is also consistent with what would be expected for an unaffected sample. Sample NA108514 is indicative of 2 copies of the CFHR3-1 deletion, evident in the lack of signal observed in the two CFHR3 and 3n copy number detected in the assay detecting CFHR4.

FIG. 12B illustrates the results of three H2/H2 homozygous samples revealing the expected 2n number of alleles in CFHR3. Two of the samples also appear to show differences in expected copy number observed in the CFHR4 assay. FIG. 12C illustrates a novel copy deletion polymorphism in exon 2 of CFHR3 in all 3 samples typed as H3/H3 homozygous. All three reveal the expected 2n copy number in exon 6 of CFHR3 while the results for the exon 5 assay of CFHR4 show pronounced increases (3n-4-n copy number) in the CFHR4 gene.

FIG. 12D illustrates results from multiple H1/H1 homozygous samples. The following samples were previously identified as having duplications in CNV1 and CNV2: NA11994, NA12716, NA07051, NA07357, NA07034, and NA10863. Results from the digital PCR assay demonstrated that there were differences in copy number in the exon 2 CFHR3 assay revealing differences in samples that were previously characterized as H1 haplotypes. In all cases, the samples previously identified as having more pronounced short read sequencing signal detected in the Depth of Coverage analysis (DOC) had higher signals in the assay detecting CFHR3 exon 2. These data indicate there appear to be different subtypes of H1 alleles that can be differentiated on the basis of copy number differences observed in the assay detecting exon 2 CFHR3. FIG. 12E illustrates results from 2 samples identified as hybrid haplotypes (H3/H1) that appear to behave similarly to H1/H1 homozygous samples. The two samples reveal expected copy number in CFHR3 (2n) and duplications in CFHR4 (3n).

SNP Allele Ratios

SNP allele ratio assays described herein measure the signal observed in heterozygous samples containing 1 copy each of a single nucleotide polymorphism variant located in regions defined as CNV 1 and CNV 2. The SNP assay distinguished various haplotype combinations that revealed differences in allele ratios that were greater or less than 1:1 in samples containing a duplication across the CHF-CHFR region.

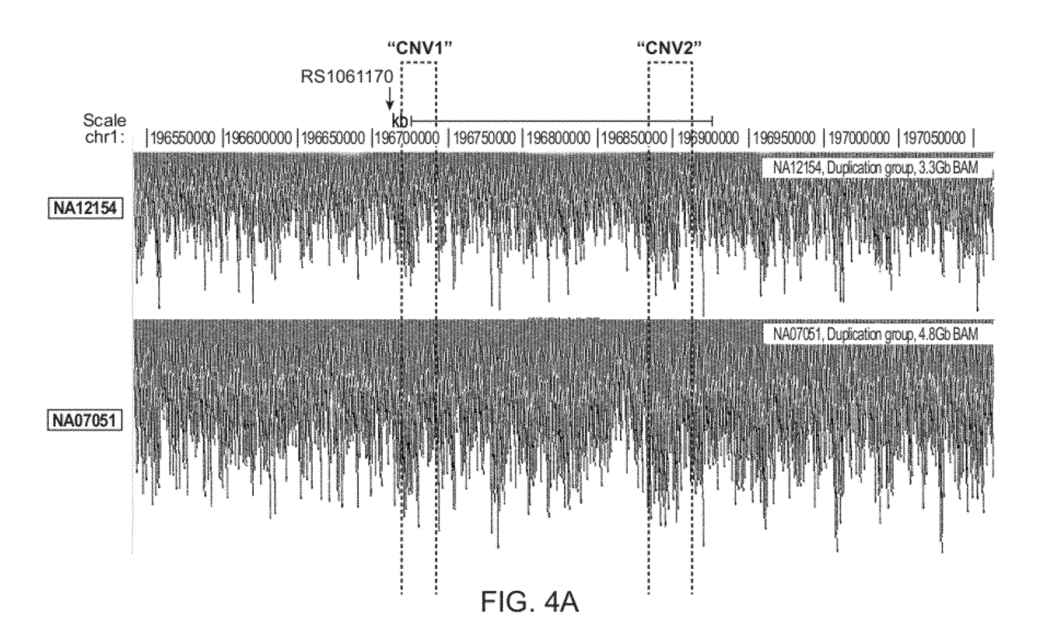

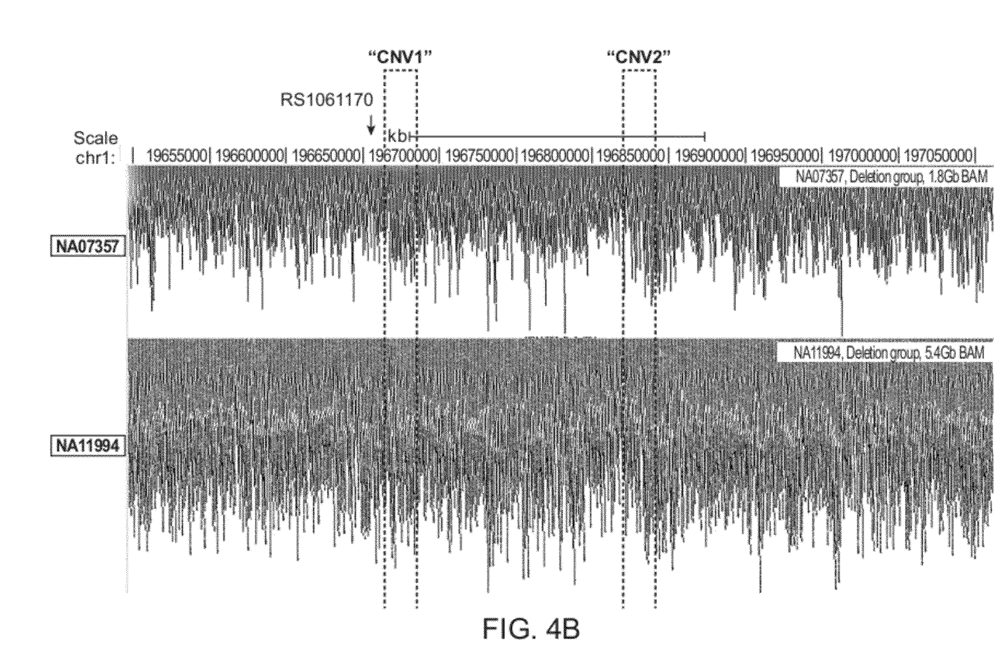

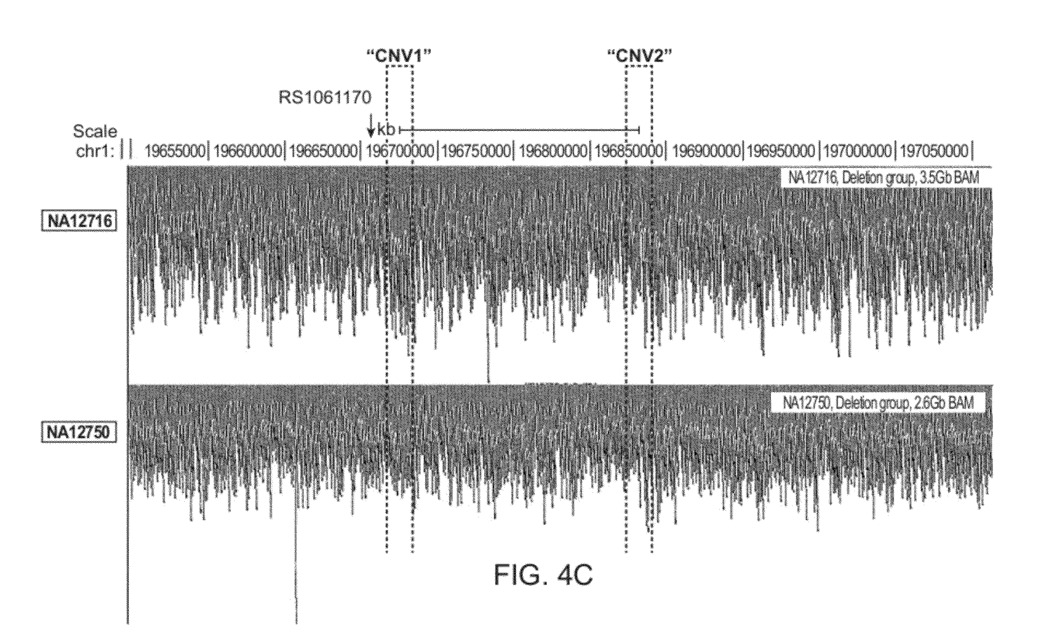

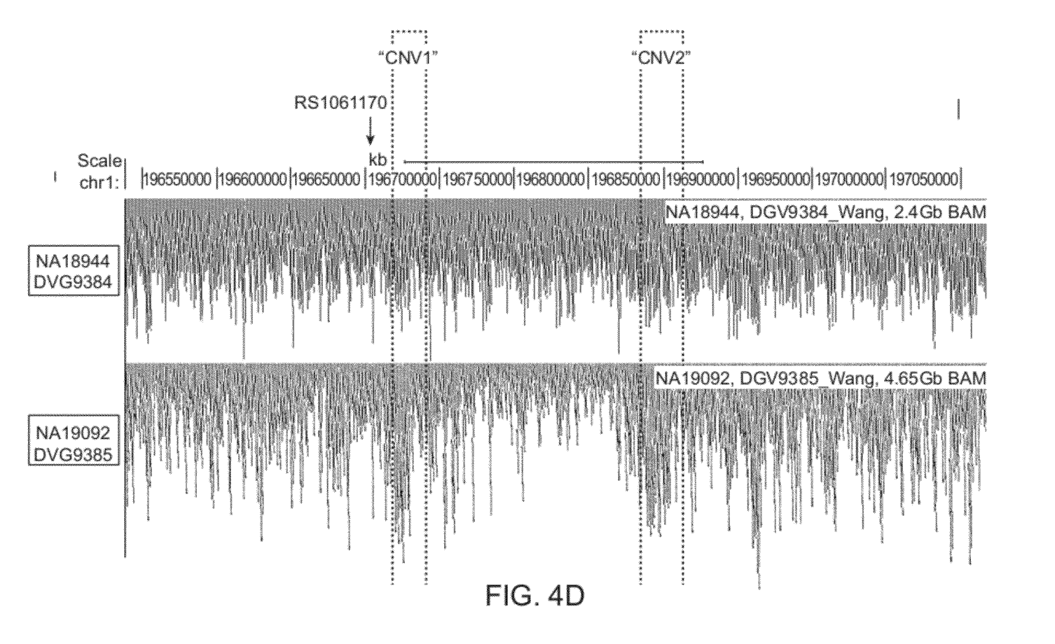

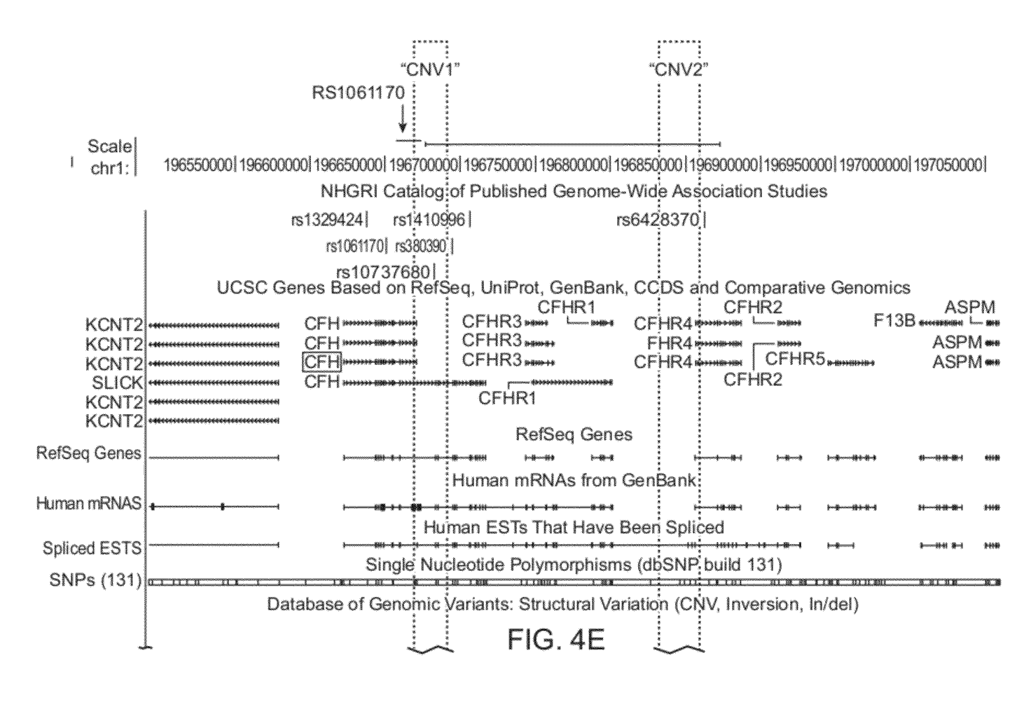

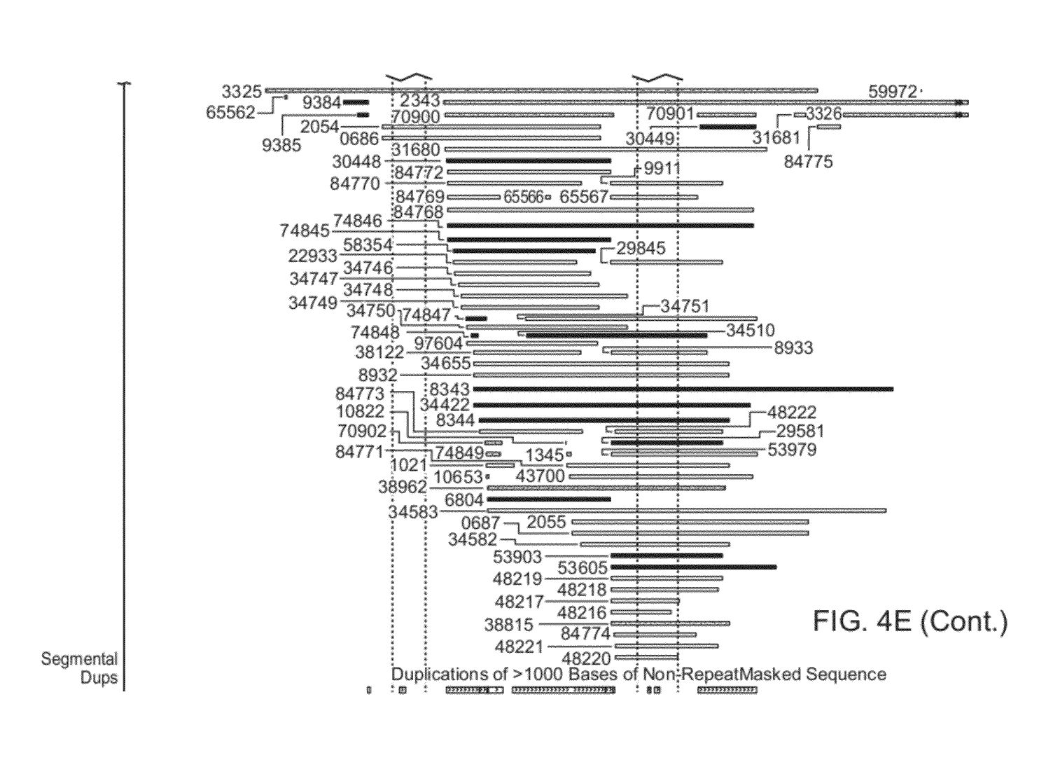

FIG. 13 illustrates the results of 26 SNPs (e.g., listed along the x-axis) tested on HapMap samples to evaluate ratio differences reflective of copy number polymorphisms in CNV2. A similar analysis also was performed for CNV1 (e.g., figure not shown). Two samples. NA 10854 (see FIG. 4a) and NA11840, revealed the most significant differences in allele ratios reflective of a duplication of the entire region spanning CFHR3-rs445207 through CFHR4-rs1409153 (SEQ ID NO: 18).

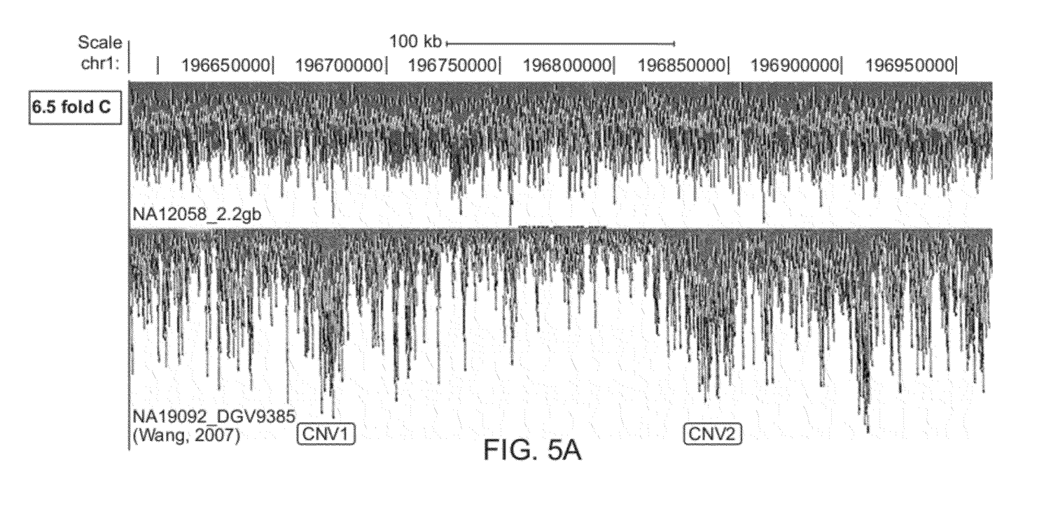

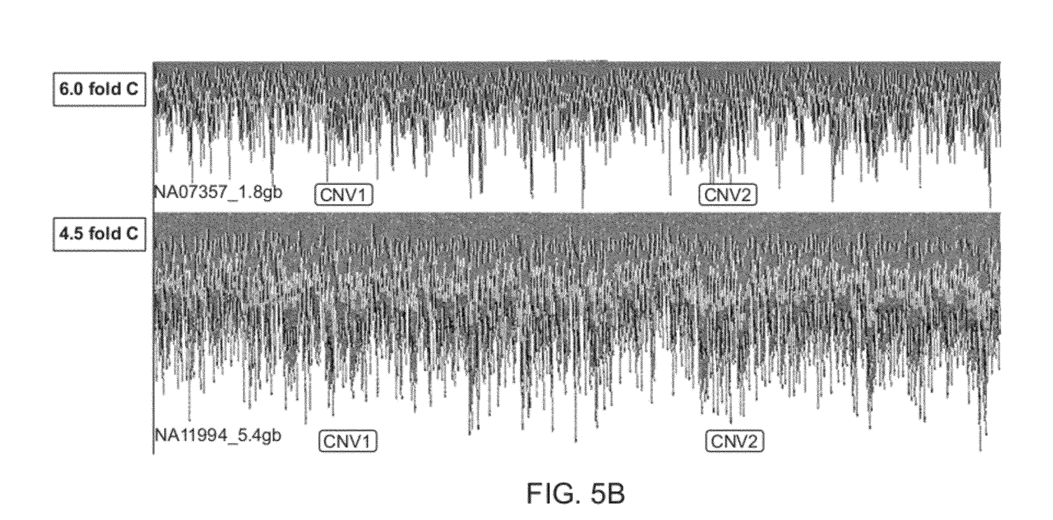

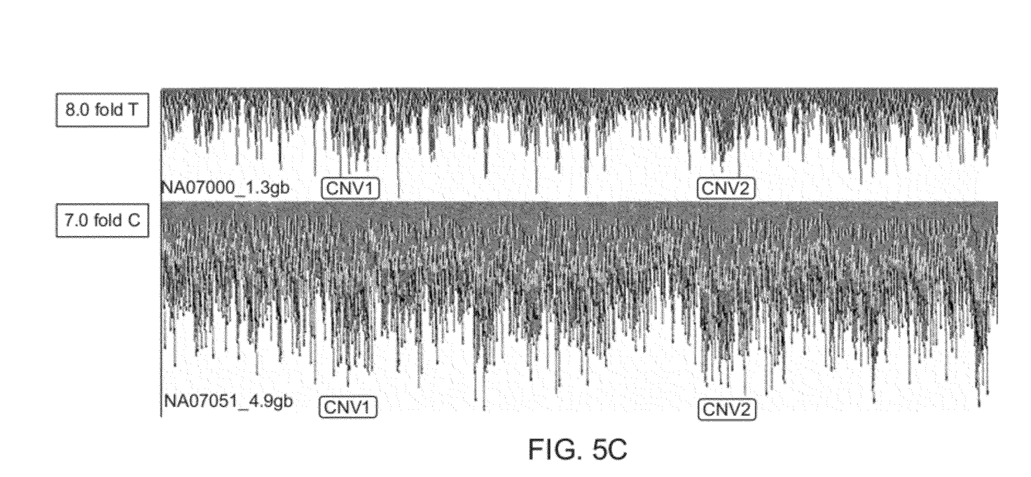

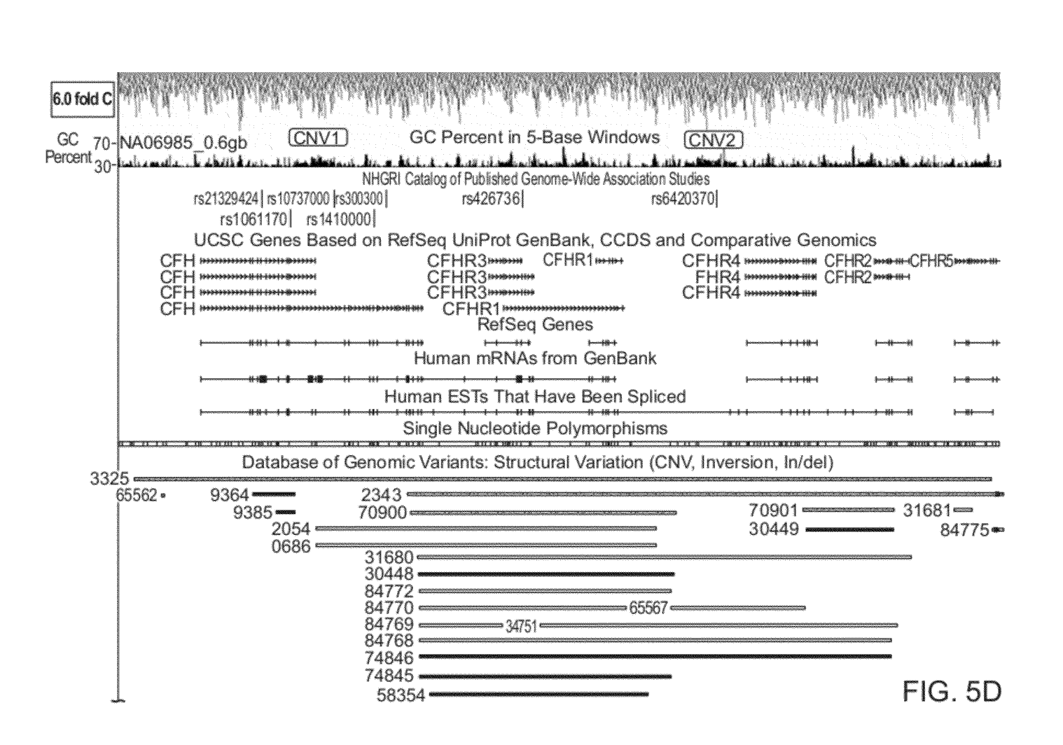

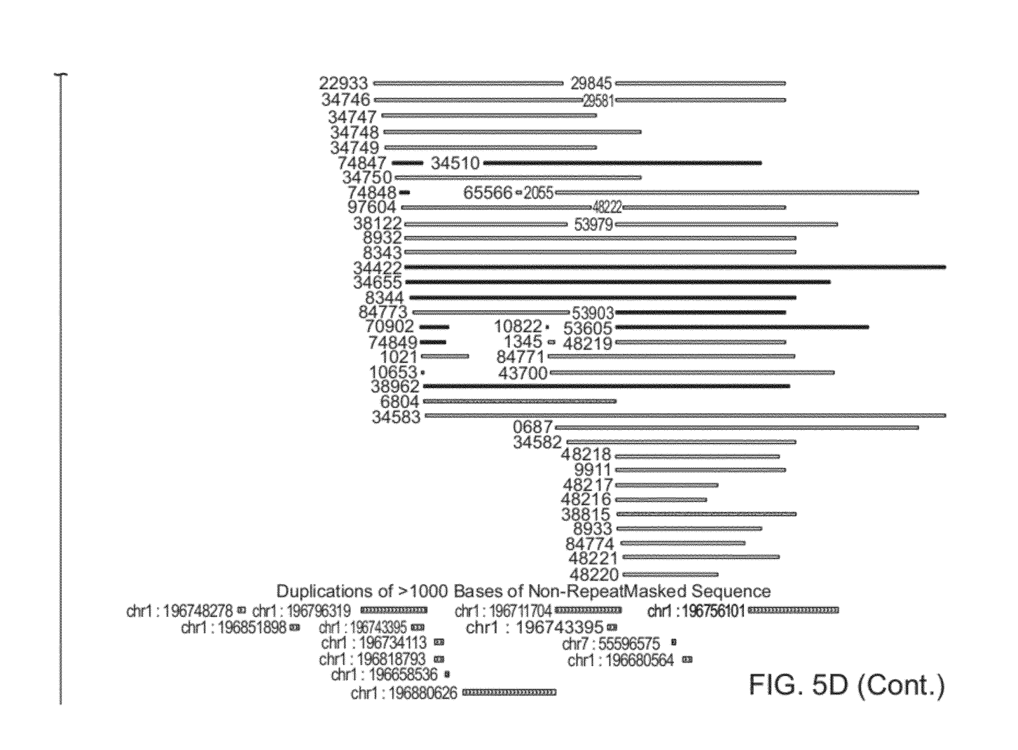

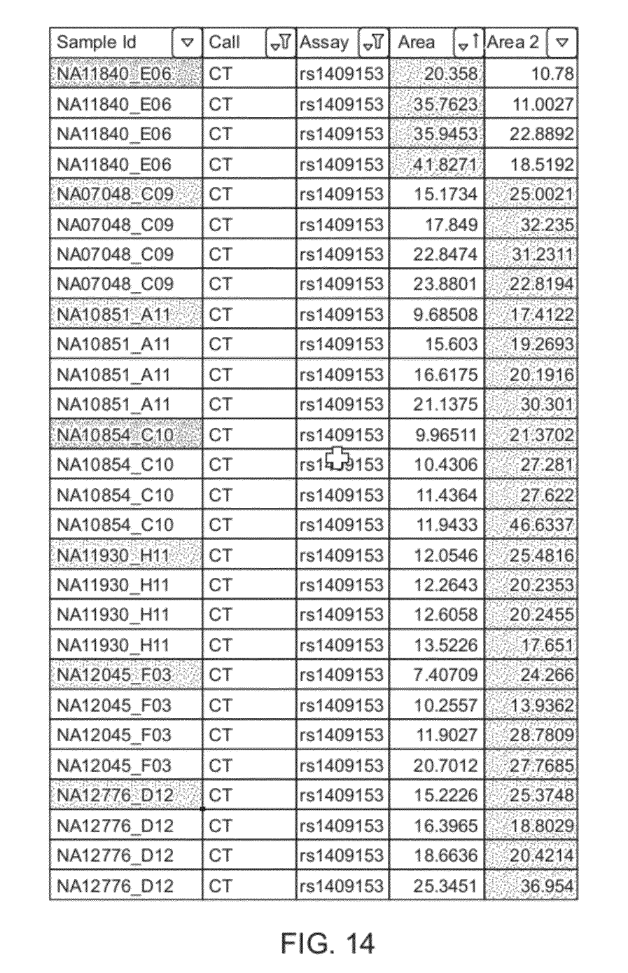

FIG. 14 illustrates the results of experiments performed to show copy number differences in samples NA10854 and NA11840 (both highlighted in dark gray) identified using multiple SNP ratio assays. SNP ratio assays measure the signal of 2 alleles in heterozygous samples, in some embodiments. Additional samples (highlighted in light gray) depicted the individual SNP assays illustrated in FIG. 5 showed ratio differences that were not as pronounced as the ratios seen for NA11840 and NA10854 but were still reflective of smaller copy number variances. The more robust differences may reflect more significant duplication while the samples revealing smaller differences may represent combinations of duplications and or deletions in this region.

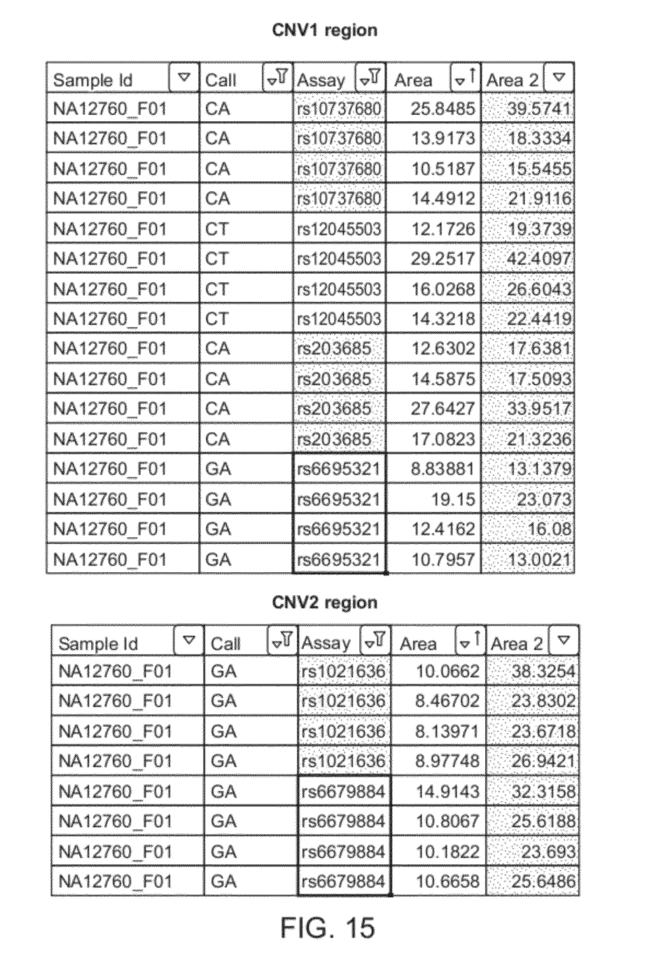

The SNP allele ratio assay also could be used to identify samples that revealed differences in allele ratios observed across multiple SNPs in both CNV1 and CNV2 regions. The samples that revealed difference in allele ratios across multiple SNPs in CNV1 and CNV2 may be indicative of duplications that involve a larger segment spanning the region between CNV1 and CNV2. Without being limited by theory, there may be some duplications that are limited to the CNV2 region while others involve a more significant section of duplication extending to the region near exon 9 of CFH. FIG. 15 below illustrates an example of a sample (NA12760) that demonstrates ratio differences observed across multiple SNPS covering both CNV1 and CNV2 regions.

Table 14 below provides relevant SNPs in CNV 2 region that detect duplication using sample NA11840 as an example. Grey highlight shows duplicated allele. Alleles are listed in column 2 “call”, SNP name is in column 3 and signal from first and second nucleotide respectively are in column 4 and 5. In Table 14 “rs1409153” is disclosed as SEQ ID NO: 18.

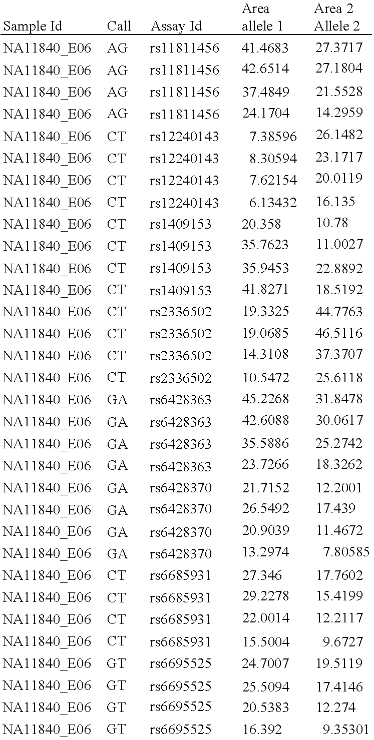

Table 15 below provides relevant SNPs in CNV 2 region that detect duplication using sample NA10864 as an example. Grey highlight shows duplicated allele. Alleles are listed in column 2 “call”, SNP name is in column 3 and signal from first and second nucleotide respectively are in column 4 and 5. In Table 15 “rs1409153” is disclosed as SEQ ID NO: 18.

TABLE 15

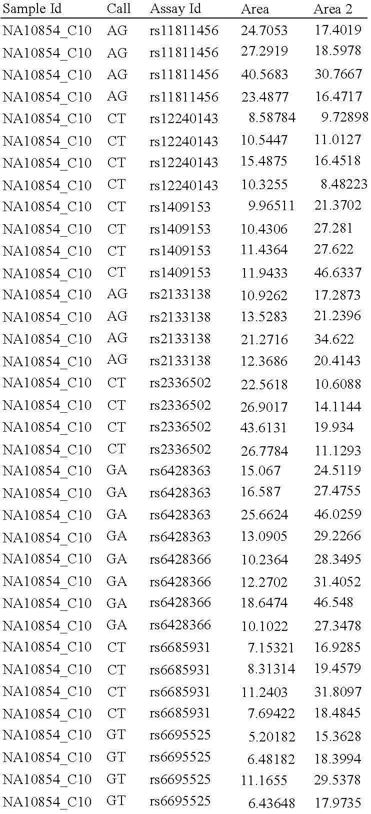

Table 16 below provides relevant SNPs in CNV 1 region that detect duplication using sample NA11840 as example. Grey highlight shows duplicated allele. Alleles are listed in column 2 “call”, SNP name is in column 3 and signal from first and second nucleotide respectively are in column 4 and 5. Note duplication as a function of signal difference is not as pronounced in CNV1 region as observed in CNV2 region for this sample. In Table 16 “rs10733086” is disclosed as SEQ ID NO: 44, “rs10737680” is disclosed as SEQ ID NO: 48, “rs10922094” is disclosed as SEQ ID NO: 21, “rs12045503” is disclosed as SEQ ID NO: 34, “rs1887973” is disclosed as SEQ ID NO: 40, “rs2019724” is disclosed as SEQ ID NO: 39, “rs2019727” is disclosed as SEQ ID NO: 38, “rs203685” is disclosed as SEQ ID NO: 46, “rs203687” is disclosed as SEQ ID NO: 37, “rs2860102” is disclosed as SEQ ID NO: 29, “rs4658046” is disclosed as SEQ ID NO: 30, “rs514943” is disclosed as SEQ ID NO: 26 and “r56428357” is disclosed as SEQ ID NO: 41.

TABLE 16

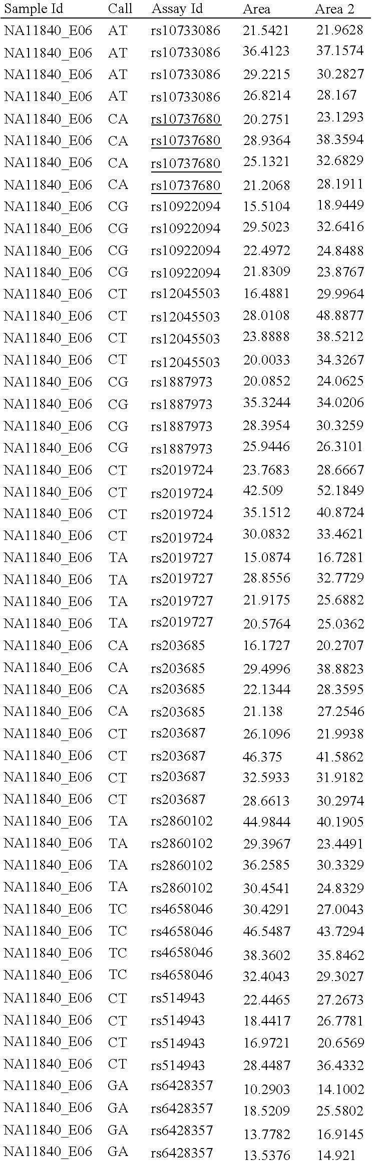

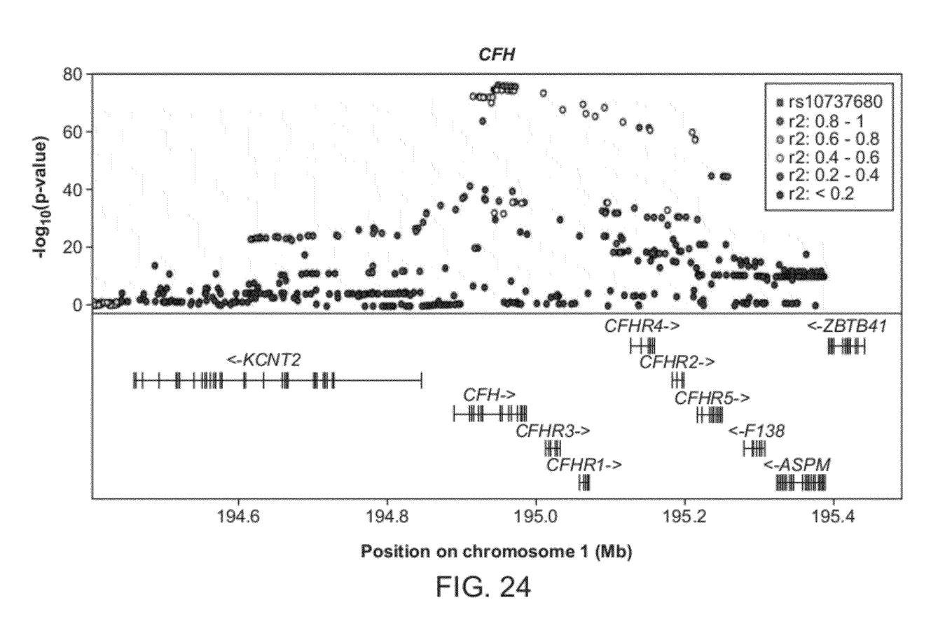

Studies have shown a consistently strong association with CFH at the missense Tyr402His variant (rs1061170 (SEQ ID NO: 16)), however a recent high density association study (Chen et al 2010), repeated association at rs1061170 (SEQ ID NO: 16), but showed strongest association with rs10737680 (SEQ ID NO: 48) (underlined in above table) in intron 10 of the CFH gene (odds ratio (OR)=3.11 (2.76, 3.51), with P<1.6×10-75). FIG. 24 illustrates a regional ARMD4 association plot for CFH (Chen et al. 2010).

Identification of Haplotypes in Clinical Samples

Clinical samples were examined for the presence of haplotypes that contained SNPs that showed a significant departure from linkage disequilibrium values expected across the highly conserved regions comprising CFH through CFHR5. A full panel of haplotypes was imputed from about 1900 clinical samples with late stage CNV AMD (Choroidal neovascular AMD) and age matched controls. These haplotypes were further evaluated in clinical samples with known disease (AMD) to identify haplotype combinations that would reflect copy number polymorphism across the CFH region.

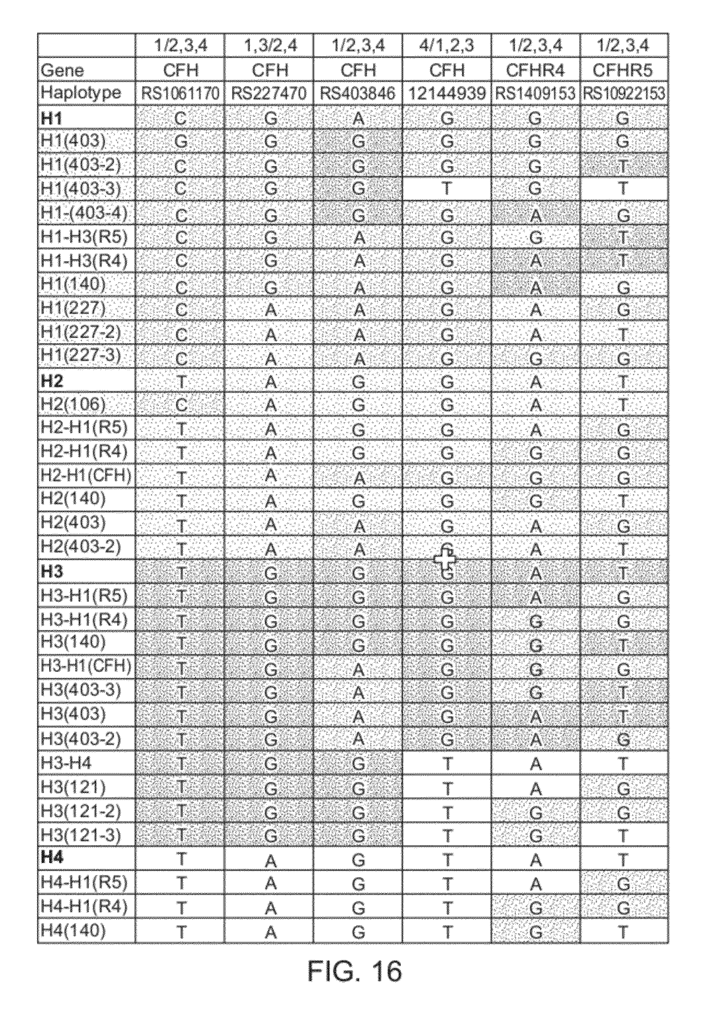

FIG. 16 illustrates the different haplotypes imputed from a collection of about 1900 clinical samples with late stage AMD (CNV) and age matched controls. The SNPs that distinguish different haplotype combinations were effective at revealing a large number of haplotypes beyond those that were reported in 2005 (H1, H2, H3, H4). The haplotypes with the most significant frequency of combination were H1 and H3, the two most significant risk haplotypes associated with AMD.

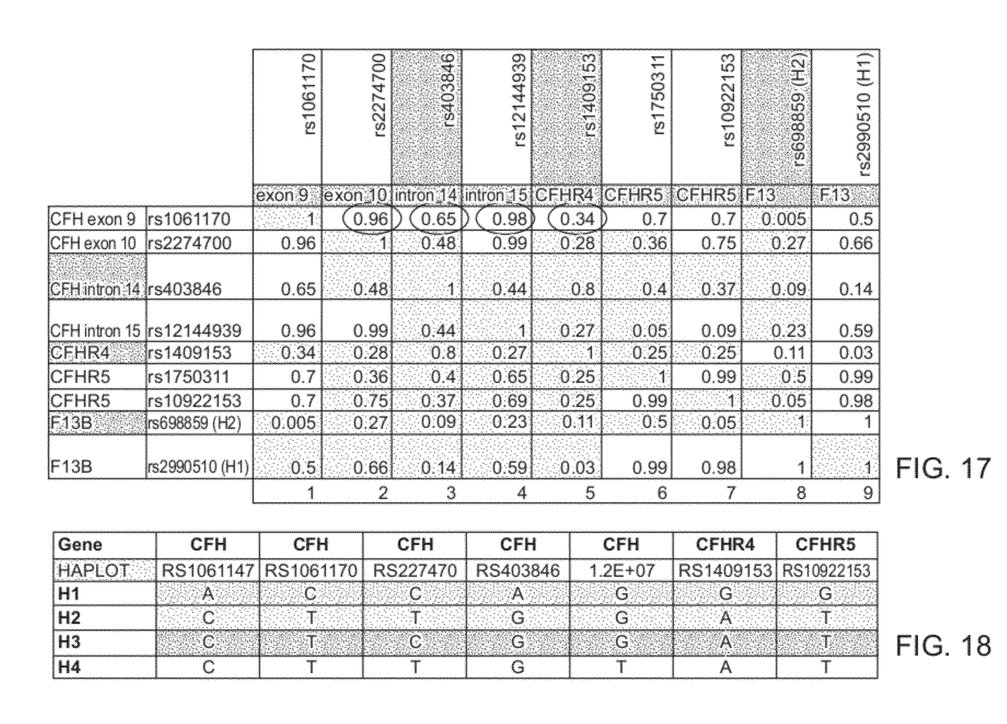

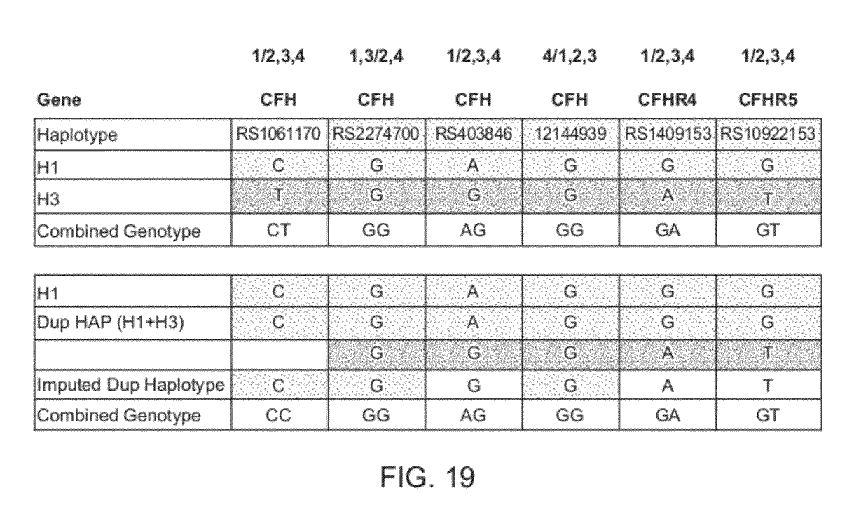

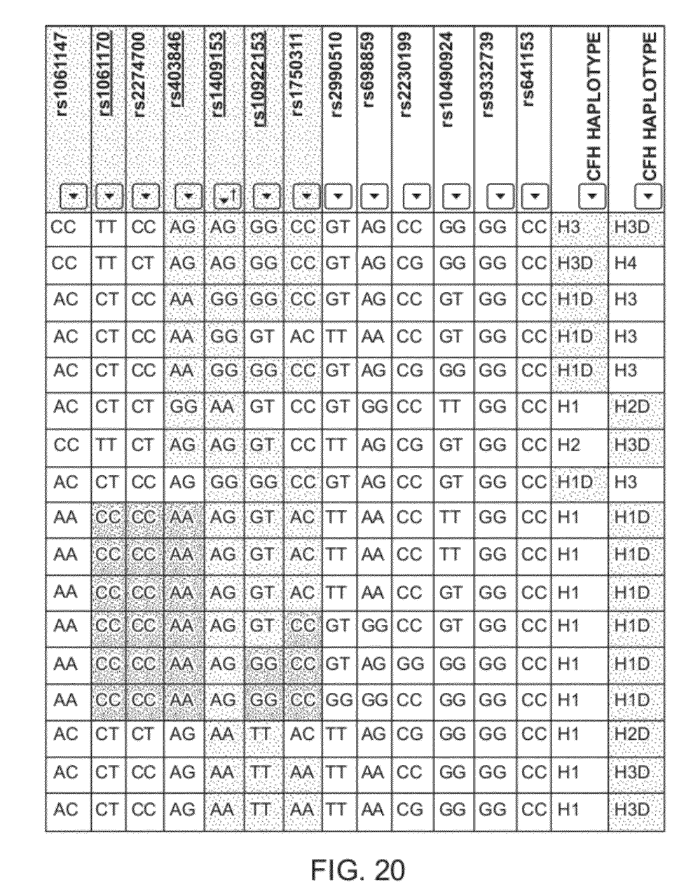

SNPs were examined for departure from expected linkage disequilibrium based on observed conserved sequences across the region. FIG. 17 reveals an unexpected drop off in LD across neighboring SNPs across the CFH and CFHR region. The SNP rs2274700 (exon 10 CFH) and rs12144939 (intron 15) are in close LD ˜0.96, 0.98 respectively with rs1061170 (SEQ ID NO: 16) (exon 9 CFH) while rs403846 (SEQ ID NO: 17) in intron 14 shows significant departure. SNP rs403846 (SEQ ID NO: 17) distinguishes H1 from H2, H3, H4 similar to the performance of rs1061170 (SEQ ID NO: 16), rs1409153 (SEQ ID NO: 18) and rs10922153 (SEQ ID NO: 19). The departure from LD cannot be explained by distance as the intron 15 SNP is further downstream. A possible explanation can be based on rs403846 (SEQ ID NO: 17) detecting the most frequent duplication involving an H3 with an H1. The LD observed for rs2274700 remains high as the presence of a H1 or H3 duplication would go undetected as this SNP distinguishes H1 and H3 from H2 and H4 (see FIG. 18). FIG. 18 illustrates SNPs useful for distinguishing haplotype combinations. By using SNPs that detect an unexpected presence of a variant originating from haplotypes H1 and H3 (see FIG. 19) it was possible to identify patterns of potential duplication in clinical samples shown in FIG. 20. The SNP's shown in FIG. 19 can be used to detect a duplication that occurs in genotypes generated by SNP's that distinguish the 2 most frequent duplications (H1/H3) observed in clinical samples.

FIG. 20 illustrates SNP patterns in clinical samples reflective of a duplication in the CFH-CFHR region. Four SNPs that distinguish H1/H2, H3, H4 haplotypes (rs1061170 (SEQ ID NO: 16), rs403846 (SEQ ID NO: 17), rs1409153 (SEQ ID NO: 18) and rs10922153 (SEQ ID NO: 19)) can be used to identify samples that potentially contain a duplicated segment of the CFH/CFHR region. Samples highlighted in light grey are indicative of duplication. Evidence to Support hot spot region near exon 9 CFH for recombination/duplication/deletion

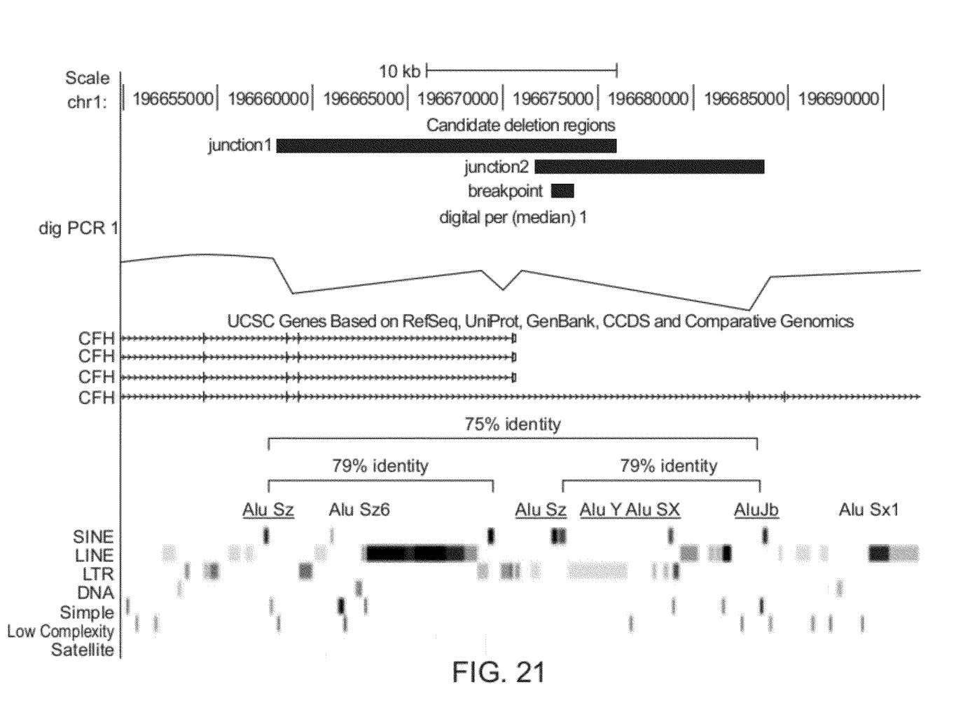

AluSz and Alu Sx elements are primate specific and often known to mediate recombination. Several possible recombination sites have been observed in the CFH-CFHR region that may result in non-homologous events mediated by AluSz and AluSx. The higher density of these elements in CNV1 might explain the higher than expected recombination/duplication observed. FIG. 21 illustrates the position of AluSz and AluSx sites in the CFH-CFHR region downstream of exon 9.

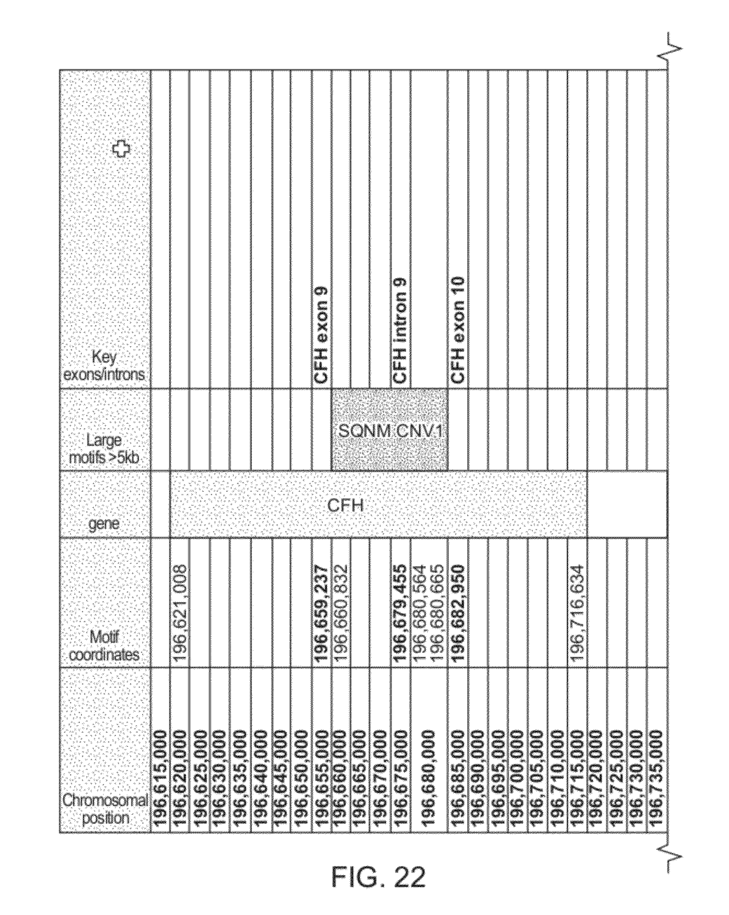

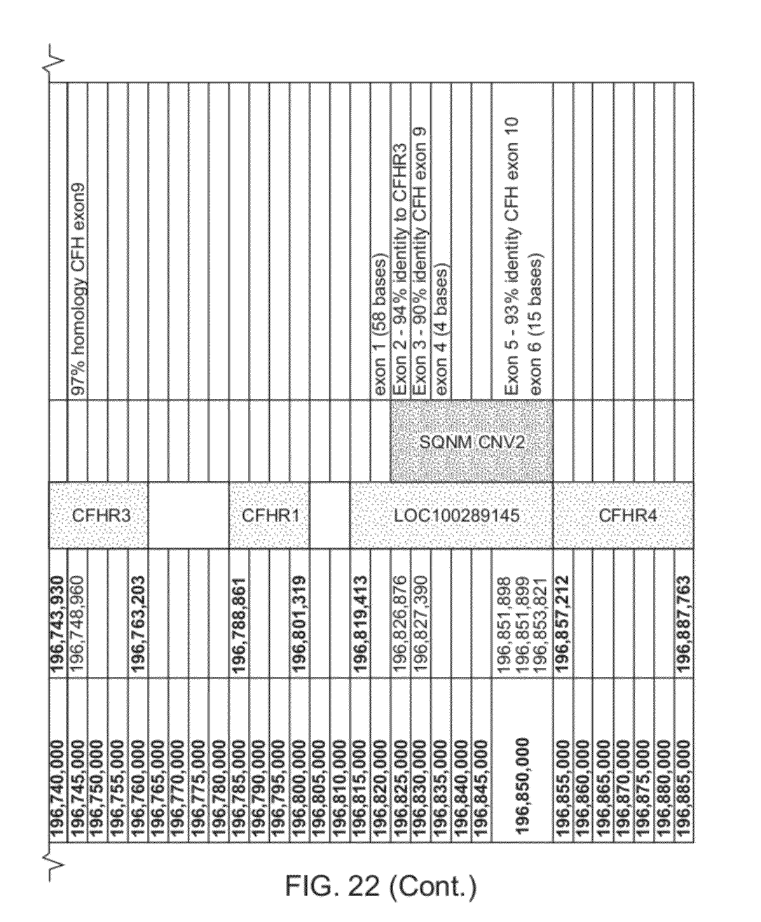

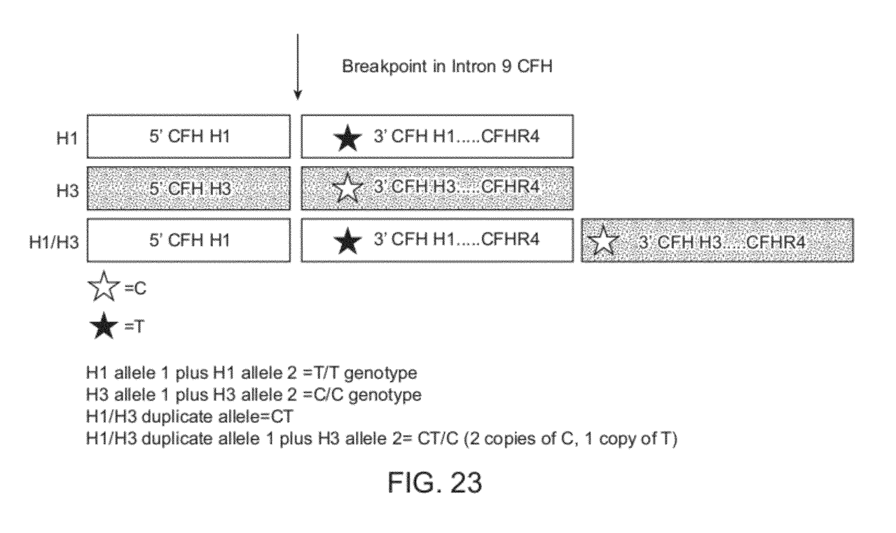

FIG. 22 provides a schematic illustration of the CFH-CFHR region and nucleotide positions for 5′ and 3′ end of various exons and introns in the locus.

### Example 6

### SNPs that Detect Copy Number Variation in the CFH-CFHR Region

| | Chromosome | | |
|---|---|---|---|
| | position | Nucleotide | Nucleotide |
| | (NCBI build | for | for |
| RS# | #36.3) | Allele 1 | Allele 2 |
| 1061170 | 194925860 | C | T |
| (SEQ ID | | | |
| NO: 16) | | | |
| 403846 | 194963360 | A | G |
| (SEQ ID | | | |
| NO: 17) | | | |
| 1409153 | 195146628 | C/G | T/A |
| (SEQ ID | | | |
| NO: 18) | | | |
| 10922153 | 195245238 | G | T |
| (SEQ ID | | | |
| NO: 19) | | | |
| 1750311 | 195220848 | C | A |
| (SEQ ID | | | |
| NO: 20) | | | |
| 10922094 | 194928128 | C | G |
| (SEQ ID | | | |
| NO: 21) | | | |
| 12124794 | 194928161 | A | T |
| (SEQ ID | | | |
| NO: 22) | | | |
| 12405238 | 194928236 | G | T |
| (SEQ ID | | | |
| NO: 23) | | | |
| 10922096 | 194929082 | C | T |
| (SEQ ID | | | |
| NO: 24) | | | |
| 12041668 | 194929670 | C | T |
| (SEQ ID | | | |
| NO: 25) | | | |
| 514943 | 194930536 | A/C | G/T |
| (SEQ ID | | | |
| NO: 26) | | | |
| 579745 | 194931199 | A | C/G |
| (SEQ ID | | | |
| NO: 27) | | | |
| 10922102 | 194934910 | C | T |
| (SEQ ID | | | |
| NO: 28) | | | |
| 2860102 | 194934942 | T | A |
| (SEQ ID | | | |
| NO: 29) | | | |
| 4658046 | 194937380 | C | T |
| (SEQ ID | | | |
| NO: 30) | | | |
| 10754199 | 194937462 | A/C | G/T |
| (SEQ ID | | | |
| NO: 31) | | | |
| 12565418 | 194938532 | C | T |
| (SEQ ID | | | |
| NO: 32) | | | |
| 12038333 | 194939077 | A | G |
| (SEQ ID | | | |
| NO: 33) | | | |
| 12045503 | 194939096 | C | T |
| (SEQ ID | | | |
| NO: 34) | | | |
| 9970784 | 194940425 | C | T |
| (SEQ ID | | | |
| NO: 35) | | | |
| 1831282 | 194940616 | G/A | T/C |
| (SEQ ID | | | |
| NO: 36) | | | |
| 203687 | 194940893 | C/G | T/A |
| (SEQ ID | | | |
| NO: 37) | | | |
| 2019727 | 194941337 | T | A |
| (SEQ ID | | | |
| NO: 38) | | | |
| 2019724 | 194941540 | C/G | T/A |
| (SEQ ID | | | |
| NO: 39) | | | |
| 1887973 | 194941802 | C | G |
| (SEQ ID | | | |
| NO: 40) | | | |
| 6428357 | 194942194 | G | A |
| (SEQ ID | | | |
| NO: 41) | | | |
| 7513157 | 194942303 | A | G |
| (SEQ ID | | | |
| NO: 42) | | | |
| 6695321 | 194942484 | A | G |
| (SEQ ID | | | |
| NO: 43) | | | |
| 10733086 | 194943558 | A | T |
| (SEQ ID | | | |
| NO: 44) | | | |
| 1410997 | 194943786 | G/A | T/C |
| (SEQ ID | | | |
| NO: 45) | | | |
| 203685 | 194944568 | C/G | A/T |
| (SEQ ID | | | |
| NO: 46) | | | |
| 203684 | 194944632 | A/C | G/T |
| (SEQ ID | | | |
| NO: 47) | | | |
| 10737680 | 194946078 | C | A |
| (SEQ ID | | | |
| NO: 48) | | | |
| 11811456 | 195114034 | A | G |
| 12240143 | 195111640 | C | T |
| 2336502 | 195109197 | C | T |
| 6428363 | 195110334 | G | A |
| 6428370 | 195111216 | G | A |
| 6685931 | 195133856 | C | T |
| 6695525 | 195112144 | G | T |
| 2133138 | 195109794 | A/C | G/T |
| 6428366 | 195110790 | G/T | A/C |

### Example 7

### Examples of Certain Embodiments

Provided hereafter are non-limiting examples of certain embodiments.

1. A method for identifying the presence or absence of a duplicated or multiplied Complement Factor H(CFH) allele in sample nucleic acid, comprising:

(a) detecting one or more nucleotides at one or more single nucleotide polymorphism (SNP) positions chosen from rs1061170 (SEQ ID NO: 16), rs403846 (SEQ ID NO: 17), rs1409153 (SEQ ID NO: 18), rs10922153 (SEQ ID NO: 19) and rs1750311 (SEQ ID NO: 20) in a nucleic acid containing a CFH allele from a biological sample, thereby providing a genotype; and

(b) identifying the presence or absence of a duplicated or multiplied CFH allele based on the genotype.

2. The method of embodiment 1, wherein the one or more SNP positions further are chosen from rs10922094 (SEQ ID NO: 21); rs12124794 (SEQ ID NO: 22); rs12405238 (SEQ ID NO: 23); rs10922096 (SEQ ID NO: 24); rs12041668 (SEQ ID NO: 25); rs514943 (SEQ ID NO: 26); rs579745 (SEQ ID NO: 27); rs10922102 (SEQ ID NO: 28); rs2860102 (SEQ ID NO: 29); rs4658046 (SEQ ID NO: 30); rs10754199 (SEQ ID NO: 31); rs12565418 (SEQ ID NO: 32); rs12038333 (SEQ ID NO: 33); rs12045503 (SEQ ID NO: 34); rs9970784 (SEQ ID NO: 35); rs1831282 (SEQ ID NO: 36); rs203687 (SEQ ID NO: 37); rs2019727 (SEQ ID NO: 38); rs2019724 (SEQ ID NO: 39); rs1887973 (SEQ ID NO: 40); rs6428357 (SEQ ID NO: 41); rs7513157 (SEQ ID NO: 42); rs6695321 (SEQ ID NO: 43); rs10733086 (SEQ ID NO: 44); rs1410997 (SEQ ID NO: 45); rs203685 (SEQ ID NO: 46); rs203684 (SEQ ID NO: 47); rs10737680 (SEQ ID NO: 48); rs11811456; rs12240143; rs2336502; rs6428363; rs6428370; rs6685931; rs6695525, rs2133138, rs6428366. rs10733086 (SEQ ID NO: 44), rs10922094 (SEQ ID NO: 21), and rs1887973 (SEQ ID NO: 40).

3. The method of embodiment 1 or 2, wherein the genotype includes two or more copies of a nucleotide at each SNP position.

4. The method of embodiment 3, wherein the genotype includes a ratio between two of the two or more copies of the nucleotide at each SNP position.

5. The method of any one of embodiments 1 to 4, comprising determining whether the subject from which the sample was obtained is homozygous or heterozygous for a nucleotide at each of the one or more SNP positions.

6. The method of any one of embodiments 1 to 5, comprising detecting the one or more nucleotides at the one or more SNP positions on a single strand of the nucleic acid.

7. The method of any one of embodiments 1 to 6, comprising detecting the presence or absence of an increased risk, decreased risk, or changed or altered risk of developing a severe form of a complement-pathway associated condition or disease based on the identification of the presence or absence of the duplicated or multiplied CFH allele.

8. The method of any one of embodiments 1 to 7, comprising detecting the presence or absence of age-related macular degeneration (AMD) based on the identification of the presence or absence of the duplicated or multiplied CFH allele.

9. The method of any one of embodiments 1 to 8, comprising obtaining from a subject the biological sample that contains the nucleic acid comprising the CFH allele.

10. The method of any one of embodiments 1 to 9, wherein the nucleic acid is double-stranded.

11. The method of any one of embodiments 1 to 9, wherein the nucleic acid is deoxyribonucleic acid (DNA).

12. The method of any one of embodiments 1 to 11, comprising amplifying the nucleic acid from the biological sample and detecting the one or more nucleotides at the one or more SNP positions in the amplified nucleic acid.

13. A method for identifying the presence or absence of a duplicated or multiplied Complement Factor H(CFH) allele in sample nucleic acid, comprising:

(a) analyzing a polynucleotide comprising a CFH allele in a nucleic acid from a biological sample, thereby providing an analyzed polynucleotide; and

(b) determining from the analyzed polynucleotide whether the CFH allele is present or absent in multiple copies on one chromosome in a region that includes one or more single nucleotide polymorphism (SNP) positions chosen from rs1061170 (SEQ ID NO: 16), rs403846 (SEQ ID NO: 17), rs1409153 (SEQ ID NO: 18), rs10922153 (SEQ ID NO: 19) and rs1750311 (SEQ ID NO: 20).

14. A method for identifying the presence or absence of a duplicated or multiplied Complement Factor H(CFH) allele in sample nucleic acid, comprising:

(a) analyzing a polynucleotide comprising a CFH allele in a nucleic acid from a biological sample, thereby providing an analyzed polynucleotide; and

(b) determining from the analyzed polynucleotide whether the CFH allele is present or absent in multiple copies on one chromosome in a region spanning about chr1: 196,621,008 to about chr1:196,887,763, which chromosome positions are according to NCBI Build 37.

15. The method of embodiment 14, which comprises determining from the analyzed polynucleotide whether the CFH allele is present or absent in multiple copies on one chromosome in a region spanning about chr1: 196,659,237 to about chr1:196,887,763, which chromosome positions are according to NCBI Build 37.

16. The method of embodiment 14, which comprises determining from the analyzed polynucleotide whether the CFH allele is present or absent in multiple copies on one chromosome in a region spanning about chr1: 196,679,455 to about chr1:196,887,763, which chromosome positions are according to NCBI Build 37.

17. The method of embodiment 14, which comprises determining from the analyzed polynucleotide whether the CFH allele is present or absent in multiple copies on one chromosome in a region spanning about chr1:196,743,930 to about chr1:196,887,763, which chromosome positions are according to NCBI Build 37.

18. A method for identifying the presence or absence of a duplicated or multiplied Complement Factor H(CFH) allele in sample nucleic acid, comprising:

(a) analyzing a polynucleotide comprising a CFH allele in a nucleic acid from a biological sample, thereby providing an analyzed polynucleotide; and

(b) determining from the analyzed polynucleotide whether the CFH allele is present or absent in multiple copies on one chromosome in a region surrounding exon 10 of the CFH allele.

19. A method for identifying the presence or absence of a duplicated or multiplied Complement Factor H(CFH) allele in sample nucleic acid, comprising:

(a) analyzing a polynucleotide comprising a CFH allele in a nucleic acid from a biological sample, thereby providing an analyzed polynucleotide; and

(b) determining from the analyzed polynucleotide whether the CFH allele is present or absent in multiple copies on one chromosome in a region in proximity to coding variant Y402H and extending through intron 9 and intron 14 of the CFH allele.

20. A method for identifying the presence or absence of a duplicated or multiplied Complement Factor H(CFH) allele in sample nucleic acid, comprising:

(a) analyzing a polynucleotide comprising a CFH allele in a nucleic acid from a biological sample, thereby providing an analyzed polynucleotide; and

(b) determining from the analyzed polynucleotide whether the CFH allele is present or absent in multiple copies on one chromosome in a region in proximity to coding variant Y402H and extending through CFHR4.

21. The method of any one of embodiments 13 to 20, wherein the analyzing in (a) comprises determining the presence or absence of one or more genetic markers associated with the multiple copies on the one chromosome.

22. The method of embodiment 21, wherein the analyzing in (a) comprises detecting one or more nucleotides at one or more single nucleotide polymorphism (SNP) positions chosen from rs1061170 (SEQ ID NO: 16), rs403846 (SEQ ID NO: 17), rs1409153 (SEQ ID NO: 18), rs10922153 (SEQ ID NO: 19) and rs1750311 (SEQ ID NO: 20) in the amplified CFH allele, thereby providing a genotype.

23. The method of embodiment 22, wherein the one or more SNP positions further are chosen from rs10922094 (SEQ ID NO: 21); rs12124794 (SEQ ID NO: 22); rs12405238 (SEQ ID NO: 23); rs10922096 (SEQ ID NO: 24); rs12041668 (SEQ ID NO: 25); rs514943 (SEQ ID NO: 26); rs579745 (SEQ ID NO: 27); rs10922102 (SEQ ID NO: 28); rs2860102 (SEQ ID NO: 29); rs4658046 (SEQ ID NO: 30); rs10754199 (SEQ ID NO: 31); rs12565418 (SEQ ID NO: 32); rs12038333 (SEQ ID NO: 33); rs12045503 (SEQ ID NO: 34); rs9970784 (SEQ ID NO: 35); rs1831282 (SEQ ID NO: 36); rs203687 (SEQ ID NO: 37); rs2019727 (SEQ ID NO: 38); rs2019724 (SEQ ID NO: 39); rs1887973 (SEQ ID NO: 40); rs6428357 (SEQ ID NO: 41); rs7513157 (SEQ ID NO: 42); rs6695321 (SEQ ID NO: 43); rs10733086 (SEQ ID NO: 44); rs1410997 (SEQ ID NO: 45); rs203685 (SEQ ID NO: 46); rs203684 (SEQ ID NO: 47); rs10737680 (SEQ ID NO: 48); rs11811456; rs12240143; rs2336502; rs6428363; rs6428370; rs6685931; rs6695525, rs2133138, rs6428366. rs10733086 (SEQ ID NO: 44), rs10922094 (SEQ ID NO: 21), and rs1887973 (SEQ ID NO: 40).

24. The method of embodiment 22 or 23, wherein the genotype includes two or more copies of a nucleotide at each SNP position.

25. The method of embodiment 24, wherein the genotype includes a ratio between two of the two or more copies of the nucleotide at each SNP position.

26. The method of any one of embodiments 22 to 25, comprising determining whether the subject from which the sample was obtained is homozygous or heterozygous for a nucleotide at each of the one or more SNP positions.

27. The method of any one of embodiments 22 to 26, comprising detecting the one or more nucleotides at the one or more SNP positions on a single strand of the nucleic acid.

28. The method of any one of embodiments 13 to 27, comprising obtaining from a subject the biological sample that contains the nucleic acid comprising the CFH allele.

29. The method of any one of embodiments 13 to 28, wherein the nucleic acid is double-stranded.

30. The method of any one of embodiments 13 to 29, wherein the nucleic acid is deoxyribonucleic acid (DNA).

31. The method of any one of embodiments 13 to 30, comprising detecting the presence or absence of an increased risk, decreased risk, or changed or altered risk of developing a complement-pathway associated condition or disease based on whether the CFH allele is present or absent in multiple copies on one chromosome.

32. The method of any one of embodiments 13 to 31, comprising detecting the presence or absence of age-related macular degeneration (AMD) based on whether the CFH allele is present or absent in multiple copies on one chromosome.

33. The method of embodiment 31, comprising detecting the presence or absence of an increased risk, decreased risk, or changed or altered risk of developing a severe form of a complement-pathway associated condition or disease based on whether the CFH allele is present or absent in multiple copies on one chromosome.

34. The method of embodiment 33, comprising detecting the presence or absence of wet age-related macular degeneration (AMD) based on whether the CFH allele is present or absent in multiple copies on one chromosome.

35. The method of any one of embodiments 13 to 34, comprising determining the risk of progressing from a less severe to a more severe form of a complement-pathway associated condition or disease based on whether the CFH allele is present or absent in multiple copies on one chromosome.

36. The method of embodiment 35, wherein the more severe form of the complement-pathway associated condition or disease is wet age-related macular degeneration (AMD).

37. The method of any one of embodiments 13 to 36, comprising amplifying the nucleic acid from the biological sample and analyzing the amplified nucleic acid in (a).

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, these modifications and improvements are within the scope and spirit of the technology.

The technology illustratively described herein suitably may be practiced in the absence of any element(s) not specifically disclosed herein. Thus, for example, the term “comprising” in each instance may be substituted by the term “consisting essentially of” or “consisting of:” The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. Use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). In certain instances units and formatting are expressed in HyperText Markup Language (HTML) format, which can be translated to another conventional format by those skilled in the art (e.g., “.sup.” refers to superscript formatting). Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claim(s) that follow(s).

1. A method for identifying the presence or absence of a duplicated or multiplied Complement Factor H(CFH) allele in sample nucleic acid, comprising:
(a) detecting one or more nucleotides at one or more single nucleotide polymorphism (SNP) positions chosen from rs1061170 (SEQ ID NO: 16), rs403846 (SEQ ID NO: 17), rs1409153 (SEQ ID NO: 18), rs10922153 (SEQ ID NO: 19) and rs1750311 (SEQ ID NO: 20) in a nucleic acid containing a CFH allele from a biological sample, thereby providing a genotype; and
(b) identifying the presence or absence of a duplicated or multiplied CFH allele based on the genotype.

2. The method of claim 1, wherein the one or more SNP positions further are chosen from rs10922094 (SEQ ID NO: 21); rs12124794 (SEQ ID NO: 22); rs12405238 (SEQ ID NO: 23); rs10922096 (SEQ ID NO: 24); rs12041668 (SEQ ID NO: 25; rs514943 (SEQ ID NO: 26); rs579745 (SEQ ID NO: 27); rs10922102 (SEQ ID NO: 28; rs2860102 (SEQ ID NO: 29); rs4658046 (SEQ ID NO: 30); rs10754199 (SEQ ID NO: 31); rs12565418 (SEQ ID NO: 32); rs12038333 (SEQ ID NO: 33); rs12045503 (SEQ ID NO: 34); rs9970784 (SEQ ID NO: 35); rs1831282 (SEQ ID NO: 36); rs203687 (SEQ ID NO: 37); rs2019727 (SEQ ID NO: 38); rs2019724 (SEQ ID NO: 39); rs1887973 (SEQ ID NO: 40); rs6428357 (SEQ ID NO: 41); rs7513157 (SEQ ID NO: 42); rs6695321 (SEQ ID NO: 43); rs10733086 (SEQ ID NO: 44); rs1410997SEQ ID NO: 45); rs203685 (SEQ ID NO: 46); rs203684 (SEQ ID NO: 47); and rs10737680 (SEQ ID NO: 48).

3. The method of claim 1, wherein the genotype includes two or more copies of a nucleotide at each SNP position.

4. The method of claim 3, wherein the genotype includes a ratio between two of the two or more copies of the nucleotide at each SNP position.

5. The method of claim 1, comprising determining whether the subject from which the sample was obtained is homozygous or heterozygous for a nucleotide at each of the one or more SNP positions.

6. The method of claim 1, comprising detecting the one or more nucleotides at the one or more SNP positions on a single strand of the nucleic acid.

7. The method of claim 1, comprising detecting the presence or absence of an increased risk, decreased risk, or changed or altered risk of developing a complement-pathway associated condition or disease based on the identification of the presence or absence of the duplicated or multiplied CFH allele.

8. The method of claim 1, comprising detecting the presence or absence of age-related macular degeneration (AMD) based on the identification of the presence or absence of the duplicated or multiplied CFH allele.

9. The method of claim 1, comprising obtaining from a subject the biological sample that contains the nucleic acid comprising the CFH allele.

10. The method of claim 1, wherein the nucleic acid is double-stranded.

11. The method of claim 1, wherein the nucleic acid is deoxyribonucleic acid (DNA).

12. The method of claim 1, comprising amplifying the nucleic acid from the biological sample and detecting the one or more nucleotides at the one or more SNP positions in the amplified nucleic acid.

13. A method for identifying the presence or absence of a duplicated or multiplied Complement Factor H(CFH) allele in sample nucleic acid, comprising:
(a) amplifying a polynucleotide comprising a CFH allele in a nucleic acid from a biological sample, thereby providing an amplified CFH allele; and
(b) determining from the amplified CFH allele whether the CFH allele is present or absent in multiple copies on one chromosome in a region containing one or more single nucleotide polymorphisms (SNPs) chosen from rs1061170 (SEQ ID NO: 16), rs403846 (SEQ ID NO: 17), rs1409153 (SEQ ID NO: 18), rs10922153 (SEQ ID NO: 19) and rs1750311 (SEQ ID NO: 20).

14. The method of claim 13, wherein the region spans about chr1:196,620,000 to about chr1:196,887,763, which chromosome positions are according to NCBI Build 37.

15. The method of claim 13, wherein the region spans about chr1:196,659,237 to about chr1:196,887,763, which chromosome positions are according to NCBI Build 37.

16. The method of claim 13, wherein the region spans about chr1:196,679,455 to about chr1:196,887,763, which chromosome positions are according to NCBI Build 37.

17. The method of claim 13, wherein the region spans about chr1:196,743,930 to about chr1:196,887,763, which chromosome positions are according to NCBI Build 37.

18. The method of claim 13, comprising detecting one or more nucleotides at one or more single nucleotide polymorphism (SNP) positions chosen from rs1061170 (SEQ ID NO: 16), rs403846 (SEQ ID NO: 17), rs1409153 (SEQ ID NO: 18), rs10922153 (SEQ ID NO: 19) and rs1750311 (SEQ ID NO: 20) in the amplified CFH allele, thereby providing a genotype.

19. The method of claim 18, wherein the one or more SNP positions further are chosen from rs10922094 (SEQ ID NO: 21); rs12124794SEQ ID NO: 22); rs12405238 (SEQ ID NO: 23); rs10922096 (SEQ ID NO: 24); rs12041668 (SEQ ID NO: 25); rs514943 (SEQ ID NO: 26); rs579745 (SEQ ID NO: 27); rs10922102 (SEQ ID NO: 28); rs2860102 (SEQ ID NO: 29); rs4658046 (SEQ ID NO: 30); rs10754199 (SEQ ID NO: 31); rs12565418 (SEQ ID NO: 32); rs12038333 (SEQ ID NO: 33); rs12045503 (SEQ ID NO: 34); rs9970784 (SEQ ID NO: 35); rs1831282SEQ ID NO: 36); rs203687 (SEQ ID NO: 37); rs2019727 (SEQ ID NO: 38); rs2019724 (SEQ ID NO: 39); rs1887973 (SEQ ID NO: 40); rs6428357 (SEQ ID NO: 41); rs7513157 (SEQ ID NO: 42); rs6695321 (SEQ ID NO: 43); rs10733086 (SEQ ID NO: 44); rs1410997 (SEQ ID NO: 45); rs203685 (SEQ ID NO: 46); rs203684 (SEQ ID NO: 47); and rs10737680 (SEQ ID NO: 48).

20. The method of claim 18, wherein the genotype includes two or more copies of a nucleotide at each SNP position.

21. The method of claim 20, wherein the genotype includes a ratio between two of the two or more copies of the nucleotide at each SNP position.

22. The method of claim 18, comprising determining whether the subject from which the sample was obtained is homozygous or heterozygous for a nucleotide at each of the one or more SNP positions.

23. The method of claim 18, comprising detecting the one or more nucleotides at the one or more SNP positions on a single strand of the nucleic acid.

24. The method of claim 13, comprising obtaining from a subject the biological sample that contains the nucleic acid comprising the CFH allele.

25. The method of claim 13, wherein the nucleic acid is double-stranded.

26. The method of claim 13, wherein the nucleic acid is deoxyribonucleic acid (DNA).

27. The method of claim 13, comprising detecting the presence or absence of an increased risk, decreased risk, or changed or altered risk of developing a complement-pathway associated condition or disease based on whether the CFH allele is present or absent in multiple copies on one chromosome.

28. The method of claim 27, comprising detecting the presence or absence of age-related macular degeneration (AMD) based on whether the CFH allele is present or absent in multiple copies on one chromosome.

29. The method of claim 13, comprising determining the risk of progressing from a less severe to a more severe form of a complement-pathway associated condition or disease based on whether the CFH allele is present or absent in multiple copies on one chromosome.

30. The method of claim 29, wherein the more severe form of the complement-pathway associated condition or disease is wet age-related macular degeneration (AMD).
